(19) United States
(12) Patent Application Publication
(10) Pub. No.: US 2020/0347386 A1
(43) Pub. Date: Nov. 5, 2020

(54) COMBINATION GENE TARGETS FOR IMPROVED IMMUNOTHERAPY

(71) Applicant: KSQ Therapeutics, Inc., Cambridge, MA (US)

(72) Inventors: Micah BENSON, Cambridge, MA (US); Michael SCHLABACH, Cambridge, MA (US); Gregory KRYUKOV, Cambridge, MA (US); Anne Louise CADZOW, Cambridge, MA (US); Isabelle Fleur LE MERCIER, Cambridge, MA (US); Frank STEGMEIER, Cambridge, MA (US)

(21) Appl. No.: 16/781,732

(22) Filed: Feb. 4, 2020

Related U.S. Application Data
(60) Provisional application No. 62/818,677, filed on Mar. 14, 2019.
(60) Provisional application No. 62/800,999, filed on Feb. 4, 2019.

(51) Int. Cl. C12N 15/11; C12N 9/22; C12N 15/86; A61K 35/17; A61K 49/00; C12N 5/0783

(52) U.S. Cl. CPC C12N 15/11; C12N 9/22; C12N 15/86; A61K 35/17; A61K 49/0004; C12N 2510/00; C12N 2310/14; C12N 2310/531; C12N 2800/80; C12N 2310/20; C12N 2740/15043; C12N 5/0636

(57) ABSTRACT

The present disclosure provides methods and compositions related to the modification of immune effector cells to increase therapeutic efficacy. In some embodiments, immune effector cells modified to reduce expression of one or more endogenous target genes, or to reduce one or more functions of an endogenous protein to enhance effector functions of the immune cells are provided. In some embodiments, immune effector cells further modified by introduction of transgenes conferring antigen specificity, such as exogenous T cell receptors (TCRs) or chimeric antigen receptors (CARs) are provided. Methods of treating a cell proliferative disorder, such as a cancer, using the modified immune effector cells described herein are also provided.

### CROSS-REFERENCE TO RELATED APPLICATIONS

This application claims priority to U.S. Provisional Patent Application No. 62/800,999, filed on Feb. 4, 2019; and U.S. Provisional Patent Application No. 62/818,677, filed on Mar. 14, 2019, both of which are incorporated herein by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: 700061_KSQW-014_ST25.txt; date recorded: Feb. 4, 2020; file size: 945 kilobytes).

### FIELD

The disclosure relates to methods, compositions, and components for editing a target nucleic acid sequence, or modulating expression of a target nucleic acid sequence, and applications thereof in connection with immunotherapy, including use with receptor-engineered immune effector cells, in the treatment of cell proliferative diseases, inflammatory diseases, and/or infectious diseases.

### BACKGROUND

Adoptive cell transfer utilizing genetically modified T cells, in particular CAR-T cells, has entered clinical testing as a therapeutic for solid and hematologic malignancies. Results to date have been mixed. In hematologic malignancies (especially lymphoma, CLL and ALL), the majority of patients in several Phase 1 and 2 trials exhibited at least a partial response, with some exhibiting complete responses (Kochenderfer et al., 2012 Blood 1 19, 2709-2720). In 2017, the FDA approved two CAR-T therapies, Kymriah™ and Yescarta™, both for the treatment of hematological cancers. However, in most tumor types (including melanoma, renal cell carcinoma and colorectal cancer), fewer responses have been observed (Johnson et al., 2009 Blood 1 14, 535-546; Lamers et al., 2013 Mol. Ther. 21, 904-912; Warren et al., 1998 Cancer Gene Ther. 5, S1-S2). As such, there is considerable room for improvement with adoptive T cell therapies, as success has largely been limited to CAR-T cells approaches targeting hematological malignancies of the B cell lineage.

### SUMMARY

There exists a need to improve the efficacy of adoptive transfer of modified immune cells in cancer treatment, in particular increasing the efficacy of adoptive cell therapies against solid malignancies, as reduced responses have been observed in these tumor types (melanoma, renal cell carcinoma and colorectal cancer; Yong, 2017, Imm Cell Biol., 95:356-363). In addition, even in hematological malignancies where a benefit of adoptive transfer has been observed, not all patients respond and relapses occur with a greater than desired frequency, likely as a result of diminished function of the adoptively transferred T cells.

Factors limiting the efficacy of genetically modified immune cells as cancer therapeutics include: (1) cell proliferation, e.g., limited proliferation of T cells following adoptive transfer; (2) cell survival, e.g., induction of T cell apoptosis by factors in the tumor environment; and (3) cell function, e.g., inhibition of cytotoxic T cell function by inhibitory factors secreted by host immune cells and cancer cells and exhaustion of immune cells during manufacturing processes and/or after transfer.

Particular features thought to increase the anti-tumor effects of an immune cell include a cell's ability to: 1) proliferate in the host following adoptive transfer; 2) infiltrate a tumor; 3) persist in the host and/or exhibit resistance to immune cell exhaustion; and 4) function in a manner capable of killing tumor cells. The present disclosure provides immune cells comprising decreased expression and/or function of one or more endogenous target genes wherein the modified immune cells demonstrate an enhancement of one or more effector functions including increased proliferation, increased infiltration into tumors, persistence of the immune cells in a subject, and/or increased resistance to immune cell exhaustion. The present disclosure also provides methods and compositions for modification of immune effector cells to elicit enhanced immune cell activity towards a tumor cell, as well as methods and compositions suitable for use in the context of adoptive immune cell transfer therapy.

In some embodiments, the present disclosure provides a modified immune effector cell comprising a gene-regulating system capable of reducing the expression and/or function of at least two endogenous target genes selected from SOCS1, PTPN2, and ZC3H12A, wherein the reduced expression and/or function of the at least two endogenous target genes enhances an effector function of the immune effector cell. In some embodiments, the at least two target genes are SOCS1 and PTPN2. In some embodiments, the at least two target genes are SOCS1 and ZC3H12A. In some embodiments, the at least two target genes are PTPN2 and ZC3H12A. In some embodiments, the gene regulating system is further capable of reducing the expression and/or function of CBLB.

In some embodiments, the gene-regulating system comprises (i) a nucleic acid molecule; (ii) an enzymatic protein; or (iii) a nucleic acid molecule and an enzymatic protein. In some embodiments, the gene-regulating system comprises a nucleic acid molecule selected from an siRNA, an shRNA, a microRNA (miR), an antagomiR, or an antisense RNA. In some embodiments, the gene-regulating system comprises an enzymatic protein, and wherein the enzymatic protein has been engineered to specifically bind to a target sequence in one or more of the endogenous genes. In some embodiments, the protein is a Transcription activator-like effector nuclease (TALEN), a zinc-finger nuclease, or a meganuclease. In some embodiments, the gene-regulating system comprises a nucleic acid molecule and an enzymatic protein, wherein the nucleic acid molecule is a guide RNA (gRNA) molecule and the enzymatic protein is a Cas protein or Cas ortholog. In some embodiments, the Cas protein is a Cas9 protein. In some embodiments, the Cas protein is a wild-type Cas protein comprising two enzymatically active domains, and capable of inducing double stranded DNA breaks. In some embodiments, the Cas protein is a Cas nickase mutant comprising one enzymatically active domain and capable of inducing single stranded DNA breaks. In some embodiments, the Cas protein is a deactivated Cas protein (dCas) and is associated with a heterologous protein capable of modulating the expression of the one or more endogenous target genes. In some embodiments, the heterologous protein is selected from the group consisting of MAX-interacting protein 1 (MXI1), Krüppel-associated box (KRAB) domain, methyl-CpG binding protein 2 (MECP2), and four concatenated mSin3 domains (SID4X).

In some embodiments, the at least two endogenous genes are SOCS1 and PTPN2, and wherein the gene-regulating system comprises at least one SOCS1-targeting gRNA molecule comprising a targeting domain sequence complementary to a nucleic acid sequence defined by any one of the set of genome coordinates shown in Tables 3 and 4 and at least one PTPN2-targeting gRNA molecule comprising a targeting domain sequence complementary to a nucleic acid sequence defined by any one of the set of genome coordinates shown in Tables 5 and 6. In some embodiments, the at least two endogenous genes are SOCS1 and PTPN2, and wherein the gene-regulating system comprises at least one SOCS1-targeting gRNA molecule comprising a targeting domain sequence that binds to a nucleic acid sequence defined by any one of the set of genome coordinates shown in Tables 3 and 4 and at least one PTPN2-targeting gRNA molecule comprising a targeting domain sequence that binds to a nucleic acid sequence defined by any one of the set of genome coordinates shown in Tables 5 and 6. In some embodiments, the at least two endogenous genes are SOCS1 and PTPN2, and wherein the gene-regulating system comprises at least one SOCS1-targeting gRNA molecule comprising a targeting domain sequence that binds to a target DNA sequence selected from the group consisting of SEQ ID NOs: 7-151 and at least one PTPN2-targeting gRNA molecule comprising a targeting domain sequence that binds to a target DNA sequence selected from the group consisting of SEQ ID NOs: 185-207. In some embodiments, the at least two endogenous genes are SOCS1 and PTPN2, and wherein the gene-regulating system comprises at least one SOCS1-targeting gRNA molecule comprising a targeting domain sequence encoded by a nucleic acid sequence selected from the group consisting of SEQ ID NOs: 7-151 and at least one PTPN2-targeting gRNA molecule comprising a targeting domain sequence encoded by a nucleic acid sequence selected from the group consisting of SEQ ID NOs: 185-207.

In some embodiments, the at least two endogenous genes are SOCS1 and ZC3H12A, and wherein the gene-regulating system comprises at least one SOCS1-targeting gRNA molecule comprising a targeting domain sequence complementary to a nucleic acid sequence defined by any one of the set of genome coordinates shown in Tables 3 and 4, and at least one ZC3H12A-targeting gRNA molecule comprising a targeting domain sequence complementary to a nucleic acid sequence defined by any one of the set of genome coordinates shown in Tables 7 and 8. In some embodiments, the at least two endogenous genes are SOCS1 and ZC3H12A, and wherein the gene-regulating system comprises at least one SOCS1-targeting gRNA molecule comprising a targeting domain sequence that binds to a nucleic acid sequence defined by any one of the set of genome coordinates shown in Tables 3 and 4 and at least one ZC3H12A-targeting gRNA molecule comprising a targeting domain sequence that binds to a nucleic acid sequence defined by any one of the set of genome coordinates shown in Tables 7 and 8. In some embodiments, the at least two endogenous genes are SOCS1 and ZC3H12A, and wherein the gene-regulating system comprises at least one SOCS1-targeting gRNA molecule comprising a targeting domain sequence that binds to a target DNA sequence selected from the group consisting of SEQ ID NOs: 7-151 and at least one ZC3H12A-targeting gRNA molecule comprising a targeting domain sequence that binds to a target DNA sequence selected from the group consisting of SEQ ID NOs: 208-230. In some embodiments, the at least two endogenous genes are SOCS1 and ZC3H12A, and wherein the gene-regulating system comprises at least one SOCS1-targeting gRNA molecule comprising a targeting domain sequence encoded by a nucleic acid sequence selected from the group consisting of SEQ ID NOs: 7-151 and at least one ZC3H12A-targeting gRNA molecule comprising a targeting domain sequence encoded by a nucleic acid sequence selected from the group consisting of SEQ ID NOs: 208-230.

In some embodiments, the at least two endogenous genes are PTPN2 and ZC3H12A, and wherein the gene-regulating system comprises at least one PTPN2-targeting gRNA molecule comprising a targeting domain sequence complementary to a nucleic acid sequence defined by any one of the set of genome coordinates shown in Tables 5 and 6 and at least one ZC3H12A-targeting gRNA molecule comprising a targeting domain sequence complementary to a nucleic acid sequence defined by any one of the set of genome coordinates shown in Tables 7 and 8. In some embodiments, the at least two endogenous genes are PTPN2 and ZC3H12A, and wherein the gene-regulating system comprises at least one PTPN2-targeting gRNA molecule comprising a targeting domain sequence that binds to a nucleic acid sequence defined by any one of the set of genome coordinates shown in Tables 5 and 6 and at least one ZC3H12A-targeting gRNA molecule comprising a targeting domain sequence that binds to a nucleic acid sequence defined by any one of the set of genome coordinates shown in Tables 7 and 8. In some embodiments, the at least two endogenous genes are PTPN2 and ZC3H12A, and wherein the gene-regulating system comprises at least one PTPN2-targeting gRNA molecule comprising a targeting domain sequence that binds to a target DNA sequence selected from the group consisting of SEQ ID NOs: 185-207 and at least one ZC3H12A-targeting gRNA molecule comprising a targeting domain sequence that binds to a target DNA sequence selected from the group consisting of SEQ ID NOs: 208-230. In some embodiments, the at least two endogenous genes are PTPN2 and ZC3H12A, and wherein the gene-regulating system comprises at least one PTPN2-targeting gRNA molecule comprising a targeting domain sequence encoded by a nucleic acid sequence selected from the group consisting of SEQ ID NOs: 185-207 and at least one ZC3H12A-targeting gRNA molecule comprising a targeting domain sequence encoded by a nucleic acid sequence selected from the group consisting of SEQ ID NOs: 208-230.

In some embodiments, the at least two endogenous genes are SOCS1 and PTPN2, and wherein the gene-regulating system comprises at least one SOCS1-targeting siRNA or shRNA molecule comprising about 19-30 nucleotides that are complementary to an RNA sequence encoded by a DNA sequence defined by a set of genome coordinates shown in Tables 3 and 4 and at least one PTPN2-targeting siRNA or shRNA molecule comprising about 19-30 nucleotides that are complementary to an RNA sequence encoded by a DNA sequence defined by a set of genome coordinates shown in Tables 5 and 6. In some embodiments, the at least two endogenous genes are SOCS1 and PTPN2, and wherein the gene-regulating system comprises at least one SOCS1-targeting siRNA or shRNA molecule comprising about 19-30 nucleotides that binds to an RNA sequence encoded by a DNA sequence defined by a set of genome coordinates shown in Tables 3 and 4 and at least one PTPN2-targeting siRNA or shRNA molecule comprising about 19-30 nucleotides that binds to an RNA sequence encoded by a DNA sequence defined by a set of genome coordinates shown in Tables 5 and 6. In some embodiments, the at least two endogenous genes are SOCS1 and PTPN2, wherein the gene-regulating system comprises at least one SOCS1-targeting siRNA or shRNA comprising about 19-30 nucleotides that bind to an RNA sequence encoded by a DNA sequence selected from the group consisting of SEQ ID NOs: 7-151 and at least one PTPN2-targeting siRNA or shRNA comprising about 19-30 nucleotides that bind to an RNA sequence encoded by a DNA sequence selected from the group consisting of SEQ ID NOs: 185-207.

In some embodiments, the at least two endogenous genes are SOCS1 and ZC3H12A, wherein the gene-regulating system comprises at least one SOCS1-targeting siRNA or shRNA molecule comprising about 19-30 nucleotides that are complementary to an RNA sequence encoded by a DNA sequence defined by a set of genome coordinates shown in Tables 3 and 4 and at least one ZC3H12A-targeting siRNA or shRNA molecule comprising about 19-30 nucleotides that are complementary to an RNA sequence encoded by a DNA sequence defined by a set of genome coordinates shown in Tables 7 and 8. In some embodiments, the at least two endogenous genes are SOCS1 and ZC3H12A, wherein the gene-regulating system comprises at least one SOCS1-targeting siRNA or shRNA molecule comprising about 19-30 nucleotides that binds to an RNA sequence encoded by a DNA sequence defined by a set of genome coordinates shown in Table 3 and 4 and at least one ZC3H12A-targeting siRNA or shRNA molecule comprising about 19-30 nucleotides that binds to an RNA sequence encoded by a DNA sequence defined by a set of genome coordinates shown in Tables 7 and 8. In some embodiments, the at least two endogenous genes are SOCS1 and ZC3H12A, wherein the gene-regulating system comprises at least one SOCS1-targeting siRNA or shRNA comprising about 19-30 nucleotides that binds to an RNA sequence encoded by a DNA sequence selected from the group consisting of SEQ ID NOs: 7-151 and at least one ZC3H12A-targeting siRNA or shRNA comprising about 19-30 nucleotides that binds to an RNA sequence encoded by a DNA sequence selected from the group consisting of SEQ ID NOs: 208-230.

In some embodiments, the at least two endogenous genes are PTPN2 and ZC3H12A, wherein the gene-regulating system comprises at least one PTPN2-targeting siRNA or shRNA molecule comprising about 19-30 nucleotides that are complementary to an RNA sequence encoded by a DNA sequence defined by a set of genome coordinates shown in Tables 5 and 6 and at least one ZC3H12A-targeting siRNA or shRNA molecule comprising about 19-30 nucleotides that are complementary to an RNA sequence encoded by a DNA sequence defined by a set of genome coordinates shown in Tables 7 and 8. In some embodiments, the at least two endogenous genes are PTPN2 and ZC3H12A, wherein the gene-regulating system comprises at least one PTPN2-targeting siRNA or shRNA molecule comprising about 19-30 nucleotides that binds to an RNA sequence encoded by a DNA sequence defined by a set of genome coordinates shown in Tables 5 and 6 and at least one ZC3H12A-targeting siRNA or shRNA molecule comprising about 19-30 nucleotides that binds to an RNA sequence encoded by a DNA sequence defined by a set of genome coordinates shown in Tables 7 and 8. In some embodiments, the at least two endogenous genes are PTPN2 and ZC3H12A, wherein the gene-regulating system comprises at least one PTPN2-targeting siRNA or shRNA comprising about 19-30 nucleotides that binds to an RNA sequence encoded by a DNA sequence selected from the group consisting of SEQ ID NOs: 185-207 and at least one ZC3H12A-targeting siRNA or shRNA comprising about 19-30 nucleotides that binds to an RNA sequence encoded by a DNA sequence selected from the group consisting of SEQ ID NOs: 208-230.

In some embodiments, the at least two endogenous genes are SOCS1 and PTPN2, and wherein the gene-regulating system comprises at least one SOCS1-targeting TALEN, zinc finger, or meganuclease protein that binds to a target DNA sequence defined by a set of genome coordinates shown in Tables 3 and 4 and at least one PTPN2-targeting TALEN, zinc finger, or meganuclease protein that binds to a target DNA sequence defined by a set of genome coordinates shown in Tables 5 and 6. In some embodiments, the at least two endogenous genes are SOCS1 and PTPN2, wherein the gene-regulating system comprises at least one SOCS1-targeting TALEN, zinc finger, or meganuclease protein that binds to a DNA sequence selected from SEQ ID NOs: 7-151 and at least one PTPN2-targeting TALEN, zinc finger, or meganuclease protein that binds to a DNA sequence selected from SEQ ID NOs: 185-207.

In some embodiments, the at least two endogenous genes are SOCS1 and ZC3H12A, wherein the gene-regulating system comprises at least one SOCS1-targeting TALEN, zinc finger, or meganuclease protein that binds to a target DNA sequence defined by a set of genome coordinates shown in Tables 3 and 4 and at least one ZC3H12A-targeting TALEN, zinc finger, or meganuclease protein that binds to a target DNA sequence defined by a set of genome coordinates shown in Tables 7 and 8. In some embodiments, the at least two endogenous genes are SOCS1 and ZC3H12A, wherein the gene-regulating system comprises at least one SOCS1-targeting TALEN, zinc finger, or meganuclease protein that binds to a DNA sequence selected from SEQ ID NOs: 7-151 and at least one ZC3H12A-targeting TALEN, zinc finger, or meganuclease protein that binds to a DNA sequence selected from SEQ ID NOs: 208-230.

In some embodiments, the at least two endogenous genes are PTPN2 and ZC3H12A, wherein the gene-regulating system comprises at least one PTPN2-targeting TALEN, zinc finger, or meganuclease protein that binds to a target DNA sequence defined by a set of genome coordinates shown in Tables 5 and 6 and at least one ZC3H12A-targeting TALEN, zinc finger, or meganuclease protein that binds to a target DNA sequence defined by a set of genome coordinates shown in Tables 7 and 8. In some embodiments, the at least two endogenous genes are PTPN2 and ZC3H12A, wherein the gene-regulating system comprises at least one PTPN2-targeting TALEN, zinc finger, or meganuclease protein that binds to a DNA sequence selected from SEQ ID NOs: 185-207 and at least one ZC3H12A-targeting TALEN, zinc finger, or meganuclease protein that binds to a DNA sequence selected from SEQ ID NOs: 208-230.

In some embodiments, the gene-regulating system is introduced to the immune effector cell by transfection, transduction, electroporation, or physical disruption of the cell membrane by a microfluidics device. In some embodiments, the gene-regulating system is introduced as a polynucleotide encoding one or more components of the system, a protein, or a ribonucleoprotein (RNP) complex.

In some embodiments, the present disclosure provides a modified immune effector cell, comprising reduced expression and/or function of at least two endogenous genes selected from SOCS1, PTPN2, and ZC3H12A, wherein the reduced expression and/or function of the at least two endogenous genes enhances an effector function of the immune effector cell. In some embodiments, the at least two target genes are SOCS1 and PTPN2. In some embodiments, the at least two target genes are SOCS1 and ZC3H12A. In some embodiments, the at least two target genes are PTPN2 and ZC3H12A.

In some embodiments, the present disclosure provides a modified immune effector cell, comprising an inactivating mutation in at least two endogenous genes selected from SOCS1, PTPN2, and ZC3H12A. In some embodiments, the immune effector cell is a tumor infiltrating lymphocyte (TIL) or a CAR-T cell. In some embodiments, the at least two target genes are SOCS1 and PTPN2. In some embodiments, the at least two target genes are SOCS1 and ZC3H12A. In some embodiments, the at least two target genes are PTPN2 and ZC3H12A. In some embodiments, the inactivating mutation comprises a deletion, substitution, or insertion of one or more nucleotides in the genomic sequences of the two or more endogenous genes. In some embodiments, the deletion is a partial or complete deletion of the two or more endogenous target genes. In some embodiments, the inactivating mutation is a frame shift mutation. In some embodiments, the inactivating mutation reduces the expression and/or function of the two or more endogenous target genes.

In some embodiments, the present disclosure provides a modified immune effector cell, comprising one or more exogenous polynucleotides encoding at least two nucleic acid inhibitors of at least two endogenous target genes selected from SOCS1, PTPN2, and ZC3H12A. In some embodiments, the immune effector cell is a tumor infiltrating lymphocyte (TIL) or a CAR-T cell. In some embodiments, the at least two target genes are SOCS1 and PTPN2. In some embodiments, the at least two target genes are SOCS1 and ZC3H12A. In some embodiments, the at least two target genes are PTPN2 and ZC3H12A. In some embodiments, the at least two nucleic acid inhibitors reduce the expression and/or function of the two or more endogenous target genes. In some embodiments, the expression of the two or more endogenous target genes is reduced by at least 50%, at least 60%, at least 70%, at least 80%, or at least 90% compared to an un-modified or control immune effector cell. In some embodiments, the function of the two or more endogenous target genes is reduced by at least 50%, at least 60%, at least 70%, at least 80%, or at least 90% compared to an un-modified or control immune effector cell. In some embodiments, the inactivating mutation or nucleic acid inhibitors substantially inhibits the expression of the two or more endogenous target genes. In some embodiments, the inactivating mutation or nucleic acid inhibitors substantially inhibits the function of the two or more endogenous target genes. In some embodiments, the inactivating mutation or nucleic acid inhibitors enhances one or more effector functions of the modified immune effector cell. In some embodiments, the one or more effector functions are enhanced compared to an un-modified or control immune effector cell.

In some embodiments, the immune effector cell is a T cell, a natural killer (NK) cell, an NKT cell, or a tumor infiltrating lymphocyte (TIL). In some embodiments, the modified immune effector cell further comprises an exogenous transgene expressing an immune activating molecule. In some embodiments, the immune activating molecule is selected from the group consisting of a cytokine, a chemokine, a co-stimulatory molecule, an activating peptide, an antibody, or an antigen-binding fragment thereof.

In some embodiments, the effector function is selected from cell proliferation, cell viability, tumor infiltration, cytotoxicity, anti-tumor immune responses, and/or resistance to exhaustion.

In some embodiments, the modified immune effector cell further comprises an engineered immune receptor displayed on the cell surface. In some embodiments, the engineered immune receptor is a chimeric antigen receptor (CAR) comprising an antigen-binding domain, a transmembrane domain, and an intracellular signaling domain. In some embodiments, the engineered immune receptor is an engineered T cell receptor (TCR). In some embodiments, the engineered immune receptor is capable of specifically binding to an antigen expressed on the surface of a target cell, wherein the antigen is a tumor-associated antigen.

In some embodiments, the present disclosure provides a composition comprising a modified immune effector cell described herein. In some embodiments, the composition comprises at least 1×10⁴, 1×10⁵, 1×10⁶, 1×10⁷, 1×10⁸, 1×10⁹, 1×10¹⁰, 1×10¹¹, or more modified immune effector cells. In some embodiments, the composition comprises a pharmaceutically acceptable carrier or diluent. In some embodiments, the composition comprises autologous immune effector cells. In some embodiments, the composition comprises allogeneic immune effector cells.

In some embodiments, the present disclosure provides a gene-regulating system capable of reducing expression of at least two endogenous target genes in a cell selected from SOCS1, PTPN2, and ZC3H12A, comprising (i) a nucleic acid molecule; (ii) an enzymatic protein; or (iii) a nucleic acid molecule and an enzymatic protein. In some embodiments, the at least two target genes are SOCS1 and PTPN2. In some embodiments, the at least two target genes are SOCS1 and ZC3H12A. In some embodiments, the at least two endogenous genes are PTPN2 and ZC3H12A.

In some embodiments, the system comprises at least two guide RNA (gRNA) nucleic acid molecules and a Cas endonuclease. In some embodiments, the at least two target genes are SOCS1 and PTPN2, and wherein the system comprises at least one SOCS1-targeting guide RNA (gRNA) molecule, at least one PTPN2-targeting gRNA molecule, and a Cas endonuclease. In some embodiments, the at least one SOCS1-targeting gRNA molecule comprises a targeting domain sequence complementary to a nucleic acid sequence defined by a set of genome coordinates shown in Tables 3 and 4 and the at least one PTPN2-targeting gRNA molecule comprises a targeting domain sequence complementary to a nucleic acid sequence defined by a set of genome coordinates shown in Tables 5 and 6. In some embodiments, the at least one SOCS1-targeting gRNA molecule comprises a targeting domain sequence that binds to a nucleic acid sequence defined by a set of genome coordinates shown in Tables 3 and 4 and the at least one PTPN2-targeting gRNA molecule comprises a targeting domain sequence that binds to a nucleic acid sequence defined by a set of genome coordinates shown in Tables 5 and 6. In some embodiments, the at least one SOCS1-targeting gRNA molecule comprises a targeting domain sequence that binds to a target DNA sequence selected from SEQ ID NOs: 7-151 and the at least one PTPN2-targeting gRNA molecule comprises a targeting domain sequence that binds to a target DNA sequence selected from SEQ ID NOs: 185-207. In some embodiments, the at least one SOCS1-targeting gRNA molecule comprises a targeting domain sequence encoded by a nucleic acid sequence selected from SEQ ID NOs: 7-151 and the at least one PTPN2-targeting gRNA molecule comprises a targeting domain sequence encoded by a nucleic acid sequence selected from SEQ ID NOs: 185-207.

In some embodiments, the at least two target genes are SOCS1 and ZC3H12A, and wherein the system comprises at least one SOCS1-targeting gRNA molecule, at least one ZC3H12A-targeting gRNA molecule, and a Cas endonuclease. In some embodiments, the at least one SOCS1-targeting gRNA molecule comprises a targeting domain sequence complementary to a nucleic acid sequence defined by a set of genome coordinates shown in Tables 3 and 4 and the at least one ZC3H12A-targeting gRNA molecule comprises a targeting domain sequence complementary to a nucleic acid sequence defined by a set of genome coordinates shown in Tables 7 and 8. In some embodiments, the at least one SOCS1-targeting gRNA molecule comprises a targeting domain sequence that binds to a nucleic acid sequence defined by a set of genome coordinates shown in Tables 3 and 4 and the at least one ZC3H12A-targeting gRNA molecule comprises a targeting domain sequence that binds to a nucleic acid sequence defined by a set of genome coordinates shown in Tables 7 and 8. In some embodiments, the at least one SOCS1-targeting gRNA molecule comprises a targeting domain sequence that binds to a target DNA sequence selected from SEQ ID NOs: 7-151 and the at least one ZC3H12A-targeting gRNA molecule comprises a targeting domain sequence that binds to a target DNA sequence selected from SEQ ID NOs: 208-230. In some embodiments, the at least one SOCS1-targeting gRNA molecule comprises a targeting domain sequence encoded by a nucleic acid sequence selected from SEQ ID NOs: 7-151 and the at least one ZC3H12A-targeting gRNA molecule comprises a targeting domain sequence encoded by a nucleic acid sequence selected from SEQ ID NOs: 208-230.

In some embodiments, the at least two endogenous genes are PTPN2 and ZC3H12A, wherein the system comprises at least one PTNP2-targeting gRNA molecule, at least one ZC3H12A-targeting gRNA molecule, and a Cas endonuclease. In some embodiments, the at least one PTPN2-targeting gRNA molecule comprises a targeting domain sequence complementary to a nucleic acid sequence defined by a set of genome coordinates shown in Tables 5 and 6 and the at least one ZC3H12A-targeting gRNA molecule comprises a targeting domain sequence complementary to a nucleic acid sequence defined by a set of genome coordinates shown in Tables 7 and 8. In some embodiments, the at least one PTPN2-targeting gRNA molecule comprises a targeting domain sequence that binds to a nucleic acid sequence defined by a set of genome coordinates shown in Tables 5 and 6 and the at least one ZC3H/2A-targeting gRNA molecule comprises a targeting domain sequence that binds to a nucleic acid sequence defined by a set of genome coordinates shown in Tables 7 and 8. In some embodiments, the at least one PTPN2-targeting gRNA molecule comprises a targeting domain sequence that binds to a target DNA sequence selected from SEQ ID NOs: 185-207 and the at least one ZC3H/2A-targeting gRNA molecule comprises a targeting domain sequence that binds to a target DNA sequence selected from SEQ ID NOs: 208-230. In some embodiments, the at least one PTPN2-targeting gRNA molecule comprises a targeting domain sequence encoded by a nucleic acid sequence selected from SEQ ID NOs: 185-207 and the at least one ZC3H/2A-targeting gRNA molecule comprises a targeting domain sequence encoded by a nucleic acid sequence selected from SEQ ID NOs: 208-230.

In some embodiments, the Cas protein is a Cas9 protein. In some embodiments, the Cas protein is a wild-type Cas protein comprising two enzymatically active domains, and capable of inducing double stranded DNA breaks. In some embodiments, the Cas protein is a Cas nickase mutant comprising one enzymatically active domain and capable of inducing single stranded DNA breaks. In some embodiments, the Cas protein is a deactivated Cas protein (dCas) and is associated with a heterologous protein capable of modulating the expression of the one or more endogenous target genes. In some embodiments, the heterologous protein is selected from the group consisting of MAX-interacting protein 1 (MXI1), Krüppel-associated box (KRAB) domain, and four concatenated mSin3 domains (SID4X).

In some embodiments, the system comprises at least two nucleic acid molecules and wherein the at least two nucleic acid molecules are selected from an siRNA, an shRNA, a microRNA (miR), an antagomiR, or an antisense RNA. In some embodiments, the at least two target genes are SOCS1 and PTPN2, and wherein the system comprises at least one SOCS1-targeting guide siRNA or shRNA molecule and at least one PTPN2-targeting siRNA or shRNA molecule. In some embodiments, the SOCS1-targeting siRNA or shRNA molecule comprises about 19-30 nucleotides that are complementary to an RNA sequence encoded by a DNA sequence defined by a set of genome coordinates shown in Tables 3 and 4 and the at least one PTPN2-targeting siRNA or shRNA molecule comprises about 19-30 nucleotides that are complementary to an RNA sequence encoded by a DNA sequence defined by a set of genome coordinates shown in Tables 5 and 6. In some embodiments, the at least one SOCS1-targeting siRNA or shRNA molecule comprises about 19-30 nucleotides that binds to an RNA sequence encoded by a DNA sequence defined by a set of genome coordinates shown in Tables 3 and 4 and the at least one PTPN2-targeting siRNA or shRNA molecule comprises about 19-30 nucleotides that binds to an RNA sequence encoded by a DNA sequence defined by a set of genome coordinates shown in Tables 5 and 6. In some embodiments, the at least one SOCS1-targeting siRNA or shRNA comprises about 19-30 nucleotides that bind to an RNA sequence encoded by a DNA sequence selected from SEQ ID NOs: 7-151 and the at least one PTPN2-targeting siRNA or shRNA comprises about 19-30 nucleotides that bind to an RNA sequence encoded by a DNA sequence selected from SEQ ID NOs: 185-207.

In some embodiments, the at least two target genes are SOCS1 and ZC3H12A, and wherein the system comprises at least one SOCS1-targeting guide siRNA or shRNA molecule and at least one ZC3H12A-targeting siRNA or shRNA molecule. In some embodiments, the at least one SOCS1-targeting siRNA or shRNA molecule comprises about 19-30 nucleotides that are complementary to an RNA sequence encoded by a DNA sequence defined by a set of genome coordinates shown in Tables 3 and 4 and the at least one ZC3H12A-targeting siRNA or shRNA molecule comprises about 19-30 nucleotides that are complementary to an RNA sequence encoded by a DNA sequence defined by a set of genome coordinates shown in Tables 7 and 8. In some embodiments, the at least one SOCS1-targeting siRNA or shRNA molecule comprises about 19-30 nucleotides that binds to an RNA sequence encoded by a DNA sequence defined by a set of genome coordinates shown in Tables 3 and 4 and the at least one ZC3H12A-targeting siRNA or shRNA molecule comprises about 19-30 nucleotides and binds to an RNA sequence encoded by a DNA sequence defined by a set of genome coordinates shown in Tables 7 and 8. In some embodiments, the at least one SOCS1-targeting siRNA or shRNA comprises about 19-30 nucleotides that bind to an RNA sequence encoded by a DNA sequence selected from SEQ ID NOs: 7-151 and the at least one ZC3H12A-targeting siRNA or shRNA comprises about 19-30 nucleotides that bind to an RNA sequence encoded by a DNA sequence selected from SEQ ID NOs: 208-230.

In some embodiments, the at least two target genes are PTPN2 and ZC3H12A, and wherein the system comprises at least one PTPN2-targeting guide siRNA or shRNA molecule and at least one ZC3H12A-targeting siRNA or shRNA molecule. In some embodiments, the at least one PTPN2-targeting siRNA or shRNA molecule comprises about 19-30 nucleotides that are complementary to an RNA sequence encoded by a DNA sequence defined by a set of genome coordinates shown in Tables 5 and 6 and the at least one ZC3H12A-targeting siRNA or shRNA molecule comprises about 19-30 nucleotides that are complementary to an RNA sequence encoded by a DNA sequence defined by a set of genome coordinates shown in Tables 7 and 8. In some embodiments, the at least one PTPN2-targeting siRNA or shRNA molecule comprises about 19-30 nucleotides that binds to an RNA sequence encoded by a DNA sequence defined by a set of genome coordinates shown in Tables 5 and 6 and the at least one ZC3H/2A-targeting siRNA or shRNA molecule comprises about 19-30 nucleotides that binds to an RNA sequence encoded by a DNA sequence defined by a set of genome coordinates shown in Tables 7 and 8. In some embodiments, the at least one PTPN2-targeting siRNA or shRNA comprises about 19-30 nucleotides that bind to an RNA sequence encoded by a DNA sequence selected from SEQ ID NOs: 185-207 and the at least one ZC3H/2A-targeting siRNA or shRNA comprises about 19-30 nucleotides that bind to an RNA sequence encoded by a DNA sequence selected from SEQ ID NOs: 208-230.

In some embodiments, the gene-regulating system comprises an enzymatic protein, and wherein the enzymatic protein has been engineered to specifically bind to a target sequence in one or more of the endogenous genes. In some embodiments, the system comprises a protein comprising a DNA binding domain and an enzymatic domain and is selected from a zinc finger nuclease and a transcription-activator-like effector nuclease (TALEN). In some embodiments, the system comprises one or more vectors encoding at least one gRNA targeting a first target gene, at least one gRNA targeting a second target gene, and a Cas endonuclease protein, wherein the first target gene is SOCS1 and the least one SOCS1-targeting gRNA comprises a targeting domain sequence encoded by a nucleic acid sequence selected from SEQ ID NOs: 7-151, and wherein the second target gene is PTPN2 and the at least one PTPN2-targeting gRNA comprises a targeting domain sequence encoded by a nucleic acid sequence selected from SEQ ID NOs: 185-207.

In some embodiments, the gene-regulating system comprises one or more vectors encoding at least one gRNA targeting a first target gene, at least one gRNA targeting a second target gene, and a Cas endonuclease protein, wherein the first target gene is SOCS1 and the least one SOCS1-targeting gRNA comprises a targeting domain sequence encoded by a nucleic acid sequence selected from SEQ ID NOs: 7-151, and wherein the second target gene is ZC3H12A and the at least one ZC3H/2A-targeting gRNA comprises a targeting domain sequence encoded by a nucleic acid sequence selected from SEQ ID NOs: 208-230.

In some embodiments, the gene-regulating system comprises one or more vectors encoding at least one gRNA targeting a first target gene, at least one gRNA targeting a second target gene, and a Cas endonuclease protein, wherein the first target gene is PTPN2 and the PTPN2-targeting gRNA comprises a targeting domain sequence encoded by a nucleic acid sequence selected from SEQ ID NOs: 185-207, and wherein the second target gene is ZC3H12A and the at least one ZC3H12A-targeting gRNA comprises a targeting domain sequence encoded by a nucleic acid sequence selected from SEQ ID NOs: 208-230.

In some embodiments, the at least one gRNA targeting the first target gene, the at least one gRNA targeting the second target gene, and the Cas endonuclease protein are encoded by one vector. In some embodiments, the at least one gRNA targeting the first target gene and the at least one gRNA targeting the second target gene are encoded by a first vector, and the Cas endonuclease protein is encoded by a second vector. In some embodiments, the at least one gRNA targeting the first target gene is encoded by a first vector, the at least one gRNA targeting the second target gene is encoded by a second vector, and the Cas endonuclease protein is encoded by a third vector.

In some embodiments, the gene-regulating system comprises (i) one or more vectors encoding at least one SOCS1-targeting gRNA comprising a targeting domain sequence encoded by a nucleic acid sequence selected from SEQ ID NOs: 7-151 and at least one PTPN2-targeting gRNA comprising a targeting domain sequence encoded by a nucleic acid sequence selected from SEQ ID NOs: 185-207; and (ii) an mRNA molecule encoding the Cas endonuclease protein.

In some embodiments, the gene-regulating system comprises (i) one or more vectors encoding at least one SOCS1-targeting gRNA comprising a targeting domain sequence encoded by a nucleic acid sequence selected from SEQ ID NOs: 7-151 and at least one ZC3H12A-targeting gRNA comprising a targeting domain sequence encoded by a nucleic acid sequence selected from SEQ ID NOs: 208-230; and (ii) an mRNA molecule encoding the Cas endonuclease protein.

In some embodiments, the gene-regulating system comprises (i) one or more vectors encoding at least one PTPN2-targeting gRNA comprising a targeting domain sequence encoded by a nucleic acid sequence selected from SEQ ID NOs: 185-207 and at least one ZC3H/2A-targeting gRNA comprising a targeting domain sequence encoded by a nucleic acid sequence selected from SEQ ID NOs: 208-230; and (ii) an mRNA molecule encoding the Cas endonuclease protein.

In some embodiments, the at least one gRNA targeting the first target gene and the at least one gRNA targeting the second target gene are encoded by one vector. In some embodiments, the at least one gRNA targeting the first target gene is encoded by a first vector and the at least one gRNA targeting the second target gene is encoded by a second vector.

In some embodiments, the gene-regulating system comprises (i) at least one SOCS1-targeting gRNA comprising a targeting domain sequence encoded by a nucleic acid sequence selected from SEQ ID NOs: 7-151 complexed to a first Cas endonuclease protein to form a first ribonucleoprotein (RNP) complex; and (ii) at least one PTPN2-targeting gRNA comprising a targeting domain sequence encoded by a nucleic acid sequence selected from SEQ ID NOs: 185-207 complexed to a second Cas endonuclease protein to form a second RNP complex.

In some embodiments, the gene-regulating system comprises (i) at least one SOCS1-targeting gRNA comprising a targeting domain sequence encoded by a nucleic acid sequence selected from SEQ ID NOs: 7-151 complexed to a first Cas endonuclease protein to form a first RNP complex; and (ii) at least one ZC3H/2A-targeting gRNA comprising a targeting domain sequence encoded by a nucleic acid sequence selected from SEQ ID NOs: 208-230 complexed to a second Cas endonuclease protein to form a second RNP complex.

In some embodiments, the gene-regulating system comprises (i) at least one PTPN2-targeting gRNA comprising a targeting domain sequence encoded by a nucleic acid sequence selected from SEQ ID NOs: 185-207 complexed to a first Cas endonuclease protein to form a first RNP complex; and (ii) at least one ZC3H/2A-targeting gRNA comprising a targeting domain sequence encoded by a nucleic acid sequence selected from SEQ ID NOs: 208-230 complexed to a second Cas endonuclease protein to form a second RNP complex.

In some embodiments, the present disclosure provides a kit comprising a gene-regulating system described herein.

In some embodiments, the present disclosure provides a composition comprising a plurality of gRNA molecules, wherein the plurality of gRNA molecules comprises at least one gRNA molecule targeting a first target gene and at least one gRNA molecule targeting a second target gene, wherein the first and second target gene are selected from SOCS1, PTPN2, and ZC3H12A. In some embodiments, the first target gene is SOCS1 and the second target gene is PTPN2. In some embodiments, the plurality of gRNA molecules comprises at least one SOCS1-targeting gRNA molecule comprising a targeting domain sequence complementary to a nucleic acid sequence defined by a set of genome coordinates shown in Tables 3 and 4 and at least one PTPN2-targeting gRNA molecule comprising a targeting domain sequence complementary to a nucleic acid sequence defined by a set of genome coordinates shown in Tables 5 and 6. In some embodiments, the plurality of gRNA molecules comprises at least one SOCS1-targeting gRNA molecule comprises a targeting domain sequence that binds to a nucleic acid sequence defined by a set of genome coordinates shown in Tables 3 and 4 and the at least one PTPN2-targeting gRNA molecule comprises a targeting domain sequence that binds to a nucleic acid sequence defined by a set of genome coordinates shown in Tables 5 and 6. In some embodiments, the plurality of gRNA molecules comprises at least one SOCS1-targeting gRNA molecule comprises a targeting domain sequence that binds to a target DNA sequence selected from SEQ ID NOs: 7-151 and the at least one PTPN2-targeting gRNA molecule comprises a targeting domain sequence that binds to a target DNA sequence selected from SEQ ID NOs: 185-207. In some embodiments, the plurality of gRNA molecules comprises at least one SOCS1-targeting gRNA molecule comprises a targeting domain sequence encoded by a nucleic acid sequence selected from SEQ ID NOs: 7-151 and the at least one PTPN2-targeting gRNA molecule comprises a targeting domain sequence encoded by a nucleic acid sequence selected from SEQ ID NOs: 185-207.

In some embodiments, the first target gene is SOCS1 and the second target gene is ZC3H12A. In some embodiments, the at least one SOCS1-targeting gRNA molecule comprises a targeting domain sequence complementary to a nucleic acid sequence defined by a set of genome coordinates shown in Tables 3 and 4 and the at least one ZC3H12A-targeting gRNA molecule comprises a targeting domain sequence complementary to a nucleic acid sequence defined by a set of genome coordinates shown in Tables 7 and 8. In some embodiments, the at least one SOCS1-targeting gRNA molecule comprises a targeting domain sequence that binds to a nucleic acid sequence defined by a set of genome coordinates shown in Tables 3 and 4 and the at least one ZC3H12A-targeting gRNA molecule comprises a targeting domain sequence that binds to a nucleic acid sequence defined by a set of genome coordinates shown in Tables 7 and 8. In some embodiments, the at least one SOCS1-targeting gRNA molecule comprises a targeting domain sequence that binds to a target DNA sequence selected from SEQ ID NOs: 7-151 and the at least one ZC3H12A-targeting gRNA molecule comprises a targeting domain sequence that binds to a target DNA sequence selected from SEQ ID NOs: 208-230. In some embodiments, the at least one SOCS1-targeting gRNA molecule comprises a targeting domain sequence encoded by a nucleic acid sequence selected from SEQ ID NOs: 7-151 and the at least one ZC3H12A-targeting gRNA molecule comprises a targeting domain sequence encoded by a nucleic acid sequence selected from SEQ ID NOs: 208-230.

In some embodiments, the first target gene is PTPN2 and the second target gene is ZC3H12A. In some embodiments, the at least one PTPN2-targeting gRNA molecule comprises a targeting domain sequence complementary to a nucleic acid sequence defined by a set of genome coordinates shown in Tables 5 and 6 and the at least one ZC3H12A-targeting gRNA molecule comprises a targeting domain sequence complementary to a nucleic acid sequence defined by a set of genome coordinates shown in Tables 7 and 8. In some embodiments, the at least one PTPN2-targeting gRNA molecule comprises a targeting domain sequence that binds to a nucleic acid sequence defined by a set of genome coordinates shown in Tables 5 and 6 and the at least one ZC3H12A-targeting gRNA molecule comprises a targeting domain sequence that binds to a nucleic acid sequence defined by a set of genome coordinates shown in Tables 7 and 8. In some embodiments, the at least one PTPN2-targeting gRNA molecule comprises a targeting domain sequence that binds to a target DNA sequence selected from SEQ ID NOs: 185-207 and the at least one ZC3H/2A-targeting gRNA molecule comprises a targeting domain sequence that binds to a target DNA sequence selected from SEQ ID NOs: 208-230. In some embodiments, the at least one PTPN2-targeting gRNA molecule comprises a targeting domain sequence encoded by a nucleic acid sequence selected from SEQ ID NOs: 185-207 and the at least one ZC3H/2A-targeting gRNA molecule comprises a targeting domain sequence encoded by a nucleic acid sequence selected from SEQ ID NOs: 208-230.

In some embodiments, the gRNAs are modular gRNA molecules. In some embodiments, the gRNAs are dual gRNA molecules. In some embodiments, the gRNA targeting domains are 16, 17, 18, 19, 20, 21, 22, 23, 24, 25, 26 or more nucleotides in length. In some embodiments, the gRNAs comprise a modification at or near the 5′ end (e.g., within 1-10, 1-5, or 1-2 nucleotides of the 5′ end) and/or a modification at or near the 3′ end (e.g., within 1-10, 1-5, or 1-2 nucleotides of the 3′ end). In some embodiments, the modified gRNAs exhibit increased resistance to nucleases when introduced into an immune effector cell. In some embodiments, the modified gRNAs do not induce an innate immune response when introduced into an immune effector cell or induce a decreased innate immune response compared to an unmodified gRNA when introduced into an immune effector cell.

In some embodiments, the present disclosure provides a polynucleotide molecule encoding a plurality of gRNA molecules, wherein the plurality of gRNA molecules comprises at least one gRNA molecule targeting a first target gene and at least one gRNA molecule targeting a second target gene, wherein the first and second target gene are selected from SOCS1, PTPN2, and ZC3H12A.

In some embodiments, the first target gene is SOCS1 and the second target gene is PTPN2. In some embodiments, the plurality of gRNA molecules comprises at least one SOCS1-targeting gRNA molecule comprising a targeting domain sequence complementary to a nucleic acid sequence defined by a set of genome coordinates shown in Tables 3 and 4 and at least one PTPN2-targeting gRNA molecule comprising a targeting domain sequence complementary to a nucleic acid sequence defined by a set of genome coordinates shown in Tables 5 and 6. In some embodiments, the plurality of gRNA molecules comprises at least one SOCS1-targeting gRNA molecule comprises a targeting domain sequence that binds to a nucleic acid sequence defined by a set of genome coordinates shown in Tables 3 and 4 and the at least one PTPN2-targeting gRNA molecule comprises a targeting domain sequence that binds to a nucleic acid sequence defined by a set of genome coordinates shown in Tables 5 and 6. In some embodiments, the plurality of gRNA molecules comprises at least one SOCS1-targeting gRNA molecule comprises a targeting domain sequence that binds to a target DNA sequence selected from SEQ ID NOs: 7-151 and the at least one PTPN2-targeting gRNA molecule comprises a targeting domain sequence that binds to a target DNA sequence selected from SEQ ID NOs: 185-207. In some embodiments, the plurality of gRNA molecules comprises at least one SOCS1-targeting gRNA molecule comprises a targeting domain sequence encoded by a nucleic acid sequence selected from SEQ ID NOs: 7-151 and the at least one PTPN2-targeting gRNA molecule comprises a targeting domain sequence encoded by a nucleic acid sequence selected from SEQ ID NOs: 185-207.

In some embodiments, the first target gene is SOCS1 and the second target gene is ZC3H12A. In some embodiments, the at least one SOCS1-targeting gRNA molecule comprises a targeting domain sequence complementary to a nucleic acid sequence defined by a set of genome coordinates shown in Tables 3 and 4 and the at least one ZC3H/2A-targeting gRNA molecule comprises a targeting domain sequence complementary to a nucleic acid sequence defined by a set of genome coordinates shown in Tables 7 and 8. In some embodiments, the at least one SOCS1-targeting gRNA molecule comprises a targeting domain sequence that binds to a nucleic acid sequence defined by a set of genome coordinates shown in Tables 3 and 4 and the at least one ZC3H12A-targeting gRNA molecule comprises a targeting domain sequence that binds to a nucleic acid sequence defined by a set of genome coordinates shown in Tables 7 and 8. In some embodiments, the at least one SOCS1-targeting gRNA molecule comprises a targeting domain sequence that binds to a target DNA sequence selected from SEQ ID NOs: 7-151 and the at least one ZC3H12A-targeting gRNA molecule comprises a targeting domain sequence that binds to a target DNA sequence selected from SEQ ID NOs: 208-230. In some embodiments, the at least one SOCS1-targeting gRNA molecule comprises a targeting domain sequence encoded by a nucleic acid sequence selected from SEQ ID NOs: 7-151 and the at least one ZC3H/2A-targeting gRNA molecule comprises a targeting domain sequence encoded by a nucleic acid sequence selected from SEQ ID NOs: 208-230.

In some embodiments, the first target gene is PTPN2 and the second target gene is ZC3H12A. In some embodiments, the at least one PTPN2-targeting gRNA molecule comprises a targeting domain sequence complementary to a nucleic acid sequence defined by a set of genome coordinates shown in Tables 5 and 6 and the at least one ZC3H12A-targeting gRNA molecule comprises a targeting domain sequence complementary to a nucleic acid sequence defined by a set of genome coordinates shown in Tables 7 and 8. In some embodiments, the at least one PTPN2-targeting gRNA molecule comprises a targeting domain sequence that binds to a nucleic acid sequence defined by a set of genome coordinates shown in Tables 5 and 6 and the at least one ZC3H12A-targeting gRNA molecule comprises a targeting domain sequence that binds to a nucleic acid sequence defined by a set of genome coordinates shown in Tables 7 and 8. In some embodiments, the at least one PTPN2-targeting gRNA molecule comprises a targeting domain sequence that binds to a target DNA sequence selected from SEQ ID NOs: 185-207 and the at least one ZC3H12A-targeting gRNA molecule comprises a targeting domain sequence that binds to a target DNA sequence selected from SEQ ID NOs: 208-230. In some embodiments, at least one PTPN2-targeting gRNA molecule comprises a targeting domain sequence encoded by a nucleic acid sequence selected from SEQ ID NOs: 185-207 and the at least one ZC3H12A-targeting gRNA molecule comprises a targeting domain sequence encoded by a nucleic acid sequence selected from SEQ ID NOs: 208-230.

In some embodiments, the present disclosure provides a polynucleotide molecule encoding a plurality of siRNA or shRNA molecules, wherein the plurality of siRNA or shRNA molecules comprises at least one siRNA or shRNA molecule targeting a first target gene and at least one siRNA or shRNA molecule targeting a second target gene, wherein the first and second target gene are selected from SOCS1, PTPN2, and ZC3H12A.

In some embodiments, the first target gene is SOCS1 and the second target gene is PTPN2. In some embodiments, the plurality of siRNA or shRNA molecules comprises at least one SOCS1-targeting siRNA or shRNA molecule comprising a targeting domain sequence that binds to an RNA sequence encoded by a DNA sequence defined by a set of genome coordinates shown in Tables 3 and 4 and at least one PTPN2-targeting siRNA or shRNA molecule comprising a targeting domain sequence complementary to an RNA sequence encoded by a DNA sequence defined by a set of genome coordinates shown in Tables 5 and 6. In some embodiments, the plurality of siRNA or shRNA molecules comprises at least one SOCS1-targeting siRNA or shRNA molecule comprising a targeting domain sequence t that binds to an RNA sequence encoded by a DNA sequence selected from SEQ ID NOs: 7-151 and the at least one PTPN2-targeting siRNA or shRNA molecule comprising a targeting domain sequence that binds to an RNA sequence encoded by a DNA sequence selected from SEQ ID NOs: 185-207.

In some embodiments, the first target gene is SOCS1 and the second target gene is ZC3H12A. In some embodiments, the at least one SOCS1-targeting siRNA or shRNA molecule comprising a targeting domain sequence that binds to an RNA sequence encoded by a DNA sequence defined by a set of genome coordinates shown in Tables 3 and 4 and the at least one ZC3H12A-targeting siRNA or shRNA molecule comprising a targeting domain sequence that binds to an RNA sequence encoded by a DNA sequence defined by a set of genome coordinates shown in Tables 7 and 8. In some embodiments, the at least one SOCS1-targeting siRNA or shRNA molecule comprising a targeting domain sequence that binds to an RNA sequence encoded by a DNA sequence selected from SEQ ID NOs: 7-151 and the at least one ZC3H12A-targeting siRNA or shRNA molecule comprising a targeting domain sequence that binds to an RNA sequence encoded by a DNA sequence selected from SEQ ID NOs: 208-230.

In some embodiments, the first target gene is PTPN2 and the second target gene is ZC3H12A. In some embodiments, the at least one PTPN2-targeting siRNA or shRNA molecule comprising a targeting domain sequence that binds to an RNA sequence encoded by a DNA sequence defined by a set of genome coordinates shown in Tables 5 and 6 and the at least one ZC3H12A-targeting siRNA or shRNA molecule comprising a targeting domain sequence that binds to an RNA sequence encoded by a DNA sequence defined by a set of genome coordinates shown in Tables 7 and 8. In some embodiments, the at least one PTPN2-targeting siRNA or shRNA molecule comprising a targeting domain sequence that binds to an RNA sequence encoded by a DNA sequence selected from SEQ ID NOs: 185-207 and the at least one ZC3H12A-targeting siRNA or shRNA molecule comprising a targeting domain sequence that binds to an RNA sequence encoded by a DNA sequence selected from SEQ ID NOs: 208-230.

In some embodiments, the present disclosure provides a polynucleotide molecule encoding at least one TALEN, zinc finger, or meganuclease protein targeting a first target gene and at least one TALEN, zinc finger, or meganuclease protein targeting a second target gene, wherein the first and second target gene are selected from SOCS1, PTPN2, and ZC3H12A.

In some embodiments, the first target gene is SOCS1 and the second target gene is PTPN2. In some embodiments, the polynucleotide encodes at least one SOCS1-targeting TALEN, zinc finger, or meganuclease protein comprising a targeting domain sequence that binds to a DNA sequence defined by a set of genome coordinates shown in Tables 3 and 4 and at least one PTPN2-targeting TALEN, zinc finger, or meganuclease protein comprising a targeting domain sequence that binds to a DNA sequence defined by a set of genome coordinates shown in Tables 5 and 6. In some embodiments, the polynucleotide encodes at least one SOCS1-targeting TALEN, zinc finger, or meganuclease protein comprising a targeting domain sequence that binds to a DNA sequence selected from SEQ ID NOs: 7-151 and at least one PTPN2-targeting TALEN, zinc finger, or meganuclease protein comprising a targeting domain sequence that binds to a DNA sequence selected from SEQ ID NOs: 185-207.

In some embodiments, the first target gene is SOCS1 and the second target gene is ZC3H12A. In some embodiments, the polynucleotide encodes at least one SOCS1-targeting TALEN, zinc finger, or meganuclease protein comprising a targeting domain sequence that binds to a DNA sequence defined by a set of genome coordinates shown in Tables 3 and 4 and the at least one ZC3H12A-targeting TALEN, zinc finger, or meganuclease protein comprising a targeting domain sequence that binds to a DNA sequence defined by a set of genome coordinates shown in Tables 7 and 8. In some embodiments, the polynucleotide encodes at least one SOCS1-targeting TALEN, zinc finger, or meganuclease protein comprising a targeting domain sequence that binds to a DNA sequence selected from SEQ ID NOs: 7-151 and at least one ZC3H12A-targeting TALEN, zinc finger, or meganuclease protein comprising a targeting domain sequence that binds to a DNA sequence selected from SEQ ID NOs: 208-230.

In some embodiments, the first target gene is PTPN2 and the second target gene is ZC3H12A. In some embodiments, the polynucleotide encodes least one PTPN2-targeting TALEN, zinc finger, or meganuclease protein comprising a targeting domain sequence that binds to a DNA sequence defined by a set of genome coordinates shown in Tables 5 and 6 and the at least one ZC3H12A-targeting TALEN, zinc finger, or meganuclease protein comprising a targeting domain sequence that binds to a DNA sequence defined by a set of genome coordinates shown in Tables 7 and 8. In some embodiments, the polynucleotide encodes at least one PTPN2-targeting TALEN, zinc finger, or meganuclease protein comprising a targeting domain sequence that binds to a DNA sequence selected from SEQ ID NOs: 185-207 and at least one ZC3H12A-targeting TALEN, zinc finger, or meganuclease protein comprising a targeting domain sequence that binds to a DNA sequence selected from SEQ ID NOs: 208-230.

In some embodiments, the present disclosure provides a composition comprising the polynucleotide described herein.

In some embodiments, the present disclosure provides a kit comprising the polynucleotide or composition described herein.

In some embodiments, the present disclosure provides a method of producing a modified immune effector cell comprising: introducing a gene-regulating system into the immune effector cell, wherein the gene-regulating system is capable of reducing expression and/or function of at least two endogenous target genes selected from SOCS1, PTPN2, and ZC3H12A.

In some embodiments, the present disclosure provides a method of producing a modified immune effector cell comprising: obtaining an immune effector cell from a subject; introducing a gene-regulating system into the immune effector cell, wherein the gene-regulating system is capable of reducing expression and/or function of at least two endogenous target genes selected from SOCS1, PTPN2, and ZC3H12A; and culturing the immune effector cell such that the expression and/or function of one or more endogenous target genes is reduced compared to an immune effector cell that has not been modified. In some embodiments, the gene-regulating system is one selected from those described herein. In some embodiments, the method further comprises introducing a polynucleotide sequence encoding an engineered immune receptor selected from a CAR and a TCR. In some embodiments, the gene-regulating system and/or the polynucleotide encoding the engineered immune receptor are introduced to the immune effector cell by transfection, transduction, electroporation, or physical disruption of the cell membrane by a microfluidics device. In some embodiments, the gene-regulating system is introduced as a polynucleotide sequence encoding one or more components of the system, as a protein, or as a ribonucleoprotein (RNP) complex.

In some embodiments, the present disclosure provides a method of producing a modified immune effector cell comprising: expanding a population of immune effector cells in a first round expansion and a second round of expansion; and introducing a gene-regulating system into the population of immune effector cells, wherein the gene-regulating system is capable of reducing expression and/or function of at least two endogenous target genes selected from SOCS1, PTPN2, and ZC3H12A.

In some embodiments, the present disclosure provides a method of producing a modified immune effector cell comprising: obtaining a population of immune effector cells; expanding the population of immune effector cells in a first round expansion and a second round of expansion; introducing a gene-regulating system into the population of immune effector cells, wherein the gene-regulating system is capable of reducing expression and/or function of at least two endogenous target genes selected from SOCS1, PTPN2, and ZC3H12A; and culturing the immune effector cell such that the expression and/or function of one or more endogenous target genes is reduced compared to an immune effector cell that has not been modified. In some embodiments, the gene-regulating system is introduced to the population of immune effector cells prior to the first and second rounds of expansion. In some embodiments, the gene-regulating system is introduced to the population of immune effector cells after the first round of expansion and prior to the second round of expansion. In some embodiments, the gene-regulating system is introduced to the population of immune effector cells after the first and second rounds of expansion. In some embodiments, the gene-regulating system is one selected from those described herein.

In some embodiments, the present disclosure provides a method of treating a disease or disorder in a subject in need thereof comprising administering to the subject an effective amount of the modified immune effector cells described herein or composition thereof. In some embodiments, the disease or disorder is a cell proliferative disorder, an inflammatory disorder, or an infectious disease. In some embodiments, the disease or disorder is a cancer or a viral infection. In some embodiments, the cancer is selected from a leukemia, a lymphoma, or a solid tumor. In some embodiments, the solid tumor is a melanoma, a pancreatic tumor, a bladder tumor, or a lung tumor or metastasis. In some embodiments, the cancer is a PD1 resistant or insensitive cancer. In some embodiments, the subject has previously been treated with a PD1 inhibitor or a PDL1 inhibitor. In some embodiments, the modified immune effector cells are autologous to the subject. In some embodiments, the modified immune effector cells are allogenic to the subject.

In some embodiments, the method further comprises administering to the subject an antibody or binding fragment thereof that specifically binds to and inhibits the function of the protein encoded by NRP1, HAVCR2, LAG3, TIGIT, CTLA4, or PDCD1. In some embodiments, the subject has not undergone lymphodepletion prior to administration of the modified immune effector cells or compositions thereof. In some embodiments, administration of the modified immune effector cells or compositions thereof to the subject is not accompanied by high dose IL-2 treatment. In some embodiments, administration of the modified immune effector cells or compositions thereof to the subject is not accompanied by any IL-2 treatment. In some embodiments, the subject has not undergone any IL-2 treatment prior to administration of the modified immune effector cells or compositions thereof. In some embodiments, the subject has not undergone any high dose IL-2 treatment prior to administration of the modified immune effector cells or compositions thereof. In some embodiments, the subject has not undergone lymphodepletion prior to administration of the modified immune effector cells or compositions thereof and the administration of modified immune effector cells or compositions thereof to the subject is not accompanied by high dose IL-2 treatment. In some embodiments, the subject has not undergone lymphodepletion prior to administration of the modified immune effector cells or compositions thereof and the administration of modified immune effector cells or compositions thereof to the subject is not accompanied by any IL-2 treatment. In some embodiments, the subject has not undergone lymphodepletion or high dose IL-2 treatment prior to administration of the modified immune effector cells or compositions thereof. In some embodiments, the subject has not undergone lymphodepletion or any IL-2 treatment prior to administration of the modified immune effector cells or compositions thereof.

In some embodiments, the present disclosure provides a method of killing a cancerous cell comprising exposing the cancerous cell to a modified immune effector cell described herein or composition thereof, wherein exposure to the modified immune effector cell results in increased killing of the cancerous cells compared to exposure to an immune effector cell that has not been modified. In some embodiments, the exposure is in vitro, in vivo, or ex vivo.

In some embodiments, the present disclosure provides a method of enhancing one or more effector functions of an immune effector cell comprising introducing a gene-regulating system into the immune effector cell, wherein the gene-regulating system is capable of reducing the expression and/or function of at least two endogenous target genes selected from SOCS1, PTPN2, and ZC3H12A.

In some embodiments, the present disclosure provides a method of enhancing one or more effector functions of an immune effector cell comprising: introducing a gene-regulating system into the immune effector cell, wherein the gene-regulating system is capable of reducing the expression and/or function of at least two endogenous target genes selected from SOCS1, PTPN2, and ZC3H12A; and culturing the immune effector cell such that the expression and/or function of one or more endogenous target genes is reduced compared to an immune effector cell that has not been modified, wherein the modified immune effector cell demonstrates one or more enhanced effector functions compared to the immune effector cell that has not been modified. In some embodiments, the one or more effector functions are selected from cell proliferation, cell viability, cytotoxicity, tumor infiltration, increased cytokine production, anti-tumor immune responses, and/or resistance to exhaustion. In some embodiments, the gene-regulating system is one described herein.

In some embodiments, the present disclosure provides a method of producing a modified immune effector cell comprising introducing an inactivating mutation in at least two endogenous target genes in an immune effector cell, wherein the at least two endogenous target genes are selected from SOCS1, PTPN2, and ZC3H12A.

In some embodiments, the present disclosure provides a method of producing a modified immune effector cell comprising: expanding a population of immune effector cells in a first round expansion and a second round of expansion; and introducing an inactivating mutation in at least two endogenous target genes in the population of immune effector cells, wherein the at least two endogenous target genes are selected from SOCS1, PTPN2, and ZC3H12A.

In some embodiments, the present disclosure provides a method of producing a modified immune effector cell comprising introducing one or more exogenous polynucleotides encoding at least two nucleic acid inhibitors of at least two endogenous target genes in an immune effector cell, wherein the at least two endogenous target genes are selected from SOCS1, PTPN2, and ZC3H12A.

In some embodiments, the present disclosure provides a method of producing a modified immune effector cell comprising: expanding a population of immune effector cells in a first round expansion and a second round of expansion; and introducing one or more exogenous polynucleotides encoding at least two nucleic acid inhibitors of at least two endogenous target genes in the population of immune effector cells, wherein the at least two endogenous target genes are selected from SOCS1, PTPN2, and ZC3H12A.

In some embodiments, the methods further comprise introducing a polynucleotide sequence encoding an engineered immune receptor selected from a CAR and a TCR. In some embodiments, the inactivating mutation is introduced by the nucleic acid gene-regulating system of any one of the preceding claims. In some embodiments, the at least two nucleic acid inhibitors are comprised in a gene-regulating system described herein.

In some embodiments, the disclosure provides a method of killing a cancerous cell in a subject in need thereof comprising administering to the subject a therapeutically effective amount of modified immune effector cell according to any one of claims 1-57 or the composition of any one of claims 79-83, wherein exposure to the modified immune effector cell results in increased killing of the cancerous cells compared to exposure to an immune effector cell that has not been modified, wherein the number of modified immune effector cells necessary to comprise a therapeutically effective amount is at least ten fold less or at least 100 fold less than the number of non-modified immune effector cells necessary to comprise a therapeutically effective amount. In some embodiments, the number of modified immune effector cells necessary to comprise a therapeutically effective amount is at least 1×10³, 5×10³, 1×10⁴, 5×10⁴, 1×10⁵, 5×10⁵, 1×10⁶, 2×10⁶, 3×10⁶, 4×10⁶, 5×10⁶, 1×10⁷, 5×10⁷, 1×10⁸, 5×10⁸, 1×10⁹ cells.

### BRIEF DESCRIPTION OF THE FIGURES

FIG. 1 shows the anti-tumor efficacy of Pptn2/Socs1 dual-edited transgenic T cells in a B16-Ova murine tumor model.

FIG. 2 shows the anti-tumor efficacy of Zc3h12a/Socs1 dual-edited transgenic T cells in a B16-Ova murine tumor model.

FIG. 3 shows the anti-tumor efficacy of Zc3h12a/Socs1 dual-edited TILs in a B16-Ova murine tumor model.

FIG. 4 shows the anti-tumor efficacy of Pd1/Lag3 dual-edited transgenic T cells in a B16-Ova murine tumor model.

FIG. 5 shows the increase in pSTAT5 levels in primary human CD8 T cells in response to IL-2 signaling after deletion of SOCS1.

FIG. 6 shows the increase pSTAT1 levels in Jurkat T cells in response to IFNγ stimulation after genetic knockdown of PTPN2.

FIGS. 7A-7D show the production of IFNγ (FIG. 7A) and TNFα (FIG. 7B) by dual SOCS1/PTPN2-edited human TILs as well as the ability of CD8 T cells within TIL populations to degranulate as measured by positivity (FIG. 7C) and intensity (FIG. 7D) of CD107a staining upon stimulation with phorbol 12-myristate 13-acetate (PMA) and ionomycin.

FIGS. 8A-8D show the ability of Ptpn2^{-/-}/Socs1^{-/-} OT1s to completely regress 100 mm³ B16Ova tumors in seventeen out of eighteen mice. (FIG. 8A). Also shown is the ability of the mice who previously regressed a B16Ova tumor to completely resist subsequent inoculation with B16Ova and partially resist subsequent parental B16F10 tumor inoculation. (FIG. 8B). The OT1 population in peripheral blood was tracked over the course of the rechallenge study (FIG. 8C) along with their memory phenotype (FIG. 8D).

FIGS. 9A-9E show the ability of Ptpn2^{-/-}/Socs1^{-/-} OT1s to completely regress larger 343 mm³B16Ova tumors in eight out of eight mice. (FIG. 9A). In a separate cohort of mice total OT1 infiltration into the tumor (FIG. 9B) and their ability to produce granzyme B (FIG. 9C) is presented. Also shown is the ability of the mice who previously regressed a B16Ova tumor to completely resist subsequent inoculation with B16Ova and partially resist subsequent parental B16F10 tumor inoculation. (FIG. 9D). The OT1 population in peripheral blood was tracked over the course of the rechallenge study along with their memory phenotype. (FIG. 9E).

FIGS. 10A-10H show tumor volume measurements after increasing doses of Ptpn2/Socs1 dual-edited or control edited mouse OT1 CD8+ T cells adoptively transferred into the large tumor B16Ova model. FIGS. 10A-10D show control cells administered at 4.1×10⁴, 4.1×10⁵, 4.1×10⁶ and 4.1×10⁷, respectively. FIGS. 10E-10H show Ptpn2/Socs1 dual-edited cells administered at 4.1×10⁴, 4.1×10⁵, 4.1×10⁶ and 4.1×10⁷, respectively. Each line represents the results for a separate animal.

### DETAILED DESCRIPTION

The present disclosure provides methods and compositions related to the modification of immune effector cells to increase their therapeutic efficacy in the context of immunotherapy. In some embodiments, immune effector cells are modified by the methods of the present disclosure to reduce the expression and/or function of two or more endogenous target genes selected from SOCS1, PTPN2, and ZC3H12A such that one or more effector functions of the immune cells are enhanced. In some embodiments, the immune effector cells are further modified by introduction of transgenes conferring antigen specificity, such as introduction of T cell receptor (TCR) or chimeric antigen receptor (CAR) expression constructs. In some embodiments, the present disclosure provides compositions and methods for modifying the immune effector cells, such as compositions of gene-regulating systems capable of reducing the expression and/or function of two or more endogenous target genes selected from SOCS1, PTPN2, and ZC3H12A. In some embodiments, the present disclosure provides methods of treating a cell proliferative disorder, such as a cancer, comprising administration of the modified immune effector cells described herein to a subject in need thereof.

### I. Definitions

As used in this specification and the appended claims, the singular forms “a,” “an” and “the” include plural references unless the content clearly dictates otherwise.

As used in this specification, the term “and/or” is used in this disclosure to mean either “and” or “or” unless indicated otherwise.

Throughout this specification, unless the context requires otherwise, the words “comprise”, or variations such as “comprises” or “comprising”, will be understood to imply the inclusion of a stated element or integer or group of elements or integers but not the exclusion of any other element or integer or group of elements or integers.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Decrease” or “reduce” refers to a decrease or a reduction in a particular value of at least 5%, for example, a 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100% decrease as compared to a reference value. A decrease or reduction in a particular value may also be represented as a fold-change in the value compared to a reference value, for example, at least a 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold, or more, decrease as compared to a reference value.

“Increase” refers to an increase in a particular value of at least 5%, for example, a 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, 100%, 200%, 300%, 400%, 500%, or more increase as compared to a reference value. An increase in a particular value may also be represented as a fold-change in the value compared to a reference value, for example, at least a 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 200, 500, 1000-fold or more, increase as compared to the level of a reference value.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

“Fragment” refers to a portion of a polypeptide or polynucleotide molecule containing less than the entire polypeptide or polynucleotide sequence. In some embodiments, a fragment of a polypeptide or polynucleotide comprises at least 10%, 20%, 30%, 40%, 50%, 60%, 70%, 80%, 90%, 95%, 96%, 97%, 98%, or 99% of the entire length of the reference polypeptide or polynucleotide. In some embodiments, a polypeptide or polynucleotide fragment may contain 5, 10, 15, 20, 25, 30, 35, 40, 45, 50, 60, 70, 80, 90, 100, 200, 300, 400, 500, 600, 700, 800, 900, 1000, or more nucleotides or amino acids.

The term “sequence identity” refers to the percentage of bases or amino acids between two polynucleotide or polypeptide sequences that are the same, and in the same relative position. As such one polynucleotide or polypeptide sequence has a certain percentage of sequence identity compared to another polynucleotide or polypeptide sequence. For sequence comparison, typically one sequence acts as a reference sequence, to which test sequences are compared. The term “reference sequence” refers to a molecule to which a test sequence is compared.

“Complementary” refers to the capacity for pairing, through base stacking and specific hydrogen bonding, between two sequences comprising naturally or non-naturally occurring bases or analogs thereof. For example, if a base at one position of a nucleic acid is capable of hydrogen bonding with a base at the corresponding position of a target, then the bases are considered to be complementary to each other at that position. Nucleic acids can comprise universal bases, or inert a basic spacers that provide no positive or negative contribution to hydrogen bonding. Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as such as 3-nitropyrrole or 5-nitroindole can hybridize to and are considered complementary to any A, C, U, or T. Nichols et al., Nature, 1994; 369:492-493 and Loakes et al., Nucleic Acids Res., 1994; 22:4039-4043. Inosine (I) has also been considered in the art to be a universal base and is considered complementary to any A, C, U, or T. See Watkins and SantaLucia, Nucl. Acids Research, 2005; 33 (19): 6258-6267.

As referred to herein, a “complementary nucleic acid sequence” is a nucleic acid sequence comprising a sequence of nucleotides that enables it to non-covalently bind to another nucleic acid in a sequence-specific, antiparallel, manner (i.e., a nucleic acid specifically binds to a complementary nucleic acid) under the appropriate in vitro and/or in vivo conditions of temperature and solution ionic strength.

Methods of sequence alignment for comparison and determination of percent sequence identity and percent complementarity are well known in the art. Optimal alignment of sequences for comparison can be conducted, e.g., by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), by manual alignment and visual inspection (see, e.g., Brent et al., (2003) Current Protocols in Molecular Biology), by use of algorithms know in the art including the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

Herein, the term “hybridize” refers to pairing between complementary nucleotide bases (e.g., adenine (A) forms a base pair with thymine (T) in a DNA molecule and with uracil (U) in an RNA molecule, and guanine (G) forms a base pair with cytosine (C) in both DNA and RNA molecules) to form a double-stranded nucleic acid molecule. (See, e.g., Wahl and Berger (1987) Methods Enzymol. 152:399; Kimmel, (1987) Methods Enzymol. 152:507). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a guide RNA molecule is considered complementary to a uracil (U), and vice versa. As such, when a G/U base-pair can be made at a given nucleotide position a protein-binding segment (dsRNA duplex) of a guide RNA molecule, the position is not considered to be non-complementary, but is instead considered to be complementary. It is understood in the art that the sequence of polynucleotide need not be 100% complementary to that of its target nucleic acid to be specifically hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted.

The term “modified” refers to a substance or compound (e.g., a cell, a polynucleotide sequence, and/or a polypeptide sequence) that has been altered or changed as compared to the corresponding unmodified substance or compound.

The term “naturally-occurring” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

“Isolated” refers to a material that is free to varying degrees from components which normally accompany it as found in its native state.

An “expression cassette” or “expression construct” refers to a DNA polynucleotide sequence operably linked to a promoter. “Operably linked” refers to a juxtaposition wherein the components so described are in a relationship permitting them to function in their intended manner. For instance, a promoter is operably linked to a polynucleotide sequence if the promoter affects the transcription or expression of the polynucleotide sequence.

The term “recombinant vector” as used herein refers to a polynucleotide molecule capable transferring or transporting another polynucleotide inserted into the vector. The inserted polynucleotide may be an expression cassette. In some embodiments, a recombinant vector may be viral vector or a non-viral vector (e.g., a plasmid).

The term “sample” refers to a biological composition (e.g., a cell or a portion of a tissue) that is subjected to analysis and/or genetic modification. In some embodiments, a sample is a “primary sample” in that it is obtained directly from a subject; in some embodiments, a “sample” is the result of processing of a primary sample, for example to remove certain components and/or to isolate or purify certain components of interest.

The term “subject” includes animals, such as e.g. mammals. In some embodiments, the mammal is a primate. In some embodiments, the mammal is a human. In some embodiments, subjects are livestock such as cattle, sheep, goats, cows, swine, and the like; or domesticated animals such as dogs and cats. In some embodiments (e.g., particularly in research contexts) subjects are rodents (e.g., mice, rats, hamsters), rabbits, primates, or swine such as inbred pigs and the like. The terms “subject” and “patient” are used interchangeably herein.

“Administration” refers herein to introducing an agent or composition into a subject.

“Treating” as used herein refers to delivering an agent or composition to a subject to affect a physiologic outcome.

As used herein, the term “effective amount” refers to the minimum amount of an agent or composition required to result in a particular physiological effect. The effective amount of a particular agent may be represented in a variety of ways based on the nature of the agent, such as mass/volume, # of cells/volume, particles/volume, (mass of the agent)/(mass of the subject), # of cells/(mass of subject), or particles/(mass of subject). The effective amount of a particular agent may also be expressed as the half-maximal effective concentration (EC₅₀), which refers to the concentration of an agent that results in a magnitude of a particular physiological response that is half-way between a reference level and a maximum response level.

“Population” of cells refers to any number of cells greater than 1, but is preferably at least 1×10³ cells, at least 1×10⁴ cells, at least 1×10⁵ cells, at least 1×10⁶ cells, at least 1×10⁷ cells, at least 1×10⁸ cells, at least 1×10⁹ cells, at least 1×10¹⁰ cells, or more cells. A population of cells may refer to an in vitro population (e.g., a population of cells in culture) or an in vivo population (e.g., a population of cells residing in a particular tissue).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### II. Modified Immune Effector Cells

In some embodiments, the present disclosure provides modified immune effector cells comprising reduced expression and/or function of two or more endogenous target genes selected from SOCS1, PTPN2, and ZC3H12A. In some embodiments, the present disclosure provides modified immune effector cells comprising a gene-regulating system capable of reducing the expression and/or function of two or more endogenous target genes selected from SOCS1, PTPN2, and ZC3H12A. Herein, the term “modified immune effector cells” encompasses immune effector cells comprising one or more genomic modifications resulting in the reduced expression and/or function of two or more endogenous target genes as well as immune effector cells comprising a gene-regulating system capable of reducing the expression and/or function of two or more endogenous target genes. Herein, an “un-modified immune effector cell” or “control immune effector cell” refers to a cell or population of cells wherein the genomes have not been modified and that does not comprise a gene-regulating system or comprises a control gene-regulating system (e.g., an empty vector control, a non-targeting gRNA, a scrambled siRNA, etc.).

The term “immune effector cell” refers to cells involved in mounting innate and adaptive immune responses, including but not limited to lymphocytes (such as T-cells (including thymocytes) and B-cells), natural killer (NK) cells, NKT cells, macrophages, monocytes, eosinophils, basophils, neutrophils, dendritic cells, and mast cells. In some embodiments, the modified immune effector cell is a T cell, such as a CD4+ T cell, a CD8+ T cell (also referred to as a cytotoxic T cell or CTL), a regulatory T cell (Treg), a Th1 cell, a Th2 cell, or a Th17 cell.

In some embodiments, the modified immune effector cell is a T cell that has been isolated from a tumor sample (referred to herein as “tumor infiltrating lymphocytes” or “TILs”). Without wishing to be bound by theory, it is thought that TILs possess increased specificity to tumor antigens (Radvanyi et al., 2012 Clin Canc Res 18:6758-6770) and can therefore mediate tumor antigen-specific immune response (e.g., activation, proliferation, and cytotoxic activity against the cancer cell) leading to cancer cell destruction (Brudno et al., 2018 Nat Rev Clin Onc 15:31-46)) without the introduction of an exogenous engineered receptor. Therefore, in some embodiments, TILs are isolated from a tumor in a subject, expanded ex vivo, and re-infused into a subject. In some embodiments, TILs are modified to express one or more exogenous receptors specific for an autologous tumor antigen, expanded ex vivo, and re-infused into the subject. Such embodiments can be modeled using in vivo mouse models wherein mice have been transplanted with a cancer cell line expressing a cancer antigen (e.g., CD19) and are treated with modified T cells that express an exogenous receptor that is specific for the cancer antigen (See e.g., Examples 6-9).

In some embodiments, the modified immune effector cell is an animal cell or is derived from an animal cell, including invertebrate animals and vertebrate animals (e.g., fish, amphibian, reptile, bird, or mammal). In some embodiments, the modified immune effector cell is a mammalian cell or is derived from a mammalian cell (e.g., a pig, a cow, a goat, a sheep, a rodent, a non-human primate, a human, etc.). In some embodiments, the modified immune effector cell is a rodent cell or is derived from a rodent cell (e.g., a rat or a mouse). In some embodiments, the modified immune effector cell is a human cell or is derived from a human cell.

In some embodiments, the modified immune effector cells comprise one or more modifications (e.g., insertions, deletions, or mutations of one or more nucleic acids) in the genomic DNA sequence of an endogenous target gene resulting in the reduced expression and/or function the endogenous gene. In such embodiments, the modified immune effector cells comprise a “modified endogenous target gene.” In some embodiments, the modifications in the genomic DNA sequence reduce or inhibit mRNA transcription, thereby reducing the expression level of the encoded mRNA transcript and protein. In some embodiments, the modifications in the genomic DNA sequence reduce or inhibit mRNA translation, thereby reducing the expression level of the encoded protein. In some embodiments, the modifications in the genomic DNA sequence encode a modified endogenous protein with reduced or altered function compared to the unmodified (i.e., wild-type) version of the endogenous protein (e.g., a dominant-negative mutant, described infra). In some embodiments, the modified immune effector cells comprise at least two modified endogenous target genes selected from SOCS1, PTPN2, and ZC3H12A.

In some embodiments, the modified immune effector cells comprise one or more genomic modifications at a genomic location other than an endogenous target gene that result in the reduced expression and/or function of the endogenous target gene or that result in the expression of a modified version of an endogenous protein. For example, in some embodiments, a polynucleotide sequence encoding a gene regulating system is inserted into one or more locations in the genome, thereby reducing the expression and/or function of an endogenous target gene upon the expression of the gene-regulating system. In some embodiments, a polynucleotide sequence encoding a modified version of an endogenous protein is inserted at one or more locations in the genome, wherein the function of the modified version of the protein is reduced compared to the un-modified or wild-type version of the protein (e.g., a dominant-negative mutant, described infra).

In some embodiments, the modified immune effector cells described herein comprise two or more modified endogenous target genes, wherein the one or more modifications result in a reduced expression and/or function of a gene product (i.e., an mRNA transcript or a protein) encoded by the endogenous target gene compared to an unmodified immune effector cell. For example, in some embodiments, a modified immune effector cell demonstrates reduced expression of an mRNA transcript and/or reduced expression of a protein. In some embodiments, the expression of the gene product in a modified immune effector cell is reduced by at least 5% compared to the expression of the gene product in an unmodified immune effector cell. In some embodiments, the expression of the gene product in a modified immune effector cell is reduced by at least 10%, 20%, 30%, 40%, 50%, 60%, 70%, 80%, 90%, or more compared to the expression of the gene product in an unmodified immune effector cell. In some embodiments, the modified immune effector cells described herein demonstrate reduced expression and/or function of gene products encoded by a plurality (e.g., two or more) of endogenous target genes compared to the expression of the gene products in an unmodified immune effector cell. For example, in some embodiments, a modified immune effector cell demonstrates reduced expression and/or function of gene products from 2, 3, 4, 5, 6, 7, 8, 9, 10, or more endogenous target genes compared to the expression of the gene products in an unmodified immune effector cell.

In some embodiments, the present disclosure provides a modified immune effector cell wherein two or more endogenous target genes, or a portion thereof, are deleted (i.e., “knocked-out”) such that the modified immune effector cell does not express the mRNA transcript or protein. In some embodiments, a modified immune effector cell comprises deletion of a plurality of endogenous target genes, or portions thereof. In some embodiments, a modified immune effector cell comprises deletion of 2, 3, 4, 5, 6, 7, 8, 9, 10, or more endogenous target genes.

In some embodiments, the modified immune effector cells described herein comprise one or more modified endogenous target genes, wherein the one or more modifications to the target DNA sequence result in expression of a protein with reduced or altered function (e.g., a “modified endogenous protein”) compared to the function of the corresponding protein expressed in an unmodified immune effector cell (e.g., a “unmodified endogenous protein”). In some embodiments, the modified immune effector cells described herein comprise 2, 3, 4, 5, 6, 7, 8, 9, 10, or more modified endogenous target genes encoding 2, 3, 4, 5, 6, 7, 8, 9, 10, or more modified endogenous proteins. In some embodiments, the modified endogenous protein demonstrates reduced or altered binding affinity for another protein expressed by the modified immune effector cell or expressed by another cell; reduced or altered signaling capacity; reduced or altered enzymatic activity; reduced or altered DNA-binding activity; or reduced or altered ability to function as a scaffolding protein.

In some embodiments, the modified endogenous target gene comprises one or more dominant negative mutations. As used herein, a “dominant-negative mutation” refers to a substitution, deletion, or insertion of one or more nucleotides of a target gene such that the encoded protein acts antagonistically to the protein encoded by the unmodified target gene. The mutation is dominant-negative because the negative phenotype confers genic dominance over the positive phenotype of the corresponding unmodified gene. A gene comprising one or more dominant-negative mutations and the protein encoded thereby are referred to as a “dominant-negative mutants”, e.g. dominant-negative genes and dominant-negative proteins. In some embodiments, the dominant negative mutant protein is encoded by an exogenous transgene inserted at one or more locations in the genome of the immune effector cell.

Various mechanisms for dominant negativity are known. Typically, the gene product of a dominant negative mutant retains some functions of the unmodified gene product but lacks one or more crucial other functions of the unmodified gene product. This causes the dominant-negative mutant to antagonize the unmodified gene product. For example, as an illustrative embodiment, a dominant-negative mutant of a transcription factor may lack a functional activation domain but retain a functional DNA binding domain. In this example, the dominant-negative transcription factor cannot activate transcription of the DNA as the unmodified transcription factor does, but the dominant-negative transcription factor can indirectly inhibit gene expression by preventing the unmodified transcription factor from binding to the transcription-factor binding site. As another illustrative embodiment, dominant-negative mutations of proteins that function as dimers are known. Dominant-negative mutants of such dimeric proteins may retain the ability to dimerize with unmodified protein but be unable to function otherwise. The dominant-negative monomers, by dimerizing with unmodified monomers to form heterodimers, prevent formation of functional homodimers of the unmodified monomers. Dominant negative mutations of the SOCS1 gene are known in the art and include the murine F59D mutant (See e.g., Hanada et al., J Biol Chem, 276:44:2 (2001), 40746-40754; and Suzuki et al., J Exp Med, 193:4 (2001), 471-482), and the human F58D mutant, identified by sequence alignments of the human and murine SOCS1 amino acid sequences.

In some embodiments, the modified immune effector cells comprise a gene-regulating system capable of reducing the expression and/or function of two or more endogenous target genes selected from SOCS1, PTPN2, and ZC3H12A. The gene-regulating system can reduce the expression and/or function of the endogenous target genes modifications by a variety of mechanisms including by modifying the genomic DNA sequence of the endogenous target gene (e.g., by insertion, deletion, or mutation of one or more nucleic acids in the genomic DNA sequence); by regulating transcription of the endogenous target gene (e.g., inhibition or repression of mRNA transcription); and/or by regulating translation of the endogenous target gene (e.g., by mRNA degradation).

In some embodiments, the modified immune effector cells described herein comprise a gene-regulating system comprising:

(a) two or more nucleic acid molecules capable of reducing the expression and/or modifying the function of a gene product encoded by two or more endogenous target genes selected from SOCS1, PTPN2, and ZC3H12A;

(b) one or more polynucleotides encoding two or more nucleic acid molecules that are capable of reducing the expression and/or modifying the function of the gene products encoded by two or more endogenous target genes selected from SOCS1, PTPN2, and ZC3H12A;

(c) two or more proteins capable of reducing the expression and/or modifying the function of the gene products encoded by two or more endogenous target genes selected from SOCS1, PTPN2, and ZC3H12A;

(d) one or more polynucleotides encoding two or more proteins that are capable of reducing the expression and/or modifying the function of a gene product encoded by two or more endogenous target genes selected from SOCS1, PTPN2, and ZC3H12A;

(e) two or more guide RNAs (gRNAs) capable of binding to a target DNA sequence in two or more endogenous genes selected from SOCS1, PTPN2, and ZC3H12A;

(f) one or more polynucleotides encoding two or more gRNAs capable of binding to a target DNA sequence in two or more endogenous genes selected from SOCS1, PTPN2, and ZC3H12A;

(g) one or more site-directed modifying polypeptides capable of interacting with a gRNA and modifying a target DNA sequence in an endogenous gene selected from SOCS1, PTPN2, and ZC3H12A;

(h) one or more polynucleotides encoding a site-directed modifying polypeptide capable of interacting with a gRNA and modifying a target DNA sequence in an endogenous gene selected from SOCS1, PTPN2, and ZC3H12A;

(i) two or more guide DNAs (gDNAs) capable of binding to a target DNA sequence in two or more endogenous genes selected from SOCS1, PTPN2, and ZC3H12A;

(j) one or more polynucleotides encoding two or more gDNAs capable of binding to a target DNA sequence in two or more endogenous genes selected from SOCS1, PTPN2, and ZC3H12A;

(k) one or more site-directed modifying polypeptides capable of interacting with a gDNA and modifying a target DNA sequence in an endogenous gene selected from SOCS1, PTPN2, and ZC3H12A;

(l) one or more polynucleotides encoding a site-directed modifying polypeptide capable of interacting with a gDNA and modifying a target DNA sequence in an endogenous gene selected from SOCS1, PTPN2, and ZC3H12A;

(m) two or more gRNAs capable of binding to a target mRNA sequence encoded by two or more endogenous genes selected from SOCS1, PTPN2, and ZC3H12A;

(n) one or more polynucleotides encoding two or more gRNAs capable of binding to a target mRNA sequence encoded by two or more endogenous genes selected from SOCS1, PTPN2, and ZC3H12A;

(o) one or more site-directed modifying polypeptides capable of interacting with a gRNA and modifying a target mRNA sequence encoded by an endogenous gene selected from SOCS1, PTPN2, and ZC3H12A;

(p) one or more polynucleotides encoding a site-directed modifying polypeptide capable of interacting with a gRNA and modifying a target mRNA sequence encoded by an endogenous gene selected from SOCS1, PTPN2, and ZC3H12A; or

(q) any combination of the above.

In some embodiments, one or more polynucleotides encoding the gene-regulating system are inserted into the genome of the immune effector cell. In some embodiments, one or more polynucleotides encoding the gene-regulating system are expressed episomaly and are not inserted into the genome of the immune effector cell.

In some embodiments, the modified immune effector cells described herein comprise reduced expression and/or function of two or more endogenous target genes and further comprise one or more exogenous transgenes inserted at one or more genomic loci (e.g., a genetic “knock-in”). In some embodiments, the one or more exogenous transgenes encode detectable tags, safety-switch systems, chimeric switch receptors, and/or engineered antigen-specific receptors.

In some embodiments, the modified immune effector cells described herein further comprise an exogenous transgene encoding a detectable tag. Examples of detectable tags include but are not limited to, FLAG tags, poly-histidine tags (e.g. 6xHis), SNAP tags, Halo tags, cMyc tags, glutathione-S-transferase tags, avidin, enzymes, fluorescent proteins, luminescent proteins, chemiluminescent proteins, bioluminescent proteins, and phosphorescent proteins. In some embodiments the fluorescent protein is selected from the group consisting of blue/UV proteins (such as BFP, TagBFP, mTagBFP2, Azurite, EBFP2, mKalama1, Sirius, Sapphire, and T-Sapphire); cyan proteins (such as CFP, eCFP, Cerulean, SCFP3A, mTurquoise, mTurquoise2, monomeric Midoriishi-Cyan, TagCFP, and mTFP1); green proteins (such as: GFP, eGFP, meGFP (A208K mutation), Emerald, Superfolder GFP, Monomeric Azami Green, TagGFP2, mUKG, mWasabi, Clover, and mNeonGreen); yellow proteins (such as YFP, eYFP, Citrine, Venus, SYFP2, and TagYFP); orange proteins (such as Monomeric Kusabira-Orange, mKOK, mKO2, mOrange, and mOrange2); red proteins (such as RFP, mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP); long stokes shift proteins (such as mKeima Red, LSS-mKate1, LSS-mKate2, and mBeRFP); photoactivatible proteins (such as PA-GFP, PAmCherryl, and PATagRFP); photoconvertible proteins (such as Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the detectable tag can be selected from AmCyan, AsRed, DsRed2, DsRed Express, E2-Crimson, HcRed, ZsGreen, ZsYellow, mCherry, mStrawberry, mOrange, mBanana, mPlum, mRasberry, tdTomato, DsRed Monomer, and/or AcGFP, all of which are available from Clontech.

In some embodiments, the modified immune effector cells described herein further comprise an exogenous transgene encoding a safety-switch system. Safety-switch systems (also referred to in the art as suicide gene systems) comprise exogenous transgenes encoding for one or more proteins that enable the elimination of a modified immune effector cell after the cell has been administered to a subject. Examples of safety-switch systems are known in the art. For example, safety-switch systems include genes encoding for proteins that convert non-toxic pro-drugs into toxic compounds such as the Herpes simplex thymidine kinase (Hsv-tk) and ganciclovir (GCV) system (Hsv-tk/GCV). Hsv-tk converts non-toxic GCV into a cytotoxic compound that leads to cellular apoptosis. As such, administration of GCV to a subject that has been treated with modified immune effector cells comprising a transgene encoding the Hsv-tk protein can selectively eliminate the modified immune effector cells while sparing endogenous immune effector cells. (See e.g., Bonini et al., Science, 1997, 276(5319):1719-1724; Ciceri et al., Blood, 2007, 109(11):1828-1836; Bondanza et al., Blood 2006, 107(5):1828-1836).

Additional safety-switch systems include genes encoding for cell-surface markers, enabling elimination of modified immune effector cells by administration of a monoclonal antibody specific for the cell-surface marker via ADCC. In some embodiments, the cell-surface marker is CD20 and the modified immune effector cells can be eliminated by administration of an anti-CD20 monoclonal antibody such as Rituximab (See e.g., Introna et al., Hum Gene Ther, 2000, 11(4):611-620; Serafini et al., Hum Gene Ther, 2004, 14, 63-76; van Meerten et al., Gene Ther, 2006, 13, 789-797). Similar systems using EGF-R and Cetuximab or Panitumumab are described in International PCT Publication No. WO 2018006880. Additional safety-switch systems include transgenes encoding pro-apoptotic molecules comprising one or more binding sites for a chemical inducer of dimerization (CID), enabling elimination of modified immune effector cells by administration of a CID which induces oligomerization of the pro-apoptotic molecules and activation of the apoptosis pathway. In some embodiments, the pro-apoptotic molecule is Fas (also known as CD95) (Thomis et al., Blood, 2001, 97(5), 1249-1257). In some embodiments, the pro-apoptotic molecule is caspase-9 (Straathof et al., Blood, 2005, 105(11), 4247-4254).

In some embodiments, the modified immune effector cells described herein further comprise an exogenous transgene encoding a chimeric switch receptor. Chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor. In some embodiments, the chimeric switch receptor comprises the extracellular domain of an inhibitory cell-surface receptor fused to an intracellular domain that leads to the transmission of an activating signal rather than the inhibitory signal normally transduced by the inhibitory cell-surface receptor. In particular embodiments, extracellular domains derived from cell-surface receptors known to inhibit immune effector cell activation can be fused to activating intracellular domains. Engagement of the corresponding ligand will then activate signaling cascades that increase, rather than inhibit, the activation of the immune effector cell. For example, in some embodiments, the modified immune effector cells described herein comprise a transgene encoding a PD1-CD28 switch receptor, wherein the extracellular domain of PD1 is fused to the intracellular signaling domain of CD28 (See e.g., Liu et al., Cancer Res 76:6 (2016), 1578-1590 and Moon et al., Molecular Therapy 22 (2014), S201). In some embodiments, the modified immune effector cells described herein comprise a transgene encoding the extracellular domain of CD200R and the intracellular signaling domain of CD28 (See Oda et al., Blood 130:22 (2017), 2410-2419).

In some embodiments, the modified immune effector cells described herein further comprise an engineered antigen-specific receptor recognizing a protein target expressed by a target cell, such as a tumor cell or an antigen presenting cell (APC), referred to herein as “modified receptor-engineered cells” or “modified RE-cells”. The term “engineered antigen receptor” refers to a non-naturally occurring antigen-specific receptor such as a chimeric antigen receptor (CAR) or a recombinant T cell receptor (TCR). In some embodiments, the engineered antigen receptor is a CAR comprising an extracellular antigen binding domain fused via hinge and transmembrane domains to a cytoplasmic domain comprising a signaling domain. In some embodiments, the CAR extracellular domain binds to an antigen expressed by a target cell in an MHC-independent manner leading to activation and proliferation of the RE cell. In some embodiments, the extracellular domain of a CAR recognizes a tag fused to an antibody or antigen-binding fragment thereof. In such embodiments, the antigen-specificity of the CAR is dependent on the antigen-specificity of the labeled antibody, such that a single CAR construct can be used to target multiple different antigens by substituting one antibody for another (See e.g., U.S. Pat. Nos. 9,233,125 and 9,624,279; US Patent Application Publication Nos. 20150238631 and 20180104354). In some embodiments, the extracellular domain of a CAR may comprise an antigen binding fragment derived from an antibody. Antigen binding domains that are useful in the present disclosure include, for example, scFvs; antibodies; antigen binding regions of antibodies; variable regions of the heavy/light chains; and single chain antibodies.

In some embodiments, the intracellular signaling domain of a CAR may be derived from the TCR complex zeta chain (such as CD3 signaling domains), FcγRIII, FccRI, or the T-lymphocyte activation domain. In some embodiments, the intracellular signaling domain of a CAR further comprises a costimulatory domain, for example a 4-1BB, CD28, CD40, MyD88, or CD70 domain. In some embodiments, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US 20130287748; WO 2015/142675; WO 2014/055657; and WO 2015/090229, incorporated herein by reference).

CARs specific for a variety of tumor antigens are known in the art, for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J Natl Cancer Inst (2014) 107(1):364), carbonic anhydrase K-specific CARs (Lamers et al., Biochem Soc Trans (2016) 44(3):951-959), FR-α-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL13Rα2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2):154-166), MSLN-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta®) and Tisagenlecleucel (Kymriah®). See also, Li et al., J Hematol and Oncol (2018) 11(22), reviewing clinical trials of tumor-specific CARs. Exemplary CARs suitable for use according to the present disclosure are described below in Table 1.

TABLE 1
Exemplary CAR constructs

| | | Ag- | Intra- | Trans- | AA | NA |
|---|---|---|---|---|---|---|
| | | binding | cellular | membrane | SEQ | SEQ |
| CAR Ref ID | Target | domain | Domain | Domain | ID | ID |
| KSQCAR017 | human | Cetuximab | CD3 | CD8a | 264 | 265 |
| | EGFR | H225 scFv | zeta | hinge | | |
| KSQCAR1909 | human | FMC63 | CD3 | CD8a | 266 | 267 |
| | CD19 | scFv | zeta | hinge | | |
| KSQCAR010 | human | Herceptin | CD3 | CD8a | 268 | 269 |
| | HER2 | scFv | zeta | hinge | | |

In some embodiments, the engineered antigen receptor is a recombinant TCR. Recombinant TCRs comprise TCRα and/or TCRβ chains that have been isolated and cloned from T cell populations recognizing a particular target antigen. For example, TCRα and/or TCRβ genes (i.e., TRAC and TRBC) can be cloned from T cell populations isolated from individuals with particular malignancies or T cell populations that have been isolated from humanized mice immunized with specific tumor antigens or tumor cells. Recombinant TCRs recognize antigen through the same mechanisms as their endogenous counterparts (e.g., by recognition of their cognate antigen presented in the context of major histocompatibility complex (MHC) proteins expressed on the surface of a target cell). This antigen engagement stimulates endogenous signal transduction pathways leading to activation and proliferation of the TCR-engineered cells.

Recombinant TCRs specific for tumor antigens are known in the art, for example WT1-specific TCRs (JTCR016, Juno Therapeutics; WT1-TCRc4, described in US Patent Application Publication No. 20160083449), MART-1 specific TCRs (including the DMF4T clone, described in Morgan et al., Science 314 (2006) 126-129); the DMF5T clone, described in Johnson et al., Blood 114 (2009) 535-546); and the ID3T clone, described in van den Berg et al., Mol. Ther. 23 (2015) 1541-1550), gp100-specific TCRs (Johnson et al., Blood 114 (2009) 535-546), CEA-specific TCRs (Parkhurst et al., Mol Ther. 19 (2011) 620-626), NY-ESO and LAGE-1 specific TCRs (1G4T clone, described in Robbins et al., J Clin Oncol 26 (2011) 917-924; Robbins et al., Clin Cancer Res 21 (2015) 1019-1027; and Rapoport et al., Nature Medicine 21 (2015) 914-921), and MAGE-A3-specific TCRs (Morgan et al., J Immunother 36 (2013) 133-151) and Linette et al., Blood 122 (2013) 227-242). (See also, Debets et al., Seminars in Immunology 23 (2016) 10-21).

To generate the recombinant TCRs, the native TRAC (SEQ ID NO: 260) and TRBC (SEQ ID NOs: 262) protein sequences are fused to the C-terminal ends of TCR-α and TCR-β chain variable regions specific for a protein or peptide of interest. For example, the engineered TCR can recognize an amino acid sequence comprising the NY-ESO peptide (SLLMWITQC, SEQ ID NO: 239), such as the 1G4 TCR or the 95:LY TCR (Robbins et al, Journal of Immunology 2008 180:6116-6131). In such illustrative embodiments, the paired 1G4-TCR α/βchains comprise SEQ ID NOs: 249 and 248, respectively and the paired 95:LY-TCR α/βchains comprise SEQ ID NOs: 252 and 251, respectively. The recombinant TCR can recognize the MART-1 peptide (AAGIGILTV, SEQ ID NO: 240), such as the DMF4 and DMF5 TCRs (Robbins et al, Journal of Immunology 2008 180:6116-6131). In such illustrative embodiments, the paired DMF4-TCR α/β chains comprise SEQ ID NOs: 255 and 254, respectively and the paired DMF5-TCR α/βchains comprise SEQ ID NOs: 258 and 257, respectively. The recombinant TCR can recognize the WT-1 peptide (RMFPNAPYL, SEQ ID NO: 241), such as the DLT TCR (Robbins et al, Journal of Immunology 2008 180:6116-6131). In such illustrative embodiments, the paired high-affinity DLT-TCR α/β chains comprise SEQ ID NOs: 246 and 245, respectively.

Codon-optimized DNA sequences encoding the recombinant TCRα and TCRβ chain proteins can be generated such that expression of both TCR chains is driven off of a single promoter in a stoichiometric fashion. In such embodiment, the P2A sequence (SEQ ID NO: 238) can be inserted between the DNA sequences encoding the TCRβ and the TCRα chain, such that the expression cassettes encoding the recombinant TCR chains comprise the following format: TCRβ-P2A-TCRα. As an illustrative embodiment, the protein sequence of the 1G4 NY-ESO-specific TCR expressed from such a cassette would comprise SEQ ID NO: 250, the protein sequence of the 95:LY NY-ESO-specific TCR expressed from such a cassette would comprise SEQ ID NO: 23, the protein sequence of the DMF4 MART1-specific TCR expressed from such a cassette would comprise SEQ ID NO: 256, the protein sequence of the DMF5 MART1-specific TCR expressed from such a cassette would comprise SEQ ID NO: 259, and the protein sequence of the DLT WT1-specific TCR expressed from such a cassette would comprise SEQ ID NO: 247.

In some embodiments, the engineered antigen receptor is directed against a target antigen selected from a cluster of differentiation molecule, such as CD3, CD4, CD8, CD16, CD24, CD25, CD33, CD34, CD45, CD64, CD71, CD78, CD80 (also known as B7-1), CD86 (also known as B7-2), CD96, CD116, CD117, CD123, CD133, and CD138, CD371 (also known as CLL1); a tumor-associated surface antigen, such as 5T4, BCMA (also known as CD269 and TNFRSF17, UniProt # Q02223), carcinoembryonic antigen (CEA), carbonic anhydrase 9 (CAIX or MN/CAIX), CD19, CD20, CD22, CD30, CD40, disialogangliosides such as GD2, ELF2M, ductal-epithelial mucin, ephrin B2, epithelial cell adhesion molecule (EpCAM), ErbB2 (HER2/neu), FCRL5 (UniProt # Q68SN8), FKBP11 (UniProt # Q9NYL4), glioma-associated antigen, glycosphingolipids, gp36, GPRC5D (UniProt # Q9NZD1), mut hsp70-2, intestinal carboxyl esterase, IGF-I receptor, ITGA8 (UniProt # P53708), KAMP3, LAGE-1a, MAGE, mesothelin, neutrophil elastase, NKG2D, Nkp30, NY-ESO-1, PAP, prostase, prostate-carcinoma tumor antigen-1 (PCTA-1), prostate specific antigen (PSA), PSMA, prostein, RAGE-1, ROR1, RU1 (SFMBT1), RU2 (DCDC2), SLAMF7 (UniProt # Q9NQ25), survivin, TAG-72, and telomerase; a major histocompatibility complex (MHC) molecule presenting a tumor-specific peptide epitope; tumor stromal antigens, such as the extra domain A (EDA) and extra domain B (EDB) of fibronectin; the A1 domain of tenascin-C(TnC A1) and fibroblast associated protein (FAP); cytokine receptors, such as epidermal growth factor receptor (EGFR), EGFR variant III (EGFRvIII), TFGβ-R or components thereof such as endoglin; a major histocompatibility complex (MHC) molecule; a virus-specific surface antigen such as an HIV-specific antigen (such as HIV gp120); an EBV-specific antigen, a CMV-specific antigen, a HPV-specific antigen, a Lassa virus-specific antigen, an Influenza virus-specific antigen as well as any derivate or variant of these surface antigens.

In some embodiments, the present disclosure provides modified immune effector cells comprising reduced expression and/or function of SOCS1 and PTPN2 or a gene-regulating system capable of reducing the expression and/or function of SOCS1 and PTPN2 and further comprising a CAR or recombinant TCR expressed on the cell surface. In some embodiments, the modified immune effector cells comprise reduced expression and/or function of SOCS1 and PTPN2 or a gene-regulating system capable of reducing the expression and/or function of SOCS1 and PTPN2 and further comprising a recombinant expression vector encoding a CAR or a recombinant TCR.

In some embodiments, the present disclosure provides modified immune effector cells comprising reduced expression and/or function of SOCS1 and ZC3H12A or a gene-regulating system capable of reducing the expression and/or function of SOCS1 and ZC3H12A and further comprising a CAR or recombinant TCR expressed on the cell surface. In some embodiments, the modified immune effector cells comprise reduced expression and/or function of SOCS1 and ZC3H12A or a gene-regulating system capable of reducing the expression and/or function of SOCS1 and ZC3H12A and further comprising a recombinant expression vector encoding a CAR or a recombinant TCR.

In some embodiments, the present disclosure provides modified immune effector cells comprising reduced expression and/or function of PTPN2 and ZC3H12A or a gene-regulating system capable of reducing the expression and/or function of PTPN2 and ZC3H12A and further comprising a CAR or recombinant TCR expressed on the cell surface. In some embodiments, the modified immune effector cells comprise reduced expression and/or function of PTPN2 and ZC3H12A or a gene-regulating system capable of reducing the expression and/or function of PTPN2 and ZC3H12A and further comprising a recombinant expression vector encoding a CAR or a recombinant TCR.

In some embodiments, the present disclosure provides modified immune effector cells comprising reduced expression and/or function of SOCS1 and PTPN2 or a gene-regulating system capable of reducing the expression and/or function of SOCS1 and PTPN2, wherein the immune effector cell is a TIL. In some embodiments, the present disclosure provides modified immune effector cells comprising reduced expression and/or function of SOCS1 and ZC3H12A or a gene-regulating system capable of reducing the expression and/or function of SOCS1 and ZC3H12A, wherein the immune effector cell is a TIL. In some embodiments, the present disclosure provides modified immune effector cells comprising reduced expression and/or function of PTPN2 and ZC3H12A or a gene-regulating system capable of reducing the expression and/or function of PTPN2 and ZC3H12A, wherein the immune effector cell is a TIL.

### A. Effector Functions

In some embodiments, the modified immune effector cells described herein comprise reduced expression and/or function (or a gene-regulating system capable of reducing the expression and/or function) of two or more endogenous target genes selected from SOCS1, PTPN2, and ZC3H12A and demonstrate an increase in one or more immune cell effector functions. Herein, the term “effector function” refers to functions of an immune cell related to the generation, maintenance, and/or enhancement of an immune response against a target cell or target antigen. In some embodiments, the modified immune effector cells described herein demonstrate one or more of the following characteristics compared to an unmodified immune effector cell: increased infiltration or migration in to a tumor, increased proliferation, increased or prolonged cell viability, increased resistance to inhibitory factors in the surrounding microenvironment such that the activation state of the cell is prolonged or increased, increased production of pro-inflammatory immune factors (e.g., pro-inflammatory cytokines, chemokines, and/or enzymes), increased cytotoxicity, and/or increased resistance to exhaustion.

In some embodiments, the modified immune effector cells described herein demonstrate increased infiltration into a tumor compared to an unmodified immune effector cell. In some embodiments, increased tumor infiltration by modified immune effector cells refers to an increase the number of modified immune effector cells infiltrating into a tumor during a given period of time compared to the number of unmodified immune effector cells that infiltrate into a tumor during the same period of time. In some embodiments, the modified immune effector cells demonstrate a 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100, or more fold increase in tumor infiltration compared to an unmodified immune cell. Tumor infiltration can be measured by isolating one or more tumors from a subject and assessing the number of modified immune cells in the sample by flow cytometry, immunohistochemistry, and/or immunofluorescence.

In some embodiments, the modified immune effector cells described herein demonstrate an increase in cell proliferation compared to an unmodified immune effector cell. In these embodiments, the result is an increase in the number of modified immune effector cells present compared to unmodified immune effector cells after a given period of time. For example, in some embodiments, modified immune effector cells demonstrate increased rates of proliferation compared to unmodified immune effector cells, wherein the modified immune effector cells divide at a more rapid rate than unmodified immune effector cells. In some embodiments, the modified immune effector cells demonstrate a 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100, or more fold increase in the rate of proliferation compared to an unmodified immune cell. In some embodiments, modified immune effector cells demonstrate prolonged periods of proliferation compared to unmodified immune effector cells, wherein the modified immune effector cells and unmodified immune effector cells divide at similar rates, but wherein the modified immune effector cells maintain the proliferative state for a longer period of time. In some embodiments, the modified immune effector cells maintain a proliferative state for 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100, or more times longer than an unmodified immune cell.

In some embodiments, the modified immune effector cells described herein demonstrate increased or prolonged cell viability compared to an unmodified immune effector cell. In such embodiments, the result is an increase in the number of modified immune effector cells or present compared to unmodified immune effector cells after a given period of time. For example, in some embodiments, modified immune effector cells described herein remain viable and persist for 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 25, 30, 35, 40, 45, 50, 60, 70, 80, 90, 100, or more times longer than an unmodified immune cell.

In some embodiments, the modified immune effector cells described herein demonstrate increased resistance to inhibitory factors compared to an unmodified immune effector cell. Exemplary inhibitory factors include signaling by immune checkpoint molecules (e.g., PD1, PDL1, CTLA4, LAG3, IDO) and/or inhibitory cytokines (e.g., IL-10, TGFβ).

In some embodiments, the modified T cells described herein demonstrate increased resistance to T cell exhaustion compared to an unmodified T cell. T cell exhaustion is a state of antigen-specific T cell dysfunction characterized by decreased effector function and leading to subsequent deletion of the antigen-specific T cells. In some embodiments, exhausted T cells lack the ability to proliferate in response to antigen, demonstrate decreased cytokine production, and/or demonstrate decreased cytotoxicity against target cells such as tumor cells. In some embodiments, exhausted T cells are identified by altered expression of cell surface markers and transcription factors, such as decreased cell surface expression of CD122 and CD127; increased expression of inhibitory cell surface markers such as PD1, LAG3, CD244, CD160, TIM3, and/or CTLA4; and/or increased expression of transcription factors such as Blimp1, NFAT, and/or BATF. In some embodiments, exhausted T cells demonstrate altered sensitivity cytokine signaling, such as increased sensitivity to TGFβ signaling and/or decreased sensitivity to IL-7 and IL-15 signaling. T cell exhaustion can be determined, for example, by co-culturing the T cells with a population of target cells and measuring T cell proliferation, cytokine production, and/or lysis of the target cells. In some embodiments, the modified immune effector cells described herein are co-cultured with a population of target cells (e.g., autologous tumor cells or cell lines that have been engineered to express a target tumor antigen) and effector cell proliferation, cytokine production, and/or target cell lysis is measured. These results are then compared to the results obtained from co-culture of target cells with a control population of immune cells (such as unmodified immune effector cells or immune effector cells that have a control modification).

In some embodiments, resistance to T cell exhaustion is demonstrated by increased production of one or more cytokines (e.g., IFNγ, TNFα, or IL-2) from the modified immune effector cells compared to the cytokine production observed from the control population of immune cells. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in cytokine production from the modified immune effector cells compared to the cytokine production from the control population of immune cells is indicative of an increased resistance to T cell exhaustion. In some embodiments, resistance to T cell exhaustion is demonstrated by increased proliferation of the modified immune effector cells compared to the proliferation observed from the control population of immune cells. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in proliferation of the modified immune effector cells compared to the proliferation of the control population of immune cells is indicative of an increased resistance to T cell exhaustion. In some embodiments, resistance to T cell exhaustion is demonstrated by increased target cell lysis by the modified immune effector cells compared to the target cell lysis observed by the control population of immune cells. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in target cell lysis by the modified immune effector cells compared to the target cell lysis by the control population of immune cells is indicative of an increased resistance to T cell exhaustion.

In some embodiments, exhaustion of the modified immune effector cells compared to control populations of immune cells is measured during the in vitro or ex vivo manufacturing process. For example, in some embodiments, TILs isolated from tumor fragments are modified according to the methods described herein and then expanded in one or more rounds of expansion to produce a population of modified TILs. In such embodiments, the exhaustion of the modified TILs can be determined immediately after harvest and prior to a first round of expansion, after the first round of expansion but prior to a second round of expansion, and/or after the first and the second round of expansion. In some embodiments, exhaustion of the modified immune effector cells compared to control populations of immune cells is measured at one or more time points after transfer of the modified immune effector cells into a subject. For example, in some embodiments, the modified cells are produced according to the methods described herein and administered to a subject. Samples can then be taken from the subject at various time points after the transfer to determine exhaustion of the modified immune effector cells in vivo over time.

In some embodiments, the modified immune effector cells described herein demonstrate increased expression or production of pro-inflammatory immune factors compared to an unmodified immune effector cell. Examples of pro-inflammatory immune factors include cytolytic factors, such as granzyme B, perforin, and granulysin; and pro-inflammatory cytokines such as interferons (IFNα, IFNβ, IFNγ), TNFα, IL-10, IL-12, IL-2, IL-17, CXCL8, and/or IL-6.

In some embodiments, the modified immune effector cells described herein demonstrate increased cytotoxicity against a target cell compared to an unmodified immune effector cell. In some embodiments, the modified immune effector cells demonstrate a 1, 2, 3, 4, 5, 6, 7, 8, 9, 10, 20, 30, 40, 50, 60, 70, 80, 90, 100, or more fold increase in cytotoxicity against a target cell compared to an unmodified immune cell.

In some embodiments, the modified immune effector cells described herein produce a TIL population that persists with both the central memory phenotype T_{cm} cells) and effector memory phenotype (Tₑₘ cells). These phenotypes provide durable anti-tumor memory and invoke eptitope spreading.

Assays for measuring immune effector function are known in the art. For example, tumor infiltration can be measured by isolating tumors from a subject and determining the total number and/or phenotype of the lymphocytes present in the tumor by flow cytometry, immunohistochemistry, and/or immunofluorescence. Cell-surface receptor expression can be determined by flow cytometry, immunohistochemistry, immunofluorescence, Western blot, and/or qPCR. Cytokine and chemokine expression and production can be measured by flow cytometry, immunohistochemistry, immunofluorescence, Western blot, ELISA, and/or qPCR. Responsiveness or sensitivity to extracellular stimuli (e.g., cytokines, inhibitory ligands, or antigen) can be measured by assaying cellular proliferation and/or activation of downstream signaling pathways (e.g., phosphorylation of downstream signaling intermediates) in response to the stimuli. Cytotoxicity can be measured by target-cell lysis assays known in the art, including in vitro or ex vivo co-culture of the modified immune effector cells with target cells and in vivo murine tumor models, such as those described throughout the Examples.

### B. Regulation of Endogenous Pathways and Genes

In some embodiments, the modified immune effector cells described herein demonstrate a reduced expression and/or function of two or more endogenous target genes selected from SOCS1, PTPN2, and ZC3H12A. Further details on the endogenous target genes are provided below in Table 2. In such embodiments, the reduced expression or function of the two or more endogenous target genes enhances one or more effector functions of the immune cell.

In some embodiments, the modified effector cells described herein comprise reduced expression and/or function of the Suppressors of cytokine signaling SOCS 1 (SOCS1) gene. The SOCS1 protein comprises C-terminal SOCS box motifs, an SH2-domain, an ESS domain, and an N-terminal KIR domain. The 12 amino-acid residues called the kinase inhibitory region (KIR) has been found to be critical in the ability of SOCS1 to negatively regulate JAK1, TYK2 and JAK2 tyrosine kinase function.

In some embodiments, the modified effector cells described herein comprise reduced expression and/or function of the PTPN2 gene. The protein tyrosine phosphatase family (PTP) dephosphorylate phospho-tyrosine residues by their phosphatase catalytic domain. PTPN2 functions as a brake on both TCRs and cytokines, which signal through JAK/STAT signaling complexes, and thus serves as a checkpoint on both Signals 1 and 3. Following T Cell engagement with antigen and activation of the TCR, positive signals are amplified downstream by the kinases Lck and Fyn by phosphorylation of tyrosine residues. PTPN2 serves to dephosphorylate both Lck and Fyn and thus attenuate TCR signaling. In addition, following T cell encounter with cytokines and signaling through common γ chain receptor complex, which transmit positive signals though JAK/STAT signaling, PTPN2 also attenuates by dephosphorylation of STAT1 and STAT3. The sum functional impact of PTPN2 loss on T cell function is a lowering of the activation threshold needed for fulminant T cell activation through the TCR, and a hypersensitivity to growth and differentiation-enhancing cytokines The protein tyrosine phosphatase family (PTP) dephosphorylate phospho-tyrosine residues by their phosphatase catalytic domain. PTPN2 functions as a brake on both TCRs and cytokines, which signal through JAK/STAT signaling complexes, and thus serves as a checkpoint on both Signals 1 and 3. Following T Cell engagement with antigen and activation of the TCR, positive signals are amplified downstream by the kinases Lck and Fyn by phosphorylation of tyrosine residues. PTPN2 serves to dephosphorylate both Lck and Fyn and thus attenuate TCR signaling. In addition, following T cell encounter with cytokines and signaling through common γc chain receptor complex, which transmit positive signals though JAK/STAT signaling, PTPN2 also attenuates by dephosphorylation of STAT1 and STAT3. The sum functional impact of PTPN2 loss on T cell function is a lowering of the activation threshold needed for fulminant T cell activation through the TCR, and a hypersensitivity to growth and differentiation-enhancing cytokines.

In addition, in genetically engineered mouse (GEM) models, deletion of PTPN2 in the whole mouse increases cytokine levels, lymphocytic infiltration in nonlymphoid tissues and early signs of rheumatoid arthritis-like symptoms; these mice do not survive past 5 weeks of age. Thus, PTPN2 has been identified as critical for postnatal development in mice. Consistent with this autoimmune phenotype, deletion of Ptpn2 in the T cell lineage from birth also results in an increase in lymphocytic infiltration in non-lymphoid tissues. Importantly, an inducible knockout of Ptpn2 in adult mouse T cells did not result in any autoimmune manifestations of its role in autoimmunity, Ptpn2 deletion was identified to associate with a small percentage of T cell acute lymphoblastic leukemia in humans (ALL); and to enhance skin tumor development in a two-stage chemically-induced carcinogenicity mouse model. These data have led some to postulate that PTPN2 may be a tumor suppressor protein.

In some embodiments, the modified effector cells described herein comprise reduced expression and/or function of the ZC3H12A gene. ZC3H12A, also referred to as MCPIP1 and REGNASE-1, is an RNase that possesses a RNase domain just upstream of a CCCH-type zinc-finger motif. Through its nuclease activity, ZC3H12A targets and destabilizes the mRNAs of transcripts, such as IL-6, by binding a conserved stem loop structure within the 3′ UTR of these genes. In T cells, ZC3H12A controls the transcript levels of a number of pro-inflammatory genes, including c-Rel, OX40 and IL-2. REGNASE-lactivation is transient and is subject to negative feedback mechanisms including proteasome-mediated degradation or mucosa-associated lymphoid tissue 1 (MALT1) mediated cleavage. The major function of REGNASE-1 is promoting mRNA decay via its ribonuclease activity by specifically targeting a subset of genes in different cell types. In monocytes, REGNASE-1 downregulates IL-6 and IL-12B mRNAs, thus mitigating inflammation, whereas in T cells, it restricts T-cell activation by targeting c-Rel, Ox40 and IL-2 transcripts. In cancer cells, REGNASE-1promotes apoptosis by inhibiting anti-apoptotic genes including BCL2L1, BCL2A1, RELB and BCL3.

TABLE 2
Endogenous target genes

| | | Human | Human | Murine | Murine |
|---|---|---|---|---|---|
| Gene | | UniProt | NCBI | UniProt | NCBI |
| Symbol | Gene Name | Ref. | Ref | Ref. | Ref |
| SOCS1 | suppressor of | O15524 | 8651 | O35716 | 12703 |
| | cytokine | | (SEQ ID | | (SEQ ID |
| | signaling 1 | | NO: 1) | | NO: 2) |
| PTPN2 | protein | P17706 | 5771 | Q06180 | 19255 |
| | tyrosine | | (SEQ ID | | (SEQ ID |
| | phosphatase, | | NO: 3) | | NO: 4) |
| | non-receptor | | | | |
| | type 2 | | | | |
| ZC3H12A | Endoribo- | Q5D1E8 | 80149 | Q5D1E7 | 230738 |
| | nuclease | | (SEQ ID | | (SEQ ID |
| | ZC3H12A | | NO: 5) | | NO: 6) |
| CBLB | Cbl proto- | Q13191 | 868 | Q3TTA7 | 208650 |
| | oncogene B | | (SEQ ID | | (SEQ ID |
| | | | NO: 823) | | NO: 824) |

In some embodiments, the modified immune effector cells comprise reduced expression and/or function of SOCS1 and reduced expression and/or function of PTPN2. In some embodiments, the modified immune effector cells comprise reduced expression and/or function of SOCS1 and reduced expression and/or function of ZC3H12A. In some embodiments, the modified immune effector cells comprise reduced expression and/or function of PTPN2 and reduced expression and/or function of ZC3H12A. In some embodiments, the modified immune effector cells comprise reduced expression and/or function of at least two genes endogenous target genes selected from SOCS1, PTPN2, and ZC3H12A and further comprise reduced expression and/or function of CBLB.

### III. Gene-Regulating Systems

Herein, the term “gene-regulating system” refers to a protein, nucleic acid, or combination thereof that is capable of modifying an endogenous target DNA sequence when introduced into a cell, thereby regulating the expression or function of the encoded gene product. Numerous gene editing systems suitable for use in the methods of the present disclosure are known in the art including, but not limited to, shRNAs, siRNAs, zinc-finger nuclease systems, TALEN systems, and CRISPR/Cas systems.

As used herein, “regulate,” when used in reference to the effect of a gene-regulating system on an endogenous target gene encompasses any change in the sequence of the endogenous target gene, any change in the epigenetic state of the endogenous target gene, and/or any change in the expression or function of the protein encoded by the endogenous target gene.

In some embodiments, the gene-regulating system may mediate a change in the sequence of the endogenous target gene, for example, by introducing one or more mutations into the endogenous target sequence, such as by insertion or deletion of one or more nucleic acids in the endogenous target sequence. Exemplary mechanisms that can mediate alterations of the endogenous target sequence include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion.

In some embodiments, the gene-regulating system may mediate a change in the epigenetic state of the endogenous target sequence. For example, in some embodiments, the gene-regulating system may mediate covalent modifications of the endogenous target gene DNA (e.g., cytosine methylation and hydroxymethylation) or of associated histone proteins (e.g. lysine acetylation, lysine and arginine methylation, serine and threonine phosphorylation, and lysine ubiquitination and sumoylation).

In some embodiments, the gene-regulating system may mediate a change in the expression of the protein encoded by the endogenous target gene. In such embodiments, the gene-regulating system may regulate the expression of the encoded protein by modifications of the endogenous target DNA sequence, or by acting on the mRNA product encoded by the DNA sequence. In some embodiments, the gene-regulating system may result in the expression of a modified endogenous protein. In such embodiments, the modifications to the endogenous DNA sequence mediated by the gene-regulating system result in the expression of an endogenous protein demonstrating a reduced function as compared to the corresponding endogenous protein in an unmodified immune effector cell. In such embodiments, the expression level of the modified endogenous protein may be increased, decreased or may be the same, or substantially similar to, the expression level of the corresponding endogenous protein in an unmodified immune cell.

### A. Nucleic Acid-Based Gene-Regulating Systems

In some embodiments, the present disclosure provides nucleic acid gene-regulating systems comprising two or more nucleic acids capable of reducing the expression and/or function of at least two endogenous genes selected from SOCS1, PTPN2, and ZC3H12A. In some embodiments, the present disclosure provides modified immune effector cells comprising such gene-regulating systems. As used herein, a nucleic acid-based gene-regulating system is a system comprising one or more nucleic acid molecules that is capable of regulating the expression of an endogenous target gene without the requirement for an exogenous protein. In some embodiments, the nucleic acid-based gene-regulating system comprises an RNA interference molecule or antisense RNA molecule that is complementary to a target nucleic acid sequence.

An “antisense RNA molecule” refers to an RNA molecule, regardless of length, that is complementary to an mRNA transcript. Antisense RNA molecules refer to single stranded RNA molecules that can be introduced to a cell, tissue, or subject and result in decreased expression of an endogenous target gene product through mechanisms that do not rely on endogenous gene silencing pathways, but rather rely on RNaseH-mediated degradation of the target mRNA transcript. In some embodiments, an antisense nucleic acid comprises a modified backbone, for example, phosphorothioate, phosphorodithioate, or others known in the art, or may comprise non-natural internucleoside linkages. In some embodiments, an antisense nucleic acid can comprise locked nucleic acids (LNA).

“RNA interference molecule” as used herein refers to an RNA polynucleotide that mediates the decreased the expression of an endogenous target gene product by degradation of a target mRNA through endogenous gene silencing pathways (e.g., Dicer and RNA-induced silencing complex (RISC)). Exemplary RNA interference agents include micro RNAs (also referred to herein as “miRNAs”), short hair-pin RNAs (shRNAs), small interfering RNAs (siRNAs), RNA aptamers, and morpholinos.

In some embodiments, the nucleic acid-based gene-regulating system comprises one or more miRNAs. miRNAs are naturally occurring, small non-coding RNA molecules of about 21-25 nucleotides in length. miRNAs are at least partially complementary to one or more target mRNA molecules. miRNAs can downregulate (e.g., decrease) expression of an endogenous target gene product through translational repression, cleavage of the mRNA, and/or deadenylation.

In some embodiments, the nucleic acid-based gene-regulating system comprises one or more shRNAs. shRNAs are single stranded RNA molecules of about 50-70 nucleotides in length that form stem-loop structures and result in degradation of complementary mRNA sequences. shRNAs can be cloned in plasmids or in non-replicating recombinant viral vectors to be introduced intracellularly and result in the integration of the shRNA-encoding sequence into the genome. As such, an shRNA can provide stable and consistent repression of endogenous target gene translation and expression.

In some embodiments, nucleic acid-based gene-regulating system comprises one or more siRNAs. siRNAs refer to double stranded RNA molecules typically about 21-23 nucleotides in length. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. Optimally, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences. siRNAs and shRNAs are further described in Fire et al., Nature, 391:19, 1998 and U.S. Pat. Nos. 7,732,417; 8,202,846; and 8,383,599.

In some embodiments, the nucleic acid-based gene-regulating system comprises one or more morpholinos. “Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

In some embodiments, the nucleic acid-based gene-regulating system comprises a nucleic acid molecule (e.g., an siRNA, an shRNA, an RNA aptamer, or a morpholino) that binds to a target RNA sequence that is at least 90% identical to a RNA sequence encoded by a DNA sequence defined by a set of genomic coordinates shown in Tables 3-8. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org web site.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2). In some embodiments, the least one SOCS1-targeting nucleic acid molecule is an siRNA or an shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 3 or Table 4. In some embodiments, the at least one SOCS1-targeting nucleic acid molecule binds to a target RNA sequence that is 100% identical to a RNA sequence encoded by a DNA sequence defined by a set of genomic coordinates shown in Table 3 or Table 4. In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 7-151. In some embodiments, the at least one SOCS1-targeting nucleic acid molecule binds to a target RNA sequence that is 100% identical to an RNA sequence encoded by one of SEQ ID NOs: 7-151.

In some embodiments, the at least one SOCS1-targeting nucleic acid molecule is a SOCS1-targeting shRNA or siRNA molecule. In some embodiments, the at least one SOCS1-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 3 or Table 4. In some embodiments, the at least one SOCS1-targeting shRNA or siRNA molecule binds to a target RNA sequence that is 100% identical to a RNA sequence encoded by a DNA sequence defined by a set of genomic coordinates shown in Table 3 or Table 4. In some embodiments, the at least one SOCS1-targeting shRNA or siRNA molecule binds to a target RNA sequence that is at least 95%, 96%, 97%, 98%, or 99% identical to an RNA sequence encoded by one of SEQ ID NOs: 7-151. In some embodiments, the at least one SOCS1-targeting shRNA or siRNA molecule binds to a target RNA sequence that is 100% identical to an RNA sequence encoded by one of SEQ ID NOs: 7-151.

In some embodiments, the nucleic acid-based gene-regulating system comprises at least one SOCS1-targeting siRNA molecule or shRNA molecule selected from those known in the art. For example, in some embodiments, the SOCS1-targeting nucleic acid molecule is a SOCS1-targeting shRNA molecule that binds to a target sequence selected from SEQ ID NOs: 152-171 shown in Table A (See U.S. Pat. No. 9,944,931). In some embodiments, the SOCS1-targeting shRNA molecule is encoded by a nucleic acid sequence selected from SEQ ID NOs: 172-174 shown in Table A (See U.S. Pat. No. 8,324,369). In some embodiments, the SOCS1-targeting nucleic acid molecule is a SOCS1-targeting siRNA comprising a nucleic acid sequence selected from SEQ ID NOs: 175-184 shown in Table B (See International PCT Publication Nos. WO 2017120996; WO 2018137295; WO 2017120998; and WO 2018137293).

TABLE 3
SOCS1 Human Genome Coordinates

| Target | Coordinates |
|---|---|
| SOCS1 | chr16: 11255187-11255206 |
| SOCS1 | chr16: 11255238-11255257 |
| SOCS1 | chr16: 11255058-11255077 |
| SOCS1 | chr16: 11255158-11255177 |
| SOCS1 | chr16: 11255239-11255258 |
| SOCS1 | chr16: 11255237-11255256 |
| SOCS1 | chr16: 11255019-11255038 |
| SOCS1 | chr16: 11255066-11255085 |
| SOCS1 | chr16: 11255238-11255257 |
| SOCS1 | chr16: 11255168-11255187 |
| SOCS1 | chr16: 11255079-11255098 |
| SOCS1 | chr16: 11255287-11255306 |
| SOCS1 | chr16: 11255249-11255268 |
| SOCS1 | chr16: 11255186-11255205 |
| SOCS1 | chr16: 11255236-11255255 |
| SOCS1 | chr16: 11255116-11255135 |
| SOCS1 | chr16: 11255070-11255089 |
| SOCS1 | chr16: 11255117-11255136 |
| SOCS1 | chr16: 11255283-11255302 |
| SOCS1 | chr16: 11255442-11255461 |
| SOCS1 | chr16: 11255209-11255228 |
| SOCS1 | chr16: 11254932-11254951 |
| SOCS1 | chr16: 11254966-11254985 |
| SOCS1 | chr16: 11254950-11254969 |
| SOCS1 | chr16: 11255049-11255068 |
| SOCS1 | chr16: 11255155-11255174 |
| SOCS1 | chr16: 11255460-11255479 |
| SOCS1 | chr16: 11255037-11255056 |
| SOCS1 | chr16: 11255154-11255173 |
| SOCS1 | chr16: 11255115-11255134 |
| SOCS1 | chr16: 11254985-11255004 |
| SOCS1 | chr16: 11255013-11255032 |
| SOCS1 | chr16: 11255016-11255035 |
| SOCS1 | chr16: 11255139-11255158 |
| SOCS1 | chr16: 11255248-11255267 |
| SOCS1 | chr16: 11255217-11255236 |
| SOCS1 | chr16: 11254994-11255013 |
| SOCS1 | chr16: 11254965-11254984 |
| SOCS1 | chr16: 11255219-11255238 |
| SOCS1 | chr16: 11255173-11255192 |
| SOCS1 | chr16: 11255210-11255229 |
| SOCS1 | chr16: 11255062-11255081 |
| SOCS1 | chr16: 11255259-11255278 |
| SOCS1 | chr16: 11255230-11255249 |
| SOCS1 | chr16: 11255084-11255103 |
| SOCS1 | chr16: 11255175-11255194 |
| SOCS1 | chr16: 11255419-11255438 |
| SOCS1 | chr16: 11254903-11254922 |
| SOCS1 | chr16: 11255089-11255108 |
| SOCS1 | chr16: 11255379-11255398 |
| SOCS1 | chr16: 11255206-11255225 |
| SOCS1 | chr16: 11255090-11255109 |
| SOCS1 | chr16: 11255208-11255227 |
| SOCS1 | chr16: 11254956-11254975 |
| SOCS1 | chr16: 11255118-11255137 |
| SOCS1 | chr16: 11254906-11254925 |
| SOCS1 | chr16: 11255167-11255186 |
| SOCS1 | chr16: 11254835-11254854 |
| SOCS1 | chr16: 11255292-11255311 |
| SOCS1 | chr16: 11255416-11255435 |
| SOCS1 | chr16: 11255136-11255155 |
| SOCS1 | chr16: 11254964-11254983 |
| SOCS1 | chr16: 11254896-11254915 |
| SOCS1 | chr16: 11254940-11254959 |
| SOCS1 | chr16: 11255349-11255368 |
| SOCS1 | chr16: 11254992-11255011 |
| SOCS1 | chr16: 11254923-11254942 |
| SOCS1 | chr16: 11255431-11255450 |
| SOCS1 | chr16: 11255463-11255482 |
| SOCS1 | chr16: 11255343-11255362 |
| SOCS1 | chr16: 11255088-11255107 |
| SOCS1 | chr16: 11254834-11254853 |
| SOCS1 | chr16: 11254922-11254941 |
| SOCS1 | chr16: 11255098-11255117 |
| SOCS1 | chr16: 11254993-11255012 |
| SOCS1 | chr16: 11254840-11254859 |
| SOCS1 | chr16: 11255400-11255419 |
| SOCS1 | chr16: 11254920-11254939 |
| SOCS1 | chr16: 11254966-11254985 |
| SOCS1 | chr16: 11254860-11254879 |
| SOCS1 | chr16: 11254980-11254999 |
| SOCS1 | chr16: 11254857-11254876 |
| SOCS1 | chr16: 11254874-11254893 |
| SOCS1 | chr16: 11255028-11255047 |
| SOCS1 | chr16: 11254956-11254975 |
| SOCS1 | chr16: 11254908-11254927 |
| SOCS1 | chr16: 11255337-11255356 |
| SOCS1 | chr16: 11254836-11254855 |
| SOCS1 | chr16: 11254842-11254861 |
| SOCS1 | chr16: 11254865-11254884 |
| SOCS1 | chr16: 11254830-11254849 |
| SOCS1 | chr16: 11255401-11255420 |
| SOCS1 | chr16: 11254864-11254883 |
| SOCS1 | chr16: 11255311-11255330 |
| SOCS1 | chr16: 11255343-11255362 |
| SOCS1 | chr16: 11255342-11255361 |
| SOCS1 | chr16: 11255272-11255291 |
| SOCS1 | chr16: 11254866-11254885 |
| SOCS1 | chr16: 11255310-11255329 |
| SOCS1 | chr16: 11255336-11255355 |
| SOCS1 | chr16: 11255416-11255435 |
| SOCS1 | chr16: 11255402-11255421 |
| SOCS1 | chr16: 11255467-11255486 |
| SOCS1 | chr16: 11254873-11254892 |
| SOCS1 | chr16: 11255265-11255284 |
| SOCS1 | chr16: 11254820-11254839 |
| SOCS1 | chr16: 11254848-11254867 |
| SOCS1 | chr16: 11255317-11255336 |
| SOCS1 | chr16: 11255351-11255370 |
| SOCS1 | chr16: 11254811-11254830 |
| SOCS1 | chr16: 11255353-11255372 |
| SOCS1 | chr16: 11255350-11255369 |
| SOCS1 | chr16: 11255309-11255328 |
| SOCS1 | chr16: 11255390-11255409 |
| SOCS1 | chr16: 11255478-11255497 |
| SOCS1 | chr16: 11255330-11255349 |
| SOCS1 | chr16: 11254875-11254894 |
| SOCS1 | chr16: 11255124-11255143 |
| SOCS1 | chr16: 11255352-11255371 |
| SOCS1 | chr16: 11254872-11254891 |
| SOCS1 | chr16: 11255331-11255350 |
| SOCS1 | chr16: 11255315-11255334 |
| SOCS1 | chr16: 11255482-11255501 |
| SOCS1 | chr16: 11254995-11255014 |
| SOCS1 | chr16: 11255316-11255335 |
| SOCS1 | chr16: 11255308-11255327 |
| SOCS1 | chr16: 11255321-11255340 |
| SOCS1 | chr16: 11255322-11255341 |
| SOCS1 | chr16: 11255330-11255349 |
| SOCS1 | chr16: 11255368-11255387 |
| SOCS1 | chr16: 11255377-11255396 |
| SOCS1 | chr16: 11255380-11255399 |

TABLE 4
Socs1 Murine Genome Coordinates

| Target | Coordinates |
|---|---|
| Socs1 | chr16: 10784479-10784498 |
| Socs1 | chr16: 10784409-10784428 |
| Socs1 | chr16: 10784456-10784475 |
| Socs1 | chr16: 10784322-10784341 |
| Socs1 | chr16: 10784548-10784567 |
| Socs1 | chr16: 10784596-10784615 |
| Socs1 | chr16: 10784264-10784283 |
| Socs1 | chr16: 10784628-10784647 |
| Socs1 | chr16: 10784526-10784545 |
| Socs1 | chr16: 10784508-10784527 |
| Socs1 | chr16: 10784565-10784584 |
| Socs1 | chr16: 10784474-10784493 |
| Socs1 | chr16: 10784293-10784312 |

TABLE A
Exemplary shRNA Target Sequences

| Sequence | SEQ ID |
|---|---|
| TTTCGAGCTGCTGGAGCACTA | 152 |
| TCGAGCTGCTGGAGCACTACG | 153 |
| TCGCCAACGGAACTGCTTCTT | 154 |
| ACTTCTGGCTGGAGACCTCAT | 155 |
| GCGAGACCTTCGACTGCCTTT | 156 |
| CGACACTCACTTCCGCACCTT | 157 |
| CTACCTGAGTTCCTTCCCCTT | 158 |
| TTCCGCTCCCACTCCGATTAC | 159 |
| TAACCCGGTACTCCGTGACTA | 160 |
| TACTCCGTGACTACCTGAGTT | 161 |
| CTTCCGCTCCCACTCCGATTA | 162 |
| GCGCGACAGTCGCCAACGGAA | 163 |
| TGGACGCCTGCGGCTTCTATT | 164 |
| CGCATCCCTCTTAACCCGGTA | 165 |
| TACATATTCCCAGTATCTTTG | 166 |
| GCGCCTTATTATTTCTTATTA | 167 |
| CCGTGACTACCTGAGTTCCTT | 168 |
| GGAGGGTCTCTGGCTTCATTT | 169 |
| TTCGCGCTCAGCGTGAAGATG | 170 |
| ATCCCTCTTAACCCGGTACTC | 171 |
| CACGCACTTCCGCACATTC | 172 |
| TTCCGTTCGCACGCCGATT | 173 |
| GAGCTTCGACTGCCTCTTC | 174 |

TABLE B
Exemplary siRNA Target Sequences

| Sequence | SEQ ID |
|---|---|
| CGCACUUCCGCACAUUCCGUUCG | 175 |
| GGGGAGGGUCUCUGGCUUUAUUU | 176 |
| CAGCAUUAACUGGGAUGCCGUGU | 177 |
| CCAGGACCUGAACUCGCACCUCC | 178 |
| UACAUAUACCCAGUAUCUUUGCA | 179 |
| GCCGACAAUGCAGUCUCCACAGC | 180 |
| CCCCUGGUUGUUGUAGCAGCUUA | 181 |
| CUGCUGUGCAGAAUCCUAUUUUA | 182 |
| UGGGAUGCCGUGUUAUUUUGUUA | 183 |
| UCGCACCUCCUACCUCUUCAUGU | 184 |

In some embodiments, the nucleic acid-based gene-regulating system comprises at least two nucleic acid molecules (e.g., an siRNA, an shRNA, an RNA aptamer, or a morpholino), wherein at least one nucleic acid molecule is a PTPN2-targeting nucleic acid molecule. In some embodiments, the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4). In some embodiments, the least one PTPN2-targeting nucleic acid molecule is an siRNA or an shRNA molecule. In some embodiments, the at least one PTPN2-targeting siRNA or an shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one PTPN2-targeting siRNA or an shRNA molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 5 or Table 6. In some embodiments, the at least one PTPN2-targeting nucleic acid molecule binds to a target RNA sequence that is 100% identical to a RNA sequence encoded by a DNA sequence defined by a set of genomic coordinates shown in Table 5 or Table 6. In some embodiments, the at least one PTPN2-targeting nucleic acid molecule binds to a target RNA sequence that is at least 95%, 96%, 97%, 98%, or 99% identical to an RNA sequence encoded by one of SEQ ID NOs: 185-207. In some embodiments, the at least one PTPN2-targeting nucleic acid molecule binds to a target RNA sequence that is 100% identical to an RNA sequence encoded by one of SEQ ID NOs: 185-207.

In some embodiments, the at least one PTPN2-targeting nucleic acid molecule is a SOCS1-targeting shRNA or siRNA molecule. In some embodiments, the at least one PTPN2-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 5 or Table 6. In some embodiments, the at least one PTPN2-targeting shRNA or siRNA molecule binds to a target RNA sequence that is 100% identical to a RNA sequence encoded by a DNA sequence defined by a set of genomic coordinates shown in Table 5 or Table 6. In some embodiments, the at least one PTPN2-targeting shRNA or siRNA molecule binds to a target RNA sequence that is at least 95%, 96%, 97%, 98%, or 99% identical to an RNA sequence encoded by one of SEQ ID NOs: 185-207. In some embodiments, the at least one PTPN2-targeting shRNA or siRNA molecule binds to a target RNA sequence that is 100% identical to an RNA sequence encoded by one of SEQ ID NOs: 185-207.

TABLE 5
PTPN2 Human Genome Coordinates

| | |
|---|---|
| PTPN2 | Chr18: 12859218-12859237 |
| PTPN2 | Chr18: 12884109-12884128 |
| PTPN2 | Chr18: 12817227-12817246 |
| PTPN2 | Chr18: 12817234-12817253 |
| PTPN2 | Chr18: 12884091-12884110 |
| PTPN2 | Chr18: 12884121-12884140 |
| PTPN2 | Chr18: 12831010-12831029 |
| PTPN2 | Chr18: 12817208-12817227 |
| PTPN2 | Chr18: 12817158-12817177 |
| PTPN2 | Chr18: 12831016-12831035 |
| PTPN2 | Chr18: 12817228-12817247 |
| PTPN2 | Chr18: 12830964-12830983 |
| PTPN2 | Chr18: 12801972-12801991 |
| PTPN2 | Chr18: 12836818-12836837 |
| PTPN2 | Chr18: 12817215-12817234 |
| PTPN2 | Chr18: 12802018-12802037 |
| PTPN2 | Chr18: 12884116-12884135 |
| PTPN2 | Chr18: 12840739-12840758 |
| PTPN2 | Chr18: 12802004-12802023 |
| PTPN2 | Chr18: 12840767-12840786 |
| PTPN2 | Chr18: 12817197-12817216 |
| PTPN2 | Chr18: 12884108-12884127 |
| PTPN2 | Chr18: 12817221-12817240 |
| PTPN2 | Chr18: 12836820-12836839 |
| PTPN2 | Chr18: 12884124-12884143 |
| PTPN2 | Chr18: 12830996-12831015 |
| PTPN2 | Chr18: 12830942-12830961 |
| PTPN2 | Chr18: 12884112-12884131 |
| PTPN2 | Chr18: 12817193-12817212 |
| PTPN2 | Chr18: 12859205-12859224 |
| PTPN2 | Chr18: 12817202-12817221 |
| PTPN2 | Chr18: 12859216-12859235 |
| PTPN2 | Chr18: 12859215-12859234 |
| PTPN2 | Chr18: 12817201-12817220 |
| PTPN2 | Chr18: 12802134-12802153 |
| PTPN2 | Chr18: 12884075-12884094 |
| PTPN2 | Chr18: 12884115-12884134 |
| PTPN2 | Chr18: 12840757-12840776 |
| PTPN2 | Chr18: 12814205-12814224 |
| PTPN2 | Chr18: 12840777-12840796 |
| PTPN2 | Chr18: 12814277-12814296 |
| PTPN2 | Chr18: 12840746-12840765 |
| PTPN2 | Chr18: 12801989-12802008 |
| PTPN2 | Chr18: 12819237-12819256 |
| PTPN2 | Chr18: 12814348-12814367 |
| PTPN2 | Chr18: 12794428-12794447 |
| PTPN2 | Chr18: 12831005-12831024 |
| PTPN2 | Chr18: 12825890-12825909 |
| PTPN2 | Chr18: 12840723-12840742 |
| PTPN2 | Chr18: 12840747-12840766 |
| PTPN2 | Chr18: 12802068-12802087 |
| PTPN2 | Chr18: 12840716-12840735 |
| PTPN2 | Chr18: 12840773-12840792 |
| PTPN2 | Chr18: 12831012-12831031 |
| PTPN2 | Chr18: 12814240-12814259 |
| PTPN2 | Chr18: 12802130-12802149 |
| PTPN2 | Chr18: 12794454-12794473 |
| PTPN2 | Chr18: 12817208-12817227 |
| PTPN2 | Chr18: 12819226-12819245 |
| PTPN2 | Chr18: 12825889-12825908 |
| PTPN2 | Chr18: 12840782-12840801 |
| PTPN2 | Chr18: 12836812-12836831 |
| PTPN2 | Chr18: 12817298-12817317 |
| PTPN2 | Chr18: 12817324-12817343 |
| PTPN2 | Chr18: 12819268-12819287 |
| PTPN2 | Chr18: 12817303-12817322 |
| PTPN2 | Chr18: 12825927-12825946 |
| PTPN2 | Chr18: 12817220-12817239 |
| PTPN2 | Chr18: 12825901-12825920 |
| PTPN2 | Chr18: 12814222-12814241 |
| PTPN2 | Chr18: 12831000-12831019 |
| PTPN2 | Chr18: 12840738-12840757 |
| PTPN2 | Chr18: 12802057-12802076 |
| PTPN2 | Chr18: 12802069-12802088 |
| PTPN2 | Chr18: 12884123-12884142 |
| PTPN2 | Chr18: 12814294-12814313 |
| PTPN2 | Chr18: 12817283-12817302 |
| PTPN2 | Chr18: 12830945-12830964 |
| PTPN2 | Chr18: 12817284-12817303 |
| PTPN2 | Chr18: 12817256-12817275 |
| PTPN2 | Chr18: 12884062-12884081 |
| PTPN2 | Chr18: 12814295-12814314 |
| PTPN2 | Chr18: 12817313-12817332 |
| PTPN2 | Chr18: 12814255-12814274 |
| PTPN2 | Chr18: 12814253-12814272 |
| PTPN2 | Chr18: 12814257-12814276 |
| PTPN2 | Chr18: 12814256-12814275 |
| PTPN2 | Chr18: 12840753-12840772 |
| PTPN2 | Chr18: 12830957-12830976 |
| PTPN2 | Chr18: 12802093-12802112 |
| PTPN2 | Chr18: 12817333-12817352 |
| PTPN2 | Chr18: 12794479-12794498 |
| PTPN2 | Chr18: 12814223-12814242 |
| PTPN2 | Chr18: 12802089-12802108 |
| PTPN2 | Chr18: 12794463-12794482 |
| PTPN2 | Chr18: 12794436-12794455 |
| PTPN2 | Chr18: 12794416-12794435 |
| PTPN2 | Chr18: 12817235-12817254 |
| PTPN2 | Chr18: 12836793-12836812 |
| PTPN2 | Chr18: 12801986-12802005 |
| PTPN2 | Chr18: 12817165-12817184 |
| PTPN2 | Chr18: 12817179-12817198 |
| PTPN2 | Chr18: 12794425-12794444 |
| PTPN2 | Chr18: 12802146-12802165 |

TABLE 6
Ptpn2 Murine Genome Coordinates

| Target | Coordinates |
|---|---|
| Ptpn2 | Chr18: 67680998-67681017 |
| Ptpn2 | Chr18: 67677801-67677820 |
| Ptpn2 | Chr18: 67680904-67680923 |
| Ptpn2 | Chr18: 67681553-67681572 |
| Ptpn2 | Chr18: 67688965-67688984 |
| Ptpn2 | Chr18: 67680958-67680977 |
| Ptpn2 | Chr18: 67688944-67688963 |
| Ptpn2 | Chr18: 67677855-67677874 |
| Ptpn2 | Chr18: 67677734-67677753 |
| Ptpn2 | Chr18: 67680967-67680986 |
| Ptpn2 | Chr18: 67688912-67688931 |
| Ptpn2 | Chr18: 67680881-67680900 |
| Ptpn2 | Chr18: 67681529-67681548 |

In some embodiments, the nucleic acid-based gene-regulating system comprises at least two nucleic acid molecules (e.g., an siRNA, an shRNA, an RNA aptamer, or a morpholino), wherein at least one nucleic acid molecule is a ZC3H12A-targeting nucleic acid molecule. In some embodiments, the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6). In some embodiments, the least one ZC3H12A-targeting nucleic acid molecule is an siRNA or an shRNA molecule. In some embodiments, the at least one ZC3H12A-targeting siRNA or an shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one ZC3H12A-targeting siRNA or an shRNA molecule binds to a target RNA sequence that is 100% identical to an RNA sequence encoded by the ZC3H12A gene (SEQ ID NO: 5) or the Zc3h12A gene (SEQ ID NO: 6).

In some embodiments, the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 7 or Table 8. In some embodiments, the at least one ZC3H12A-targeting nucleic acid molecule binds to a target RNA sequence that is 100% identical to a RNA sequence encoded by a DNA sequence defined by a set of genomic coordinates shown in Table 7 or Table 8. In some embodiments, the at least one ZC3H12A-targeting nucleic acid molecule binds to a target RNA sequence that is at least 95%, 96%, 97%, 98%, or 99% identical to an RNA sequence encoded by one of SEQ ID NOs: 208-230. In some embodiments, the at least one ZC3H12A-targeting nucleic acid molecule binds to a target RNA sequence that is 100% identical to an RNA sequence encoded by one of SEQ ID NOs: 208-230.

In some embodiments, the at least one ZC3H12A-targeting nucleic acid molecule is a SOCS1-targeting shRNA or siRNA molecule. In some embodiments, the at least one ZC3H12A-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 7 or Table 8. In some embodiments, the at least one ZC3H12A-targeting shRNA or siRNA molecule binds to a target RNA sequence that is 100% identical to a RNA sequence encoded by a DNA sequence defined by a set of genomic coordinates shown in Table 7 or Table 8. In some embodiments, the at least one ZC3H12A-targeting shRNA or siRNA molecule binds to a target RNA sequence that is at least 95%, 96%, 97%, 98%, or 99% identical to an RNA sequence encoded by one of SEQ ID NOs: 208-230. In some embodiments, the at least one ZC3H12A-targeting shRNA or siRNA molecule binds to a target RNA sequence that is 100% identical to an RNA sequence encoded by one of SEQ ID NOs: 208-230. In some embodiments, the ZC3H12A-targeting nucleic acid molecule is a ZC3H12A-targeting shRNA molecule encoded by a nucleic acid sequence selected from SEQ ID NOs: 234-237 (See Huang et al., J Biol Chem (2015) 290(34), 20782-20792). In some embodiments, the ZC3H12A-targeting nucleic acid molecule is a ZC3H12A-targeting siRNA comprising a nucleic acid sequence selected from SEQ ID NOs: 231-233 (See Liu et al., Scientific Reports (2016), 6, Article #24073 and Mino et al., Cell (2015) 161(5), 1058-1073).

TABLE 7
ZC3H12A Human Genome Coordinates

| Target | Coordinates |
|---|---|
| ZC3H12A | Chr1: 37481708-37481727 |
| ZC3H12A | Chr1: 37475808-37475827 |
| ZC3H12A | Chr1: 37475809-37475828 |
| ZC3H12A | Chr1: 37475684-37475703 |
| ZC3H12A | Chr1: 37481823-37481842 |
| ZC3H12A | Chr1: 37480415-37480434 |
| ZC3H12A | Chr1: 37475756-37475775 |
| ZC3H12A | Chr1: 37481692-37481711 |
| ZC3H12A | Chr1: 37481648-37481667 |
| ZC3H12A | Chr1: 37480284-37480303 |
| ZC3H12A | Chr1: 37481779-37481798 |
| ZC3H12A | Chr1: 37475827-37475846 |
| ZC3H12A | Chr1: 37481747-37481766 |
| ZC3H12A | Chr1: 37482445-37482464 |
| ZC3H12A | Chr1: 37475631-37475650 |
| ZC3H12A | Chr1: 37480274-37480293 |
| ZC3H12A | Chr1: 37482967-37482986 |
| ZC3H12A | Chr1: 37482922-37482941 |
| ZC3H12A | Chr1: 37480273-37480292 |
| ZC3H12A | Chr1: 37482886-37482905 |
| ZC3H12A | Chr1: 37483185-37483204 |
| ZC3H12A | Chr1: 37475817-37475836 |
| ZC3H12A | Chr1: 37483033-37483052 |
| ZC3H12A | Chr1: 37480408-37480427 |
| ZC3H12A | Chr1: 37483026-37483045 |
| ZC3H12A | Chr1: 37483463-37483482 |
| ZC3H12A | Chr1: 37480362-37480381 |
| ZC3H12A | Chr1: 37482962-37482981 |
| ZC3H12A | Chr1: 37475775-37475794 |
| ZC3H12A | Chr1: 37475509-37475528 |
| ZC3H12A | Chr1: 37475722-37475741 |
| ZC3H12A | Chr1: 37475818-37475837 |
| ZC3H12A | Chr1: 37482966-37482985 |
| ZC3H12A | Chr1: 37480388-37480407 |
| ZC3H12A | Chr1: 37483142-37483161 |
| ZC3H12A | Chr1: 37482448-37482467 |
| ZC3H12A | Chr1: 37483049-37483068 |
| ZC3H12A | Chr1: 37482905-37482924 |
| ZC3H12A | Chr1: 37482733-37482752 |
| ZC3H12A | Chr1: 37480423-37480442 |
| ZC3H12A | Chr1: 37482456-37482475 |
| ZC3H12A | Chr1: 37483551-37483570 |
| ZC3H12A | Chr1: 37481767-37481786 |
| ZC3H12A | Chr1: 37475715-37475734 |
| ZC3H12A | Chr1: 37483377-37483396 |
| ZC3H12A | Chr1: 37475593-37475612 |
| ZC3H12A | Chr1: 37475875-37475894 |
| ZC3H12A | Chr1: 37475534-37475553 |
| ZC3H12A | Chr1: 37482764-37482783 |
| ZC3H12A | Chr1: 37475869-37475888 |
| ZC3H12A | Chr1: 37483437-37483456 |
| ZC3H12A | Chr1: 37475598-37475617 |
| ZC3H12A | Chr1: 37482438-37482457 |
| ZC3H12A | Chr1: 37483257-37483276 |
| ZC3H12A | Chr1: 37483263-37483282 |
| ZC3H12A | Chr1: 37482545-37482564 |
| ZC3H12A | Chr1: 37483015-37483034 |
| ZC3H12A | Chr1: 37481595-37481614 |
| ZC3H12A | Chr1: 37482923-37482942 |
| ZC3H12A | Chr1: 37483143-37483162 |
| ZC3H12A | Chr1: 37482348-37482367 |
| ZC3H12A | Chr1: 37483018-37483037 |
| ZC3H12A | Chr1: 37482612-37482631 |
| ZC3H12A | Chr1: 37475613-37475632 |
| ZC3H12A | Chr1: 37475563-37475582 |
| ZC3H12A | Chr1: 37475535-37475554 |
| ZC3H12A | Chr1: 37482843-37482862 |
| ZC3H12A | Chr1: 37480424-37480443 |
| ZC3H12A | Chr1: 37482606-37482625 |
| ZC3H12A | Chr1: 37483098-37483117 |
| ZC3H12A | Chr1: 37483508-37483527 |
| ZC3H12A | Chr1: 37483559-37483578 |
| ZC3H12A | Chr1: 37483256-37483275 |
| ZC3H12A | Chr1: 37475936-37475955 |
| ZC3H12A | Chr1: 37475607-37475626 |
| ZC3H12A | Chr1: 37475809-37475828 |
| ZC3H12A | Chr1: 37483186-37483205 |
| ZC3H12A | Chr1: 37481747-37481766 |
| ZC3H12A | Chr1: 37482734-37482753 |
| ZC3H12A | Chr1: 37483278-37483297 |
| ZC3H12A | Chr1: 37482332-37482351 |
| ZC3H12A | Chr1: 37483109-37483128 |
| ZC3H12A | Chr1: 37475633-37475652 |
| ZC3H12A | Chr1: 37482591-37482610 |
| ZC3H12A | Chr1: 37483271-37483290 |
| ZC3H12A | Chr1: 37483603-37483622 |
| ZC3H12A | Chr1: 37482504-37482523 |
| ZC3H12A | Chr1: 37483252-37483271 |
| ZC3H12A | Chr1: 37483119-37483138 |
| ZC3H12A | Chr1: 37482343-37482362 |
| ZC3H12A | Chr1: 37483144-37483163 |
| ZC3H12A | Chr1: 37483213-37483232 |
| ZC3H12A | Chr1: 37482981-37483000 |
| ZC3H12A | Chr1: 37482789-37482808 |
| ZC3H12A | Chr1: 37483159-37483178 |
| ZC3H12A | Chr1: 37482349-37482368 |
| ZC3H12A | Chr1: 37483602-37483621 |
| ZC3H12A | Chr1: 37481596-37481615 |
| ZC3H12A | Chr1: 37482537-37482556 |
| ZC3H12A | Chr1: 37482370-37482389 |
| ZC3H12A | Chr1: 37475546-37475565 |
| ZC3H12A | Chr1: 37482598-37482617 |
| ZC3H12A | Chr1: 37483146-37483165 |
| ZC3H12A | Chr1: 37475812-37475831 |
| ZC3H12A | Chr1: 37483400-37483419 |
| ZC3H12A | Chr1: 37475703-37475722 |
| ZC3H12A | Chr1: 37483418-37483437 |
| ZC3H12A | Chr1: 37480284-37480303 |
| ZC3H12A | Chr1: 37482800-37482819 |
| ZC3H12A | Chr1: 37475721-37475740 |
| ZC3H12A | Chr1: 37482715-37482734 |
| ZC3H12A | Chr1: 37480281-37480300 |
| ZC3H12A | Chr1: 37482491-37482510 |
| ZC3H12A | Chr1: 37483497-37483516 |
| ZC3H12A | Chr1: 37475899-37475918 |
| ZC3H12A | Chr1: 37475889-37475908 |
| ZC3H12A | Chr1: 37482375-37482394 |
| ZC3H12A | Chr1: 37475741-37475760 |
| ZC3H12A | Chr1: 37482900-37482919 |
| ZC3H12A | Chr1: 37482442-37482461 |
| ZC3H12A | Chr1: 37481644-37481663 |
| ZC3H12A | Chr1: 37482464-37482483 |
| ZC3H12A | Chr1: 37482994-37483013 |
| ZC3H12A | Chr1: 37483437-37483456 |
| ZC3H12A | Chr1: 37482736-37482755 |
| ZC3H12A | Chr1: 37482538-37482557 |
| ZC3H12A | Chr1: 37483515-37483534 |
| ZC3H12A | Chr1: 37475874-37475893 |
| ZC3H12A | Chr1: 37483145-37483164 |
| ZC3H12A | Chr1: 37482587-37482606 |
| ZC3H12A | Chr1: 37475482-37475501 |
| ZC3H12A | Chr1: 37475844-37475863 |
| ZC3H12A | Chr1: 37480415-37480434 |
| ZC3H12A | Chr1: 37481709-37481728 |
| ZC3H12A | Chr1: 37483366-37483385 |
| ZC3H12A | Chr1: 37475627-37475646 |
| ZC3H12A | Chr1: 37482447-37482466 |
| ZC3H12A | Chr1: 37481758-37481777 |
| ZC3H12A | Chr1: 37483560-37483579 |
| ZC3H12A | Chr1: 37475869-37475888 |
| ZC3H12A | Chr1: 37481655-37481674 |
| ZC3H12A | Chr1: 37481645-37481664 |
| ZC3H12A | Chr1: 37483016-37483035 |
| ZC3H12A | Chr1: 37475838-37475857 |
| ZC3H12A | Chr1: 37482850-37482869 |
| ZC3H12A | Chr1: 37475510-37475529 |
| ZC3H12A | Chr1: 37483510-37483529 |
| ZC3H12A | Chr1: 37483064-37483083 |
| ZC3H12A | Chr1: 37483149-37483168 |
| ZC3H12A | Chr1: 37483449-37483468 |
| ZC3H12A | Chr1: 37483264-37483283 |
| ZC3H12A | Chr1: 37475508-37475527 |
| ZC3H12A | Chr1: 37480415-37480434 |
| ZC3H12A | Chr1: 37482918-37482937 |
| ZC3H12A | Chr1: 37482474-37482493 |
| ZC3H12A | Chr1: 37483232-37483251 |
| ZC3H12A | Chr1: 37475732-37475751 |
| ZC3H12A | Chr1: 37481602-37481621 |
| ZC3H12A | Chr1: 37480289-37480308 |
| ZC3H12A | Chr1: 37483165-37483184 |
| ZC3H12A | Chr1: 37483248-37483267 |
| ZC3H12A | Chr1: 37483078-37483097 |
| ZC3H12A | Chr1: 37483017-37483036 |
| ZC3H12A | Chr1: 37483174-37483193 |
| ZC3H12A | Chr1: 37482857-37482876 |
| ZC3H12A | Chr1: 37475578-37475597 |
| ZC3H12A | Chr1: 37480329-37480348 |
| ZC3H12A | Chr1: 37480288-37480307 |
| ZC3H12A | Chr1: 37481600-37481619 |
| ZC3H12A | Chr1: 37483212-37483231 |
| ZC3H12A | Chr1: 37483337-37483356 |
| ZC3H12A | Chr1: 37475542-37475561 |
| ZC3H12A | Chr1: 37483197-37483216 |
| ZC3H12A | Chr1: 37482730-37482749 |
| ZC3H12A | Chr1: 37475599-37475618 |
| ZC3H12A | Chr1: 37483262-37483281 |
| ZC3H12A | Chr1: 37482790-37482809 |
| ZC3H12A | Chr1: 37482719-37482738 |
| ZC3H12A | Chr1: 37482860-37482879 |
| ZC3H12A | Chr1: 37483443-37483462 |
| ZC3H12A | Chr1: 37483558-37483577 |
| ZC3H12A | Chr1: 37481599-37481618 |
| ZC3H12A | Chr1: 37475845-37475864 |
| ZC3H12A | Chr1: 37475730-37475749 |
| ZC3H12A | Chr1: 37482524-37482543 |
| ZC3H12A | Chr1: 37482849-37482868 |
| ZC3H12A | Chr1: 37475529-37475548 |
| ZC3H12A | Chr1: 37475664-37475683 |
| ZC3H12A | Chr1: 37482972-37482991 |
| ZC3H12A | Chr1: 37483321-37483340 |
| ZC3H12A | Chr1: 37482984-37483003 |
| ZC3H12A | Chr1: 37475807-37475826 |
| ZC3H12A | Chr1: 37483213-37483232 |
| ZC3H12A | Chr1: 37482427-37482446 |
| ZC3H12A | Chr1: 37483104-37483123 |
| ZC3H12A | Chr1: 37482879-37482898 |
| ZC3H12A | Chr1: 37483409-37483428 |
| ZC3H12A | Chr1: 37482752-37482771 |
| ZC3H12A | Chr1: 37480391-37480410 |
| ZC3H12A | Chr1: 37475694-37475713 |
| ZC3H12A | Chr1: 37482458-37482477 |
| ZC3H12A | Chr1: 37475774-37475793 |
| ZC3H12A | Chr1: 37475574-37475593 |
| ZC3H12A | Chr1: 37475803-37475822 |
| ZC3H12A | Chr1: 37481605-37481624 |
| ZC3H12A | Chr1: 37482437-37482456 |
| ZC3H12A | Chr1: 37482825-37482844 |
| ZC3H12A | Chr1: 37483595-37483614 |
| ZC3H12A | Chr1: 37483510-37483529 |
| ZC3H12A | Chr1: 37483283-37483302 |
| ZC3H12A | Chr1: 37482446-37482465 |
| ZC3H12A | Chr1: 37475700-37475719 |
| ZC3H12A | Chr1: 37475721-37475740 |
| ZC3H12A | Chr1: 37475628-37475647 |
| ZC3H12A | Chr1: 37482848-37482867 |
| ZC3H12A | Chr1: 37483134-37483153 |
| ZC3H12A | Chr1: 37475543-37475562 |
| ZC3H12A | Chr1: 37482799-37482818 |
| ZC3H12A | Chr1: 37483296-37483315 |
| ZC3H12A | Chr1: 37483332-37483351 |
| ZC3H12A | Chr1: 37483600-37483619 |
| ZC3H12A | Chr1: 37482410-37482429 |
| ZC3H12A | Chr1: 37481718-37481737 |
| ZC3H12A | Chr1: 37483395-37483414 |
| ZC3H12A | Chr1: 37482428-37482447 |
| ZC3H12A | Chr1: 37475562-37475581 |
| ZC3H12A | Chr1: 37483500-37483519 |
| ZC3H12A | Chr1: 37475827-37475846 |
| ZC3H12A | Chr1: 37483586-37483605 |
| ZC3H12A | Chr1: 37483089-37483108 |
| ZC3H12A | Chr1: 37483419-37483438 |
| ZC3H12A | Chr1: 37480285-37480304 |
| ZC3H12A | Chr1: 37483256-37483275 |
| ZC3H12A | Chr1: 37483420-37483439 |
| ZC3H12A | Chr1: 37475691-37475710 |
| ZC3H12A | Chr1: 37483419-37483438 |
| ZC3H12A | Chr1: 37475918-37475937 |
| ZC3H12A | Chr1: 37475589-37475608 |
| ZC3H12A | Chr1: 37482362-37482381 |
| ZC3H12A | Chr1: 37482566-37482585 |
| ZC3H12A | Chr1: 37482963-37482982 |
| ZC3H12A | Chr1: 37483420-37483439 |
| ZC3H12A | Chr1: 37483139-37483158 |
| ZC3H12A | Chr1: 37483619-37483638 |
| ZC3H12A | Chr1: 37481764-37481783 |
| ZC3H12A | Chr1: 37475650-37475669 |
| ZC3H12A | Chr1: 37483405-37483424 |
| ZC3H12A | Chr1: 37483037-37483056 |
| ZC3H12A | Chr1: 37483211-37483230 |
| ZC3H12A | Chr1: 37475537-37475556 |
| ZC3H12A | Chr1: 37475756-37475775 |
| ZC3H12A | Chr1: 37482403-37482422 |
| ZC3H12A | Chr1: 37482455-37482474 |
| ZC3H12A | Chr1: 37480311-37480330 |
| ZC3H12A | Chr1: 37482586-37482605 |
| ZC3H12A | Chr1: 37483099-37483118 |
| ZC3H12A | Chr1: 37483342-37483361 |
| ZC3H12A | Chr1: 37481823-37481842 |
| ZC3H12A | Chr1: 37482777-37482796 |
| ZC3H12A | Chr1: 37482412-37482431 |
| ZC3H12A | Chr1: 37483604-37483623 |
| ZC3H12A | Chr1: 37483438-37483457 |
| ZC3H12A | Chr1: 37482445-37482464 |
| ZC3H12A | Chr1: 37483331-37483350 |
| ZC3H12A | Chr1: 37483111-37483130 |
| ZC3H12A | Chr1: 37482847-37482866 |
| ZC3H12A | Chr1: 37483249-37483268 |
| ZC3H12A | Chr1: 37481754-37481773 |
| ZC3H12A | Chr1: 37475684-37475703 |
| ZC3H12A | Chr1: 37482519-37482538 |
| ZC3H12A | Chr1: 37482475-37482494 |
| ZC3H12A | Chr1: 37482613-37482632 |
| ZC3H12A | Chr1: 37482939-37482958 |
| ZC3H12A | Chr1: 37475541-37475560 |
| ZC3H12A | Chr1: 37481763-37481782 |
| ZC3H12A | Chr1: 37483231-37483250 |
| ZC3H12A | Chr1: 37482953-37482972 |
| ZC3H12A | Chr1: 37482407-37482426 |
| ZC3H12A | Chr1: 37475808-37475827 |
| ZC3H12A | Chr1: 37481620-37481639 |
| ZC3H12A | Chr1: 37475592-37475611 |
| ZC3H12A | Chr1: 37483156-37483175 |
| ZC3H12A | Chr1: 37480329-37480348 |
| ZC3H12A | Chr1: 37475573-37475592 |
| ZC3H12A | Chr1: 37483198-37483217 |
| ZC3H12A | Chr1: 37483557-37483576 |
| ZC3H12A | Chr1: 37482892-37482911 |
| ZC3H12A | Chr1: 37483334-37483353 |
| ZC3H12A | Chr1: 37481708-37481727 |
| ZC3H12A | Chr1: 37483063-37483082 |
| ZC3H12A | Chr1: 37482998-37483017 |
| ZC3H12A | Chr1: 37482942-37482961 |
| ZC3H12A | Chr1: 37475508-37475527 |
| ZC3H12A | Chr1: 37482371-37482390 |
| ZC3H12A | Chr1: 37483119-37483138 |
| ZC3H12A | Chr1: 37482798-37482817 |
| ZC3H12A | Chr1: 37475859-37475878 |
| ZC3H12A | Chr1: 37483401-37483420 |
| ZC3H12A | Chr1: 37482851-37482870 |
| ZC3H12A | Chr1: 37475524-37475543 |
| ZC3H12A | Chr1: 37475601-37475620 |
| ZC3H12A | Chr1: 37475815-37475834 |
| ZC3H12A | Chr1: 37482801-37482820 |
| ZC3H12A | Chr1: 37475544-37475563 |
| ZC3H12A | Chr1: 37483010-37483029 |
| ZC3H12A | Chr1: 37483077-37483096 |
| ZC3H12A | Chr1: 37482404-37482423 |
| ZC3H12A | Chr1: 37475692-37475711 |
| ZC3H12A | Chr1: 37483596-37483615 |
| ZC3H12A | Chr1: 37483372-37483391 |
| ZC3H12A | Chr1: 37481596-37481615 |
| ZC3H12A | Chr1: 37480370-37480389 |
| ZC3H12A | Chr1: 37480377-37480396 |
| ZC3H12A | Chr1: 37483381-37483400 |
| ZC3H12A | Chr1: 37482899-37482918 |
| ZC3H12A | Chr1: 37480373-37480392 |
| ZC3H12A | Chr1: 37481847-37481866 |
| ZC3H12A | Chr1: 37483330-37483349 |
| ZC3H12A | Chr1: 37483065-37483084 |
| ZC3H12A | Chr1: 37482499-37482518 |
| ZC3H12A | Chr1: 37483105-37483124 |
| ZC3H12A | Chr1: 37475631-37475650 |
| ZC3H12A | Chr1: 37483530-37483549 |
| ZC3H12A | Chr1: 37483407-37483426 |
| ZC3H12A | Chr1: 37483308-37483327 |
| ZC3H12A | Chr1: 37482853-37482872 |
| ZC3H12A | Chr1: 37482934-37482953 |
| ZC3H12A | Chr1: 37475591-37475610 |
| ZC3H12A | Chr1: 37475826-37475845 |
| ZC3H12A | Chr1: 37475865-37475884 |
| ZC3H12A | Chr1: 37481784-37481803 |
| ZC3H12A | Chr1: 37480322-37480341 |
| ZC3H12A | Chr1: 37475664-37475683 |
| ZC3H12A | Chr1: 37475757-37475776 |
| ZC3H12A | Chr1: 37483385-37483404 |
| ZC3H12A | Chr1: 37482933-37482952 |
| ZC3H12A | Chr1: 37475866-37475885 |
| ZC3H12A | Chr1: 37475843-37475862 |
| ZC3H12A | Chr1: 37475797-37475816 |
| ZC3H12A | Chr1: 37475642-37475661 |
| ZC3H12A | Chr1: 37483270-37483289 |
| ZC3H12A | Chr1: 37483024-37483043 |
| ZC3H12A | Chr1: 37483201-37483220 |
| ZC3H12A | Chr1: 37482447-37482466 |
| ZC3H12A | Chr1: 37483253-37483272 |
| ZC3H12A | Chr1: 37483429-37483448 |
| ZC3H12A | Chr1: 37483195-37483214 |
| ZC3H12A | Chr1: 37481648-37481667 |
| ZC3H12A | Chr1: 37483424-37483443 |
| ZC3H12A | Chr1: 37475580-37475599 |
| ZC3H12A | Chr1: 37482980-37482999 |
| ZC3H12A | Chr1: 37480408-37480427 |
| ZC3H12A | Chr1: 37483405-37483424 |
| ZC3H12A | Chr1: 37475740-37475759 |
| ZC3H12A | Chr1: 37480387-37480406 |
| ZC3H12A | Chr1: 37483507-37483526 |
| ZC3H12A | Chr1: 37483110-37483129 |
| ZC3H12A | Chr1: 37483325-37483344 |
| ZC3H12A | Chr1: 37481692-37481711 |
| ZC3H12A | Chr1: 37475826-37475845 |
| ZC3H12A | Chr1: 37483098-37483117 |
| ZC3H12A | Chr1: 37481758-37481777 |
| ZC3H12A | Chr1: 37480320-37480339 |
| ZC3H12A | Chr1: 37483380-37483399 |
| ZC3H12A | Chr1: 37483011-37483030 |
| ZC3H12A | Chr1: 37483509-37483528 |
| ZC3H12A | Chr1: 37483509-37483528 |
| ZC3H12A | Chr1: 37482768-37482787 |
| ZC3H12A | Chr1: 37475804-37475823 |
| ZC3H12A | Chr1: 37475808-37475827 |
| ZC3H12A | Chr1: 37475859-37475878 |
| ZC3H12A | Chr1: 37482973-37482992 |
| ZC3H12A | Chr1: 37475634-37475653 |
| ZC3H12A | Chr1: 37475854-37475873 |
| ZC3H12A | Chr1: 37480334-37480353 |
| ZC3H12A | Chr1: 37480414-37480433 |
| ZC3H12A | Chr1: 37480316-37480335 |
| ZC3H12A | Chr1: 37482971-37482990 |
| ZC3H12A | Chr1: 37482781-37482800 |
| ZC3H12A | Chr1: 37483173-37483192 |
| ZC3H12A | Chr1: 37482391-37482410 |
| ZC3H12A | Chr1: 37482392-37482411 |
| ZC3H12A | Chr1: 37482936-37482955 |
| ZC3H12A | Chr1: 37483408-37483427 |
| ZC3H12A | Chr1: 37481779-37481798 |
| ZC3H12A | Chr1: 37483206-37483225 |
| ZC3H12A | Chr1: 37482561-37482580 |
| ZC3H12A | Chr1: 37481745-37481764 |
| ZC3H12A | Chr1: 37475802-37475821 |
| ZC3H12A | Chr1: 37483494-37483513 |
| ZC3H12A | Chr1: 37483371-37483390 |
| ZC3H12A | Chr1: 37482552-37482571 |
| ZC3H12A | Chr1: 37475491-37475510 |
| ZC3H12A | Chr1: 37482479-37482498 |
| ZC3H12A | Chr1: 37483140-37483159 |
| ZC3H12A | Chr1: 37483313-37483332 |
| ZC3H12A | Chr1: 37483458-37483477 |
| ZC3H12A | Chr1: 37483320-37483339 |
| ZC3H12A | Chr1: 37483204-37483223 |
| ZC3H12A | Chr1: 37475792-37475811 |
| ZC3H12A | Chr1: 37483475-37483494 |
| ZC3H12A | Chr1: 37475577-37475596 |
| ZC3H12A | Chr1: 37475787-37475806 |
| ZC3H12A | Chr1: 37483574-37483593 |
| ZC3H12A | Chr1: 37480284-37480303 |
| ZC3H12A | Chr1: 37482369-37482388 |
| ZC3H12A | Chr1: 37483384-37483403 |
| ZC3H12A | Chr1: 37483425-37483444 |
| ZC3H12A | Chr1: 37482582-37482601 |
| ZC3H12A | Chr1: 37483153-37483172 |
| ZC3H12A | Chr1: 37482935-37482954 |
| ZC3H12A | Chr1: 37483378-37483397 |
| ZC3H12A | Chr1: 37482952-37482971 |
| ZC3H12A | Chr1: 37483399-37483418 |
| ZC3H12A | Chr1: 37483309-37483328 |
| ZC3H12A | Chr1: 37483200-37483219 |
| ZC3H12A | Chr1: 37481641-37481660 |
| ZC3H12A | Chr1: 37481656-37481675 |
| ZC3H12A | Chr1: 37483036-37483055 |
| ZC3H12A | Chr1: 37483474-37483493 |
| ZC3H12A | Chr1: 37483004-37483023 |
| ZC3H12A | Chr1: 37481846-37481865 |
| ZC3H12A | Chr1: 37483205-37483224 |
| ZC3H12A | Chr1: 37483406-37483425 |
| ZC3H12A | Chr1: 37480336-37480355 |
| ZC3H12A | Chr1: 37481716-37481735 |
| ZC3H12A | Chr1: 37480335-37480354 |
| ZC3H12A | Chr1: 37481659-37481678 |
| ZC3H12A | Chr1: 37475809-37475828 |
| ZC3H12A | Chr1: 37482565-37482584 |
| ZC3H12A | Chr1: 37482491-37482510 |
| ZC3H12A | Chr1: 37483379-37483398 |
| ZC3H12A | Chr1: 37481654-37481673 |
| ZC3H12A | Chr1: 37482567-37482586 |
| ZC3H12A | Chr1: 37481614-37481633 |
| ZC3H12A | Chr1: 37482562-37482581 |
| ZC3H12A | Chr1: 37475868-37475887 |
| ZC3H12A | Chr1: 37482557-37482576 |
| ZC3H12A | Chr1: 37483511-37483530 |
| ZC3H12A | Chr1: 37475615-37475634 |
| ZC3H12A | Chr1: 37483333-37483352 |
| ZC3H12A | Chr1: 37482840-37482859 |
| ZC3H12A | Chr1: 37483545-37483564 |
| ZC3H12A | Chr1: 37482830-37482849 |
| ZC3H12A | Chr1: 37482444-37482463 |
| ZC3H12A | Chr1: 37482571-37482590 |
| ZC3H12A | Chr1: 37482553-37482572 |
| ZC3H12A | Chr1: 37483543-37483562 |
| ZC3H12A | Chr1: 37483542-37483561 |
| ZC3H12A | Chr1: 37482575-37482594 |
| ZC3H12A | Chr1: 37475855-37475874 |
| ZC3H12A | Chr1: 37482572-37482591 |

TABLE 8
Zc3h12a Murine Genome Coordinates

| Target | Coordinates |
|---|---|
| Zc3h12a | Chr1: 125122335-125122354 |
| Zc3h12a | Chr1: 125121083-125121102 |
| Zc3h12a | Chr1: 125120961-125120980 |
| Zc3h12a | Chr1: 125122390-125122409 |
| Zc3h12a | Chr1: 125120373-125120392 |
| Zc3h12a | Chr1: 125122250-125122269 |
| Zc3h12a | Chr1: 125122375-125122394 |
| Zc3h12a | Chr1: 125120975-125120994 |

In some embodiments, the at least one SOCS1-, PTPN2-, or ZC3H12A-targeting siRNA molecule or shRNA molecule is obtained from a commercial suppliers such as Sigma Aldrich®, Dharmacon®, ThermoFisher®, and the like. In some embodiments, the at least one SOCS1-, PTPN2-, or ZC3H12A-targeting siRNA molecule is one shown in Table 9. In some embodiments, the at least one SOCS1-, PTPN2-, or ZC3H12A-targeting shRNA molecule is one shown in Table 10.

TABLE 9
Exemplary SOCS1, PTPN2, and ZC3H12A siRNAs

| Target Gene | siRNA construct |
|---|---|
| SOCS1 | MISSION ® esiRNA targeting mouse Socs1 |
| | (SigmaAlrich# EMU203261) |
| | Rosetta Predictions human (SigmaAlrich# |
| | NM_003745) |
| | Rosetta Predictions murine (SigmaAlrich# |
| | NM_009896) |
| PTPN2 | MISSION ® esiRNA human PTPN2 (esiRNA1) |
| | (SigmaAldrich# EHU113971) |
| | human Rosetta Predictions (SigmaAldrich# |
| | NM_002828) |
| | human Rosetta Predictions (SigmaAldrich# |
| | NM_080422) |
| | human Rosetta Predictions (SigmaAldrich# |
| | NM_080423) |
| | murine Rosetta Predictions (SigmaAldrich# |
| | NM_001127177) |
| ZC3H12A | MISSION ® esiRNA targeting human ZC3H12A |
| | (esiRNA1) (SigmaAldrich# EHU009491) |
| | MISSION ® esiRNA targeting mouse Zc3h12a |
| | (esiRNA1) (SigmaAldrich# EMU048551) |
| | Rosetta Predictions human (SigmaAldrich# |
| | NM_025079) |
| | Rosetta Predictions mouse (SigmaAldrich# |
| | NM_153159) |

TABLE 10
Exemplary SOCS1, PTPN2, and ZC3H12A shRNAs

| Target Gene | shRNA construct |
|---|---|
| SOCS1 | MISSION ® shRNA Plasmid DNA human |
| | (SigmaAlrich# SHCLND-NM_003745) |
| | MISSION ® shRNA Plasmid DNA murine |
| | (SigmaAlrich# SHCLND-NM_009896) |
| PTPN2 | MISSION ® shRNA Plasmid human |
| | (SigmaAldrich# SHCLND-NM_002827) |
| | MISSION ® shRNA Plasmid murine |
| | (SigmaAldrich# SHCLND-NM_011201) |
| ZC3H12A | MISSION ® shRNA Plasmid DNA human |
| | (SigmaAldrich# SHCLND-NM_025079) |
| | MISSION ® shRNA Plasmid DNA mouse |
| | (SigmaAldrich# SHCLND-NM_153159) |

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule and at least one nucleic acid molecule is a PTPN2-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2) and the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 3 or Table 4 and the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 5 or Table 6. In some embodiments, the at least one SOCS1-targeting nucleic acid molecule binds to a target RNA sequence that is 100% identical to a RNA sequence encoded by a DNA sequence defined by a set of genomic coordinates shown in Table 3 or Table 4 and the at least one PTPN2-targeting nucleic acid molecule binds to a target RNA sequence that is 100% identical to a RNA sequence encoded by a DNA sequence defined by a set of genomic coordinates shown in Table 5 or Table 6.

In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 7-151 and the at least one PTPN2-targeting nucleic acid molecule binds to a target RNA sequence that is at least 95%, 96%, 97%, 98%, or 99% identical to an RNA sequence encoded by one of SEQ ID NOs: 185-207. In some embodiments, the at least one SOCS1-targeting nucleic acid molecule binds to a target RNA sequence that is 100% identical to an RNA sequence encoded by one of SEQ ID NOs: 7-151 and the at least one PTPN2-targeting nucleic acid molecule binds to a target RNA sequence that is 100% identical to an RNA sequence encoded by one of SEQ ID NOs: 185-207.

In some embodiments, the nucleic acid-based gene-regulating system comprises at least two siRNA or shRNA molecules, wherein at least one siRNA or shRNA molecule is a SOCS1-targeting siRNA or shRNA molecule and at least one siRNA or shRNA molecule is a PTPN2-targeting siRNA or shRNA molecule. In some embodiments, the least one SOCS1-targeting nucleic acid molecule is an siRNA or an shRNA molecule and at least one PTPN2-targeting nucleic acid molecule is an siRNA or shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one PTPN2-targeting siRNA or shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2) and the at least one PTPN2-targeting siRNA or shRNA molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one SOCS1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 3 or Table 4 and the at least one PTPN2-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 5 or Table 6. In some embodiments, the at least one SOCS1-targeting siRNA or shRNA molecule binds to a target RNA sequence that is 100% identical to a RNA sequence encoded by a DNA sequence defined by a set of genomic coordinates shown in Table 3 or Table 4 and the at least one PTPN2-targeting siRNA or shRNA molecule binds to a target RNA sequence that is 100% identical to a RNA sequence encoded by a DNA sequence defined by a set of genomic coordinates shown in Table 5 or Table 6.

In some embodiments, the at least one SOCS1-targeting siRNA or shRNA molecule binds to a target RNA sequence that is at least 95%, 96%, 97%, 98%, or 99% identical to an RNA sequence encoded by one of SEQ ID NOs: 7-151 and the at least one PTPN2-targeting siRNA or shRNA molecule binds to a target RNA sequence that is at least 95%, 96%, 97%, 98%, or 99% identical to an RNA sequence encoded by one of SEQ ID NOs: 185-207. In some embodiments, the at least one SOCS1-targeting siRNA or shRNA molecule binds to a target RNA sequence that is 100% identical to an RNA sequence encoded by one of SEQ ID NOs: 7-151 and the at least one PTPN2-targeting siRNA or shRNA molecule binds to a target RNA sequence that is 100% identical to an RNA sequence encoded by one of SEQ ID NOs: 185-207.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule and at least one nucleic acid molecule is a ZC3H12A-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2) and the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 3 or Table 4 and the at least one ZC3H/2A-targeting nucleic acid molecule binds to a target RNA sequence that is at least 95%, 96%, 97%, 98%, or 99% identical to a RNA sequence encoded by a DNA sequence defined by a set of genomic coordinates shown in Table 7 or Table 8. In some embodiments, the at least one SOCS1-targeting nucleic acid molecule binds to a target RNA sequence that is 100% identical to a RNA sequence encoded by a DNA sequence defined by a set of genomic coordinates shown in Table 3 or Table 4 and the at least one ZC3H/2A-targeting nucleic acid molecule binds to a target RNA sequence that is 100% identical to a RNA sequence encoded by a DNA sequence defined by a set of genomic coordinates shown in Table 7 or Table 8.

In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 7-151 and the at least one ZC3H12A-targeting nucleic acid molecule binds to a target RNA sequence that is at least 95%, 96%, 97%, 98%, or 99% identical to an RNA sequence encoded by one of SEQ ID NOs: 208-230. In some embodiments, the at least one SOCS1-targeting nucleic acid molecule binds to a target RNA sequence that is 100% identical to an RNA sequence encoded by one of SEQ ID NOs: 7-151 and the at least one ZC3H/2A-targeting nucleic acid molecule binds to a target RNA sequence that is 100% identical to an RNA sequence encoded by one of SEQ ID NOs: 208-230.

In some embodiments, the nucleic acid-based gene-regulating system comprises at least two siRNA or shRNA molecules, wherein at least one siRNA or shRNA molecule is a SOCS1-targeting siRNA or shRNA molecule and at least one siRNA or shRNA molecule is a ZC3H12A-targeting siRNA or shRNA molecule. In some embodiments, the least one SOCS1-targeting nucleic acid molecule is an siRNA or an shRNA molecule and at least one ZC3H/2A-targeting nucleic acid molecule is an siRNA or shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one ZC3H/2A-targeting siRNA or shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2) and the at least one ZC3H/2A-targeting siRNA or shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one SOCS1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 3 or Table 4 and the at least one ZC3H/2A-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 7 or Table 8. In some embodiments, the at least one SOCS1-targeting siRNA or shRNA molecule binds to a target RNA sequence that is 100% identical to a RNA sequence encoded by a DNA sequence defined by a set of genomic coordinates shown in Table 3 or Table 4 and the at least one ZC3H/2A-targeting siRNA or shRNA molecule binds to a target RNA sequence that is 100% identical to a RNA sequence encoded by a DNA sequence defined by a set of genomic coordinates shown in Table 7 or Table 8.

In some embodiments, the at least one SOCS1-targeting siRNA or shRNA molecule binds to a target RNA sequence that is at least 95%, 96%, 97%, 98%, or 99% identical to an RNA sequence encoded by one of SEQ ID NOs: 7-151 and the at least one ZC3H/2A-targeting siRNA or shRNA molecule binds to a target RNA sequence that is at least 95%, 96%, 97%, 98%, or 99% identical to an RNA sequence encoded by one of SEQ ID NOs: 208-230. In some embodiments, the at least one SOCS1-targeting siRNA or shRNA molecule binds to a target RNA sequence that is 100% identical to an RNA sequence encoded by one of SEQ ID NOs: 7-151 and the at least one ZC3H12A-targeting siRNA or shRNA molecule binds to a target RNA sequence that is 100% identical to an RNA sequence encoded by one of SEQ ID NOs: 208-230.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a PTPN2-targeting nucleic acid molecule and at least one nucleic acid molecule is a ZC3H/2A-targeting nucleic acid molecule. In some embodiments, the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4) and the at least one ZC3H/2A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4) and the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 5 or Table 6 and the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 7 or Table 8. In some embodiments, the at least one PTPN2-targeting nucleic acid molecule binds to a target RNA sequence that is 100% identical to a RNA sequence encoded by a DNA sequence defined by a set of genomic coordinates shown in Table 5 or Table 6 and the at least one ZC3H12A-targeting nucleic acid molecule binds to a target RNA sequence that is 100% identical to a RNA sequence encoded by a DNA sequence defined by a set of genomic coordinates shown in Table 7 or Table 8.

In some embodiments, the at least one PTPN2-targeting nucleic acid molecule binds to a target RNA sequence that is at least 95%, 96%, 97%, 98%, or 99% identical to an RNA sequence encoded by one of SEQ ID NOs: 185-207 and the at least one ZC3H12A-targeting nucleic acid molecule binds to a target RNA sequence that is at least 95%, 96%, 97%, 98%, or 99% identical to an RNA sequence encoded by one of SEQ ID NOs: 208-230. In some embodiments, the at least one PTPN2-targeting nucleic acid molecule binds to a target RNA sequence that is 100% identical to an RNA sequence encoded by one of SEQ ID NOs: 185-207 and the at least one ZC3H12A-targeting nucleic acid molecule binds to a target RNA sequence that is 100% identical to an RNA sequence encoded by one of SEQ ID NOs: 208-230.

In some embodiments, the nucleic acid-based gene-regulating system comprises at least two siRNA or shRNA molecules, wherein at least one siRNA or shRNA molecule is a PTPN2-targeting siRNA or shRNA molecule and at least one siRNA or shRNA molecule is a ZC3H12A-targeting siRNA or shRNA molecule. In some embodiments, the at least one PTPN2-targeting siRNA or an shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4) and the at least one ZC3H12A-targeting siRNA or shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one PTPN2-targeting siRNA or an shRNA molecule binds to a target RNA sequence that is 100% identical to an RNA sequence encoded by the PTPN2 gene (SEQ ID NO: 3) or the Ptpn2 gene (SEQ ID NO: 4) and the at least one ZC3H12A-targeting siRNA or shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one PTPN2-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 5 or Table 6 and the at least one ZC3H12A-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 7 or Table 8. In some embodiments, the at least one PTPN2-targeting siRNA or shRNA molecule binds to a target RNA sequence that is 100% identical to a RNA sequence encoded by a DNA sequence defined by a set of genomic coordinates shown in Table 5 or Table 6 and the at least one ZC3H12A-targeting siRNA or shRNA molecule binds to a target RNA sequence that is 100% identical to a RNA sequence encoded by a DNA sequence defined by a set of genomic coordinates shown in Table 7 or Table 8.

In some embodiments, the at least one PTPN2-targeting siRNA or shRNA molecule binds to a target RNA sequence that is at least 95%, 96%, 97%, 98%, or 99% identical to an RNA sequence encoded by one of SEQ ID NOs: 185-207 and the at least one ZC3H12A-targeting siRNA or shRNA molecule binds to a target RNA sequence that is at least 95%, 96%, 97%, 98%, or 99% identical to an RNA sequence encoded by one of SEQ ID NOs: 208-230. In some embodiments, the at least one PTPN2-targeting siRNA or shRNA molecule binds to a target RNA sequence that is 100% identical to an RNA sequence encoded by one of SEQ ID NOs: 185-207 and the at least one ZC3H12A-targeting siRNA or shRNA molecule binds to a target RNA sequence that is 100% identical to an RNA sequence encoded by one of SEQ ID NOs: 208-230.

### B. Protein-Based Gene-Regulating Systems

In some embodiments, the present disclosure provides protein gene-regulating systems comprising two or more proteins capable of reducing the expression and/or function of at least two endogenous genes selected from SOCS1, PTPN2, and ZC3H12A. In some embodiments, the present disclosure provides modified immune effector cells comprising such gene-regulating systems. In some embodiments, a protein-based gene-regulating system is a system comprising one or more proteins capable of regulating the expression of an endogenous target gene in a sequence specific manner without the requirement for a nucleic acid guide molecule. In some embodiments, the protein-based gene-regulating system comprises a protein comprising one or more zinc-finger binding domains and an enzymatic domain. In some embodiments, the protein-based gene-regulating system comprises a protein comprising a Transcription activator-like effector nuclease (TALEN) domain and an enzymatic domain. Such embodiments are referred to herein as “TALENs”.

### 1. Zinc Finger Systems

In some embodiments, the present disclosure provides zinc finger gene-regulating systems comprising two or more zinc finger fusion proteins capable of reducing the expression and/or function of at least two endogenous genes selected from SOCS1, PTPN2, and ZC3H12A. In some embodiments, the present disclosure provides modified immune effector cells comprising such gene-regulating systems. Herein, zinc finger-based systems comprise a fusion protein with two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired (e.g., a target locus in a target gene referenced in Tables 2 or 3), one or more zinc finger binding domains can be engineered to bind to one or more target DNA sequences in the target genetic locus. Expression of a fusion protein comprising a zinc finger binding domain and an enzymatic domain in a cell affects modification in the target genetic locus.

In some embodiments, a zinc finger binding domain comprises one or more zinc fingers. Miller et al. (1985) EMBO J. 4:1609-1614; Rhodes (1993) Scientific American Febuary:56-65; U.S. Pat. No. 6,453,242. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore, the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some embodiments, the DNA-binding domains of individual ZFNs comprise between three and six individual zinc finger repeats and can each recognize between 9 and 18 basepairs.

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection.

Selection of a target DNA sequence for binding by a zinc finger domain can be accomplished, for example, according to the methods disclosed in U.S. Pat. No. 6,453,242. It will be clear to those skilled in the art that simple visual inspection of a nucleotide sequence can also be used for selection of a target DNA sequence. Accordingly, any means for target DNA sequence selection can be used in the methods described herein. A target site generally has a length of at least 9 nucleotides and, accordingly, is bound by a zinc finger binding domain comprising at least three zinc fingers. However binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

In some embodiments, the protein-based gene-regulating system comprises at least two zinc finger fusion proteins (ZFP), wherein at least one ZFP comprises a SOCS1-targeting zinc finger binding domain. In some embodiments, the at least one SOCS1-targeting zinc finger binding domain binds to a target DNA sequence that is at least 95%, 96%, 97%, 98%, or 99% identical to a target DNA sequence in the SOCS1 gene (SEQ ID NO: 1) or the Socs1 gene (SEQ ID NO: 2). In some embodiments, the at least one SOCS1-targeting zinc finger binding domain binds to a target DNA sequence that is 100% identical to a target DNA sequence in the SOCS1 gene (SEQ ID NO: 1) or the Socs1 gene (SEQ ID NO: 2).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 3 or Table 4. In some embodiments, the at least one SOCS1-targeting zinc finger binding domain binds to a target DNA sequence that is 100% identical to a DNA sequence defined by a set of genomic coordinates shown in Table 3 or Table 4. In some embodiments, the at least one SOCS1-targeting zinc finger binding domain binds to a target DNA sequence that is at least 90%, 95%, 96%, 97%, 98%, or 99% identical to one of SEQ ID NOs: 7-151. In some embodiments, the at least one SOCS1-targeting zinc finger binding domain binds to a target DNA sequence that is 100% identical to one of SEQ ID NOs: 7-151. Exemplary SOCS1 target DNA sequences are shown in Tables 12 and 13.

In some embodiments, the protein-based gene-regulating system comprises at least two zinc finger fusion proteins (ZFP), wherein at least one ZFP comprises a PTPN2-targeting zinc finger binding domain. In some embodiments, the at least one PTPN2-targeting zinc finger binding domain binds to a target DNA sequence that is at least 95%, 96%, 97%, 98%, or 99% identical to a target DNA sequence in the PTPN2 gene (SEQ ID NO: 3) or the Ptpn2 gene (SEQ ID NO: 4). In some embodiments, the at least one PTPN2-targeting zinc finger binding domain binds to a target DNA sequence that is 100% identical to a target DNA sequence in the PTPN2 gene (SEQ ID NO: 3) or the Ptpn2 gene (SEQ ID NO: 4).

In some embodiments, the at least one PTPN2-targeting zinc finger binding domain binds to a target DNA sequence that is at least 95%, 96%, 97%, 98%, or 99% identical to a DNA sequence defined by a set of genomic coordinates shown in Table 5 or Table 6. In some embodiments, the at least one PTPN2-targeting zinc finger binding domain binds to a target DNA sequence that is 100% identical to a DNA sequence defined by a set of genomic coordinates shown in Table 5 or Table 6. In some embodiments, the at least one PTPN2-targeting zinc finger binding domain binds to a target DNA sequence that is at least 95%, 96%, 97%, 98%, or 99% identical to one of SEQ ID NOs: 185-207. In some embodiments, the at least one PTPN2-targeting zinc finger binding domain binds to a target DNA sequence that is 100% identical to one of SEQ ID NOs: 185-207. Exemplary PTPN2 target DNA sequences are shown in Tables 14 and 15.

In some embodiments, the protein-based gene-regulating system comprises at least two zinc finger fusion proteins (ZFP), wherein at least one ZFP comprises a ZC3H12A-targeting zinc finger binding domain. In some embodiments, the at least one ZC3H12A-targeting zinc finger binding domain binds to a target DNA sequence that is at least 95%, 96%, 97%, 98%, or 99% identical to a target DNA sequence in the ZC3H12A gene (SEQ ID NO: 5) or the Zc3h12a gene (SEQ ID NO: 6). In some embodiments, the at least one ZC3H12A-targeting zinc finger binding domain binds to a target DNA sequence that is 100% identical to a target DNA sequence in the ZC3H12A gene (SEQ ID NO: 5) or the Zc3h12a gene (SEQ ID NO: 6).

In some embodiments, the at least one ZC3H12A-targeting zinc finger binding domain binds to a target DNA sequence that is at least 95%, 96%, 97%, 98%, or 99% identical to a DNA sequence defined by a set of genomic coordinates shown in Table 7 or Table 8. In some embodiments, the at least one ZC3H12A-targeting zinc finger binding domain binds to a target DNA sequence that is 100% identical to a DNA sequence defined by a set of genomic coordinates shown in Table 7 or Table 8. In some embodiments, the at least one ZC3H12A-targeting zinc finger binding domain binds to a target DNA sequence that is at least 95%, 96%, 97%, 98%, or 99% identical to one of SEQ ID NOs: 208-230. In some embodiments, the at least one ZC3H12A-targeting zinc finger binding domain binds to a target DNA sequence that is 100% identical to one of SEQ ID NOs: 208-230. Exemplary ZC3H12A target DNA sequences are shown in Tables 16 and 17.

In some embodiments, the at least one SOCS1-, PTPN2-, or ZC3H12A-targeting ZFP is obtained from a commercial suppliers such as Sigma Aldrich, Dharmacon, ThermoFisher, and the like. For example, in some embodiments, the at least one SOCS1, PTPN2, or ZC3H12A ZFP is one shown in Table 11.

TABLE 11
Exemplary SOCS1, PTPN2, and ZC3H12A Zinc Finger Systems

| Target Gene | Zinc Finger System |
|---|---|
| SOCS1 | CompoZr ® Knockout ZFN plasmid Human |
| | SOCS1 (NM_003745) (SigmaAldrich# |
| | CKOZFND20320) |
| | CompoZr ® Knockout ZFN plasmid Mouse |
| | Socs1 (NM_009896.2) (SigmaAldrich# |
| | CKOZFND41801) |
| PTPN2 | PTPN2 CompoZr ® Knockout ZFN human |
| | plasmid PTPN2 (NM_002828) |
| | (SigmaAldrich# CKOZFND17697) |
| | CompoZr ® Knockout ZFN murine |
| | plasmid Ptpn2 (NM_008977.3) |
| | (SigmaAldrich# CKOZFND39632) |
| ZC3H12A | CompoZr ® Knockout ZFN Kit, ZFN |
| | plasmid Human ZC3H12A (NM_25079) |
| | (SigmaAldrich# CKOZFND23094) |
| | CompoZr ® Knockout ZFN Kit, ZFN |
| | plasmid mouse Zc3h12a (NM_153159.2) |
| | (SigmaAldrich# CKOZFND44851) |

In some embodiments, the protein-based gene-regulating system comprises at least two ZFPs, wherein at least one ZFP comprises a SOCS1-targeting zinc finger binding domain and at least one ZFP comprises a PTPN2-targeting zinc finger binding domain. In some embodiments, the at least one SOCS1-targeting zinc finger binding domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one PTPN2-targeting zinc finger binding domain binds to a target DNA sequence that is at least 95%, 96%, 97%, 98%, or 99% identical to a DNA sequence the PTPN2 gene (SEQ ID NO: 3) or the Ptpn2 gene (SEQ ID NO: 4). In some embodiments, the at least one SOCS1-targeting zinc finger binding domain binds to a target DNA sequence that is 100% identical to a DNA sequence in the SOCS1 gene (SEQ ID NO: 1) or the Socs1 gene (SEQ ID NO: 2) and the at least one PTPN2-targeting zinc finger binding domain binds to a target DNA sequence that is 100% identical to a DNA sequence in the PTPN2 gene (SEQ ID NO: 3) or the Ptpn2 gene (SEQ ID NO: 4).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 3 or Table 4 and the at least one PTPN2-targeting zinc finger binding domain binds to a target DNA sequence that is at least 95%, 96%, 97%, 98%, or 99% identical to a DNA sequence defined by a set of genomic coordinates shown in Table 5 or Table 6. In some embodiments, the at least one SOCS1-targeting zinc finger binding domain binds to a target DNA sequence that is 100% identical to a DNA sequence defined by a set of genomic coordinates shown in Table 3 or Table 4 and the at least one PTPN2-targeting zinc finger binding domain binds to a target DNA sequence that is 100% identical to a DNA sequence defined by a set of genomic coordinates shown in Table 5 or Table 6.

In some embodiments, the at least one SOCS1-targeting zinc finger binding domain binds to a target DNA sequence that is at least 95%, 96%, 97%, 98%, or 99% identical to one of SEQ ID NOs: 7-151 and the at least one PTPN2-targeting zinc finger binding domain binds to a target DNA sequence that is at least 95%, 96%, 97%, 98%, or 99% identical to one of SEQ ID NOs: 185-207. In some embodiments, the at least one SOCS1-targeting zinc finger binding domain binds to a target DNA sequence that is 100% identical to one of SEQ ID NOs: 7-151 and the at least one PTPN2-targeting zinc finger binding domain binds to a target DNA sequence that is 100% identical to one of SEQ ID NOs: 185-207.

In some embodiments, the protein-based gene-regulating system comprises at least two ZFPs, wherein at least one ZFP comprises a SOCS1-targeting zinc finger binding domain and at least one ZFP comprises a ZC3H12A-targeting zinc finger binding domain. In some embodiments, the at least one SOCS1-targeting zinc finger binding domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one ZC3H12A-targeting zinc finger binding domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one SOCS1-targeting zinc finger binding domain binds to a target DNA sequence that is 100% identical to a DNA sequence in the SOCS1 gene (SEQ ID NO: 1) or the Socs1 gene (SEQ ID NO: 2) and the at least one ZC3H12A-targeting zinc finger binding domain binds to a target DNA sequence that is 100% identical to a DNA sequence in the ZC3H12A gene (SEQ ID NO: 5) or the Zc3h12a gene (SEQ ID NO: 6).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 3 or Table 4 and the at least one ZC3H12A-targeting zinc finger binding domain binds to a target DNA sequence that is at least 95%, 96%, 97%, 98%, or 99% identical to a DNA sequence defined by a set of genomic coordinates shown in Table 7 or Table 8. In some embodiments, the at least one SOCS1-targeting zinc finger binding domain binds to a target DNA sequence that is 100% identical to a DNA sequence defined by a set of genomic coordinates shown in Table 3 or Table 4 and the at least one ZC3H12A-targeting zinc finger binding domain binds to a target DNA sequence that is 100% identical to a DNA sequence defined by a set of genomic coordinates shown in Table 7 or Table 8.

In some embodiments, the at least one SOCS1-targeting zinc finger binding domain binds to a target DNA sequence that is at least 95%, 96%, 97%, 98%, or 99% identical to one of SEQ ID NOs: 7-151 and the at least one ZC3H12A-targeting zinc finger binding domain binds to a target DNA sequence that is at least 95%, 96%, 97%, 98%, or 99% identical to one of SEQ ID NOs: 208-230. In some embodiments, the at least one SOCS1-targeting zinc finger binding domain binds to a target DNA sequence that is 100% identical to one of SEQ ID NOs: 7-151 and the at least one ZC3H12A-targeting zinc finger binding domain binds to a target DNA sequence that is 100% identical to one of SEQ ID NOs: 208-230.

In some embodiments, the protein-based gene-regulating system comprises at least two ZFPs, wherein at least one ZFP comprises a PTPN2-targeting zinc finger binding domain and at least one ZFP comprises a ZC3H12A-targeting zinc finger binding domain. In some embodiments, the at least one PTPN2-targeting zinc finger binding domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4) and the at least one ZC3H12A-targeting zinc finger binding domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one PTPN2-targeting zinc finger binding domain binds to a target DNA sequence that is 100% identical to a DNA sequence in the PTPN2 gene (SEQ ID NO: 3) or the Ptpn2 gene (SEQ ID NO: 4) and the at least one ZC3H12A-targeting zinc finger binding domain binds to a target DNA sequence that is 100% identical to a DNA sequence in the ZC3H12A gene (SEQ ID NO: 5) or the Zc3h12a gene (SEQ ID NO: 6).

In some embodiments, the at least one PTPN2-targeting zinc finger binding domain binds to a target DNA sequence that is at least 95%, 96%, 97%, 98%, or 99% identical to a DNA sequence defined by a set of genomic coordinates shown in Table 5 or Table 6 and the at least one ZC3H12A-targeting zinc finger binding domain binds to a target DNA sequence that is at least 95%, 96%, 97%, 98%, or 99% identical to a DNA sequence defined by a set of genomic coordinates shown in Table 7 or Table 8. In some embodiments, the at least one PTPN2-targeting zinc finger binding domain binds to a target DNA sequence that is 100% identical to a DNA sequence defined by a set of genomic coordinates shown in Table 5 or Table 6 and the at least one ZC3H12A-targeting zinc finger binding domain binds to a target DNA sequence that is 100% identical to a DNA sequence defined by a set of genomic coordinates shown in Table 7 or Table 8.

In some embodiments, the at least one PTPN2-targeting zinc finger binding domain binds to a target DNA sequence that is at least 95%, 96%, 97%, 98%, or 99% identical to one of SEQ ID NOs: 185-207 and the at least one ZC3H12A-targeting zinc finger binding domain binds to a target DNA sequence that is at least 95%, 96%, 97%, 98%, or 99% identical to one of SEQ ID NOs: 208-230. In some embodiments, the at least one PTPN2-targeting zinc finger binding domain binds to a target DNA sequence that is 100% identical to one of SEQ ID NOs: 185-207 and the at least one ZC3H12A-targeting zinc finger binding domain binds to a target DNA sequence that is 100% identical to one of SEQ ID NOs: 208-230.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNasel; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains.

Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the enzymatic domain from at least one Type IIS restriction enzyme and one or more zinc finger binding domains.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Thus, for targeted double-stranded DNA cleavage using zinc finger-FokI fusions, two fusion proteins, each comprising a FokI enzymatic domain, can be used to reconstitute a catalytically active cleavage domain. Alternatively, a single polypeptide molecule containing a zinc finger binding domain and two FokI enzymatic domains can also be used. Exemplary ZFPs comprising FokI enzymatic domains are described in U.S. Pat. No. 9,782,437.

### 2. TALEN Systems

In some embodiments, the present disclosure provides TALEN gene-regulating systems comprising two or more TALEN fusion proteins capable of reducing the expression and/or function of at least two endogenous genes selected from SOCS1, PTPN2, and ZC3H12A. In some embodiments, the present disclosure provides modified immune effector cells comprising such gene-regulating systems. TALEN-based systems comprise a TALEN fusion protein comprising a TAL effector DNA binding domain and an enzymatic domain. They are made by fusing a TAL effector DNA-binding domain to a DNA cleavage domain (a nuclease which cuts DNA strands). The FokI restriction enzyme described above is an exemplary enzymatic domain suitable for use in TALEN-based gene-regulating systems.

TAL effectors are proteins that are secreted by Xanthomonas bacteria via their type III secretion system when they infect plants. The DNA binding domain contains a repeated, highly conserved, 33-34 amino acid sequence with divergent 12th and 13th amino acids. These two positions, referred to as the Repeat Variable Diresidue (RVD), are highly variable and strongly correlated with specific nucleotide recognition. Therefore, the TAL effector domains can be engineered to bind specific target DNA sequences by selecting a combination of repeat segments containing the appropriate RVDs. The nucleic acid specificity for RVD combinations is as follows: HD targets cytosine, NI targets adenine, NG targets thymine, and NN targets guanine (though, in some embodiments, NN can also bind adenenine with lower specificity).

Methods and compositions for assembling the TAL-effector repeats are known in the art. See e.g., Cermak et al, Nucleic Acids Research, 39:12, 2011, e82. Plasmids for constructions of the TAL-effector repeats are commercially available from Addgene.

In some embodiments, the protein-based gene-regulating system comprises at least two TALEN fusion proteins, wherein at least one TALEN fusion protein comprises a SOCS1-targeting TAL effector domain. In some embodiments, the at least one SOCS1-targeting TAL effector domain binds to a target DNA sequence that is at least 95%, 96%, 97%, 98%, or 99% identical to a target DNA sequence in the SOCS1 gene (SEQ ID NO: 1) or the Socs1 gene (SEQ ID NO: 2). In some embodiments, the at least one SOCS1-targeting TAL effector domain binds to a target DNA sequence that is 100% identical to a target DNA sequence in the SOCS1 gene (SEQ ID NO: 1) or the Socs1 gene (SEQ ID NO: 1).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 3 or Table 4. In some embodiments, the at least one SOCS1-targeting TAL effector domain binds to a target DNA sequence that is 100% identical to a DNA sequence defined by a set of genomic coordinates shown in Table 3 or Table 4. In some embodiments, the at least one SOCS1-targeting TAL effector domain binds to a target DNA sequence that is at least 95%, 96%, 97%, 98%, or 99% identical to one of SEQ ID NOs: 7-151. In some embodiments, the at least one SOCS1-targeting TAL effector domain binds to a target DNA sequence that is 100% identical to one of SEQ ID NOs: 7-151. Exemplary SOCS1 target DNA sequences are shown in Tables 12 and 13.

In some embodiments, the protein-based gene-regulating system comprises at least two TALEN fusion proteins, wherein at least one TALEN fusion protein comprises a PTPN2-targeting TAL effector domain. In some embodiments, the at least one PTPN2-targeting TAL effector domain binds to a target DNA sequence that is at least 95%, 96%, 97%, 98%, or 99% identical to a target DNA sequence in the PTPN2 gene (SEQ ID NO: 3) or the Ptpn2 gene (SEQ ID NO: 4). In some embodiments, the at least one PTPN2-targeting TAL effector domain binds to a target DNA sequence that is 100% identical to a target DNA sequence in the PTPN2 gene (SEQ ID NO: 3) or the Ptpn2 gene (SEQ ID NO: 4).

In some embodiments, the at least one PTPN2-targeting TAL effector domain binds to a target DNA sequence that is at least 95%, 96%, 97%, 98%, or 99% identical to a DNA sequence defined by a set of genomic coordinates shown in Table 5 or Table 6. In some embodiments, the at least one PTPN2-targeting TAL effector domain binds to a target DNA sequence that is 100% identical to a DNA sequence defined by a set of genomic coordinates shown in Table 5 or Table 6. In some embodiments, the at least one PTPN2-targeting TAL effector domain binds to a target DNA sequence that is at least 95%, 96%, 97%, 98%, or 99% identical to one of SEQ ID NOs: 185-207. In some embodiments, the at least one PTPN2-targeting TAL effector domain binds to a target DNA sequence that is 100% identical to one of SEQ ID NOs: 185-207. Exemplary PTPN2 target DNA sequences are shown in Tables 14 and 15.

In some embodiments, the protein-based gene-regulating system comprises at least two TALEN fusion proteins, wherein at least one TALEN fusion protein comprises a ZC3H12A-targeting TAL effector domain. In some embodiments, the at least one ZC3H12A-targeting TAL effector domain binds to a target DNA sequence that is at least 95%, 96%, 97%, 98%, or 99% identical to a target DNA sequence in the ZC3H12A gene (SEQ ID NO: 5) or the Zc3h12a gene (SEQ ID NO: 6). In some embodiments, the at least one ZC3H12A-targeting TAL effector domain binds to a target DNA sequence that is 100% identical to a target DNA sequence in the ZC3H12A gene (SEQ ID NO: 5) or the Zc3h12a gene (SEQ ID NO: 6).

In some embodiments, the at least one ZC3H12A-targeting TAL effector domain binds to a target DNA sequence that is at least 95%, 96%, 97%, 98%, or 99% identical to a DNA sequence defined by a set of genomic coordinates shown in Table 7 or Table 8. In some embodiments, the at least one ZC3H12A-targeting TAL effector domain binds to a target DNA sequence that is 100% identical to a DNA sequence defined by a set of genomic coordinates shown in Table 7 or Table 8. In some embodiments, the at least one ZC3H12A-targeting TAL effector domain binds to a target DNA sequence that is at least 95%, 96%, 97%, 98%, or 99% identical to one of SEQ ID NOs: 208-230. In some embodiments, the at least one ZC3H12A-targeting TAL effector domain binds to a target DNA sequence that is 100% identical to one of SEQ ID NOs: 208-230. Exemplary ZC3H12A target DNA sequences are shown in Tables 16 and 17.

In some embodiments, the protein-based gene-regulating system comprises at least two TAL fusion proteins, wherein at least one TALEN fusion protein comprises a SOCS1-targeting TAL effector domain and at least one TALEN fusion protein comprises a PTPN2-targeting TAL effector domain. In some embodiments, the at least one SOCS1-targeting TAL effector domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one PTPN2-targeting TAL effector domain binds to a target DNA sequence that is at least 95%, 96%, 97%, 98%, or 99% identical to a DNA sequence the PTPN2 gene (SEQ ID NO: 3) or the Ptpn2 gene (SEQ ID NO: 4). In some embodiments, the at least one SOCS1-targeting TAL effector domain binds to a target DNA sequence that is 100% identical to a DNA sequence in the SOCS1 gene (SEQ ID NO: 1) or the Socs1 gene (SEQ ID NO: 2) and the at least one PTPN2-targeting TAL effector domain binds to a target DNA sequence that is 100% identical to a DNA sequence in the PTPN2 gene (SEQ ID NO: 3) or the Ptpn2 gene (SEQ ID NO: 4).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 3 or Table 4 and the at least one PTPN2-targeting TAL effector domain binds to a target DNA sequence that is at least 95%, 96%, 97%, 98%, or 99% identical to a DNA sequence defined by a set of genomic coordinates shown in Table 5 or Table 6. In some embodiments, the at least one SOCS1-targeting TAL effector domain binds to a target DNA sequence that is 100% identical to a DNA sequence defined by a set of genomic coordinates shown in Table 3 or Table 4 and the at least one PTPN2-targeting TAL effector domain binds to a target DNA sequence that is 100% identical to a DNA sequence defined by a set of genomic coordinates shown in Table 5 or Table 6.

In some embodiments, the at least one SOCS1-targeting TAL effector domain binds to a target DNA sequence that is at least 95%, 96%, 97%, 98%, or 99% identical to one of SEQ ID NOs: 7-151 and the at least one PTPN2-targeting TAL effector domain binds to a target DNA sequence that is at least 95%, 96%, 97%, 98%, or 99% identical to one of SEQ ID NOs: 185-207. In some embodiments, the at least one SOCS1-targeting TAL effector domain binds to a target DNA sequence that is 100% identical to one of SEQ ID NOs: 7-151 and the at least one PTPN2-targeting TAL effector domain binds to a target DNA sequence that is 100% identical to one of SEQ ID NOs: 185-207.

In some embodiments, the protein-based gene-regulating system comprises at least two TALEN fusion proteins, wherein at least one TALEN fusion protein comprises a SOCS1-targeting TAL effector domain and at least one TALEN fusion protein comprises a ZC3H12A-targeting TAL effector domain. In some embodiments, the at least one SOCS1-targeting TAL effector domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one ZC3H12A-targeting TAL effector domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one SOCS1-targeting TAL effector domain binds to a target DNA sequence that is 100% identical to a DNA sequence in the SOCS1 gene (SEQ ID NO: 1) or the Socs1 gene (SEQ ID NO: 2) and the at least one ZC3H12A-targeting TAL effector domain binds to a target DNA sequence that is 100% identical to a DNA sequence in the ZC3H12A gene (SEQ ID NO: 5) or the Zc3h12a gene (SEQ ID NO: 6).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 3 or Table 4 and the at least one ZC3H12A-targeting TAL effector domain binds to a target DNA sequence that is at least 95%, 96%, 97%, 98%, or 99% identical to a DNA sequence defined by a set of genomic coordinates shown in Table 7 or Table 8. In some embodiments, the at least one SOCS1-targeting TAL effector domain binds to a target DNA sequence that is 100% identical to a DNA sequence defined by a set of genomic coordinates shown in Table 3 or Table 4 and the at least one ZC3H12A-targeting TAL effector domain binds to a target DNA sequence that is 100% identical to a DNA sequence defined by a set of genomic coordinates shown in Table 7 or Table 8.

In some embodiments, the at least one SOCS1-targeting TAL effector domain binds to a target DNA sequence that is at least 95%, 96%, 97%, 98%, or 99% identical to one of SEQ ID NOs: 7-151 and the at least one ZC3H12A-targeting TAL effector domain binds to a target DNA sequence that is at least 95%, 96%, 97%, 98%, or 99% identical to one of SEQ ID NOs: 208-230. In some embodiments, the at least one SOCS1-targeting TAL effector domain binds to a target DNA sequence that is 100% identical to one of SEQ ID NOs: 7-151 and the at least one ZC3H12A-targeting TAL effector domain binds to a target DNA sequence that is 100% identical to one of SEQ ID NOs: 208-230.

In some embodiments, the protein-based gene-regulating system comprises at least two TALEN fusion proteins, wherein at least one TALEN fusion protein comprises a PTPN2-targeting TAL effector domain and at least one TALEN fusion protein comprises a ZC3H12A-targeting TAL effector domain. In some embodiments, the at least one PTPN2-targeting TAL effector domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4) and the at least one ZC3H12A-targeting TAL effector domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one PTPN2-targeting TAL effector domain binds to a target DNA sequence that is 100% identical to a DNA sequence in the PTPN2 gene (SEQ ID NO: 3) or the Ptpn2 gene (SEQ ID NO: 4) and the at least one ZC3H12A-targeting TAL effector domain binds to a target DNA sequence that is 100% identical to a DNA sequence in the ZC3H12A gene (SEQ ID NO: 5) or the Zc3h12a gene (SEQ ID NO: 6).

In some embodiments, the at least one PTPN2-targeting TAL effector domain binds to a target DNA sequence that is at least 95%, 96%, 97%, 98%, or 99% identical to a DNA sequence defined by a set of genomic coordinates shown in Table 5 or Table 6 and the at least one ZC3H12A-targeting TAL effector domain binds to a target DNA sequence that is at least 95%, 96%, 97%, 98%, or 99% identical to a DNA sequence defined by a set of genomic coordinates shown in Table 7 or Table 8. In some embodiments, the at least one PTPN2-targeting TAL effector domain binds to a target DNA sequence that is 100% identical to a DNA sequence defined by a set of genomic coordinates shown in Table 5 or Table 6 and the at least one ZC3H12A-targeting TAL effector domain binds to a target DNA sequence that is 100% identical to a DNA sequence defined by a set of genomic coordinates shown in Table 7 or Table 8.

In some embodiments, the at least one PTPN2-targeting TAL effector domain binds to a target DNA sequence that is at least 95%, 96%, 97%, 98%, or 99% identical to one of SEQ ID NOs: 185-207 and the at least one ZC3H12A-targeting TAL effector domain binds to a target DNA sequence that is at least 95%, 96%, 97%, 98%, or 99% identical to one of SEQ ID NOs: 208-230. In some embodiments, the at least one PTPN2-targeting TAL effector domain binds to a target DNA sequence that is 100% identical to one of SEQ ID NOs: 185-207 and the at least one ZC3H/2A-targeting TAL effector domain binds to a target DNA sequence that is 100% identical to one of SEQ ID NOs: 208-230.

### C. Combination Nucleic Acid/Protein-Based Gene-Regulating Systems

Combination gene-regulating systems comprise a site-directed modifying polypeptide and a nucleic acid guide molecule. Herein, a “site-directed modifying polypeptide” refers to a polypeptide that binds to a nucleic acid guide molecule, is targeted to a target nucleic acid sequence, (for example, an endogenous target DNA or RNA sequence) by the nucleic acid guide molecule to which it is bound, and modifies the target nucleic acid sequence (e.g., cleavage, mutation, or methylation of a target nucleic acid sequence).

A site-directed modifying polypeptide comprises two portions, a portion that binds the nucleic acid guide and an activity portion. In some embodiments, a site-directed modifying polypeptide comprises an activity portion that exhibits site-directed enzymatic activity (e.g., DNA methylation, DNA or RNA cleavage, histone acetylation, histone methylation, etc.), wherein the site of enzymatic activity is determined by the guide nucleic acid. In some cases, a site-directed modifying polypeptide comprises an activity portion that has enzymatic activity that modifies the endogenous target nucleic acid sequence (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, a site-directed modifying polypeptide comprises an activity portion that has enzymatic activity that modifies a polypeptide (e.g., a histone) associated with the endogenous target nucleic acid sequence (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). In some embodiments, a site-directed modifying polypeptide comprises an activity portion that modulates transcription of a target DNA sequence (e.g., to increase or decrease transcription). In some embodiments, a site-directed modifying polypeptide comprises an activity portion that modulates expression or translation of a target RNA sequence (e.g., to increase or decrease transcription).

The nucleic acid guide comprises two portions: a first portion that is complementary to, and capable of binding with, an endogenous target nucleic sequence (referred to herein as a “nucleic acid-binding segment”), and a second portion that is capable of interacting with the site-directed modifying polypeptide (referred to herein as a “protein-binding segment”). In some embodiments, the nucleic acid-binding segment and protein-binding segment of a nucleic acid guide are comprised within a single polynucleotide molecule. In some embodiments, the nucleic acid-binding segment and protein-binding segment of a nucleic acid guide are each comprised within separate polynucleotide molecules, such that the nucleic acid guide comprises two polynucleotide molecules that associate with each other to form the functional guide.

The nucleic acid guide mediates the target specificity of the combined protein/nucleic acid gene-regulating systems by specifically hybridizing with a target nucleic acid sequence. In some embodiments, the target nucleic acid sequence is an RNA sequence, such as an RNA sequence comprised within an mRNA transcript of a target gene. In some embodiments, the target nucleic acid sequence is a DNA sequence comprised within the DNA sequence of a target gene. Reference herein to a target gene encompasses the full-length DNA sequence for that particular gene which comprises a plurality of target genetic loci (i.e., portions of a particular target gene sequence (e.g., an exon or an intron)). Within each target genetic loci are shorter stretches of DNA sequences referred to herein as “target DNA sequences” that can be modified by the gene-regulating systems described herein. Further, each target genetic loci comprises a “target modification site,” which refers to the precise location of the modification induced by the gene-regulating system (e.g., the location of an insertion, a deletion, or mutation, the location of a DNA break, or the location of an epigenetic modification). The gene-regulating systems described herein may comprise 2 or more nucleic acid guides (e.g., 2, 3, 4, 5, 6, 7, 8, 9, 10, or more nucleic acid guides).

In some embodiments, the combined protein/nucleic acid gene-regulating systems comprise site-directed modifying polypeptides derived from Argonaute (Ago) proteins (e.g., T. thermophiles Ago or TtAgo). In such embodiments, the site-directed modifying polypeptide is a T. thermophiles Ago DNA endonuclease and the nucleic acid guide is a guide DNA (gDNA) (See, Swarts et al., Nature 507 (2014), 258-261). In some embodiments, the present disclosure provides a polynucleotide encoding a gDNA. In some embodiments, a gDNA-encoding nucleic acid is comprised in an expression vector, e.g., a recombinant expression vector. In some embodiments, the present disclosure provides a polynucleotide encoding a TtAgo site-directed modifying polypeptide or variant thereof. In some embodiments, the polynucleotide encoding a TtAgo site-directed modifying polypeptide is comprised in an expression vector, e.g., a recombinant expression vector.

In some embodiments, the gene editing systems described herein are CRISPR (Clustered Regularly Interspaced Short Palindromic Repeats)/Cas (CRISPR Associated) nuclease systems. In some embodiments, the CRISPR/Cas system is a Class 2 system. Class 2 CRISPR/Cas systems are divided into three types: Type II, Type V, and Type VI systems. In some embodiments, the CRISPR/Cas system is a Class 2 Type II system, utilizing the Cas9 protein. In such embodiments, the site-directed modifying polypeptide is a Cas9 DNA endonuclease (or variant thereof) and the nucleic acid guide molecule is a guide RNA (gRNA). In some embodiments, the CRISPR/Cas system is a Class 2 Type V system, utilizing the Cas12 proteins (e.g., Cas12a (also known as Cpf1), Cas12b (also known as C2c1), Cas12c (also known as C2c3), Cas12d (also known as CasY), and Cas12e (also known as CasX)). In such embodiments, the site-directed modifying polypeptide is a Cas12 DNA endonuclease (or variant thereof) and the nucleic acid guide molecule is a gRNA. In some embodiments, the CRISPR/Cas system is a Class 2 and Type VI system, utilizing the Cas13 proteins (e.g., Cas13a (also known as C2c2), Cas13b, and Cas13c). (See, Pyzocha et al., ACS Chemical Biology, 13(2), 347-356). In such embodiments, the site-directed modifying polypeptide is a Cas13 RNA riboendonuclease and the nucleic acid guide molecule is a gRNA.

A Cas polypeptide refers to a polypeptide that can interact with a gRNA molecule and, in concert with the gRNA molecule, home or localize to a target DNA or target RNA sequence. Cas polypeptides include naturally occurring Cas proteins and engineered, altered, or otherwise modified Cas proteins that differ by one or more amino acid residues from a naturally-occurring Cas sequence.

A guide RNA (gRNA) comprises two segments, a DNA-binding segment and a protein-binding segment. In some embodiments, the protein-binding segment of a gRNA is comprised in one RNA molecule and the DNA-binding segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The protein-binding segment of a gRNA comprises, in part, two complementary stretches of nucleotides that hybridize to one another to form a double stranded RNA duplex (dsRNA duplex), which facilitates binding to the Cas protein. The nucleic acid-binding segment (or “nucleic acid-binding sequence”) of a gRNA comprises a nucleotide sequence that is complementary to and capable of binding to a specific target nucleic acid sequence. The protein-binding segment of the gRNA interacts with a Cas polypeptide and the interaction of the gRNA molecule and site-directed modifying polypeptide results in Cas binding to the endogenous nucleic acid sequence and produces one or more modifications within or around the target nucleic acid sequence. The precise location of the target modification site is determined by both (i) base-pairing complementarity between the gRNA and the target nucleic acid sequence; and (ii) the location of a short motif, referred to as the protospacer adjacent motif (PAM), in the target DNA sequence (referred to as a protospacer flanking sequence (PFS) in target RNA sequences). The PAM/PFS sequence is required for Cas binding to the target nucleic acid sequence. A variety of PAM/PFS sequences are known in the art and are suitable for use with a particular Cas endonuclease (e.g., a Cas9 endonuclease). (See e.g., Nat Methods. 2013 November; 10(11): 1116-1121 and Sci Rep. 2014; 4: 5405). In some embodiments, the PAM sequence is located within 50 base pairs of the target modification site in a target DNA sequence. In some embodiments, the PAM sequence is located within 10 base pairs of the target modification site in a target DNA sequence. The DNA sequences that can be targeted by this method are limited only by the relative distance of the PAM sequence to the target modification site and the presence of a unique 20 base pair sequence to mediate sequence-specific, gRNA-mediated Cas binding. In some embodiments, the PFS sequence is located at the 3′ end of the target RNA sequence. In some embodiments, the target modification site is located at the 5′ terminus of the target locus. In some embodiments, the target modification site is located at the 3′ end of the target locus. In some embodiments, the target modification site is located within an intron or an exon of the target locus.

In some embodiments, the present disclosure provides a polynucleotide encoding a gRNA. In some embodiments, a gRNA-encoding nucleic acid is comprised in an expression vector, e.g., a recombinant expression vector. In some embodiments, the present disclosure provides a polynucleotide encoding a site-directed modifying polypeptide. In some embodiments, the polynucleotide encoding a site-directed modifying polypeptide is comprised in an expression vector, e.g., a recombinant expression vector.

### 1. Cas Proteins

In some embodiments, the site-directed modifying polypeptide is a Cas protein. Cas molecules of a variety of species can be used in the methods and compositions described herein, including Cas molecules derived from S. pyogenes, S. aureus, N. meningitidis, S. thermophiles, Acidovorax avenae, Actinobacillus pleuropneumonias, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

In some embodiments, the Cas protein is a naturally-occurring Cas protein. In some embodiments, the Cas endonuclease is selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas protein is an endoribonuclease such as a Cas13 protein. In some embodiments, the Cas13 protein is a Cas13a (Abudayyeh et al., Nature 550 (2017), 280-284), Cas13b (Cox et al., Science (2017) 358:6336, 1019-1027), Cas13c (Cox et al., Science (2017) 358:6336, 1019-1027), or Cas13d (Zhang et al., Cell 175 (2018), 212-223) protein.

In some embodiments, the Cas protein is a wild-type or naturally occurring Cas9 protein or a Cas9 ortholog. Wild-type Cas9 is a multi-domain enzyme that uses an HNH nuclease domain to cleave the target strand of DNA and a RuvC-like domain to cleave the non-target strand. Binding of WT Cas9 to DNA based on gRNA specificity results in double-stranded DNA breaks that can be repaired by non-homologous end joining (NHEJ) or homology-directed repair (HDR). Exemplary naturally occurring Cas9 molecules are described in Chylinski et al., RNA Biology 2013 10:5, 727-737 and additional Cas9 orthologs are described in International PCT Publication No. WO 2015/071474. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

In some embodiments, the naturally occurring Cas9 polypeptide is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some embodiments, the Cas polypeptide comprises one or more of the following activities:

(a) a nickase activity, i.e., the ability to cleave a single strand, e.g., the non-complementary strand or the complementary strand, of a nucleic acid molecule;

(b) a double stranded nuclease activity, i.e., the ability to cleave both strands of a double stranded nucleic acid and create a double stranded break, which in an embodiment is the presence of two nickase activities;

(c) an endonuclease activity;

(d) an exonuclease activity; and/or

(e) a helicase activity, i.e., the ability to unwind the helical structure of a double stranded nucleic acid.

In some embodiments, the Cas polypeptide is fused to heterologous proteins that recruit DNA-damage signaling proteins, exonucleases, or phosphatases to further increase the likelihood or the rate of repair of the target sequence by one repair mechanism or another. In some embodiments, a WT Cas polypeptide is co-expressed with a nucleic acid repair template to facilitate the incorporation of an exogenous nucleic acid sequence by homology-directed repair.

In some embodiments, different Cas proteins (i.e., Cas9 proteins from various species) may be advantageous to use in the various provided methods in order to capitalize on various enzymatic characteristics of the different Cas proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some embodiments, the Cas protein is a Cas9 protein derived from S. pyogenes and recognizes the PAM sequence motif NGG, NAG, NGA (Mali et al, Science 2013; 339(6121): 823-826). In some embodiments, the Cas protein is a Cas9 protein derived from S. thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the Cas protein is a Cas9 protein derived from S. mutans and recognizes the PAM sequence motif NGG and/or NAAR (R=A or G) (See, e.g., Deveau et al, J BACTERIOL 2008; 190(4): 1390-1400). In some embodiments, the Cas protein is a Cas9 protein derived from S. aureus and recognizes the PAM sequence motif NNGRR (R=A or G). In some embodiments, the Cas protein is a Cas9 protein derived from S. aureus and recognizes the PAM sequence motif N GRRT (R=A or G). In some embodiments, the Cas protein is a Cas9 protein derived from S. aureus and recognizes the PAM sequence motif N GRRV (R=A or G). In some embodiments, the Cas protein is a Cas9 protein derived from N. meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the Cas protein is a Cas13a protein derived from Leptotrichia shahii and recognizes the PFS sequence motif of a single 3′ A, U, or C.

In some embodiments, a polynucleotide encoding a Cas protein is provided. In some embodiments, the polynucleotide encodes a Cas protein that is at least 90% identical to a Cas protein described in International PCT Publication No. WO 2015/071474 or Chylinski et al., RNA Biology 2013 10:5, 727-737. In some embodiments, the polynucleotide encodes a Cas protein that is at least 95%, 96%, 97%, 98%, or 99% identical to a Cas protein described in International PCT Publication No. WO 2015/071474 or Chylinski et al., RNA Biology 2013 10:5, 727-737. In some embodiments, the polynucleotide encodes a Cas protein that is 100% identical to a Cas protein described in International PCT Publication No. WO 2015/071474 or Chylinski et al., RNA Biology 2013 10:5, 727-737.

### 2. Cas Mutants

In some embodiments, the Cas polypeptides are engineered to alter one or more properties of the Cas polypeptide. For example, in some embodiments, the Cas polypeptide comprises altered enzymatic properties, e.g., altered nuclease activity, (as compared with a naturally occurring or other reference Cas molecule) or altered helicase activity. In some embodiments, an engineered Cas polypeptide can have an alteration that alters its size, e.g., a deletion of amino acid sequence that reduces its size without significant effect on another property of the Cas polypeptide. In some embodiments, an engineered Cas polypeptide comprises an alteration that affects PAM recognition. For example, an engineered Cas polypeptide can be altered to recognize a PAM sequence other than the PAM sequence recognized by the corresponding wild-type Cas protein.

Cas polypeptides with desired properties can be made in a number of ways, including alteration of a naturally occurring Cas polypeptide or parental Cas polypeptide, to provide a mutant or altered Cas polypeptide having a desired property. For example, one or more mutations can be introduced into the sequence of a parental Cas polypeptide (e.g., a naturally occurring or engineered Cas polypeptide). Such mutations and differences may comprise substitutions (e.g., conservative substitutions or substitutions of non-essential amino acids); insertions; or deletions. In some embodiments, a mutant Cas polypeptide comprises one or more mutations (e.g., at least 1, 2, 3, 4, 5, 10, 15, 20, 30, 40 or 50 mutations) relative to a parental Cas polypeptide.

In an embodiment, a mutant Cas polypeptide comprises a cleavage property that differs from a naturally occurring Cas polypeptide. In some embodiments, the Cas is a deactivated Cas (dCas) mutant. In such embodiments, the Cas polypeptide does not comprise any intrinsic enzymatic activity and is unable to mediate target nucleic acid cleavage. In such embodiments, the dCas may be fused with a heterologous protein that is capable of modifying the target nucleic acid in a non-cleavage based manner. For example, in some embodiments, a dCas protein is fused to transcription activator or transcription repressor domains (e.g., the Kruppel associated box (KRAB or SKD); the Mad mSIN3 interaction domain (SID or SID4X); the ERF repressor domain (ERD); the MAX-interacting protein 1 (MXI1); methyl-CpG binding protein 2 (MECP2); etc.). In some such cases, the dCas fusion protein is targeted by the gRNA to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target DNA or modifies a polypeptide associated with the target DNA). In some cases, the changes are transient (e.g., transcription repression or activation). In some cases, the changes are inheritable (e.g., when epigenetic modifications are made to the target DNA or to proteins associated with the target DNA, e.g., nucleosomal histones).

In some embodiments, the dCas is a dCas13 mutant (Konermann et al., Cell 173 (2018), 665-676). These dCas13 mutants can then be fused to enzymes that modify RNA, including adenosine deaminases (e.g., ADAR1 and ADAR2). Adenosine deaminases convert adenine to inosine, which the translational machinery treats like guanine, thereby creating a functional A→G change in the RNA sequence. In some embodiments, the dCas is a dCas9 mutant.

In some embodiments, the mutant Cas9 is a Cas9 nickase mutant. Cas9 nickase mutants comprise only one catalytically active domain (either the HNH domain or the RuvC domain). The Cas9 nickase mutants retain DNA binding based on gRNA specificity, but are capable of cutting only one strand of DNA resulting in a single-strand break (e.g. a “nick”). In some embodiments, two complementary Cas9 nickase mutants (e.g., one Cas9 nickase mutant with an inactivated RuvC domain, and one Cas9 nickase mutant with an inactivated HNH domain) are expressed in the same cell with two gRNAs corresponding to two respective target sequences; one target sequence on the sense DNA strand, and one on the antisense DNA strand. This dual-nickase system results in staggered double stranded breaks and can increase target specificity, as it is unlikely that two off-target nicks will be generated close enough to generate a double stranded break. In some embodiments, a Cas9 nickase mutant is co-expressed with a nucleic acid repair template to facilitate the incorporation of an exogenous nucleic acid sequence by homology-directed repair.

In some embodiments, the Cas polypeptides described herein can be engineered to alter the PAM/PFS specificity of the Cas polypeptide. In some embodiments, a mutant Cas polypeptide has a PAM/PFS specificity that is different from the PAM/PFS specificity of the parental Cas polypeptide. For example, a naturally occurring Cas protein can be modified to alter the PAM/PFS sequence that the mutant Cas polypeptide recognizes to decrease off target sites, improve specificity, or eliminate a PAM/PFS recognition requirement. In some embodiments, a Cas protein can be modified to increase the length of the PAM/PFS recognition sequence. In some embodiments, the length of the PAM recognition sequence is at least 4, 5, 6, 7, 8, 9, 10 or 15 amino acids in length. Cas polypeptides that recognize different PAM/PFS sequences and/or have reduced off-target activity can be generated using directed evolution. Exemplary methods and systems that can be used for directed evolution of Cas polypeptides are described, e.g., in Esvelt et al. Nature 2011, 472(7344): 499-503.

Exemplary Cas mutants are described in International PCT Publication No. WO 2015/161276 and Konermann et al., Cell 173 (2018), 665-676 which are incorporated herein by reference in their entireties.

3. gRNAs

The present disclosure provides guide RNAs (gRNAs) that direct a site-directed modifying polypeptide to a specific target nucleic acid sequence. A gRNA comprises a nucleic acid-targeting segment and protein-binding segment. The nucleic acid-targeting segment of a gRNA comprises a nucleotide sequence that is complementary to a sequence in the target nucleic acid sequence. As such, the nucleic acid-targeting segment of a gRNA interacts with a target nucleic acid in a sequence-specific manner via hybridization (i.e., base pairing), and the nucleotide sequence of the nucleic acid-targeting segment determines the location within the target nucleic acid that the gRNA will bind. The nucleic acid-targeting segment of a gRNA can be modified (e.g., by genetic engineering) to hybridize to any desired sequence within a target nucleic acid sequence.

The protein-binding segment of a guide RNA interacts with a site-directed modifying polypeptide (e.g. a Cas protein) to form a complex. The guide RNA guides the bound polypeptide to a specific nucleotide sequence within target nucleic acid via the above-described nucleic acid-targeting segment. The protein-binding segment of a guide RNA comprises two stretches of nucleotides that are complementary to one another and which form a double stranded RNA duplex.

In some embodiments, a gRNA comprises two separate RNA molecules. In such embodiments, each of the two RNA molecules comprises a stretch of nucleotides that are complementary to one another such that the complementary nucleotides of the two RNA molecules hybridize to form the double-stranded RNA duplex of the protein-binding segment. In some embodiments, a gRNA comprises a single RNA molecule (sgRNA).

The specificity of a gRNA for a target locus is mediated by the sequence of the nucleic acid-binding segment, which comprises about 20 nucleotides that are complementary to a target nucleic acid sequence within the target locus. In some embodiments, the corresponding target nucleic acid sequence is approximately 20 nucleotides in length. In some embodiments, the nucleic acid-binding segments of the gRNA sequences of the present disclosure are at least 90% complementary to a target nucleic acid sequence within a target locus. In some embodiments, the nucleic acid-binding segments of the gRNA sequences of the present disclosure are at least 95%, 96%, 97%, 98%, or 99% complementary to a target nucleic acid sequence within a target locus. In some embodiments, the nucleic acid-binding segments of the gRNA sequences of the present disclosure are 100% complementary to a target nucleic acid sequence within a target locus. In some embodiments, the target nucleic acid sequence is an RNA target sequence. In some embodiments, the target nucleic acid sequence is a DNA target sequence.

In some embodiments, the target nucleic acid sequence within the target locus must be changed. For example, changes in the target nucleic acid sequence may occur because the Cas protein being used is changed and the new Cas protein has a different PAM. The description provides many examples of target nucleic acid sequences for gRNAs in the specification and tables provided herein. Any of these target nucleic acid sequences can be changed by moving the target nucleic acid sequence 5′ or 3′ within the target locus within a given gene. In some embodiments, the target nucleic acid sequence is moved, at most, 100 bp 5′ or 3′ within the target locus within a given gene. In other embodiments, the target nucleic acid sequence is moved, at most, 1, 2, 3, 4, 5, 6, 7, 8, 9, 10, 11, 12, 13, 14, 15, 16, 17, 18, 19 20, 21, 22, 23, 24, 25, 30, 35, 40, 45, 50, 55, 60, 65, 70, 75, 80, 85, 90 or 95 bp 5′ or 3′ within the target locus within a given gene.

In some embodiments, the gene-regulating system comprises at least two gRNA molecules, wherein at least one gRNA molecule comprises a SOCS1-targeting nucleic acid-binding segment (i.e., a SOCS1-targeting gRNA). In some embodiments, the nucleic acid-binding segment of the at least one SOCS1-targeting gRNA molecules binds to a target DNA sequence that is at least 95%, 96%, 97%, 98%, or 99% identical to a DNA sequence encoded by the SOCS1 gene (SEQ ID NO: 1) or the Socs1 gene (SEQ ID NO: 2). In some embodiments, the nucleic acid-binding segment of the at least one SOCS1-targeting gRNA molecule binds to a target DNA sequence that is 100% identical to a DNA sequence encoded by the SOCS1 gene (SEQ ID NO: 1) or the Socs1 gene (SEQ ID NO: 2).

In some embodiments, the nucleic acid-binding segment of the at least one SOCS1-targeting gRNA molecules binds to a target DNA sequence that is at least 95%, 96%, 97%, 98%, or 99% identical to a DNA sequence defined by a set of genomic coordinates shown in Table 3 or Table 4. In some embodiments, the nucleic acid-binding segment of the at least one SOCS1-targeting gRNA molecules binds to a target DNA sequence that is 100% identical to a DNA sequence defined by a set of genomic coordinates shown in Table 3 or Table 4. In some embodiments, the nucleic acid-binding segment of the at least one SOCS1-targeting gRNA molecules binds to a target DNA sequence that is at least 95%, 96%, 97%, 98%, or 99% identical to one of SEQ ID NOs: 7-151. In some embodiments, the nucleic acid-binding segment of the at least one SOCS1-targeting gRNA molecules binds to a target DNA sequence that is 100% identical to one of SEQ ID NOs: 7-151. Exemplary SOCS1 target DNA sequences are shown in Tables 12 and 13.

In some embodiments, the nucleic acid-binding segment of the at least one SOCS1-targeting gRNA molecules is encoded by a DNA sequence that is at least 95%, 96%, 97%, 98%, or 99% identical to one of SEQ ID NOs: 7-151. In some embodiments, the nucleic acid-binding segment of the at least one SOCS1-targeting gRNA molecules is encoded by a DNA sequence that is 100% identical to one of SEQ ID NOs: 7-151. Exemplary DNA sequences encoding the nucleic acid-binding segment of the SOCS1-targeting gRNAs are shown in Tables 12 and 13.

TABLE 12
Exemplary human SOCS1 gRNA sequences

| Target | Sequence | SEQ ID |
|---|---|---|
| hSOCS1_gRNA_1 | GCGGCTGCGCGCCGAGCCCG | 20 |
| hSOCS1_gRNA_2 | GGACGCCTGCGGATTCTACT | 21 |
| hSOCS1_gRNA_3 | GGCTGCCATCCAGGTGAAAG | 22 |
| hSOCS1_gRNA_4 | GCGGCTGTCGCGCACCAGGA | 23 |
| hSOCS1_gRNA_5 | TGGACGCCTGCGGATTCTAC | 24 |
| hSOCS1_gRNA_6 | GACGCCTGCGGATTCTACTG | 25 |
| hSOCS1_gRNA_7 | AGTGCTCCAGCAGCTCGAAG | 26 |
| hSOCS1_gRNA_8 | GCCGGCCGCTTTCACCTGGA | 27 |
| hSOCS1_gRNA_9 | AGTAGAATCCGCAGGCGTCC | 28 |
| hSOCS1_gRNA_10 | CGCACCAGGAAGGTGCCCAC | 29 |
| hSOCS1_gRNA_11 | GGCCGGCCTGAAAGTGCACG | 30 |
| hSOCS1_gRNA_12 | TCCGTTCGCACGCCGATTAC | 31 |
| hSOCS1_gRNA_13 | AGCGCGCTCCTGGACGCCTG | 32 |
| hSOCS1_gRNA_14 | CGGCTGCGCGCCGAGCCCGT | 33 |
| hSOCS1_gRNA_15 | ACGCCTGCGGATTCTACTGG | 34 |
| hSOCS1_gRNA_16 | CGAGGCCATCTTCACGCTAA | 35 |
| hSOCS1_gRNA_17 | TCAGGCCGGCCGCTTTCACC | 36 |
| hSOCS1_gRNA_18 | CTTAGCGTGAAGATGGCCTC | 37 |
| hSOCS1_gRNA_19 | GCCGGTAATCGGCGTGCGAA | 38 |
| hSOCS1_gRNA_20 | CTGCATTGTCGGCTGCCACC | 39 |
| hSOCS1_gRNA_21 | GTGCGCCCCGTGCACGCTCA | 40 |
| hSOCS1_gRNA_22 | GCTGTGCCGCCAGCGCATCG | 41 |
| hSOCS1_gRNA_23 | CACGCGGCGCTGGCGCAGCG | 42 |
| hSOCS1_gRNA_24 | GCTCCTGCAGCGGCCGCACG | 43 |
| hSOCS1_gRNA_25 | AGCTCTCGCGGCTGCCATCC | 44 |
| hSOCS1_gRNA_26 | TGGTGCGCGACAGCCGCCAG | 45 |
| hSOCS1_gRNA_27 | GATGGTAGCACACAACCAGG | 46 |
| hSOCS1_gRNA_28 | AGAGGCAGTCGAAGCTCTCG | 47 |
| hSOCS1_gRNA_29 | GCTGGCGGCTGTCGCGCACC | 48 |
| hSOCS1_gRNA_30 | CCGAGGCCATCTTCACGCTA | 49 |
| hSOCS1_gRNA_31 | GGGGCCCCCAGCATGCGGCG | 50 |
| hSOCS1_gRNA_32 | GCTGCTGGAGCACTACGTGG | 51 |
| hSOCS1_gRNA_33 | CGAGCTGCTGGAGCACTACG | 52 |
| hSOCS1_gRNA_34 | CGAAAAAGCAGTTCCGCTGG | 53 |
| hSOCS1_gRNA_35 | GCAGGCGTCCAGGAGCGCGC | 54 |
| hSOCS1_gRNA_36 | GGGGCCCCTGAGCGTGCACG | 55 |
| hSOCS1_gRNA_37 | GCGGCGCCGCGCCGCATGCT | 56 |
| hSOCS1_gRNA_38 | GCACGCGGCGCTGGCGCAGC | 57 |
| hSOCS1_gRNA_39 | TGGGGGCCCCTGAGCGTGCA | 58 |
| hSOCS1_gRNA_40 | CAGGAAGGTGCCCACGGGCT | 59 |
| hSOCS1_gRNA_41 | TGCGCCCCGTGCACGCTCAG | 60 |
| hSOCS1_gRNA_42 | GCCATCCAGGTGAAAGCGGC | 61 |
| hSOCS1_gRNA_43 | CACGCGCGCCAGCGCGCTCC | 62 |
| hSOCS1_gRNA_44 | GGGCCCCCAGTAGAATCCGC | 63 |
| hSOCS1_gRNA_45 | ATCCGCGTGCACTTTCAGGC | 64 |
| hSOCS1_gRNA_46 | CGAGCCCGTGGGCACCTTCC | 65 |
| hSOCS1_gRNA_47 | CCACAGCAGCAGAGCCCCGA | 66 |
| hSOCS1_gRNA_48 | AGCCAGGTTCTCGCGGCCCA | 67 |
| hSOCS1_gRNA_49 | AAAGTGCACGCGGATGCTCG | 68 |
| hSOCS1_gRNA_50 | CTCTTCCTCCTCCTCGCCCG | 69 |
| hSOCS1_gRNA_51 | GCGTGCACGGGGCGCACGAG | 70 |
| hSOCS1_gRNA_52 | AAGTGCACGCGGATGCTCGT | 71 |
| hSOCS1_gRNA_53 | CGTGCGCCCCGTGCACGCTC | 72 |
| hSOCS1_gRNA_54 | GCAGCGGCCGCACGCGGCGC | 73 |
| hSOCSi gRNA_55 | CCTTAGCGTGAAGATGGCCT | 74 |
| hSOCS1_gRNA_56 | CAGGTTCTCGCGGCCCACGG | 75 |
| hSOCS1_gRNA_57 | GCGCACCAGGAAGGTGCCCA | 76 |
| hSOCS1_gRNA_58 | GCTGCCGGTCAAATCTGGAA | 77 |
| hSOCS1_gRNA_59 | CGGCGTGCGAACGGAATGTG | 78 |
| hSOCS1_gRNA_60 | CAGCAGCAGAGCCCCGACGG | 79 |
| hSOCS1_gRNA_61 | GGGCGAAAAAGCAGTTCCGC | 80 |
| hSOCS1_gRNA_62 | CGCACGCGGCGCTGGCGCAG | 81 |
| hSOCS1_gRNA_63 | GGATGCGAGCCAGGTTCTCG | 82 |
| hSOCS1_gRNA_64 | TGGCGGCACAGCTCCTGCAG | 83 |
| hSOCS1_gRNA_65 | GCGCCCGCGGCCGTGCCCCG | 84 |
| hSOCS1_gRNA_66 | GGCGCCGCGCCGCATGCTGG | 85 |
| hSOCS1_gRNA_67 | CGGTGGCCACGATGCGCTGG | 86 |
| hSOCS1_gRNA_68 | TGCTGTGGAGACTGCATTGT | 87 |
| hSOCS1_gRNA_69 | TAGGATGGTAGCACACAACC | 88 |
| hSOCS1_gRNA_70 | GCGGCCGTGCCCCGCGGTCC | 89 |
| hSOCS1_gRNA_71 | GAGCATCCGCGTGCACTTTC | 90 |
| hSOCS1_gRNA_72 | CGCTGCCGGTCAAATCTGGA | 91 |
| hSOCS1_gRNA_73 | CAGCGCATCGTGGCCACCGT | 92 |
| hSOCS1_gRNA_74 | GCGGATGCTCGTGGGTCCCG | 93 |
| hSOCS1_gRNA_75 | CGGCGCCGCGCCGCATGCTG | 94 |
| hSOCS1_gRNA_76 | CGGTCAAATCTGGAAGGGGA | 95 |
| hSOCS1_gRNA_77 | AGGAAGGTTCTGGCCGCCGT | 96 |
| hSOCS1_gRNA_78 | CCACGGTGGCCACGATGCGC | 97 |
| hSOCS1_gRNA_79 | CGCTGCGCCAGCGCCGCGTG | 98 |
| hSOCS1_gRNA_80 | AGGAGCTCAGGTAGTCGCGG | 99 |
| hSOCS1_gRNA_81 | GCAGCGGGGCCCCCAGCATG | 100 |
| hSOCS1_gRNA_82 | GGAAGGAGCTCAGGTAGTCG | 101 |
| hSOCS1_gRNA_83 | TCGCGGAGGACGGGGTTGAG | 102 |
| hSOCS1_gRNA_84 | CGACTGCCTCTTCGAGCTGC | 103 |
| hSOCS1_gRNA_85 | GCGCCGCGTGCGGCCGCTGC | 104 |
| hSOCS1_gRNA_86 | CACCGTGGGCCGCGAGAACC | 105 |
| hSOCS1_gRNA_87 | GTGCCCCGCGGTCCCGGCCC | 106 |
| hSOCS1_gRNA_88 | CTGCCGGTCAAATCTGGAAG | 107 |
| hSOCS1_gRNA_89 | CTTCCCCTTCCAGATTTGAC | 108 |
| hSOCS1_gRNA_90 | CTCAGGTAGTCGCGGAGGAC | 109 |
| hSOCS1_gRNA_91 | CGGGCGCTGCCGGTCAAATC | 110 |
| hSOCS1_gRNA_92 | GGAAGGTTCTGGCCGCCGTC | 111 |
| hSOCS1_gRNA_93 | GCTCAGGTAGTCGCGGAGGA | 112 |
| hSOCS1_gRNA_94 | GCGGAAGTGCGTGTCGCCGG | 113 |
| hSOCS1_gRNA_95 | GGACCGCGGGGCACGGCCGC | 114 |
| hSOCS1_gRNA_96 | GGGACCGCGGGGCACGGCCG | 115 |
| hSOCS1_gRNA_97 | GCGCGTGATGCGCCGGTAAT | 116 |
| hSOCS1_gRNA_98 | TCAGGTAGTCGCGGAGGACG | 117 |
| hSOCS1_gRNA_99 | TGCGGAAGTGCGTGTCGCCG | 118 |
| hSOCS1_gRNA_100 | GGGGCCGGGACCGCGGGGCA | 119 |
| hSOCS1_gRNA_101 | CCGTCGGGGCTCTGCTGCTG | 120 |
| hSOCS1_gRNA_102 | GAAGGTTCTGGCCGCCGTCG | 121 |
| hSOCS1_gRNA_103 | GTGTGCTACCATCCTACAGA | 122 |
| hSOCS1_gRNA_104 | GTCGCGGAGGACGGGGTTGA | 123 |
| hSOCS1_gRNA_105 | CGCTGGCGCGCGTGATGCGC | 124 |
| hSOCS1_gRNA_106 | GCGTGCACGGCGGGCGCTGC | 125 |
| hSOCS1_gRNA_107 | TCTGGAAGGGGAAGGAGCTC | 126 |
| hSOCS1_gRNA_108 | GTGCGTGTCGCCGGGGGCCG | 127 |
| hSOCS1_gRNA_109 | GGGCACGGCCGCGGGCGCGC | 128 |
| hSOCS1_gRNA_110 | GTTAATGCTGCGTGCACGGC | 129 |
| hSOCS1_gRNA_111 | GCACGGCCGCGGGCGCGCGG | 130 |
| hSOCS1_gRNA_112 | GGGGCACGGCCGCGGGCGCG | 131 |
| hSOCS1_gRNA_113 | GTGCGGAAGTGCGTGTCGCC | 132 |
| hSOCS1_gRNA_114 | GAGGAAGAGGAGGAAGGTTC | 133 |
| hSOCS1_gRNA_115 | GGCTGGCCCCTTCTGTAGGA | 134 |
| hSOCS1_gRNA_116 | GGGGCCGGGGCCGGGACCGC | 135 |
| hSOCS1_gRNA_117 | CGCGGAGGACGGGGTTGAGG | 136 |
| hSOCS1_gRNA_118 | TTTCGCCCTTAGCGTGAAGA | 137 |
| hSOCS1_gRNA_119 | GGCACGGCCGCGGGCGCGCG | 138 |
| hSOCS1_gRNA_120 | AGTCGCGGAGGACGGGGTTG | 139 |
| hSOCS1_gRNA_121 | GGGCCGGGGCCGGGACCGCG | 140 |
| hSOCS1_gRNA_122 | AAGTGCGTGTCGCCGGGGGC | 141 |
| hSOCS1_gRNA_123 | CTCCGGCTGGCCCCTTCTGT | 142 |
| hSOCS1_gRNA_124 | GGCGGCGCCGCGCCGCATGC | 143 |
| hSOCS1_gRNA_125 | AGTGCGTGTCGCCGGGGGCC | 144 |
| hSOCS1_gRNA_126 | TGTGCGGAAGTGCGTGTCGC | 145 |
| hSOCS1_gRNA_127 | GTGTCGCCGGGGGCCGGGGC | 146 |
| hSOCS1_gRNA_128 | TGTCGCCGGGGGCCGGGGCC | 147 |
| hSOCS1_gRNA_129 | GCGGTCCCGGCCCCGGCCCC | 148 |
| hSOCS1_gRNA_130 | CGCGGGGGCCGCGGGCGAGG | 149 |
| hSOCS1_gRNA_131 | CGCGGGCGAGGAGGAGGAAG | 150 |
| hSOCS1_gRNA_132 | GGGCGAGGAGGAGGAAGAGG | 151 |

TABLE 13
Exemplary murine Socs1_gRNA sequences

| Target | Sequence | SEQ ID |
|---|---|---|
| mSocs1_gRNA_1 | GAAGTGCACGCGGATGCTCG | 7 |
| mSocs1_gRNA_2 | AGTGCTCCAGCAGCTCGAAA | 8 |
| mSocs1_gRNA_3 | GCCGGCCGCTTCCACTTGGA | 9 |
| mSocs1_gRNA_4 | GCTGTGTCGCCAGCGCATCG | 10 |
| mSocs1_gRNA_5 | GCGACTGTCGCGCACCAAGA | 11 |
| mSocs1_gRNA_6 | GCGTGCACGGGGCGCACGAG | 12 |
| mSocs1_gRNA_7 | TCACGGAGTACCGGGTTAAG | 13 |
| mSocs1_gRNA_8 | GGACGCCTGCGGCTTCTATT | 14 |
| mSocs1_gRNA_9 | GCGCGAAGAAGCAGTTCCGT | 15 |
| mSocs1_gRNA_10 | GCTCAGCGTGAAGATGGCTT | 16 |
| mSocs1_gRNA_11 | CGAGCCCGTGGGCACCTTCT | 17 |
| mSocs1_gRNA_12 | ATCCGCGTGCACTTCCAGGC | 18 |
| mSocs1_gRNA_13 | CGCCAGGTTCTCGCGACCCA | 19 |

In some embodiments, the gene-regulating system comprises at least two gRNA molecules, wherein at least one gRNA molecule comprises a PTPN2-targeting nucleic acid-binding segment (i.e., a PTPN2-targeting gRNA). In some embodiments, the nucleic acid-binding segment of the at least one PTPN2-targeting gRNA molecules binds to a target DNA sequence that is at least 95%, 96%, 97%, 98%, or 99% identical to a DNA sequence encoded by the PTPN2 gene (SEQ ID NO: 3) or the Ptpn2 gene (SEQ ID NO: 4). In some embodiments, the nucleic acid-binding segment of the at least one PTPN2-targeting gRNA molecules binds to a target DNA sequence that is 100% identical to a DNA sequence encoded by the PTPN2 gene (SEQ ID NO: 3) or the Ptpn2 gene (SEQ ID NO: 4).

In some embodiments, the nucleic acid-binding segment of the at least one PTPN2-targeting gRNA molecules binds to a target DNA sequence that is at least 95%, 96%, 97%, 98%, or 99% identical to a DNA sequence defined by a set of genomic coordinates shown in Table 5 or Table 6. In some embodiments, the nucleic acid-binding segment of the at least one PTPN2-targeting gRNA molecules binds to a target DNA sequence that is 100% identical to a DNA sequence defined by a set of genomic coordinates shown in Table 5 or Table 6. In some embodiments, the nucleic acid-binding segment of the at least one PTPN2-targeting gRNA molecules binds to a target DNA sequence that is at least 95%, 96%, 97%, 98%, or 99% identical to one of SEQ ID NOs: 185-207. In some embodiments, the nucleic acid-binding segment of the at least one PTPN2-targeting gRNA molecules binds to a target DNA sequence that is 100% identical to one of SEQ ID NOs: 185-207. Exemplary PTPN2 target DNA sequences are shown in Tables 14 and 15.

In some embodiments, the nucleic acid-binding segment of the at least one PTPN2-targeting gRNA molecules is encoded by a DNA sequence that is at least 95%, 96%, 97%, 98%, or 99% identical to one of SEQ ID NOs: 185-207. In some embodiments, the nucleic acid-binding segment of the at least one PTPN2-targeting gRNA molecules is encoded by a DNA sequence that is 100% identical to one of SEQ ID NOs: 185-207. In some embodiments, the nucleic acid-binding segment of the at least one PTPN2-targeting gRNA molecules is encoded by a DNA sequence that is at least 95%, 96%, 97%, 98%, or 99% identical to one of SEQ ID NOs: 272-375. In some embodiments, the nucleic acid-binding segment of the at least one PTPN2-targeting gRNA molecules is encoded by a DNA sequence that is 100% identical to one of SEQ ID NOs: 272-375. In some embodiments, the nucleic acid-binding segment of the at least one PTPN2-targeting gRNA molecules is encoded by a DNA sequence that is at least 95%, 96%, 97%, 98%, or 99% identical to one of SEQ ID NOs: 272-308. In some embodiments, the nucleic acid-binding segment of the at least one PTPN2-targeting gRNA molecules is encoded by a DNA sequence that is 100% identical to one of SEQ ID NOs: 272-308. Exemplary DNA sequences encoding the nucleic acid-binding segment of the PTPN2-targeting gRNAs are shown in Tables 14 and 15.

TABLE 14
Exemplary human PTPN2 gRNA sequences

| Target | Sequence | SEQ ID |
|---|---|---|
| hPTPN2_gRNA_1 | CCATGCCCACCACCATCGAG | 185 |
| hPTPN2_gRNA_2 | TCTACGGAAACGTATTCGAG | 186 |
| hPTPN2_gRNA_3 | TTTAGTATATTGAGAACTTG | 187 |
| hPTPN2_gRNA_4 | GCACTACAGTGGATCACCGC | 188 |
| hPTPN2_gRNA_5 | TGTCATGCTGAACCGCATTG | 189 |
| hPTPN2_gRNA_6 | GGAAACTTGGCCACTCTATG | 190 |
| hPTPN2_gRNA_7 | GTATTTGAAATTATTAATGC | 191 |
| hPTPN2_gRNA_8 | CAGTTTAGTTGACATAGAAG | 192 |
| hPTPN2_gRNA_9 | GGGTCTGAATAAGACCCATT | 193 |
| hPTPN2_gRNA_10 | CCATGACTATCCTCATAGAG | 194 |
| hPTPN2_gRNA_11 | CCATGACTATCCTCATAGAG | 272 |
| hPTPN2_gRNA_12 | CTCTTCGAACTCCCGCTCGA | 273 |
| hPTPN2_gRNA_13 | GAACCCTGACCATGGGCCTG | 274 |
| hPTPN2_gRNA_14 | GCTCCTTGAACCCTGACCAT | 275 |
| hPTPN2_gRNA_15 | AGTTGGATACTCAGCGTCGC | 276 |
| hPTPN2_gRNA_16 | CCGCTCGATGGTGGTGGGCA | 277 |
| hPTPN2_gRNA_17 | CAGAAATGGCAGCATGTGTT | 278 |
| hPTPN2_gRNA_18 | GCACTACAGTGGATCACCGC | 279 |
| hPTPN2_gRNA_19 | GGTAGACACTTGTCTTGTTT | 280 |
| hPTPN2_gRNA_20 | TGGCAGCATGTGTTAGGAAG | 281 |
| hPTPN2_gRNA_21 | AGGCCCATGGTCAGGGTTCA | 282 |
| hPTPN2_gRNA_22 | GTTCAGCATGACAACTGCTT | 283 |
| hPTPN2_gRNA_23 | CAATGGAGGAGAACAGTGAG | 284 |
| hPTPN2_gRNA_24 | CTCTTCTATGTCAACTAAAC | 285 |
| hPTPN2_gRNA_25 | AGTGGATCACCGCAGGCCCA | 286 |
| hPTPN2_gRNA_26 | CTGACAGGTGACCGATGTAC | 287 |
| hPTPN2_gRNA_27 | AACTCCCGCTCGATGGTGGT | 288 |
| hPTPN2_gRNA_28 | GTCTCCCTGATCCATCCAGT | 289 |
| hPTPN2_gRNA_29 | TAGAGGAAAGTCCTGTACAT | 290 |
| hPTPN2_gRNA_30 | ATGTATGGAAAGGATGGTAA | 291 |
| hPTPN2_gRNA_31 | GCCCAATGCCTGCACTACAG | 292 |
| hPTPN2_gRNA_32 | CGAGCGGGAGTTCGAAGAGT | 293 |
| hPTPN2_gRNA_33 | TCACCGCAGGCCCATGGTCA | 294 |
| hPTPN2_gRNA_34 | CAGTTTAGTTGACATAGAAG | 295 |
| hPTPN2_gRNA_35 | CCATGCCCACCACCATCGAG | 296 |
| hPTPN2_gRNA_36 | GCCAAACCATAAGCCAGAAA | 297 |
| hPTPN2_gRNA_37 | CCGATTCTTTCTCCACAATG | 298 |
| hPTPN2_gRNA_38 | TTCGAACTCCCGCTCGATGG | 299 |
| hPTPN2_gRNA_39 | AGTGCAGGCATTGGGCGCTC | 300 |
| hPTPN2_gRNA_40 | GGAAACTTGGCCACTCTATG | 301 |
| hPTPN2_gRNA_41 | ATCCACTGTAGTGCAGGCAT | 302 |
| hPTPN2_gRNA_42 | CACTCTATGAGGATAGTCAT | 303 |
| hPTPN2_gRNA_43 | CCACTCTATGAGGATAGTCA | 304 |
| hPTPN2_gRNA_44 | TCCACTGTAGTGCAGGCATT | 305 |
| hPTPN2_gRNA_45 | AAGTTCTTTCCATCGTTTCT | 306 |
| hPTPN2_gRNA_46 | TCGCTGGCAGCCGCTGTACT | 307 |
| hPTPN2_gRNA_47 | GAACTCCCGCTCGATGGTGG | 308 |
| hPTPN2_gRNA_48 | AGGATGGTAAAGGCACCAAC | 309 |
| hPTPN2_gRNA_49 | AAAGGGAGATTCTAGTATAC | 310 |
| hPTPN2_gRNA_50 | AGAATTTAGGATGTATGGAA | 311 |
| hPTPN2_gRNA_51 | GGGTCTGAATAAGACCCATT | 312 |
| hPTPN2_gRNA_52 | GGCACCAACTGGATGGATCA | 313 |
| hPTPN2_gRNA_53 | CTCTAAAATGCAAGATACAA | 314 |
| hPTPN2_gRNA_54 | GTATTTGAAATTATTAATGC | 315 |
| hPTPN2_gRNA_55 | CCTTTCTTGCAGATGGAAAA | 316 |
| hPTPN2_gRNA_56 | CTGCACCTTCTGAGCTGTGG | 317 |
| hPTPN2_gRNA_57 | ATGCTGCCATTTCTGGCTTA | 318 |
| hPTPN2_gRNA_58 | TTTCTTTAAACAGCATCTCT | 319 |
| hPTPN2_gRNA_59 | AGACATGGAATGCAGAATGC | 320 |
| hPTPN2_gRNA_60 | AGGCACCAACTGGATGGATC | 321 |
| hPTPN2_gRNA_61 | TAATGACTGAAAAATACAAT | 322 |
| hPTPN2_gRNA_62 | GAATGCAGAATGCAGGAAAT | 323 |
| hPTPN2_gRNA_63 | TTTAGGATGTATGGAAAGGA | 324 |
| hPTPN2_gRNA_64 | CTAACACATGCTGCCATTTC | 325 |
| hPTPN2_gRNA_65 | TCATACATGGCTATAATAGA | 326 |
| hPTPN2_gRNA_66 | ACGATGGAAAGAACTTTCTA | 327 |
| hPTPN2_gRNA_67 | ACGTATTCGAGAGGACAGAA | 328 |
| hPTPN2_gRNA_68 | GCGGTGATCCACTGTAGTGC | 329 |
| hPTPN2_gRNA_69 | TATTAATGCTGGATGTTAAA | 330 |
| hPTPN2_gRNA_70 | GAGATGCTGTTTAAAGAAAC | 331 |
| hPTPN2_gRNA_71 | CAGCAAGAATTTAGGATGTA | 332 |
| hPTPN2_gRNA_72 | TTGACATAGAAGAGGCACAA | 333 |
| hPTPN2_gRNA_73 | GATTCAGGGACTCCAAAATC | 334 |
| hPTPN2_gRNA_74 | CTCACTTTCATTATACTACC | 335 |
| hPTPN2_gRNA_75 | TTTAGTATATTGAGAACTTG | 336 |
| hPTPN2_gRNA_76 | AGGGACTCCAAAATCTGGCC | 337 |
| hPTPN2_gRNA_77 | AGGTTAAATGTGCACAGTAC | 338 |
| hPTPN2_gRNA_78 | ATCACCGCAGGCCCATGGTC | 339 |
| hPTPN2_gRNA_79 | AGCATCTCTTGGTCATCTGT | 340 |
| hPTPN2_gRNA_80 | GAAGGAGCAAAATGTATAAA | 341 |
| hPTPN2_gRNA_81 | GCCATTTCTGGCTTATGGTT | 342 |
| hPTPN2_gRNA_82 | CTGGATGGATCAGGGAGACA | 343 |
| hPTPN2_gRNA_83 | AAATACAATGGGAACAGAAT | 344 |
| hPTPN2_gRNA_84 | ATAATGACTGAAAAATACAA | 345 |
| hPTPN2_gRNA_85 | CATGCCCACCACCATCGAGC | 346 |
| hPTPN2_gRNA_86 | AACATGAGAAAATACCGAAT | 347 |
| hPTPN2_gRNA_87 | AGAAATGAAGCTGGTGATTC | 348 |
| hPTPN2_gRNA_88 | CCGCATTGTGGAGAAAGAAT | 349 |
| hPTPN2_gRNA_89 | GAAATGAAGCTGGTGATTCA | 350 |
| hPTPN2_gRNA_90 | TTGTTTAAAGTGAGAGAATC | 351 |
| hPTPN2_gRNA_91 | CCGCGACTCACCAAGTACAG | 352 |
| hPTPN2_gRNA_92 | GAACATGAGAAAATACCGAA | 353 |
| hPTPN2_gRNA_93 | TATACTACCTGGCCAGATTT | 354 |
| hPTPN2_gRNA_94 | TATGAGAATCTCAGTTGATC | 355 |
| hPTPN2_gRNA_95 | TCAACTGAGATTCTCATACA | 356 |
| hPTPN2_gRNA_96 | TGAGAATCTCAGTTGATCTG | 357 |
| hPTPN2_gRNA_97 | ATGAGAATCTCAGTTGATCT | 358 |
| hPTPN2_gRNA_98 | TGGTAAAGGCACCAACTGGA | 359 |
| hPTPN2_gRNA_99 | TGTCATGCTGAACCGCATTG | 360 |
| hPTPN2_gRNA_100 | TTTGGTGAATGATCAAAGGC | 361 |
| hPTPN2_gRNA_101 | ATGAAAGTGAGATATTGTTC | 362 |
| hPTPN2_gRNA_102 | TATTTCCTCATAGTGCTCTA | 363 |
| hPTPN2_gRNA_103 | AGAAGGAGCAAAATGTATAA | 364 |
| hPTPN2_gRNA_104 | TTTGTTTGGTGAATGATCAA | 365 |
| hPTPN2_gRNA_105 | TCTACGGAAACGTATTCGAG | 366 |
| hPTPN2_gRNA_106 | AAAGGCCACCACAGCTCAGA | 367 |
| hPTPN2_gRNA_107 | AGGTGCAGCAGATGAAACAG | 368 |
| hPTPN2_gRNA_108 | GGCTCCTTGAACCCTGACCA | 369 |
| hPTPN2_gRNA_109 | AAGGAGTTACATCTTAACAC | 370 |
| hPTPN2_gRNA_110 | TAAAATGCAAGATACAATGG | 371 |
| hPTPN2_gRNA_111 | ACAAGTGTCTACCAGAGAGA | 372 |
| hPTPN2_gRNA_112 | GCGCTCTGGCACCTTCTCTC | 373 |
| hPTPN2_gRNA_113 | CTGCTGCACCTTCTGAGCTG | 374 |
| hPTPN2_gRNA_114 | TCTTCCCTACCTAGAAACGA | 375 |

TABLE 15
Exemplary murine Ptpn2 gRNA sequences

| Target | Sequence | SEQ ID |
|---|---|---|
| mPTPN2_gRNA_1 | AATCTGGCCAGGTGGTATAA | 195 |
| mPTPN2_gRNA_2 | AATATGAGAAAGTATCGAAT | 196 |
| mPTPN2_gRNA_3 | ATCACTGCAGGTCCATGGTC | 197 |
| mPTPN2_gRNA_4 | ATGTGCACAGTACTGGCCAA | 198 |
| mPTPN2_gRNA_5 | GGCAGCATGTGTTCGGAAGT | 199 |
| mPTPN2_gRNA_6 | AAGAAGTTTAGAAATGAAGC | 200 |
| mPTPN2_gRNA_7 | GCCACACCATGAGCCAGAAA | 201 |
| mPTPN2_gRNA_8 | CCTTTCTTGCAGATGGAAAA | 202 |
| mPTPN2_gRNA_9 | GTACTTTGCTCCTTCTATTA | 203 |
| mPTPN2_gRNA_10 | AGAAATGAAGCTGGTGACTC | 204 |
| mPTPN2_gRNA_11 | GTTTAGCATGACAACTGCTT | 205 |
| mPTPN2_gRNA_12 | GCCCGATGCCCGCACTGCAA | 206 |
| mPTPN2_gRNA_13 | TGACAGAGAAATGGTGTTTA | 207 |

In some embodiments, the gene-regulating system comprises at least two gRNA molecules, wherein at least one gRNA molecule comprises a ZC3H12A-targeting nucleic acid-binding segment (i.e., a ZC3H12A-targeting gRNA). In some embodiments, the nucleic acid-binding segment of the at least one ZC3H12A-targeting gRNA molecules binds to a target DNA sequence that is at least 95%, 96%, 97%, 98%, or 99% identical to a DNA sequence encoded by the ZC3H12A gene (SEQ ID NO: 5) or the Zc3h12a gene (SEQ ID NO: 6). In some embodiments, the nucleic acid-binding segment of the at least one ZC3H12A-targeting gRNA molecules binds to a target DNA sequence that is 100% identical to a DNA sequence encoded by the ZC3H12A gene (SEQ ID NO: 5) or the Zc3h12a gene (SEQ ID NO: 6).

In some embodiments, the nucleic acid-binding segment of the at least one ZC3H12A-targeting gRNA molecules binds to a target DNA sequence that is at least 95%, 96%, 97%, 98%, or 99% identical to a DNA sequence defined by a set of genomic coordinates shown in Table 7 or Table 8. In some embodiments, the nucleic acid-binding segment of the at least one ZC3H12A-targeting gRNA molecules binds to a target DNA sequence that is 100% identical to a DNA sequence defined by a set of genomic coordinates shown in Table 7 or Table 8. In some embodiments, the nucleic acid-binding segment of the at least one ZC3H12A-targeting gRNA molecules binds to a target DNA sequence that is at least 95%, 96%, 97%, 98%, or 99% identical to one of SEQ ID NOs: 208-230. In some embodiments, the nucleic acid-binding segment of the at least one ZC3H12A-targeting gRNA molecules binds to a target DNA sequence that is 100% identical to one of SEQ ID NOs: 208-230. Exemplary ZC3H12A target DNA sequences are shown in Tables 16 and 17.

In some embodiments, the nucleic acid-binding segment of the at least one ZC3H12A-targeting gRNA molecules is encoded by a DNA sequence that is at least 95%, 96%, 97%, 98%, or 99% identical to one of SEQ ID NOs: 208-230. In some embodiments, the nucleic acid-binding segment of the at least one ZC3H12A-targeting gRNA molecules is encoded by a DNA sequence that is 100% identical to one of SEQ ID NOs: 208-230. In some embodiments, the nucleic acid-binding segment of the at least one ZC3H12A-targeting gRNA molecules is encoded by a DNA sequence that is at least 95%, 96%, 97%, 98%, or 99% identical to one of SEQ ID NOs: 376-812. In some embodiments, the nucleic acid-binding segment of the at least one ZC3H12A-targeting gRNA molecules is encoded by a DNA sequence that is 100% identical to one of SEQ ID NOs: 376-812. In some embodiments, the nucleic acid-binding segment of the at least one ZC3H12A-targeting gRNA molecules is encoded by a DNA sequence that is at least 95%, 96%, 97%, 98%, or 99% identical to one of SEQ ID NOs: 376-575. In some embodiments, the nucleic acid-binding segment of the at least one ZC3H12A-targeting gRNA molecules is encoded by a DNA sequence that is 100% identical to one of SEQ ID NOs: 376-575. Exemplary DNA sequences encoding the nucleic acid-binding segment of the ZC3H12A-targeting gRNAs are shown in Tables 16 and 17.

TABLE 16
Exemplary human ZC3H12A gRNA sequences

| Target | Sequence | SEQ ID |
|---|---|---|
| hZC3H12A_gRNA_1 | AAGCTGGCCTACGAGTCTGA | 216 |
| hZC3H12A_gRNA_2 | GCGGGACTAGAGGGAGCTGA | 217 |
| hZC3H12A_gRNA_3 | CAGCTCCCTCTAGTCCCGCG | 218 |
| hZC3H12A_gRNA_4 | CAGGACGCTGTGGATCTCCG | 219 |
| hZC3H12A_gRNA_5 | AACATACTTGTCATTGACGA | 220 |
| hZC3H12A_gRNA_6 | CTCACCTGTGATGGGCACGT | 221 |
| hZC3H12A_gRNA_7 | AACACGGGACAGCCACCGAG | 222 |
| hZC3H12A_gRNA_8 | CGACAGATTCATTGTGAAGC | 223 |
| hZC3H12A_gRNA_9 | ACACCATCACGACGCGTGGG | 224 |
| hZC3H12A_gRNA_10 | TCCCAGCCATGGGAACAAGG | 225 |
| hZC3H12A_gRNA_11 | GGAGTGGAAGCGCTTCATCG | 226 |
| hZC3H12A_gRNA_12 | TTAGGGGTGCCACCACCCCG | 227 |
| hZC3H12A_gRNA_13 | GACACATACCGTGACCTCCA | 228 |
| hZC3H12A_gRNA_14 | CCGGCCCAGTGGGTCATCAG | 229 |
| hZC3H12A_gRNA_15 | CCTGGAACTGCAGATGAAGG | 230 |
| hZC3H12A_gRNA_16 | GTCCTCTCCCTCCCAGCCAT | 376 |
| hZC3H12A_gRNA_17 | TCCCCAGGGTCCCGCCAAGA | 377 |
| hZC3H12A_gRNA_18 | AGTGAGCAGTGCAGCCTGGA | 378 |
| hZC3H12A_gRNA_19 | TGTCCTCTCCCTCCCAGCCA | 379 |
| hZC3H12A_gRNA_20 | CTGGACTGGGATGAAGGTGA | 380 |
| hZC3H12A_gRNA_21 | GGGGTGGGCCCGGCTCACCA | 381 |
| hZC3H12A_gRNA_22 | CACCACCCCGCGGGACTAGA | 382 |
| hZC3H12A_gRNA_23 | CTGCTGCCACTGCCCCCGCT | 383 |
| hZC3H12A_gRNA_24 | CGGCCCGACGTGCCCATCAC | 384 |
| hZC3H12A_gRNA_25 | CACTGCCCCCGCTAGGTGCG | 385 |
| hZC3H12A_gRNA_26 | ATACACGCTGGCCTGCTCCT | 386 |
| hZC3H12A_gRNA_27 | CAAACACTGTGATGTCTGTG | 387 |
| hZC3H12A_gRNA_28 | GCGGGACCCTGGGGATGCCT | 388 |
| hZC3H12A_gRNA_29 | GCGGGAGCGCCAGACCTCAC | 389 |
| hZC3H12A_gRNA_30 | AGGACAGGCTTCTCTCCACA | 390 |
| hZC3H12A_gRNA_31 | GCAGACACCAACACGGTGCT | 391 |
| hZC3H12A_gRNA_32 | CCACCACCCCGCGGGACTAG | 392 |
| hZC3H12A_gRNA_33 | ATCCCCAGGGTCCCGCCAAG | 393 |
| hZC3H12A_gRNA_34 | CCTGGAGGAAGGAGCAGCCT | 394 |
| hZC3H12A_gRNA_35 | AGAGCCAGATGTCGGAACTT | 395 |
| hZC3H12A_gRNA_36 | ATGACCCACTGGGCCGGCAC | 396 |
| hZC3H12A_gRNA_37 | GCAGCTTTGGGCCCACAGAC | 397 |
| hZC3H12A_gRNA_38 | ACTCTCTGTTAGCAGAGAGC | 398 |
| hZC3H12A_gRNA_39 | CCAGGAAGGAAATGCACCTA | 399 |
| hZC3H12A_gRNA_40 | AGGCACCACTCACCTGTGAT | 400 |
| hZC3H12A_gRNA_41 | CTGGGCCCGTGCCGGCCCAG | 401 |
| hZC3H12A_gRNA_42 | CAGCCAGCTGCTGGGGGTCC | 402 |
| hZC3H12A_gRNA_43 | TCCACTCCTGCCGCTCGCCT | 403 |
| hZC3H12A_gRNA_44 | CGTCCAGGCAGACACCAACA | 404 |
| hZC3H12A_gRNA_45 | CCCACCCACATCAGTCCTTC | 405 |
| hZC3H12A_gRNA_46 | GCCAGCTCTTGACCCGGCCT | 406 |
| hZC3H12A_gRNA_47 | CTGCCCTCCTTTTCCTCTTC | 407 |
| hZC3H12A_gRNA_48 | CCAGCCCCACCATGAGTCTG | 408 |
| hZC3H12A_gRNA_49 | GCCGATTCTTCCACCCAGAG | 409 |
| hZC3H12A_gRNA_50 | CTCCCAGAAGAGGAAAAGGA | 410 |
| hZC3H12A_gRNA_51 | GTGGGGCAGGGCAGGCAGCC | 411 |
| hZC3H12A_gRNA_52 | GGGTCAAGAGCTGGCCGCTG | 412 |
| hZC3H12A_gRNA_53 | ATGCCCCCTGATGACCCACT | 413 |
| hZC3H12A_gRNA_54 | AGCCTTCTCTGCCTTTGGCC | 414 |
| hZC3H12A_gRNA_55 | CTCTGCCTTTGGCCGGGCCA | 415 |
| hZC3H12A_gRNA_56 | GGAACCCAGCCTGCCCTCCC | 416 |
| hZC3H12A_gRNA_57 | GGCAGGAGCCTCGCACCTAG | 417 |
| hZC3H12A_gRNA_58 | TCCCAGACCAGCACATCCTG | 418 |
| hZC3H12A_gRNA_59 | GTGAGCAGTGCAGCCTGGAT | 419 |
| hZC3H12A_gRNA_60 | GAGCCAGATGTCGGAACTTT | 420 |
| hZC3H12A_gRNA_61 | GGCCGATGGCAAGCCTTGCT | 421 |
| hZC3H12A_gRNA_62 | AGGAGCCTCGCACCTAGCGG | 422 |
| hZC3H12A_gRNA_63 | AGGTCCCCAAGAGGAAAACA | 423 |
| hZC3H12A_gRNA_64 | CGCTGAGGAGGCCTCGGCCC | 424 |
| hZC3H12A_gRNA_65 | GAGGACAGCCACAGCCGTCA | 425 |
| hZC3H12A_gRNA_66 | CAGCCCCACCATGAGTCTGT | 426 |
| hZC3H12A_gRNA_67 | ACCCCCCAGAGCCCCAAGCA | 427 |
| hZC3H12A_gRNA_68 | GAGGCACCACTCACCTGTGA | 428 |
| hZC3H12A_gRNA_69 | CCAAGAGGAAAACAGGGCAC | 429 |
| hZC3H12A_gRNA_70 | GTACGTCTCCCAGGATTGCC | 430 |
| hZC3H12A_gRNA_71 | CACAGCCTCCACCAGGTGCG | 431 |
| hZC3H12A_gRNA_72 | GATCTCGGCAGCCAGCTGCT | 432 |
| hZC3H12A_gRNA_73 | CAGCCTTCTCTGCCTTTGGC | 433 |
| hZC3H12A_gRNA_74 | CAGAAGTGACACTTACCTCA | 434 |
| hZC3H12A_gRNA_75 | GCTGGCCGCTGAGGAGGCCT | 435 |
| hZC3H12A_gRNA_76 | CAGCTCCCTCTAGTCCCGCG | 436 |
| hZC3H12A_gRNA_77 | CGGGGTGGGCCCGGCTCACC | 437 |
| hZC3H12A_gRNA_78 | GACACATACCGTGACCTCCA | 438 |
| hZC3H12A_gRNA_79 | CAGGAAGGAAATGCACCTAT | 439 |
| hZC3H12A_gRNA_80 | AGTGGCCAGCACCCATGGCC | 440 |
| hZC3H12A_gRNA_81 | CTCTCCTATTCTTCCCAGCA | 441 |
| hZC3H12A_gRNA_82 | GCCCGAGTCCAGGCAATCCT | 442 |
| hZC3H12A_gRNA_83 | CACCTTCATCTGCAGTTCCA | 443 |
| hZC3H12A_gRNA_84 | GGCACAGGCAGACAGGTGAG | 444 |
| hZC3H12A_gRNA_85 | AGCACCCATGGCCCGGCCAA | 445 |
| hZC3H12A_gRNA_86 | CCACAGGCAGCTTACTCACT | 446 |
| hZC3H12A_gRNA_87 | TTCCTGTGCTCCAAAGTGAG | 447 |
| hZC3H12A_gRNA_88 | ACCGCAGCCTTCTCTGCCTT | 448 |
| hZC3H12A_gRNA_89 | GGGAGCCAATGCCCGAGTCC | 449 |
| hZC3H12A_gRNA_90 | TTCCCAGCAAGGCTTGCCAT | 450 |
| hZC3H12A_gRNA_91 | AGCCAGATGTCGGAACTTTG | 451 |
| hZC3H12A_gRNA_92 | TACACGGGCTACAGTCCCTA | 452 |
| hZC3H12A_gRNA_93 | TCTGTGTTAGACCCTCTTGG | 453 |
| hZC3H12A_gRNA_94 | AAGCTGCCCCCAGCGCTCTG | 454 |
| hZC3H12A_gRNA_95 | CTTTGGGGGGTTCGAGGAGG | 455 |
| hZC3H12A_gRNA_96 | GGGCCGATGGCAAGCCTTGC | 456 |
| hZC3H12A_gRNA_97 | CACAGGCAGCTTACTCACTG | 457 |
| hZC3H12A_gRNA_98 | CCCAGACCAGCACATCCTGC | 458 |
| hZC3H12A_gRNA_99 | AGGCTGGGTTCCATACCATA | 459 |
| hZC3H12A_gRNA_100 | GGACTTCTAATTGCTGAGAA | 460 |
| hZC3H12A_gRNA_101 | CTCAAATTCCCACAGACTCA | 461 |
| hZC3H12A_gRNA_102 | AAAACAGGGCACAGGCAGAC | 462 |
| hZC3H12A_gRNA_103 | CCAGATGTCGGAACTTTGGG | 463 |
| hZC3H12A_gRNA_104 | CTCCCTCTAGTCCCGCGGGG | 464 |
| hZC3H12A_gRNA_105 | AGCCCCCAGTGCAGAGCCCA | 465 |
| hZC3H12A_gRNA_106 | CCTGGACGCCCAGCTTCTGC | 466 |
| hZC3H12A_gRNA_107 | CAGGGGCTGGCAGGAGCCCG | 467 |
| hZC3H12A_gRNA_108 | CCTTGTTCCCATGGCTGGGA | 468 |
| hZC3H12A_gRNA_109 | CTCATCTGCCACAGAGCGCT | 469 |
| hZC3H12A_gRNA_110 | GGCAGACACCAACACGGTGC | 470 |
| hZC3H12A_gRNA_111 | TCCCTCTTGATTCCTCTTCC | 471 |
| hZC3H12A_gRNA_112 | CCCTCCCAGCCATGGGAACA | 472 |
| hZC3H12A_gRNA_113 | GCGTAAGAAGCCACTCACTT | 473 |
| hZC3H12A_gRNA_114 | TGTGTTTCCCCCGCACCTGG | 474 |
| hZC3H12A_gRNA_115 | CTGAGACCAGTGGTCATCGA | 475 |
| hZC3H12A_gRNA_116 | GGGCAGCGACCTGAGACCAG | 476 |
| hZC3H12A_gRNA_117 | AGCAATTAGAAGTCCCTGCA | 477 |
| hZC3H12A_gRNA_118 | TGGGTGAGCTGGTGAAACAC | 478 |
| hZC3H12A_gRNA_119 | CTGTTAGCAGAGAGCTGGAC | 479 |
| hZC3H12A_gRNA_120 | CCCCTGATGACCCACTGGGC | 480 |
| hZC3H12A_gRNA_121 | GTTCACACCATCACGACGCG | 481 |
| hZC3H12A_gRNA_122 | TGTCCAGGCTGGGCCCGTGC | 482 |
| hZC3H12A_gRNA_123 | ACACAGACCTATGCCCCATC | 483 |
| hZC3H12A_gRNA_124 | GGCTGCCTGCCCTGCCCCAC | 484 |
| hZC3H12A_gRNA_125 | CCATAGGTGCATTTCCTTCC | 485 |
| hZC3H12A_gRNA_126 | CAGGCTGGGTTCCATACCAT | 486 |
| hZC3H12A_gRNA_127 | GCCCCATCACAGCCTCCACC | 487 |
| hZC3H12A_gRNA_128 | TGCCCTCCTTTTCCTCTTCT | 488 |
| hZC3H12A_gRNA_129 | GCCAGATGTCGGAACTTTGG | 489 |
| hZC3H12A_gRNA_130 | CAGGCAGACAGGTGAGAGGA | 490 |
| hZC3H12A_gRNA_131 | CCAGGAGTCTGAGCTATGAG | 491 |
| hZC3H12A_gRNA_132 | GCTCCAGGTTGGGAGCCTTA | 492 |
| hZC3H12A_gRNA_133 | CTCACCTGTGATGGGCACGT | 493 |
| hZC3H12A_gRNA_134 | AGCTGGCCTACGAGTCTGAC | 494 |
| hZC3H12A_gRNA_135 | GTGGGTGGGGGCAGTGGGTA | 495 |
| hZC3H12A_gRNA_136 | CATCTGCAGTTCCAGGGCCG | 496 |
| hZC3H12A_gRNA_137 | GATGACCCACTGGGCCGGCA | 497 |
| hZC3H12A_gRNA_138 | TGACCTCCAAGGCGAGCGGC | 498 |
| hZC3H12A_gRNA_139 | GGATCTCGGCAGCCAGCTGC | 499 |
| hZC3H12A_gRNA_140 | TCCTTTTCCTCTTCTGGGAG | 500 |
| hZC3H12A_gRNA_141 | CACGACGCGTGGGTGGCAAG | 501 |
| hZC3H12A_gRNA_142 | TTCACACCATCACGACGCGT | 502 |
| hZC3H12A_gRNA_143 | GCAGGAGCCTCGCACCTAGC | 503 |
| hZC3H12A_gRNA_144 | CACCCCTAAGGCTCCCAACC | 504 |
| hZC3H12A_gRNA_145 | TTGTCCTTGCTTGGGGCTCT | 505 |
| hZC3H12A_gRNA_146 | CAGGACAGGCTTCTCTCCAC | 506 |
| hZC3H12A_gRNA_147 | CACCTGGTGGAGGCTGTGAT | 507 |
| hZC3H12A_gRNA_148 | CGTCTGTGGGAGCCAGTCTG | 508 |
| hZC3H12A_gRNA_149 | CCCCCCAAAGTTCCGACATC | 509 |
| hZC3H12A_gRNA_150 | AGGCAGCCTGGCCAAGGAGC | 510 |
| hZC3H12A_gRNA_151 | TCTGCCTTTGGCCGGGCCAT | 511 |
| hZC3H12A_gRNA_152 | GGACAGGCTTCTCTCCACAG | 512 |
| hZC3H12A_gRNA_153 | ACGTGCCCATCACAGGTGAG | 513 |
| hZC3H12A_gRNA_154 | AGAGAGTGAGCAGTGCAGCC | 514 |
| hZC3H12A_gRNA_155 | CGCAGGAAGTTGTCCAGGCT | 515 |
| hZC3H12A_gRNA_156 | GGCTGGGAGCTCAGATCCAT | 516 |
| hZC3H12A_gRNA_157 | CAGCTCACCCAGCACCGTGT | 517 |
| hZC3H12A_gRNA_158 | CCAGCACATCCTGCGGGAAC | 518 |
| hZC3H12A_gRNA_159 | GACCTCCTTGTTCCCATGGC | 519 |
| hZC3H12A_gRNA_160 | GGGGTTCGAGGAGGAGGCCC | 520 |
| hZC3H12A_gRNA_161 | CAGAGAAGGCTGCGGTGGCT | 521 |
| hZC3H12A_gRNA_162 | GGGAGTGAGTCCAGCGTCTG | 522 |
| hZC3H12A_gRNA_163 | CAGGAGCCTCGCACCTAGCG | 523 |
| hZC3H12A_gRNA_164 | GGAGGAGGCCCTGGTGAGCC | 524 |
| hZC3H12A_gRNA_165 | CAAGCAAGGACAAAAATGGC | 525 |
| hZC3H12A_gRNA_166 | CGTCAGGGCACCCCAAGGCC | 526 |
| hZC3H12A_gRNA_167 | GCTGGCAGTGAACTGGTTTC | 527 |
| hZC3H12A_gRNA_168 | ACCTCCTTGTTCCCATGGCT | 528 |
| hZC3H12A_gRNA_169 | TCCCGCAGGATGTGCTGGTC | 529 |
| hZC3H12A_gRNA_170 | AGGGACTGTAGCCCGTGTAA | 530 |
| hZC3H12A_gRNA_171 | CCAGTACTCTCGAGGTGGAA | 531 |
| hZC3H12A_gRNA_172 | AATTCCCACAGACTCATGGT | 532 |
| hZC3H12A_gRNA_173 | CCCACCCCGAGCCCCTTACA | 533 |
| hZC3H12A_gRNA_174 | GTGCATTTCCTTCCTGGAAG | 534 |
| hZC3H12A_gRNA_175 | TCAGCGGCCAGCTCTTGACC | 535 |
| hZC3H12A_gRNA_176 | GGCCCGGCCAAAGGCAGAGA | 536 |
| hZC3H12A_gRNA_177 | ACAGAGCGCTGGGGGCAGCT | 537 |
| hZC3H12A_gRNA_178 | TCTTGATTCCTCTTCCAGGA | 538 |
| hZC3H12A_gRNA_179 | GCAAGGACAAAAATGGCCGG | 539 |
| hZC3H12A_gRNA_180 | CAGGGCAGGCAGCCTGGCCA | 540 |
| hZC3H12A_gRNA_181 | ATCTCGGCAGCCAGCTGCTG | 541 |
| hZC3H12A_gRNA_182 | CCCGCAGGATGTGCTGGTCT | 542 |
| hZC3H12A_gRNA_183 | GGCTCCAGGTTGGGAGCCTT | 543 |
| hZC3H12A_gRNA_184 | CAACACGGTGCTGGGTGAGC | 544 |
| hZC3H12A_gRNA_185 | GCAGCCGTGTCCCTATGGTA | 545 |
| hZC3H12A_gRNA_186 | TGTCCTTGCTTGGGGCTCTG | 546 |
| hZC3H12A_gRNA_187 | TCATGGTGGGGCTGGCTTCC | 547 |
| hZC3H12A_gRNA_188 | GAAGCTGGGCTATTCATCCA | 548 |
| hZC3H12A_gRNA_189 | GACCCTCTTGGCGGGACCCT | 549 |
| hZC3H12A_gRNA_190 | GGAAAGGCAGGGGGCGCGGG | 550 |
| hZC3H12A_gRNA_191 | AGGTCTGTGTTAGACCCTCT | 551 |
| hZC3H12A_gRNA_192 | CTCAGCTCCCTCTAGTCCCG | 552 |
| hZC3H12A_gRNA_193 | TAGGGACTGTAGCCCGTGTA | 553 |
| hZC3H12A_gRNA_194 | AGGGGGCATAAACCTGCAGA | 554 |
| hZC3H12A_gRNA_195 | CTCCCAGGATTGCCTGGACT | 555 |
| hZC3H12A_gRNA_196 | GGGATGAAGGTGAAGGCCGC | 556 |
| hZC3H12A_gRNA_197 | TGCAGAGCCCAGGGGCTGGC | 557 |
| hZC3H12A_gRNA_198 | GAATCGGCACTTGATCCCAT | 558 |
| hZC3H12A_gRNA_199 | CCGAGGCTGCTCCTTCCTCC | 559 |
| hZC3H12A_gRNA_200 | CCAGCTTCTGCAGGACGCTG | 560 |
| hZC3H12A_gRNA_201 | GGGCCGGCACGGGCCCAGCC | 561 |
| hZC3H12A_gRNA_202 | TGAGGTCTGGCGCTCCCGCT | 562 |
| hZC3H12A_gRNA_203 | TTGGGGTGCCCTGACGGCTG | 563 |
| hZC3H12A_gRNA_204 | ACTAGAGGGAGCTGAGGGCA | 564 |
| hZC3H12A_gRNA_205 | CCAGTTCCCGCAGGATGTGC | 565 |
| hZC3H12A_gRNA_206 | TATGCCCCCTGATGACCCAC | 566 |
| hZC3H12A_gRNA_207 | GTGAGAGGAGAGCATTGGCA | 567 |
| hZC3H12A_gRNA_208 | AGCTTACTCACTGGGGTGCT | 568 |
| hZC3H12A_gRNA_209 | ATCACAGCCTCCACCAGGTG | 569 |
| hZC3H12A_gRNA_210 | ACTGAAGTGGCCAGCACCCA | 570 |
| hZC3H12A_gRNA_211 | GCCGGCCCAGTGGGTCATCA | 571 |
| hZC3H12A_gRNA_212 | CCTGCAGAAGCTGGGCGTCC | 572 |
| hZC3H12A_gRNA_213 | GCACCGTGTTGGTGTCTGCC | 573 |
| hZC3H12A_gRNA_214 | GGCCCTGGAACTGCAGATGA | 574 |
| hZC3H12A_gRNA_215 | GTCCTTGCTTGGGGCTCTGG | 575 |
| hZC3H12A_gRNA_216 | CTCCCTGGAGAGCCAGATGT | 576 |
| hZC3H12A_gRNA_217 | AAATTCCCACAGACTCATGG | 577 |
| hZC3H12A_gRNA_218 | TCATCTGCCACAGAGCGCTG | 578 |
| hZC3H12A_gRNA_219 | AGTCGGCAGGGACACTGAAG | 579 |
| hZC3H12A_gRNA_220 | ACTCTCGAGGTGGAAAGGCA | 580 |
| hZC3H12A_gRNA_221 | CCCAGTGAGTAAGCTGCCTG | 581 |
| hZC3H12A_gRNA_222 | AGAGGGTGCAAAGAACTCTC | 582 |
| hZC3H12A_gRNA_223 | CACGATCCCGTCAGACTCGT | 583 |
| hZC3H12A_gRNA_224 | TCTGCACTGGGGGCTCCTGA | 584 |
| hZC3H12A_gRNA_225 | CAGGGGGCATAAACCTGCAG | 585 |
| hZC3H12A_gRNA_226 | TGAGGACAGCCACAGCCGTC | 586 |
| hZC3H12A_gRNA_227 | GTTTCCCCCGCACCTGGTGG | 587 |
| hZC3H12A_gRNA_228 | TTAGGGGTGCCACCACCCCG | 588 |
| hZC3H12A_gRNA_229 | ACTGGGGTGCTGGGACTTGT | 589 |
| hZC3H12A_gRNA_230 | CTCACTCCCGTACGTCTCCC | 590 |
| hZC3H12A_gRNA_231 | AGGGGCTGGCAGGAGCCCGT | 591 |
| hZC3H12A_gRNA_232 | TCCTTGTTCCCATGGCTGGG | 592 |
| hZC3H12A_gRNA_233 | GCCAAAGGCAGAGAAGGCTG | 593 |
| hZC3H12A_gRNA_234 | CACGGGCTCCTGCCAGCCCC | 594 |
| hZC3H12A_gRNA_235 | CCACAGCGTCCTGCAGAAGC | 595 |
| hZC3H12A_gRNA_236 | ACGGGCTCCTGCCAGCCCCT | 596 |
| hZC3H12A_gRNA_237 | ATGGGAGCAACGTGGCCATG | 597 |
| hZC3H12A_gRNA_238 | CCCAAGGCCGGGTCAAGAGC | 598 |
| hZC3H12A_gRNA_239 | AATTGCTGAGAAGGGGCCGA | 599 |
| hZC3H12A_gRNA_240 | GGGCAGGAGTGAGGAGGGCC | 600 |
| hZC3H12A_gRNA_241 | GGCGGGACCCTGGGGATGCC | 601 |
| hZC3H12A_gRNA_242 | GGGGCTGGCAGGAGCCCGTG | 602 |
| hZC3H12A_gRNA_243 | TTCCGACATCTGGCTCTCCA | 603 |
| hZC3H12A_gRNA_244 | GTGCTGCCCTTGCCAGCCAC | 604 |
| hZC3H12A_gRNA_245 | ACTCCTGCCGCTCGCCTTGG | 605 |
| hZC3H12A_gRNA_246 | GTGGACTTCTTCCGGAAGCT | 606 |
| hZC3H12A_gRNA_247 | CCAGTGCAGAGCCCAGGGGC | 607 |
| hZC3H12A_gRNA_248 | GGGGCAGTGGCAGCAGCTTT | 608 |
| hZC3H12A_gRNA_249 | GGGACTGTAGCCCGTGTAAG | 609 |
| hZC3H12A_gRNA_250 | CCACAGACTCATGGTGGGGC | 610 |
| hZC3H12A_gRNA_251 | AACACGGGACAGCCACCGAG | 611 |
| hZC3H12A_gRNA_252 | GCAAAGAACTCTCTGGAGGT | 612 |
| hZC3H12A_gRNA_253 | TGGGCCCGTGCCGGCCCAGT | 613 |
| hZC3H12A_gRNA_254 | CTCCTGCCGGGGCATCCTGC | 614 |
| hZC3H12A_gRNA_255 | AGGCAGACAGGTGAGAGGAA | 615 |
| hZC3H12A_gRNA_256 | AGGCAATCCTGGGAGACGTA | 616 |
| hZC3H12A_gRNA_257 | TCAGACCAGTACTCTCGAGG | 617 |
| hZC3H12A_gRNA_258 | AACATACTTGTCATTGACGA | 618 |
| hZC3H12A_gRNA_259 | GGCAGCTTGGCCGCTCTGGG | 619 |
| hZC3H12A_gRNA_260 | GAGTTCTTTGCACCCTCTGC | 620 |
| hZC3H12A_gRNA_261 | GCCACAGGCAGCTTACTCAC | 621 |
| hZC3H12A_gRNA_262 | AGGCTGCCTGCCCTGCCCCA | 622 |
| hZC3H12A_gRNA_263 | CCGGCCCAGTGGGTCATCAG | 623 |
| hZC3H12A_gRNA_264 | CTCTCGAGGTGGAAAGGCAG | 624 |
| hZC3H12A_gRNA_265 | GATTGCCTGGACTCGGGCAT | 625 |
| hZC3H12A_gRNA_266 | TCCTTGCTTGGGGCTCTGGG | 626 |
| hZC3H12A_gRNA_267 | GCAGAGAAGGCTGCGGTGGC | 627 |
| hZC3H12A_gRNA_268 | ACCGTGACCTCCAAGGCGAG | 628 |
| hZC3H12A_gRNA_269 | CAGGACGCTGTGGATCTCCG | 629 |
| hZC3H12A_gRNA_270 | AGGAAGCAGCCGTGTCCCTA | 630 |
| hZC3H12A_gRNA_271 | ACGCAGGAAGTTGTCCAGGC | 631 |
| hZC3H12A_gRNA_272 | GAGGTCCCCAAGAGGAAAAC | 632 |
| hZC3H12A_gRNA_273 | CCCCCAGCTTCTTCCCATCC | 633 |
| hZC3H12A_gRNA_274 | ATTCCCACAGACTCATGGTG | 634 |
| hZC3H12A_gRNA_275 | TCCAAGGCGAGCGGCAGGAG | 635 |
| hZC3H12A_gRNA_276 | GCTGGGAGCTCAGATCCATA | 636 |
| hZC3H12A_gRNA_277 | TGGGGGCCCAGGCATCCCCA | 637 |
| hZC3H12A_gRNA_278 | GGGTGCAAAGAACTCTCTGG | 638 |
| hZC3H12A_gRNA_279 | GCGGGACTAGAGGGAGCTGA | 639 |
| hZC3H12A_gRNA_280 | ACTGGAGAAGAAGAAGATCC | 640 |
| hZC3H12A_gRNA_281 | CCAGCTCTTGACCCGGCCTT | 641 |
| hZC3H12A_gRNA_282 | GAACTTTGGGGGGTTCGAGG | 642 |
| hZC3H12A_gRNA_283 | GAAACCAGTTCACTGCCAGC | 643 |
| hZC3H12A_gRNA_284 | ACAGCCGTCAGGGCACCCCA | 644 |
| hZC3H12A_gRNA_285 | CCACCCCGAGCCCCTTACAC | 645 |
| hZC3H12A_gRNA_286 | TCTCGGCAGCCAGCTGCTGG | 646 |
| hZC3H12A_gRNA_287 | AGAGAGCTGGACTGGGATGA | 647 |
| hZC3H12A_gRNA_288 | CCTTTCCACCTCGAGAGTAC | 648 |
| hZC3H12A_gRNA_289 | AAGCTGGCCTACGAGTCTGA | 649 |
| hZC3H12A_gRNA_290 | GTCTGTGGGAGCCAGTCTGT | 650 |
| hZC3H12A_gRNA_291 | AGACCTATGCCCCATCAGGC | 651 |
| hZC3H12A_gRNA_292 | TGGGAAGAAGCTGGGGGCCC | 652 |
| hZC3H12A_gRNA_293 | CTGTGGAGAGAAGCCTGTCC | 653 |
| hZC3H12A_gRNA_294 | GGGACTTCTAATTGCTGAGA | 654 |
| hZC3H12A_gRNA_295 | GGACTCGGGCATTGGCTCCC | 655 |
| hZC3H12A_gRNA_296 | CATCTGCCACAGAGCGCTGG | 656 |
| hZC3H12A_gRNA_297 | CTTCTGGGAGTGGAGGCTCC | 657 |
| hZC3H12A_gRNA_298 | GCCCCCAGTGCAGAGCCCAG | 658 |
| hZC3H12A_gRNA_299 | TTTGTCCTTGCTTGGGGCTC | 659 |
| hZC3H12A_gRNA_300 | GTGGGGCTGGCTTCCAGGAC | 660 |
| hZC3H12A_gRNA_301 | TCAAGAGCTGGCCGCTGAGG | 661 |
| hZC3H12A_gRNA_302 | CCTCTAGTCCCGCGGGGTGG | 662 |
| hZC3H12A_gRNA_303 | GCTCATCTGCCACAGAGCGC | 663 |
| hZC3H12A_gRNA_304 | CATGAGTCTGTGGGAATTTG | 664 |
| hZC3H12A_gRNA_305 | TGCGAGGCTCCTGCCTGATG | 665 |
| hZC3H12A_gRNA_306 | GGAGTGAGTCCAGCGTCTGT | 666 |
| hZC3H12A_gRNA_307 | TGCAAAGAACTCTCTGGAGG | 667 |
| hZC3H12A_gRNA_308 | CACAGCGTCCTGCAGAAGCT | 668 |
| hZC3H12A_gRNA_309 | CAGCTTACTCACTGGGGTGC | 669 |
| hZC3H12A_gRNA_310 | ACTGATGTGGGTGGGGGCAG | 670 |
| hZC3H12A_gRNA_311 | GCAGGATGTGCTGGTCTGGG | 671 |
| hZC3H12A_gRNA_312 | TCACAGTGTTTGTGCCATCC | 672 |
| hZC3H12A_gRNA_313 | GTTTGTGCCATCCTGGAGGA | 673 |
| hZC3H12A_gRNA_314 | TCCTGAAGGACTGATGTGGG | 674 |
| hZC3H12A_gRNA_315 | TGTTAGCAGAGAGCTGGACT | 675 |
| hZC3H12A_gRNA_316 | CAGTGTTTGTGCCATCCTGG | 676 |
| hZC3H12A_gRNA_317 | AGTCTGTCAGGGCCTCTGGG | 677 |
| hZC3H12A_gRNA_318 | TCTCGAGGTGGAAAGGCAGG | 678 |
| hZC3H12A_gRNA_319 | AGACTGGCTCCCACAGACGC | 679 |
| hZC3H12A_gRNA_320 | AGCCACTCACTTTGGAGCAC | 680 |
| hZC3H12A_gRNA_321 | TCCCAGGATTGCCTGGACTC | 681 |
| hZC3H12A_gRNA_322 | CCTGGAACTGCAGATGAAGG | 682 |
| hZC3H12A_gRNA_323 | GGGGCGCTTCCCACAGCTCC | 683 |
| hZC3H12A_gRNA_324 | CAGCCCCTGGGCTCTGCACT | 684 |
| hZC3H12A_gRNA_325 | GCGCGGGTGGGTAGTCGGCA | 685 |
| hZC3H12A_gRNA_326 | GCCCCAAGCAAGGACAAAAA | 686 |
| hZC3H12A_gRNA_327 | AGCCTGGATGGGAAGAAGCT | 687 |
| hZC3H12A_gRNA_328 | CAGCTCTTGACCCGGCCTTG | 688 |
| hZC3H12A_gRNA_329 | TAGGGGTGCCACCACCCCGC | 689 |
| hZC3H12A_gRNA_330 | TCCACTCCCAGAAGAGGAAA | 690 |
| hZC3H12A_gRNA_331 | GGAAGCGCTTCATCGAGGAG | 691 |
| hZC3H12A_gRNA_332 | GCATCCTGCTGGCAGTGAAC | 692 |
| hZC3H12A_gRNA_333 | TGGATGAATAGCCCAGCTTC | 693 |
| hZC3H12A_gRNA_334 | ACACGGGACAGCCACCGAGC | 694 |
| hZC3H12A_gRNA_335 | GGGCTCCTGAAGGACTGATG | 695 |
| hZC3H12A_gRNA_336 | CAGCCTGGATGGGAAGAAGC | 696 |
| hZC3H12A_gRNA_337 | TTTTCCTCTTCTGGGAGTGG | 697 |
| hZC3H12A_gRNA_338 | CTCCAGGTTGGGAGCCTTAG | 698 |
| hZC3H12A_gRNA_339 | GGGAGCTGAGGGCAGGGGTC | 699 |
| hZC3H12A_gRNA_340 | AGATGAAGGTGGACTTCTTC | 700 |
| hZC3H12A_gRNA_341 | TTTGGCCGGGCCATGGGTGC | 701 |
| hZC3H12A_gRNA_342 | CTCGCACCTAGCGGGGGCAG | 702 |
| hZC3H12A_gRNA_343 | CCCGTGTAAGGGGCTCGGGG | 703 |
| hZC3H12A_gRNA_344 | TGCCGGCCCAGTGGGTCATC | 704 |
| hZC3H12A_gRNA_345 | AAAGGCAGAGAAGGCTGCGG | 705 |
| hZC3H12A_gRNA_346 | AGGAGCCCGTGGGGCAGGGC | 706 |
| hZC3H12A_gRNA_347 | TAAGGGGCTCGGGGTGGGCC | 707 |
| hZC3H12A_gRNA_348 | ACACCATCACGACGCGTGGG | 708 |
| hZC3H12A_gRNA_349 | CTGGCAGGAGCCCGTGGGGC | 709 |
| hZC3H12A_gRNA_350 | CCGGCCTTGGGGTGCCCTGA | 710 |
| hZC3H12A_gRNA_351 | CTGTGTTAGACCCTCTTGGC | 711 |
| hZC3H12A_gRNA_352 | GTGATGGGCACGTCGGGCCG | 712 |
| hZC3H12A_gRNA_353 | GCCCCTGGGCTCTGCACTGG | 713 |
| hZC3H12A_gRNA_354 | CTGGGTGAGCTGGTGAAACA | 714 |
| hZC3H12A_gRNA_355 | GGCTGCTCCTTCCTCCAGGA | 715 |
| hZC3H12A_gRNA_356 | ACAGCCTCCACCAGGTGCGG | 716 |
| hZC3H12A_gRNA_357 | TGCCCGAGTCCAGGCAATCC | 717 |
| hZC3H12A_gRNA_358 | AGGTGGAAAGGCAGGGGGCG | 718 |
| hZC3H12A_gRNA_359 | CGACAGATTCATTGTGAAGC | 719 |
| hZC3H12A_gRNA_360 | GCGGGGTGGTGGCACCCCTA | 720 |
| hZC3H12A_gRNA_361 | GGCAATCCTGGGAGACGTAC | 721 |
| hZC3H12A_gRNA_362 | GCCGCTCGCCTTGGAGGTCA | 722 |
| hZC3H12A_gRNA_363 | TCACTGCCAGCAGGATGCCC | 723 |
| hZC3H12A_gRNA_364 | CCTGAAGGACTGATGTGGGT | 724 |
| hZC3H12A_gRNA_365 | GTGCGAGGCTCCTGCCTGAT | 725 |
| hZC3H12A_gRNA_366 | GCACCTGGTGGAGGCTGTGA | 726 |
| hZC3H12A_gRNA_367 | TCACAGCCTCCACCAGGTGC | 727 |
| hZC3H12A_gRNA_368 | GCCGCTCTGGGTGGAAGAAT | 728 |
| hZC3H12A_gRNA_369 | GACTAGAGGGAGCTGAGGGC | 729 |
| hZC3H12A_gRNA_370 | TCAGCTCCCTCTAGTCCCGC | 730 |
| hZC3H12A_gRNA_371 | GGAGCCTCCACTCCCAGAAG | 731 |
| hZC3H12A_gRNA_372 | AGACCCTCTTGGCGGGACCC | 732 |
| hZC3H12A_gRNA_373 | CCACCTTCATCTGCAGTTCC | 733 |
| hZC3H12A_gRNA_374 | GGGAGTGGAGGCTCCAGGTT | 734 |
| hZC3H12A_gRNA_375 | CAGTGAACTGGTTTCTGGAG | 735 |
| hZC3H12A_gRNA_376 | TCACCTGTGATGGGCACGTC | 736 |
| hZC3H12A_gRNA_377 | TGCCAGCAGGATGCCCCGGC | 737 |
| hZC3H12A_gRNA_378 | ACCCTCTTGGCGGGACCCTG | 738 |
| hZC3H12A_gRNA_379 | TGGGGGCAGCTTGGCCGCTC | 739 |
| hZC3H12A_gRNA_380 | AGGAGGAGGCCCTGGTGAGC | 740 |
| hZC3H12A_gRNA_381 | CTGGAGGTGGGAGCCATGCA | 741 |
| hZC3H12A_gRNA_382 | TCTGGAGGTGGGAGCCATGC | 742 |
| hZC3H12A_gRNA_383 | CCTGGATGGGAAGAAGCTGG | 743 |
| hZC3H12A_gRNA_384 | CCAGCCCCTGGGCTCTGCAC | 744 |
| hZC3H12A_gRNA_385 | GGAGTGGAAGCGCTTCATCG | 745 |
| hZC3H12A_gRNA_386 | TGTAGCCCGTGTAAGGGGCT | 746 |
| hZC3H12A_gRNA_387 | GGAGTGAGGAGGGCCGGGGA | 747 |
| hZC3H12A_gRNA_388 | GAGGTCACGGTATGTGTCGT | 748 |
| hZC3H12A_gRNA_389 | CTAGAGGGAGCTGAGGGCAG | 749 |
| hZC3H12A_gRNA_390 | TGGTGTGTTTCCCCCGCACC | 750 |
| hZC3H12A_gRNA_391 | CTGATGTGGGTGGGGGCAGT | 751 |
| hZC3H12A_gRNA_392 | AGGGCCGGGGAGGGCAGGCT | 752 |
| hZC3H12A_gRNA_393 | TGAGCTATGAGTGGCCCCTG | 753 |
| hZC3H12A_gRNA_394 | TCTTACGCAGGAAGTTGTCC | 754 |
| hZC3H12A_gRNA_395 | GTTCCGACATCTGGCTCTCC | 755 |
| hZC3H12A_gRNA_396 | AGGGGGCGCGGGTGGGTAGT | 756 |
| hZC3H12A_gRNA_397 | CGCTGGCCTGCTCCTTGGCC | 757 |
| hZC3H12A_gRNA_398 | GAAAGGCAGGGGGCGCGGGT | 758 |
| hZC3H12A_gRNA_399 | TAGCCCGTGTAAGGGGCTCG | 759 |
| hZC3H12A_gRNA_400 | CTGAGGGCAGGGGTCCGGTG | 760 |
| hZC3H12A_gRNA_401 | ACACAGCTTAGTATACACGC | 761 |
| hZC3H12A_gRNA_402 | CCGTCAGGGCACCCCAAGGC | 762 |
| hZC3H12A_gRNA_403 | GGCAGGGGTCCGGTGAGGTC | 763 |
| hZC3H12A_gRNA_404 | GGACTTGTAGGAGAGGATCT | 764 |
| hZC3H12A_gRNA_405 | TCCCAGCCATGGGAACAAGG | 765 |
| hZC3H12A_gRNA_406 | GACTTCTAATTGCTGAGAAG | 766 |
| hZC3H12A_gRNA_407 | GGCTCCTGAAGGACTGATGT | 767 |
| hZC3H12A_gRNA_408 | TGGCAGGAGCCCGTGGGGCA | 768 |
| hZC3H12A_gRNA_409 | AGACAGGTGAGAGGAAGGGC | 769 |
| hZC3H12A_gRNA_410 | TCGGAACTTTGGGGGGTTCG | 770 |
| hZC3H12A_gRNA_411 | GCCTGGATGGGAAGAAGCTG | 771 |
| hZC3H12A_gRNA_412 | TGAAGGACTGATGTGGGTGG | 772 |
| hZC3H12A_gRNA_413 | CTGGGGGCCCAGGCATCCCC | 773 |
| hZC3H12A_gRNA_414 | GAGCCCCCAGTGCAGAGCCC | 774 |
| hZC3H12A_gRNA_415 | GGCGCGGGTGGGTAGTCGGC | 775 |
| hZC3H12A_gRNA_416 | CCGTGTAAGGGGCTCGGGGT | 776 |
| hZC3H12A_gRNA_417 | GTCGTGATGGTGTGAACACC | 777 |
| hZC3H12A_gRNA_418 | ACGACGCGTGGGTGGCAAGC | 778 |
| hZC3H12A_gRNA_419 | GGGGGCAGTGGCAGCAGCTT | 779 |
| hZC3H12A_gRNA_420 | AGCGTGTATACTAAGCTGTG | 780 |
| hZC3H12A_gRNA_421 | GCTCCTGCCTGATGGGGCAT | 781 |
| hZC3H12A_gRNA_422 | GTCTGTCAGGGCCTCTGGGA | 782 |
| hZC3H12A_gRNA_423 | GTAGCCCGTGTAAGGGGCTC | 783 |
| hZC3H12A_gRNA_424 | AGCCCCTGGGCTCTGCACTG | 784 |
| hZC3H12A_gRNA_425 | GTGAACTGGTTTCTGGAGCG | 785 |
| hZC3H12A_gRNA_426 | CTACGAGTCTGACGGGATCG | 786 |
| hZC3H12A_gRNA_427 | AGTGAACTGGTTTCTGGAGC | 787 |
| hZC3H12A_gRNA_428 | ACGCGTGGGTGGCAAGCGGG | 788 |
| hZC3H12A_gRNA_429 | CGCGGGACTAGAGGGAGCTG | 789 |
| hZC3H12A_gRNA_430 | GGCAGGAGTGAGGAGGGCCG | 790 |
| hZC3H12A_gRNA_431 | AAGTGAGTGGCTTCTTACGC | 791 |
| hZC3H12A_gRNA_432 | CTGAAGGACTGATGTGGGTG | 792 |
| hZC3H12A_gRNA_433 | TTGCCACCCACGCGTCGTGA | 793 |
| hZC3H12A_gRNA_434 | AGGGCAGGAGTGAGGAGGGC | 794 |
| hZC3H12A_gRNA_435 | TCTTCTTCTCCAGTTCCCGC | 795 |
| hZC3H12A_gRNA_436 | AGGAGTGAGGAGGGCCGGGG | 796 |
| hZC3H12A_gRNA_437 | ACTCCCAGAAGAGGAAAAGG | 797 |
| hZC3H12A_gRNA_438 | TGAGGAGGGCCGGGGAGGGC | 798 |
| hZC3H12A_gRNA_439 | ACCTGGTGGAGGCTGTGATG | 799 |
| hZC3H12A_gRNA_440 | CAGGGCCGAGGCCTCCTCAG | 800 |
| hZC3H12A_gRNA_441 | TACTCTCGAGGTGGAAAGGC | 801 |
| hZC3H12A_gRNA_442 | TTGGGGCTCTGGGGGGTGAG | 802 |
| hZC3H12A_gRNA_443 | GCTCCTGGACCCCCAGCAGC | 803 |
| hZC3H12A_gRNA_444 | GGGGGGTGAGAGGAGAGCAT | 804 |
| hZC3H12A_gRNA_445 | CGGCCCAGTGGGTCATCAGG | 805 |
| hZC3H12A_gRNA_446 | AGGAAGGGCAGGAGTGAGGA | 806 |
| hZC3H12A_gRNA_447 | GAGGGCCGGGGAGGGCAGGC | 807 |
| hZC3H12A_gRNA_448 | TGCTGGGGGTCCAGGAGCTG | 808 |
| hZC3H12A_gRNA_449 | GCTGGGGGTCCAGGAGCTGT | 809 |
| hZC3H12A_gRNA_450 | TGAGAGGAAGGGCAGGAGTG | 810 |
| hZC3H12A_gRNA_451 | TGGGAGTGGAGGCTCCAGGT | 811 |
| hZC3H12A_gRNA_452 | GAGGAAGGGCAGGAGTGAGG | 812 |

TABLE 17
Exemplary murine Zc3h12a gRNA sequences

| Target | Sequence | SEQ ID |
|---|---|---|
| mZc3h12a_gRNA_1 | GCTGGCTGTGAACTGGTTTC | 208 |
| mZc3h12a_gRNA_2 | CTAGTTCCCGAAGGATGTGC | 209 |
| mZc3h12a_gRNA_3 | ATTGGAGACCACCACTCCGT | 210 |
| mZc3h12a_gRNA_4 | TTCCCTCCTCTGCCAGCCAT | 211 |
| mZc3h12a_gRNA_5 | CGAAGGAAGTTGTCCAGGCT | 212 |
| mZc3h12a_gRNA_6 | ATACCTGTGATAGGCACATC | 213 |
| mZc3h12a_gRNA_7 | GACTTCCTTGTTCCCATGGC | 214 |
| mZc3h12a_gRNA_8 | GGCCTTCGAATCCGACGGAG | 215 |

In some embodiments, the gene-regulating system comprises at least two gRNA molecules, wherein at least one gRNA molecule comprises a SOCS1-targeting nucleic acid-binding segment (i.e., a SOCS1-targeting gRNA) and at least one gRNA molecule comprises a PTPN2-targeting nucleic acid-binding segment (i.e., a PTPN2-targeting gRNA). In some embodiments, the nucleic acid-binding segment of the at least one SOCS1-targeting gRNA binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the nucleic acid-binding segment of the at least one PTPN2-targeting gRNA binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the nucleic acid-binding segment of the at least one SOCS1-targeting gRNA binds to a target DNA sequence that is 100% identical to a DNA sequence in the SOCS1 gene (SEQ ID NO: 1) or the Socs1 gene (SEQ ID NO: 2) and the nucleic acid-binding segment of the at least one PTPN2-targeting gRNA binds to a target DNA sequence that is 100% identical to a DNA sequence in the PTPN2 gene (SEQ ID NO: 3) or the Ptpn2 gene (SEQ ID NO: 4).

In some embodiments, the nucleic acid-binding segment of the at least one SOCS1-targeting gRNA binds to a target DNA sequence that is at least 95%, 96%, 97%, 98%, or 99% identical to a DNA sequence defined by a set of genomic coordinates shown in Table 3 or Table 4 and the nucleic acid-binding segment of the at least one PTPN2-targeting gRNA binds to a target DNA sequence that is at least 95%, 96%, 97%, 98%, or 99% identical to a DNA sequence defined by a set of genomic coordinates shown in Table 5 or Table 6. In some embodiments, the nucleic acid-binding segment of the at least one SOCS1-targeting gRNA binds to a target DNA sequence that is 100% identical to a DNA sequence defined by a set of genomic coordinates shown in Table 3 or Table 4 and the nucleic acid-binding segment of the at least one PTPN2-targeting gRNA binds to a target DNA sequence that is 100% identical to a DNA sequence defined by a set of genomic coordinates shown in Table 5 or Table 6.

In some embodiments, the nucleic acid-binding segment of the at least one SOCS1-targeting gRNA binds to a target DNA sequence that is at least 95%, 96%, 97%, 98%, or 99% identical to one of SEQ ID NOs: 7-151 and the nucleic acid-binding segment of the at least one PTPN2-targeting gRNA binds to a target DNA sequence that is at least 95%, 96%, 97%, 98%, or 99% identical to one of SEQ ID NOs: 185-207. In some embodiments, the nucleic acid-binding segment of the at least one SOCS1-targeting gRNA binds to a target DNA sequence that is 100% identical to one of SEQ ID NOs: 7-151 and the nucleic acid-binding segment of the at least one PTPN2-targeting gRNA binds to a target DNA sequence that is 100% identical to one of SEQ ID NOs: 185-207. Exemplary SOCS1 target DNA sequences are shown in Tables 12 and 13 and exemplary PTPN2 target DNA sequences are shown in Tables 14 and 15.

In some embodiments, the nucleic acid-binding segment of the at least one SOCS1-targeting gRNA is encoded by a DNA sequence that is at least 95%, 96%, 97%, 98%, or 99% identical to one of SEQ ID NOs: 7-151 and the nucleic acid-binding segment of the at least one PTPN2-targeting gRNA is encoded by a DNA sequence that is at least 95%, 96%, 97%, 98%, or 99% identical to one of SEQ ID NOs: 185-207. In some embodiments, the nucleic acid-binding segment of the at least one SOCS1-targeting gRNA is encoded by a DNA sequence that is 100% identical to one of SEQ ID NOs: 7-151 and the nucleic acid-binding segment of the at least one PTPN2-targeting gRNA is encoded by a DNA sequence that is 100% identical to one of SEQ ID NOs: 185-207. Exemplary DNA sequences encoding the nucleic acid-binding segment of the SOCS1-targeting gRNAs are shown in Tables 12 and 13 and exemplary DNA sequences encoding the nucleic acid-binding segment of the PTPN2-targeting gRNAs are shown in Tables 14 and 15.

In some embodiments, the gene-regulating system comprises at least two gRNA molecules, wherein at least one gRNA molecule comprises a SOCS1-targeting nucleic acid-binding segment (i.e., a SOCS1-targeting gRNA) and at least one gRNA molecule comprises a ZC3H12A-targeting nucleic acid-binding segment (i.e., a ZC3H12A-targeting gRNA). In some embodiments, the nucleic acid-binding segment of the at least one SOCS1-targeting gRNA binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the nucleic acid-binding segment of the at least one ZC3H12A-targeting gRNA binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the nucleic acid-binding segment of the at least one SOCS1-targeting gRNA binds to a target DNA sequence that is 100% identical to a DNA sequence in the SOCS1 gene (SEQ ID NO: 1) or the Socs1 gene (SEQ ID NO: 2) and the nucleic acid-binding segment of the at least one ZC3H12A-targeting gRNA binds to a target DNA sequence that is 100% identical to a DNA sequence in the ZC3H12A gene (SEQ ID NO: 5) or the Zc3h12a gene (SEQ ID NO: 6).

In some embodiments, the nucleic acid-binding segment of the at least one SOCS1-targeting gRNA binds to a target DNA sequence that is at least 95%, 96%, 97%, 98%, or 99% identical to a DNA sequence defined by a set of genomic coordinates shown in Table 3 or Table 4 and the nucleic acid-binding segment of the at least one ZC3H12A-targeting gRNA binds to a target DNA sequence that is at least 95%, 96%, 97%, 98%, or 99% identical to a DNA sequence defined by a set of genomic coordinates shown in Table 7 or Table 8. In some embodiments, the nucleic acid-binding segment of the at least one SOCS1-targeting gRNA binds to a target DNA sequence that is 100% identical to a DNA sequence defined by a set of genomic coordinates shown in Table 3 or Table 4 and the nucleic acid-binding segment of the at least one ZC3H12A-targeting gRNA binds to a target DNA sequence that is 100% identical to a DNA sequence defined by a set of genomic coordinates shown in Table 7 or Table 8.

In some embodiments, the nucleic acid-binding segment of the at least one SOCS1-targeting gRNA binds to a target DNA sequence that is at least 95%, 96%, 97%, 98%, or 99% identical to one of SEQ ID NOs: 7-151 and the nucleic acid-binding segment of the at least one ZC3H12A-targeting gRNA binds to a target DNA sequence that is at least 95%, 96%, 97%, 98%, or 99% identical to one of SEQ ID NOs: 208-230. In some embodiments, the nucleic acid-binding segment of the at least one SOCS1-targeting gRNA binds to a target DNA sequence that is 100% identical to one of SEQ ID NOs: 7-151 and the nucleic acid-binding segment of the at least one ZC3H12A-targeting gRNA binds to a target DNA sequence that is 100% identical to one of SEQ ID NOs: 208-230. Exemplary SOCS1 target DNA sequences are shown in Tables 12 and 13 and exemplary ZC3H12A target DNA sequences are shown in Tables 16 and 17.

In some embodiments, the nucleic acid-binding segment of the at least one SOCS1-targeting gRNA is encoded by a DNA sequence that is at least 95%, 96%, 97%, 98%, or 99% identical to one of SEQ ID NOs: 7-151 and the nucleic acid-binding segment of the at least one ZC3H12A-targeting gRNA is encoded by a DNA sequence that is at least 95%, 96%, 97%, 98%, or 99% identical to one of SEQ ID NOs: 208-230. In some embodiments, the nucleic acid-binding segment of the at least one SOCS1-targeting gRNA is encoded by a DNA sequence that is 100% identical to one of SEQ ID NOs: 7-151 and the nucleic acid-binding segment of the at least one ZC3H12A-targeting gRNA is encoded by a DNA sequence that is 100% identical to one of SEQ ID NOs: 208-230. Exemplary DNA sequences encoding the nucleic acid-binding segment of the SOCS1-targeting gRNAs are shown in Tables 12 and 13 and exemplary DNA sequences encoding the nucleic acid-binding segment of the ZC3H12A-targeting gRNAs are shown in Tables 16 and 17.

In some embodiments, the gene-regulating system comprises at least two gRNA molecules, wherein at least one gRNA molecule comprises a PTPN2-targeting nucleic acid-binding segment (i.e., a PTPN2-targeting gRNA) and at least one gRNA molecule comprises a ZC3H12A-targeting nucleic acid-binding segment (i.e., a ZC3H12A-targeting gRNA). In some embodiments, the nucleic acid-binding segment of the at least one PTPN2-targeting gRNA binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4) and the nucleic acid-binding segment of the at least one ZC3H12A-targeting gRNA binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the nucleic acid-binding segment of the at least one PTPN2-targeting gRNA binds to a target DNA sequence that is 100% identical to a DNA sequence in the PTPN2 gene (SEQ ID NO: 3) or the Ptpn2 gene (SEQ ID NO: 4) and the nucleic acid-binding segment of the at least one ZC3H12A-targeting gRNA binds to a target DNA sequence that is 100% identical to a DNA sequence in the ZC3H12A gene (SEQ ID NO: 5) or the Zc3h12a gene (SEQ ID NO: 6).

In some embodiments, the nucleic acid-binding segment of the at least one PTPN2-targeting gRNA binds to a target DNA sequence that is at least 95%, 96%, 97%, 98%, or 99% identical to a DNA sequence defined by a set of genomic coordinates shown in Table 5 or Table 6 and the nucleic acid-binding segment of the at least one ZC3H12A-targeting gRNA binds to a target DNA sequence that is at least 95%, 96%, 97%, 98%, or 99% identical to a DNA sequence defined by a set of genomic coordinates shown in Table 7 or Table 8. In some embodiments, the nucleic acid-binding segment of the at least one PTPN2-targeting gRNA binds to a target DNA sequence that is 100% identical to a DNA sequence defined by a set of genomic coordinates shown in Table 5 or Table 6 and the nucleic acid-binding segment of the at least one ZC3H12A-targeting gRNA binds to a target DNA sequence that is 100% identical to a DNA sequence defined by a set of genomic coordinates shown in Table 7 or Table 8.

In some embodiments, the nucleic acid-binding segment of the at least one PTPN2-targeting gRNA binds to a target DNA sequence that is at least 95%, 96%, 97%, 98%, or 99% identical to one of SEQ ID NOs: 185-207 and the nucleic acid-binding segment of the at least one ZC3H12A-targeting gRNA binds to a target DNA sequence that is at least 95%, 96%, 97%, 98%, or 99% identical to one of SEQ ID NOs: 208-230. In some embodiments, the nucleic acid-binding segment of the at least one PTPN2-targeting gRNA binds to a target DNA sequence that is 100% identical to one of SEQ ID NOs: 185-207 and the nucleic acid-binding segment of the at least one ZC3H/2A-targeting gRNA binds to a target DNA sequence that is 100% identical to one of SEQ ID NOs: 208-230. Exemplary PTPN2 target DNA sequences are shown in Tables 14 and 15 and exemplary ZC3H12A target DNA sequences are shown in Tables 16 and 17.

In some embodiments, the nucleic acid-binding segment of the at least one PTPN2-targeting gRNA is encoded by a DNA sequence that is at least 95%, 96%, 97%, 98%, or 99% identical to one of SEQ ID NOs: 185-207 and the nucleic acid-binding segment of the at least one ZC3H12A-targeting gRNA is encoded by a DNA sequence that is at least 95%, 96%, 97%, 98%, or 99% identical to one of SEQ ID NOs: 208-230. In some embodiments, the nucleic acid-binding segment of the at least one PTPN2-targeting gRNA is encoded by a DNA sequence that is 100% identical to one of SEQ ID NOs: 185-207 and the nucleic acid-binding segment of the at least one ZC3H/2A-targeting gRNA is encoded by a DNA sequence that is 100% identical to one of SEQ ID NOs: 208-230. Exemplary DNA sequences encoding the nucleic acid-binding segment of the PTPN2-targeting gRNAs are shown in Tables 14 and 15 and exemplary DNA sequences encoding the nucleic acid-binding segment of the ZC3H/2A-targeting gRNAs are shown in Tables 16 and 17.

In some embodiments, the nucleic acid-binding segments of the gRNA sequences described herein are designed to minimize off-target binding using algorithms known in the art (e.g., Cas-OFF finder) to identify target sequences that are unique to a particular target locus or target gene.

In some embodiments, the gRNAs described herein can comprise one or more modified nucleosides or nucleotides which introduce stability toward nucleases. In such embodiments, these modified gRNAs may elicit a reduced innate immune as compared to a non-modified gRNA. The term “innate immune response” includes a cellular response to exogenous nucleic acids, including single stranded nucleic acids, generally of viral or bacterial origin, which involves the induction of cytokine expression and release, particularly the interferons, and cell death.

In some embodiments, the gRNAs described herein are modified at or near the 5′ end (e.g., within 1-10, 1-5, or 1-2 nucleotides of their 5′ end). In some embodiments, the 5′ end of a gRNA is modified by the inclusion of a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)). In some embodiments, an in vitro transcribed gRNA is modified by treatment with a phosphatase (e.g., calf intestinal alkaline phosphatase) to remove the 5′ triphosphate group. In some embodiments, a gRNA comprises a modification at or near its 3′ end (e.g., within 1-10, 1-5, or 1-2 nucleotides of its 3′ end). For example, in some embodiments, the 3′ end of a gRNA is modified by the addition of one or more (e.g., 25-200) adenine (A) residues.

In some embodiments, modified nucleosides and modified nucleotides can be present in a gRNA, but also may be present in other gene-regulating systems, e.g., mRNA, RNAi, or siRNA-based systems. In some embodiments, modified nucleosides and nucleotides can include one or more of:

(a) alteration, e.g., replacement, of one or both of the non-linking phosphate oxygens and/or of one or more of the linking phosphate oxygens in the phosphodiester backbone linkage;

(b) alteration, e.g., replacement, of a constituent of the ribose sugar, e.g., of the 2′ hydroxyl on the ribose sugar;

(c) wholesale replacement of the phosphate moiety with “dephospho” linkers;

(d) modification or replacement of a naturally occurring nucleobase;

(e) replacement or modification of the ribose-phosphate backbone;

(f) modification of the 3′ end or 5′ end of the oligonucleotide, e.g., removal, modification or replacement of a terminal phosphate group or conjugation of a moiety; and

(g) modification of the sugar.

In some embodiments, the modifications listed above can be combined to provide modified nucleosides and nucleotides that can have two, three, four, or more modifications. For example, in some embodiments, a modified nucleoside or nucleotide can have a modified sugar and a modified nucleobase. In some embodiments, every base of a gRNA is modified. In some embodiments, each of the phosphate groups of a gRNA molecule are replaced with phosphorothioate groups.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool.

### IV. Methods of Producing Modified Immune Effector Cells

In some embodiments, the present disclosure provides methods for producing modified immune effector cells. In some embodiments, the methods comprise introducing a gene-regulating system into a population of immune effector cells wherein the gene-regulating system is capable of reducing expression and/or function of two or more endogenous target genes selected from SOCS1, PTPN2, and ZC3H12A.

The components of the gene-regulating systems described herein, e.g., a nucleic acid-, protein-, or nucleic acid/protein-based system can be introduced into target cells in a variety of forms using a variety of delivery methods and formulations. In some embodiments, a polynucleotide encoding one or more components of the system is delivered by a recombinant vector (e.g., a viral vector or plasmid). In some embodiments, where the system comprises more than a single component, a vector may comprise a plurality of polynucleotides, each encoding a component of the system. In some embodiments, where the system comprises more than a single component, a plurality of vectors may be used, wherein each vector comprises a polynucleotide encoding a particular component of the system. In some embodiments, a vector may also comprise a sequence encoding a signal peptide (e.g., for nuclear localization, nucleolar localization, mitochondrial localization), fused to the polynucleotide encoding the one or more components of the system. For example, a vector may comprise a nuclear localization sequence (e.g., from SV40) fused to the polynucleotide encoding the one or more components of the system. In some embodiments, the introduction of the gene-regulating system to the cell occurs in vitro. In some embodiments, the introduction of the gene-regulating system to the cell occurs in vivo. In some embodiments, the introduction of the gene-regulating system to the cell occurs ex vivo.

In some embodiments, the recombinant vector comprising a polynucleotide encoding one or more components of a gene-regulating system described herein is a viral vector. Suitable viral vectors include, but are not limited to, viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., U.S. Pat. No. 7,078,387; Ali et al., Hum Gene Ther 9:81 86, 1998, Flannery et al, PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al, Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments, the recombinant vector comprising a polynucleotide encoding one or more components of a gene-regulating system described herein is a plasmid. Numerous suitable plasmid expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other plasmid vector may be used so long as it is compatible with the host cell. Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a polynucleotide sequence encoding one or more components of a gene-regulating system described herein is operably linked to a control element, e.g., a transcriptional control element, such as a promoter. The transcriptional control element may be functional in either a eukaryotic cell (e.g., a mammalian cell) or a prokaryotic cell (e.g., bacterial or archaeal cell). In some embodiments, a polynucleotide sequence encoding one or more components of a gene-regulating system described herein is operably linked to multiple control elements that allow expression of the polynucleotide in both prokaryotic and eukaryotic cells. Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-1. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6xHis tag, hemagglutinin tag, green fluorescent protein, etc.) that are fused to the site-directed modifying polypeptide, thus resulting in a chimeric polypeptide.

In some embodiments, a polynucleotide sequence encoding one or more components of a gene-regulating system described herein is operably linked to an inducible promoter. In some embodiments, a polynucleotide sequence encoding one or more components of a gene-regulating system described herein is operably linked to a constitutive promoter.

Methods of introducing polynucleotides and recombinant vectors into a host cell are known in the art, and any known method can be used to introduce components of a gene-regulating system into a cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: 50169-409X(12)00283-9), microfluidics delivery methods (See e.g., International PCT Publication No. WO 2013/059343), and the like. In some embodiments, delivery via electroporation comprises mixing the cells with the components of a gene-regulating system in a cartridge, chamber, or cuvette and applying one or more electrical impulses of defined duration and amplitude. In some embodiments, cells are mixed with components of a gene-regulating system in a vessel connected to a device (e.g., a pump) which feeds the mixture into a cartridge, chamber, or cuvette wherein one or more electrical impulses of defined duration and amplitude are applied, after which the cells are delivered to a second vessel.

In some embodiments, one or more components of a gene-regulating system, or polynucleotide sequence encoding one or more components of a gene-regulating system described herein are introduced to a cell in a non-viral delivery vehicle, such as a transposon, a nanoparticle (e.g., a lipid nanoparticle), a liposome, an exosome, an attenuated bacterium, or a virus-like particle. In some embodiments, the vehicle is an attenuated bacterium (e.g., naturally or artificially engineered to be invasive but attenuated to prevent pathogenesis including Listeria monocytogenes, certain Salmonella strains, Bifidobacterium longum, and modified Escherichia coli), bacteria having nutritional and tissue-specific tropism to target specific cells, and bacteria having modified surface proteins to alter target cell specificity. In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In some embodiments, the vehicle is a biological liposome. For example, the biological liposome is a phospholipid-based particle derived from human cells (e.g., erythrocyte ghosts, which are red blood cells broken down into spherical structures derived from the subject and wherein tissue targeting can be achieved by attachment of various tissue or cell-specific ligands), secretory exosomes, or subject derived membrane-bound nanovescicles (30-100 nm) of endocytic origin (e.g., can be produced from various cell types and can therefore be taken up by cells without the need for targeting ligands).

In some embodiments, the methods of modified immune effector cells described herein comprise obtaining a population of immune effector cells from a sample. In some embodiments, a sample comprises a tissue sample, a fluid sample, a cell sample, a protein sample, or a DNA or RNA sample. In some embodiments, a tissue sample may be derived from any tissue type including, but not limited to skin, hair (including roots), bone marrow, bone, muscle, salivary gland, esophagus, stomach, small intestine (e.g., tissue from the duodenum, jejunum, or ileum), large intestine, liver, gallbladder, pancreas, lung, kidney, bladder, uterus, ovary, vagina, placenta, testes, thyroid, adrenal gland, cardiac tissue, thymus, spleen, lymph node, spinal cord, brain, eye, ear, tongue, cartilage, white adipose tissue, or brown adipose tissue. In some embodiments, a tissue sample may be derived from a cancerous, pre-cancerous, or non-cancerous tumor. In some embodiments, a fluid sample comprises buccal swabs, blood, plasma, oral mucous, vaginal mucous, peripheral blood, cord blood, saliva, semen, urine, ascites fluid, pleural fluid, spinal fluid, pulmonary lavage, tears, sweat, semen, seminal fluid, seminal plasma, prostatic fluid, pre-ejaculatory fluid (Cowper's fluid), excreta, cerebrospinal fluid, lymph, cell culture media comprising one or more populations of cells, buffered solutions comprising one or more populations of cells, and the like.

In some embodiments, the sample is processed to enrich or isolate a particular cell type, such as an immune effector cell, from the remainder of the sample. In certain embodiments, the sample is a peripheral blood sample which is then subject to leukopheresis to separate the red blood cells and platelets and to isolate lymphocytes. In some embodiments, the sample is a leukopak from which immune effector cells can be isolated or enriched. In some embodiments, the sample is a tumor sample that is further processed to isolate lymphocytes present in the tumor (i.e., to isolate tumor infiltrating lymphocytes).

In some embodiments, the isolated immune effector cells are expanded in culture to produce an expanded population of immune effector cells. One or more activating or growth factors may be added to the culture system during the expansion process. For example, in some embodiments, one or more cytokines (such as IL-2, IL-15, and/or IL-7) can be added to the culture system to enhance or promote cell proliferation and expansion. In some embodiments, one or more activating antibodies, such as an anti-CD3 antibody, may be added to the culture system to enhance or promote cell proliferation and expansion. In some embodiments, the immune effector cells may be co-cultured with feeder cells during the expansion process. In some embodiments, the methods provided herein comprise one or more expansion phases. For example, in some embodiments, the immune effector cells can be expanded after isolation from a sample, allowed to rest, and then expanded again. In some embodiments, the immune effector cells can be expanded in one set of expansion conditions followed by a second round of expansion in a second, different, set of expansion conditions. Methods for ex vivo expansion of immune cells are known in the art, for example, as described in US Patent Application Publication Nos. 20180282694 and 20170152478 and U.S. Pat. Nos. 8,383,099 and 8,034,334.

At any point during the culture and expansion process, the gene-regulating systems described herein can be introduced to the immune effector cells to produce a population of modified immune effector cells. In some embodiments, the gene-regulating system is introduced to the population of immune effector cells immediately after enrichment from a sample. In some embodiments, the gene-regulating system is introduced to the population of immune effector cells before, during, or after the one or more expansion process. In some embodiments, the gene-regulating system is introduced to the population of immune effector cells immediately after enrichment from a sample or harvest from a subject, and prior to any expansion rounds. In some embodiments, the gene-regulating system is introduced to the population of immune effector cells after a first round of expansion and prior to a second round of expansion. In some embodiments, the gene-regulating system is introduced to the population of immune effector cells after a first and a second round of expansion.

In some embodiments, the modified immune effector cells produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

In some embodiments, the modified immune effector cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

### A. Producing Modified Immune Effector Cells Using CRISPR/Cas Systems

In some embodiments, a method of producing a modified immune effector cell involves contacting a target DNA sequence with a complex comprising a gRNA and a Cas polypeptide. As discussed above, a gRNA and Cas polypeptide form a complex, wherein the DNA-binding domain of the gRNA targets the complex to a target DNA sequence and wherein the Cas protein (or heterologous protein fused to an enzymatically inactive Cas protein) modifies target DNA sequence. In some embodiments, this complex is formed intracellularly after introduction of the gRNA and Cas protein (or polynucleotides encoding the gRNA and Cas proteins) to a cell. In some embodiments, the nucleic acid encoding the Cas protein is a DNA nucleic acid and is introduced to the cell by transduction. In some embodiments, the Cas9 and gRNA components of a CRISPR/Cas gene editing system are encoded by a single polynucleotide molecule. In some embodiments, the polynucleotide encoding the Cas protein and gRNA component are comprised in a viral vector and introduced to the cell by viral transduction. In some embodiments, the Cas9 and gRNA components of a CRISPR/Cas gene editing system are encoded by different polynucleotide molecules. In some embodiments, the polynucleotide encoding the Cas protein is comprised in a first viral vector and the polynucleotide encoding the gRNA is comprised in a second viral vector. In some aspects of this embodiment, the first viral vector is introduced to a cell prior to the second viral vector. In some aspects of this embodiment, the second viral vector is introduced to a cell prior to the first viral vector. In such embodiments, integration of the vectors results in sustained expression of the Cas9 and gRNA components. However, sustained expression of Cas9 may lead to increased off-target mutations and cutting in some cell types. Therefore, in some embodiments, an mRNA nucleic acid sequence encoding the Cas protein may be introduced to the population of cells by transfection. In such embodiments, the expression of Cas9 will decrease over time, and may reduce the number of off target mutations or cutting sites.

In some embodiments, this complex is formed in a cell-free system by mixing the gRNA molecules and Cas proteins together and incubating for a period of time sufficient to allow complex formation. This pre-formed complex, comprising the gRNA and Cas protein and referred to herein as a CRISPR-ribonucleoprotein (CRISPR-RNP) can then be introduced to a cell in order to modify a target DNA sequence.

B. Producing Modified Immune Effector Cells Using shRNA Systems

In some embodiments, a method of producing a modified immune effector cell introducing into the cell one or more DNA polynucleotides encoding one or more shRNA molecules with sequence complementary to the mRNA transcript of a target gene. The immune effector cell can be modified to produce the shRNA by introducing specific DNA sequences into the cell nucleus via a small gene cassette. Both retroviruses and lentiviruses can be used to introduce shRNA-encoding DNAs into immune effector cells. The introduced DNA can either become part of the cell's own DNA or persist in the nucleus, and instructs the cell machinery to produce shRNAs. shRNAs may be processed by Dicer or AGO2-mediated slicer activity inside the cell to induce RNAi mediated gene knockdown.

### V. Compositions and Kits

The term “composition” as used herein refers to a formulation of a gene-regulating system or a modified immune effector cell described herein that is capable of being administered or delivered to a subject or cell. Typically, formulations include all physiologically acceptable compositions including derivatives and/or prodrugs, solvates, stereoisomers, racemates, or tautomers thereof with any physiologically acceptable carriers, diluents, and/or excipients. A “therapeutic composition” or “pharmaceutical composition” (used interchangeably herein) is a composition of a gene-regulating system or a modified immune effector cell capable of being administered to a subject for the treatment of a particular disease or disorder or contacted with a cell for modification of one or more endogenous target genes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, and/or emulsifier which has been approved by the United States Food and Drug Administration as being acceptable for use in humans and/or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations. Except insofar as any conventional media and/or agent is incompatible with the agents of the present disclosure, its use in therapeutic compositions is contemplated. Supplementary active ingredients also can be incorporated into the compositions.

“Pharmaceutically acceptable salt” includes both acid and base addition salts. Pharmaceutically-acceptable salts include the acid addition salts (formed with the free amino groups of the protein) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, ptoluenesulfonic acid, trifluoroacetic acid, undecylenic acid, and the like. Salts formed with the free carboxyl groups can also be derived from inorganic bases such as, for example, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

In some embodiments, the present disclosure provides kits for carrying out a method described herein. In some embodiments, a kit can include:

(a) two or more nucleic acid molecules capable of reducing the expression or modifying the function of a gene product encoded by two or more endogenous target genes selected from SOCS1, PTPN2, and ZC3H12A;

(b) one or more polynucleotides encoding two or more nucleic acid molecules that are capable of reducing the expression or modifying the function of a gene product encoded by two or more endogenous target genes selected from SOCS1, PTPN2, and ZC3H12A;

(c) two or more proteins capable of reducing the expression or modifying the function of a gene product encoded by two or more endogenous target genes selected from SOCS1, PTPN2, and ZC3H12A;

(d) one or more polynucleotides encoding two or more modifying proteins that is capable of reducing the expression or modifying the function of a gene product encoded by two or more endogenous target genes selected from SOCS1, PTPN2, and ZC3H12A;

(e) two or more gRNAs capable of binding to a target DNA sequence in two or more endogenous target genes selected from SOCS1, PTPN2, and ZC3H12A;

(f) one or more polynucleotides encoding two or more gRNAs capable of binding to a target DNA sequence in two or more endogenous target genes selected from SOCS1, PTPN2, and ZC3H12A;

(g) one or more site-directed modifying polypeptides capable of interacting with a gRNA and modifying a target DNA sequence in an endogenous gene;

(h) one or more polynucleotides encoding a site-directed modifying polypeptide capable of interacting with a gRNA and modifying a target DNA sequence in two or more endogenous target genes selected from SOCS1, PTPN2, and ZC3H12A;

(i) two or more guide DNAs (gDNAs) capable of binding to a target DNA sequence in two or more endogenous target genes selected from SOCS1, PTPN2, and ZC3H12A;

(j) one or more polynucleotides encoding two or more gDNAs capable of binding to a target DNA sequence in two or more endogenous target genes selected from SOCS1, PTPN2, and ZC3H12A;

(k) one or more site-directed modifying polypeptides capable of interacting with a gDNA and modifying a target DNA sequence in an endogenous gene;

(l) one or more polynucleotides encoding a site-directed modifying polypeptide capable of interacting with a gDNA and modifying a target DNA sequence in an endogenous gene;

(m) two or more gRNAs capable of binding to a target mRNA sequence encoded by two or more endogenous target genes selected from SOCS1, PTPN2, and ZC3H12A;

(n) one or more polynucleotides encoding two or more gRNAs capable of binding to a target mRNA sequence encoded by two or more endogenous target genes selected from SOCS1, PTPN2, and ZC3H12A;

(o) one or more site-directed modifying polypeptides capable of interacting with a gRNA and modifying a target mRNA sequence encoded by an endogenous gene;

(p) one or more polynucleotides encoding a site-directed modifying polypeptide capable of interacting with a gRNA and modifying a target mRNA sequence encoded by an endogenous gene;

(q) a modified immune effector cell described herein; or

(r) any combination of the above.

In some embodiments, the kits described herein further comprise one or more immune checkpoint inhibitors. Several immune checkpoint inhibitors are known in the art and have received FDA approval for the treatment of one or more cancers. For example, FDA-approved PD-L1 inhibitors include Atezolizumab (Tecentriq®, Genentech), Avelumab (Bavencio®, Pfizer), and Durvalumab (Imfinzi®, AstraZeneca); FDA-approved PD-1 inhibitors include Pembrolizumab (Keytruda®, Merck) and Nivolumab (Opdivo®, Bristol-Myers Squibb); and FDA-approved CTLA4 inhibitors include Ipilimumab (Yervoy®, Bristol-Myers Squibb). Additional inhibitory immune checkpoint molecules that may be the target of future therapeutics include A2AR, B7-H3, B7-H4, BTLA, IDO, LAG3 (e.g., BMS-986016, under development by BSM), KIR (e.g., Lirilumab, under development by BSM), TIM3, TIGIT, and VISTA.

In some embodiments, the kits described herein comprise one or more components of a gene-regulating system (or one or more polynucleotides encoding the one or more components) and one or more immune checkpoint inhibitors known in the art (e.g., a PD1 inhibitor, a CTLA4 inhibitor, a PDL1 inhibitor, etc.). In some embodiments, the kits described herein comprise one or more components of a gene-regulating system (or one or more polynucleotides encoding the one or more components) and an anti-PD1 antibody (e.g., Pembrolizumab or Nivolumab). In some embodiments, the kits described herein comprise a modified immune effector cell described herein (or population thereof) and one or more immune checkpoint inhibitors known in the art (e.g., a PD1 inhibitor, a CTLA4 inhibitor, a PDL1 inhibitor, etc.). In some embodiments, the kits described herein comprise a modified immune effector cell described herein (or population thereof) and an anti-PD1 antibody (e.g., Pembrolizumab or Nivolumab).

In some embodiments, the kit comprises one or more components of a gene-regulating system (or one or more polynucleotides encoding the one or more components) and a reagent for reconstituting and/or diluting the components. In some embodiments, a kit comprising one or more components of a gene-regulating system (or one or more polynucleotides encoding the one or more components) and further comprises one or more additional reagents, where such additional reagents can be selected from: a buffer for introducing the gene-regulating system into a cell; a wash buffer; a control reagent; a control expression vector or RNA polynucleotide; a reagent for in vitro production of the gene-regulating system from DNA, and the like. Components of a kit can be in separate containers or can be combined in a single container.

In addition to above-mentioned components, in some embodiments a kit further comprises instructions for using the components of the kit to practice the methods of the present disclosure. The instructions for practicing the methods are generally recorded on a suitable recording medium. For example, the instructions may be printed on a substrate, such as paper or plastic, etc. As such, the instructions may be present in the kits as a package insert or in the labeling of the container of the kit or components thereof (i.e., associated with the packaging or sub-packaging). In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### VI. Therapeutic Methods and Applications

In some embodiments, the modified immune effector cells and gene-regulating systems described herein may be used in a variety of therapeutic applications. For example, in some embodiments the modified immune effector cells and/or gene-regulating systems described herein may be administered to a subject for purposes such as gene therapy, e.g. to treat a disease, for use as an antiviral, for use as an anti-pathogenic, for use as an anti-cancer therapeutic, or for biological research.

In some embodiments, the subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g. mice, rats, guinea pigs, hamsters, rabbits, etc.) may be used for experimental investigations.

Administration of the modified immune effector cells described herein, populations thereof, and compositions thereof can occur by injection, irrigation, inhalation, consumption, electro-osmosis, hemodialysis, iontophoresis, and other methods known in the art. In some embodiments, administration route is local or systemic. In some embodiments administration route is intraarterial, intracranial, intradermal, intraduodenal, intrammamary, intrameningeal, intraperitoneal, intrathecal, intratumoral, intravenous, intravitreal, ophthalmic, parenteral, spinal, subcutaneous, ureteral, urethral, vaginal, or intrauterine.

In some embodiments, the administration route is by infusion (e.g., continuous or bolus). Examples of methods for local administration, that is, delivery to the site of injury or disease, include through an Ommaya reservoir, e.g. for intrathecal delivery (See e.g., U.S. Pat. Nos. 5,222,982 and 5,385,582, incorporated herein by reference); by bolus injection, e.g. by a syringe, e.g. into a joint; by continuous infusion, e.g. by cannulation, such as with convection (See e.g., US Patent Application Publication No. 2007-0254842, incorporated herein by reference); or by implanting a device upon which the cells have been reversibly affixed (see e.g. US Patent Application Publication Nos. 2008-0081064 and 2009-0196903, incorporated herein by reference). In some embodiments, the administration route is by topical administration or direct injection. In some embodiments, the modified immune effector cells described herein may be provided to the subject alone or with a suitable substrate or matrix, e.g. to support their growth and/or organization in the tissue to which they are being transplanted.

In some embodiments, at least 1×10³ cells are administered to a subject. In some embodiments, at least 5×10³ cells, 1×10⁴ cells, 5×10⁴ cells, 1×10⁵ cells, 5×10⁵ cells, 1×10⁶, 2×10⁶, 3×10⁶, 4×10⁶, 5×10⁶, 1×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², or more cells are administered to a subject. In some embodiments, between about 1×10⁷ and about 1×10¹² cells are administered to a subject. In some embodiments, between about 1×10⁸ and about 1×10¹² cells are administered to a subject. In some embodiments, between about 1×10⁹ and about 1×10¹² cells are administered to a subject. In some embodiments, between about 1×10¹⁰ and about 1×10¹² cells are administered to a subject. In some embodiments, between about 1×10¹¹ and about 1×10¹² cells are administered to a subject. In some embodiments, between about 1×10⁷ and about 1×10¹¹ cells are administered to a subject. In some embodiments, between about 1×10⁷ and about 1×10¹⁰ cells are administered to a subject. In some embodiments, between about 1×10⁷ and about 1×10⁹ cells are administered to a subject. In some embodiments, between about 1×10⁷ and about 1×10⁸ cells are administered to a subject. The number of administrations of treatment to a subject may vary. In some embodiments, introducing the modified immune effector cells into the subject may be a one-time event. In some embodiments, such treatment may require an on-going series of repeated treatments. In some embodiments, multiple administrations of the modified immune effector cells may be required before an effect is observed. The exact protocols depend upon the disease or condition, the stage of the disease and parameters of the individual subject being treated.

In some embodiments, the gene-regulating systems described herein are employed to modify cellular DNA or RNA in vivo, such as for gene therapy or for biological research. In such embodiments, a gene-regulating system may be administered directly to the subject, such as by the methods described supra. In some embodiments, the gene-regulating systems described herein are employed for the ex vivo or in vitro modification of a population of immune effector cells. In such embodiments, the gene-regulating systems described herein are administered to a sample comprising immune effector cells.

In some embodiments, the modified immune effector cells described herein are administered to a subject. In some embodiments, the modified immune effector cells described herein administered to a subject are autologous immune effector cells. The term “autologous” in this context refers to cells that have been derived from the same subject to which they are administered. For example, immune effector cells may be obtained from a subject, modified ex vivo according to the methods described herein, and then administered to the same subject in order to treat a disease. In such embodiments, the cells administered to the subject are autologous immune effector cells. In some embodiments, the modified immune effector cells, or compositions thereof, administered to a subject are allogenic immune effector cells. The term “allogenic” in this context refers to cells that have been derived from one subject and are administered to another subject. For example, immune effector cells may be obtained from a first subject, modified ex vivo according to the methods described herein and then administered to a second subject in order to treat a disease. In such embodiments, the cells administered to the subject are allogenic immune effector cells.

In some embodiments, the modified immune effector cells described herein are administered to a subject in order to treat a disease. In some embodiments, treatment comprises delivering an effective amount of a population of cells (e.g., a population of modified immune effector cells) or composition thereof to a subject in need thereof. In some embodiments, treating refers to the treatment of a disease in a mammal, e.g., in a human, including (a) inhibiting the disease, i.e., arresting disease development or preventing disease progression; (b) relieving the disease, i.e., causing regression of the disease state or relieving one or more symptoms of the disease; and (c) curing the disease, i.e., remission of one or more disease symptoms. In some embodiments, treatment may refer to a short-term (e.g., temporary and/or acute) and/or a long-term (e.g., sustained) reduction in one or more disease symptoms. In some embodiments, treatment results in an improvement or remediation of the symptoms of the disease. The improvement is an observable or measurable improvement, or may be an improvement in the general feeling of well-being of the subject.

The effective amount of a modified immune effector cell administered to a particular subject will depend on a variety of factors, several of which will differ from patient to patient including the disorder being treated and the severity of the disorder; activity of the specific agent(s) employed; the age, body weight, general health, sex and diet of the patient; the timing of administration, route of administration; the duration of the treatment; drugs used in combination; the judgment of the prescribing physician; and like factors known in the medical arts.

In some embodiments, the effective amount of a modified immune effector cell may be the number of cells required to result in at least 1, 2, 3, 4, 5, 6, 7, 8, 9, 10, 20, 30, 40, 50, 60, 70, 80, 90, 100, or more fold decrease in tumor mass or volume, decrease in the number of tumor cells, or decrease in the number of metastases. In some embodiments, the effective amount of a modified immune effector cell may be the number of cells required to achieve an increase in life expectancy, an increase in progression-free or disease-free survival, or amelioration of various physiological symptoms associated with the disease being treated. In some embodiments, an effective amount of modified immune effector cells will be at least 1×10³, for example 5×10³, 1×10⁴, 5×10⁴, 1×10⁵, 5×10⁵, 1×10⁶, 2×10⁶, 3×10⁶, 4×10⁶, 5×10⁶, 1×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², or more cells.

In some embodiments, the modified immune effector cells and gene-regulating systems described herein may be used in the treatment of a cell-proliferative disorder, such as a cancer. Cancers that may be treated using the compositions and methods disclosed herein include cancers of the blood and solid tumors. For example, cancers that may be treated using the compositions and methods disclosed herein include, but are not limited to, adenoma, carcinoma, sarcoma, leukemia or lymphoma. In some embodiments, the cancer is chronic lymphocytic leukemia (CLL), B cell acute lymphocytic leukemia (B-ALL), acute lymphoblastic leukemia (ALL), acute myeloid leukemia (AML), non-Hodgkin's lymphoma (NHL), diffuse large cell lymphoma (DLCL), diffuse large B cell lymphoma (DLBCL), Hodgkin's lymphoma, multiple myeloma, renal cell carcinoma (RCC), neuroblastoma, colorectal cancer, breast cancer, ovarian cancer, melanoma, sarcoma, prostate cancer, lung cancer including but not limited to NSCLC esophageal cancer, hepatocellular carcinoma, pancreatic cancer, astrocytoma, mesothelioma, head and neck cancer, medulloblastoma, bladder cancer, and liver cancer.

As described above, several immune checkpoint inhibitors are currently approved for use in a variety of oncologic indications (e.g., CTLA4 inhibitors, PD1 inhibitors, PDL1 inhibitors, etc.). In some embodiments, administration of a modified immune effector cell comprising reduced expression and/or function of an endogenous target gene described herein results in an enhanced therapeutic effect (e.g., a more significant reduction in tumor growth, an increase in tumor infiltration by lymphocytes, an increase in the length of progression free survival, etc.) than is observed after treatment with an immune checkpoint inhibitor.

Further, some oncologic indications are non-responsive (i.e., are insensitive) to treatment with immune checkpoint inhibitors. Further still, some oncologic indications that are initially responsive (i.e., sensitive) to treatment with immune checkpoint inhibitors develop an inhibitor-resistant phenotype during the course of treatment. Therefore, in some embodiments, the modified immune effector cells described herein, or compositions thereof, are administered to treat a cancer that is resistant (or partially resistant) or insensitive (or partially insensitive) to treatment with one or more immune checkpoint inhibitors. In some embodiments, administration of the modified immune effector cells or compositions thereof to a subject suffering from a cancer that is resistant (or partially resistant) or insensitive (or partially insensitive) to treatment with one or more immune checkpoint inhibitors results in treatment of the cancer (e.g., reduction in tumor growth, an increase in the length of progression free survival, etc.). In some embodiments, the cancer is resistant (or partially resistant) or insensitive (or partially insensitive) to treatment with a PD1 inhibitor.

In some embodiments, the modified immune effector cells or compositions thereof are administered in combination with an immune checkpoint inhibitor. In some embodiments, administration of the modified immune effector cells in combination with the immune checkpoint inhibitor results in an enhanced therapeutic effect in a cancer that is resistant, refractory, or insensitive to treatment by an immune checkpoint inhibitor than is observed by treatment with either the modified immune effector cells or the immune checkpoint inhibitor alone. In some embodiments, administration of the modified immune effector cells in combination with the immune checkpoint inhibitor results in an enhanced therapeutic effect in a cancer that is partially resistant, partially refractory, or partially insensitive to treatment by an immune checkpoint inhibitor than is observed by treatment with either the modified immune effector cells or the immune checkpoint inhibitor alone. In some embodiments, the cancer is resistant (or partially resistant), refractory (or partially refractory), or insensitive (or partially insensitive) to treatment with a PD1 inhibitor.

In some embodiments, administration of a modified immune effector cell described herein or composition thereof in combination with an anti-PD1 antibody results in an enhanced therapeutic effect in a cancer that is resistant or insensitive to treatment by the anti-PD1 antibody alone. In some embodiments, administration of a modified immune effector cell described herein or composition thereof in combination with an anti-PD1 antibody results in an enhanced therapeutic effect in a cancer that is partially resistant or partially insensitive to treatment by the anti-PD1 antibody alone.

Cancers that demonstrate resistance or sensitivity to immune checkpoint inhibition are known in the art and can be tested in a variety of in vivo and in vitro models. For example, some melanomas are sensitive to treatment with an immune checkpoint inhibitor such as an anti-PD1 antibody and can be modeled in an in vivo B16-Ova tumor model (See Example 5). Further, some colorectal cancers are known to be resistant to treatment with an immune checkpoint inhibitor such as an anti-PD1 antibody and can be modeled in a PMEL/MC38-gp100 model (See Example 5). Further still, some lymphomas are known to be insensitive to treatment with an immune checkpoint inhibitor such as an anti-PD1 antibody and can be modeled in various models by adoptive transfer or subcutaneous administration of lymphoma cell lines, such as Raji cells (See Example 6-9).

Current adoptive cell therapy, including TIL therapy, includes lymphodepletion 7 days prior to TIL infusion using Cy/Flu based treatment. The lymphodepletion is believed necessary to deplete the endogenous Treg population, to boost endogenous IL-7 and IL-15 production and to create physical space for the TIL infusion. This lymphodepletion is associated with severe grade 3, 4, and sometimes 5 adverse events and can significantly impact patient outcome. In addition, current therapy includes an infusion of high dose IL-2 5 days prior to TIL infusion in order to boost function and survival of the transferred TILs. However, the high dose IL-2 infusion is associated with severe grade 3 and 4 adverse events, including capillary leak syndrome. In some embodiments, the modified immune effector cells described herein are transferred to a recipient host that has not undergone lymphodepletion treatment and/or are transferred to a recipient host in the absence of high dose IL-2 treatment. Without wishing to be bound by theory, it is possible that the modified immune effector cells described herein (e.g., modified TILs) demonstrate increased sensitivity to IL-7, IL-15 and/or IL-2, therefore allowing for increased steps enhanced competitive fitness, survival, and/or persistence of the modified cells such that lymphodepletion and/or high dose IL-2 is not required.

In some embodiments, the modified immune effector cells and gene-regulating systems described herein may be used in the treatment of a viral infection. In some embodiments, the virus is selected from one of adenoviruses, herpesviruses (including, for example, herpes simplex virus and Epstein Barr virus, and herpes zoster virus), poxviruses, papovaviruses, hepatitis viruses, (including, for example, hepatitis B virus and hepatitis C virus), papilloma viruses, orthomyxoviruses (including, for example, influenza A, influenza B, and influenza C), paramyxoviruses, coronaviruses, picornaviruses, reoviruses, togaviruses, flaviviruses, bunyaviridae, rhabdoviruses, rotavirus, respiratory syncitial virus, human immunodeficiency virus, or retroviruses.

### EXAMPLES

### Example 1: Materials and Methods

The experiments described herein utilize the CRISPR/Cas9 system to reduce expression of two or more of SOCS1, PTPN2, and ZC3H12A in different T cell populations.

gRNA-Cas9 RNPs:

Unless otherwise indicated, the following experiments use dual gRNA molecules formed by duplexing 200 μM tracrRNA (IDT Cat #1072534) with 200 μM of target-specific crRNA (IDT) in nuclease free duplex buffer (IDT Cat #11-01-03-01) for 5 min at 95° C., to form 100 μM of tracrRNA:crRNA duplex, where the tracrRNA and crRNA are present at a 1:1 ratio. Alternatively, single-molecule gRNAs (sgRNAs) (IDT) were resuspended at 100 μM in nuclease free duplex buffer (IDT). Cas9 was expressed in target cells by introduction of either Cas9 mRNA or a Cas9 protein. Unless otherwise indicated, the Cas9 protein derived from S. pyogenes (IDT Cat #1074182) was used in the following experiments. For Human RNPs, gRNA-Cas9 ribonucleoproteins (RNPs) were formed by combining 1.2 μL of 100 μM tracrRNA:crRNA duplex or gRNAs with 1 μL of 20 μM Cas9 protein and 0.8 μL of PBS. For Mouse RNPs, gRNA-Cas9 ribonucleoproteins (RNPs) were formed by combining 1 Volume of 44 μM tracrRNA:crRNA duplex or gRNA with 1 Volume of 36 μM Cas9 in Invitrogen Buffer T. For both, mixtures were incubated at RT for 20 minutes to form the RNP complexes. gRNAs used in the following experiments are provided in Table 18 below.

TABLE 18

| Target Gene | Guide ID | Sequence | SEQ ID |
|---|---|---|---|
| Pccd1 | Nm.Pdcd1 | CGGAGGATCTTATGCTGAAC | 270 |
| Cb1b | Nm.Cb1b | CCTTATCTTCAGTCACATGC | 271 |
| Zc3h12a | Nm.Zc3h12a | TTCCCTCCTCTGCCAGCCAT | 211 |
| Socs1 | Nm.Socs1 | GCCGGCCGCTTCCACTTGGA | 9 |
| Ptpn2 | Nm.Ptpn2 | CCTTTCTTGCAGATGGAAAA | 202 |

CAR Expression Constructs:

A Chimeric antigen receptor (CAR) specific for human CD19, was generated. Briefly, the 22 amino acid signal peptide of the human granulocyte-macrophage colony stimulating factor receptor subunit alpha (GMSCF-Rα) was fused to an antigen-specific scFv domain specifically binding to CD19 (clone FMC63). The human CD8α stalk was used as a transmembrane domain. The intracellular signaling domains of the CD3ξ chain were fused to the cytoplasmic end of the CD8α stalk. The full length CAR construct is provided in SEQ ID NO: 813 and nucleic acid sequences of the full length CAR constructs is provided in SEQ ID NO: 814.

Engineered TCRs Expression Constructs:

A recombinant T cell receptor (TCR) specific for the NY-ESO-1 peptide (in the context of HLA-A*02:01) was generated. Paired TCR-α:TCR-β variable region protein sequences encoding the 1G4 TCR specific for the NY-ESO-1 peptide comprising the sequence of SLLMWITQ (SEQ ID NO: 815), presented by HLA-A*02:01, were identified from the literature (Robbins et al, Journal of Immunology 2008 180:6116-6131). The NY-ESO-1 peptide can have an additional cysteine or valine at its C-terminal end. TCRα chains were composed of V and J gene segments and CDR3α sequences and TCRβ chains were composed of V, D, and J gene segment and CDR3-β sequences. The native TRAC (SEQ ID NO: 816) and TRBC (SEQ ID NOs: 817) protein sequences were fused to the C-terminal ends of the α and β chain variable regions, respectively, to produce 95:LY 1G4-TCR α/βchains (SEQ ID NOs: 818 and 819, respectively.

Codon-optimized DNA sequences encoding the engineered TCRα and TCRβ chain proteins were generated where the P2A sequence (SEQ ID NO: 820) was inserted between the DNA sequences encoding the TCRβ and the TCRα chain, such that expression of both TCR chains was driven off of a single promoter in a stoichiometric fashion. The expression cassettes encoding the engineered TCR chains therefore comprised the following format: TCRβ-P2A-TCRα. Final protein sequences for each TCR construct are provided in SEQ ID NO: 821 (95:LY 1G4). This TCR construct is referred to hereafter as “TCR2”.

Lentiviral Expression Constructs:

The CAR and engineered TCR expression constructs described above were then inserted into a plasmid comprising an SFFV promoter driving expression of the engineered receptor, a T2A sequence, and a puromycin resistance cassette. Lentiviral constructs comprising an engineered CAR expression construct may further comprise an sgRNA targeting the endogenous TRAC gene, which encodes the constant region of the α chain of the T cell receptor.

Lentiviruses encoding the engineered receptors described above were generated as follows. Briefly, 289×10⁶ of LentiX-293T cells were plated out in a 5-layer CellSTACK 24 hours prior to transfection. Serum-free OptiMEM and TransIT-293 were combined and incubated for 5 minutes before combining helper plasmids (58 μg VSVG and 115 μg PAX2-Gag-Pol) with 231 μg of an engineered receptor- and sgRNA-expressing plasmid described above. After 20 minutes, this mixture was added to the LentiX-293T cells with fresh media. Media was replaced 18 hours after transfection and viral supernatants were collected 48 hours post-transfection. Supernatants were treated with Benzonase® nuclease and passed through a 0.45 μm filter to isolate the viral particles. Virus particles were then concentrated by Tangential Flow Filtration (TFF), aliquoted, tittered, and stored at −80° C.

Human T cell Isolation and Activation:

Total human PBMCs were isolated from fresh leukopheresis by Ficoll gradient centrifugation. CD8+ T-cells were then purified from total PBMCs using a CD8+ T-cell isolation kit (Stemcell Technologies Cat #17953). For T cell activation, CD8+ T cells were plated at 2×10⁶ cells/mL in X-VIVO 15 T Cell Expansion Medium (Lonza, Cat #04-418Q) in a T175 flask, with 6.25 μL/mL of ImmunoCult T-cell activators (anti-CD3/CD28/CD2, StemCell Technologies, Vancouver BC, Canada) and 10 ng/mL human IL2. T-cells were activated for 18 hours prior to transduction with lentiviral constructs.

Lentiviral transduction of T cells: T-cells activated 18 hours prior were seeded at 5×10⁶ cells per well in a 6 well plate, in 1.5 mL volume of X-VIVO 15 media, 10 ng/mL human IL-2 and 12.5 μL Immunocult Human CD3/CD28/CD2 T-cell Activator. Lentivirus expressing the engineered receptors was added at an MOI capable of infecting 80% of all cells. 25 μL of Retronectin (1 mg/mL) was added to each well. XVIVO-15 media was added to a final volume of 2.0 mL per well. Plates were spun at 600×g for 1.5 hours at room temperature. One day later, cells were washed and seeded at 1×10⁶ cells/mL in X-VIVO 15, 10 ng/mL IL2+ T-cell activators.

Electroporation of Human PBMC-Derived T Cells:

3 days after T cell activation, T cells were harvested and resuspended in nucleofection buffer (18% supplement 1, 82% P3 buffer from the Amaxa P3 primary cell 4D-Nuclefector X kit S) at a concentration of 100×10⁶ cells/mL. 1.5 μL of sgRNA/Cas9 RNP complexes (containing 120 pmol of crRNA:tracrRNA duplex and 20 pmol of Cas9 nuclease) and 2.1 μL (100 pmol) of electroporation enhancer were added per 20 μL of cell solution. 25 μL of the cell/RNP/enhancer mixture was then added to each electroporation well. Cells were electroporated using the Lonza electroporator with the “EO-115” program. After electroporation, 80 μL of warm X-VIVO 15 media was added to each well and cells were pooled into a culture flask at a density of 2×10⁶ cells/mL in X-VIVO 15 media containing IL-2 (10 ng/mL). On Day 4, cells were washed, counted, and seeded at densities of 50-100×10⁶ cells/L in X-VIVO 15 media containing IL-2 (10 ng/mL) in G-Rex6M well plates or G-Rex100M, depending on the number of cells available. On Days 6 and 8, 10 ng/mL of fresh recombinant human IL-2 was added to the cultures.

Human TIL Isolation and Activation:

Tumor infiltrating lymphocytes can also be modified by the methods described herein. In such cases, tumors are surgically resected from human patients and diced with scalpel blades into 1 mm³ pieces, with a single piece of tumor placed into each well of a 24 plate. 2 mL of complete TIL media (RPMI+10% heat inactivated human male AB serum, 1 mM pyruvate, 20 μg/mL gentamycin, 1× glutamax) supplemented with 6000 U/mL of recombinant human IL-2 is added to each well of isolated TILs. 1 mL of media is removed from the well and replaced with fresh media and IL-2 up to 3 times a week as needed. As wells reach confluence, they are split 1:1 in new media+IL-2. After 4-5 weeks of culture, the cells are harvested for rapid expansion.

TIL Rapid Expansion:

TILs are rapidly expanded by activating 500,000 TILs with 26×10⁶ allogeneic, irradiated (5000 cGy) PBMC feeder cells in 20 mL TIL media+20 mL of Aim-V media (Invitrogen)+30 ng/mL OKT3 mAb. 48 hours later (Day 2), 6000 U/mL IL-2 is added to the cultures. On day 5, 20 mL of media is removed, and 20 mL fresh media (+30 ng/ml OKT3) is added. On Day 7, cells are counted, and reseeded at 60×10⁶ cells/L in G-Rex6M well plates (Wilson Wolf, Cat #80660M) or G-Rex100M (Wilson Wolf, Cat #81100S), depending on the number of cells available. 6000 U/mL fresh IL-2 is added on Day 9 and 3000 U/mL fresh IL-2 is added on Day 12. TILs are harvested on Day 14. Expanded cells are then slow-frozen in Cryostor CS-10 (Stemcell Technologies Cat #07930) using Coolcell Freezing containers (Corning) and stored long term in liquid nitrogen.

Mice:

Wild type CD8⁺ T cells were derived from C57BL/6J mice (The Jackson Laboratory, Bar Harbor Me.). Ovalbumin (Ova)-specific CD8⁺ T cells were derived from OT1 mice (C57BL/6-Tg(TcraTcrb) 1100Mjba; Jackson Laboratory). OT1 mice comprise a transgenic TCR that recognizes residues 257-264 of the ovalbumin (Ova) protein. gp100-specific CD8+ T cells were derived from PMEL mice (B6.Cg-Thy1<a>/CyTg(TcraTcrb) 8Rest/J; The Jackson Laboratory, Bar Harbor Me. Cat #005023). Mice constitutively expressing the Cas9 protein were obtain from Jackson labs (B6J.129(Cg)-Gt(ROSA)26Sortm1.1(CAG-cas9*,-EGFP)Fezha; The Jackson Laboratory, Bar Harbor Me. Strain #026179), TCR-transgenic mice constitutively expressing Cas9 were obtained by breeding of OT1 mice with Cas9 mice.

Murine T Cell Isolation and Activation:

Spleens from transgenic mice were harvested and reduced to a single cell suspension using the GentleMACS system, according to the manufacturer's recommendations. Purified CD8⁺ T cells were obtained using the EasySep Mouse CD8⁺ T Cell Isolation Kit (Catalog #19853). CD8 T cells were cultured at 1×10⁶ cells/mL in complete T cell media (RPMI+10% heat inactivated FBS, 20 mM HEPES, 100 U/mL Penicillin, 100 μg/mL Streptomycin, 50 μM Beta-Mercaptoethanol) supplemented with 2 ng/mL of Recombinant Mouse IL-2 (Biolegend Catalog #575406) and activated with anti-CD3/anti-CD28 beads (Dynabeads™ Mouse T-Activator CD3/CD28 for T-Cell Expansion and Activation Cat #11456D).

Electroporation of Mouse T Cells:

Murine T-cells activated 48 hours prior were harvested, activation beads were removed and cells were washed and resuspended in Neon nucleofection buffer T. Up to 20×10⁶ cells resuspended in 90 μL (for single edits) or 80 μL (for combination edits) Buffer T can be electroporated using one Neon™ 100-4, tip. 10 μL of each sgRNA/Cas9 RNP complexes and 20 μL of 10.8 μM electroporation enhancer were added per tip. The T cell/RNP/enhancer mixture was loaded into the Neon™ tips and cells were electroporated on the Neon Transfection System using a single pulse of 20 ms at 1700V. Immediately after electroporation, the cells were transferred into a culture flask at a density of 1.6×10⁶ cells/mL in warm complete T cell media supplemented with 2 ng/mL of Recombinant Mouse IL-2. Edited murine CD8 T cells were further cultured at 1×10⁶ cells/mL in complete T cell media supplemented with IL-2 for an additional 2 days. On Day 4, cells were harvested counted and resuspended in PBS for injection in vivo.

Generation and Editing of Murine TILs:

To generate TILs, donor CD45.1 Pep Boy mice (B6. SJL-Ptprc^{a} Pepc^{b}/BoyJ) were injected subcutaneously with 0.5×10⁶ B16-Ova cells. On Day 14 post-tumor cell inoculation, tumors were harvested to generate edited CD45.1 Tumor Infiltrating Lymphocytes (TILs) to infuse into the second cohort of mice. B16-OVA tumors (200-600 mm³) were harvested, diced and reduced to a single cell suspension using the GentleMACS system and mouse Tumor Dissociation Kit (Miltenyi Biotech Catalog #130-096-730), according to the manufacturer's recommendations. Tumor suspension were filtered over 70 μm cell strainers and TILs were enriched using CD4/CD8 (TIL) Microbeads (Miltenyi Biotech Catalog #130-116-480). Isolated TILs were cultured in 6 well plates at 1.5×10⁶ cells/mL in complete mTIL media (RPMI+10% heat inactivated FBS, 20 mM HEPES, 100 U/mL Penicillin, 100 μg/mL Streptomycin, 50 μM Beta-Mercaptoethanol) supplemented with 3000 U/mL of recombinant human IL-2 (Peprotech Catalog #200-02). On Day 3 cells were harvested, washed and resuspended in nucleofection buffer T and electroporated with RNPs using the Neon Transfection System. After electroporation, TILs were cultured in 6 well plates at 1.5×10⁶ cells/mL in complete mTIL media supplemented with 3000 U/mL of recombinant human IL-2. On Day 5 and 7, cells were resuspended in fresh complete mTIL media supplemented with 3000 U/mL of recombinant human IL-2 and plated in flasks at a density of 1×10⁶ cells/mL. On Day 8, cells were harvested counted and resuspended in PBS for injection in vivo.

### Example 2: Screen for Dual-Edit Combinations

A double sgRNA library was constructed in a retroviral backbone. The library consisted of two U6 promoters (one human and one mouse), each driving expression of a single guide RNA (guide+tracr, sgRNA). The guides were cloned as pools to provide random pairings between guides, such that every sgRNA would be paired with every other sgRNA. The final double guide library was transfected into Phoenix-Eco 293T cells to generate murine ecotropic retrovirus. TCR transgenic OT1 cells expressing Cas9 were infected with the sgRNA-expressing virus to edit the two loci targeted by each of the sgRNAs. The edited transgenic T-cells were then transferred into mice bearing >400 mm³ B16-Ova tumors allografts. After two weeks, the tumors were excised and digested into single cell suspension using Miltenyi Tumor Dissociation Kit. gDNA was extracted from the cell pellet using a Qiagen QIAmp DNA and blood kit and the retroviral inserts were recovered by PCR using primers corresponding to the retroviral backbone sequences. The resulting PCR product were then sequenced to identify the sgRNAs present in the tumors two weeks after transfer. The representation of guide pairs in the final isolated cell populations was compared to the initial plasmid population and the population of infected transgenic T-cells before injection into the mouse. The frequency of sgRNA pairs that improved T-cells fitness and/or tumor infiltration were expected to increase over time, while combinations that impaired fitness were expected to decrease over time. Table 19 below shows the median fold change of sgRNA frequency in the final cell population compared to the sgRNA frequency in the initial cell population transferred in vivo.

TABLE 19

| Mouse Gene 1 | Mouse Gene 2 | Avg(Tmedian.Ifoldch.all) |
|---|---|---|
| CBLB | ZAP70 | 0 |
| CBLB | LAG3 | 0.08 |
| CBLB | CBLB | 0.17 |
| CBLB | LICIT | 0.31 |
| CBLB | TGFBR1 | 0.15 |
| CBLB | CTLA4 | 0.21 |
| CBLB | PTPN2 | 1.48 |
| CBLB | TGFBR2 | 0.75 |
| CBLB | ZC3H12A | 8.9 |
| PDCD1 | ZAP70 | 0 |
| PDCD1 | LAG3 | 0.02 |
| PDCD1 | PDCD1 | 0.02 |
| PDCD1 | TIGIT | 0.02 |
| PDCD1 | TGFBR1 | 0.02 |
| PDCD1 | CTLA4 | 0.59 |
| PDCD1 | PTPN2 | 0.07 |
| PDCD1 | TGFBR2 | 0.07 |
| PDCD1 | ZC3H12A | 1.33 |
| PTPN2 | ZAP70 | 0 |
| PTPN2 | LAG3 | 0.09 |
| PTPN2 | PTPN2 | 0.04 |
| PTPN2 | TIGIT | 0.38 |
| PTPN2 | TGFBR1 | 0.22 |
| PTPN2 | CTLA4 | 0.03 |
| PTPN2 | TGFBR2 | 0.23 |
| PTPN2 | ZC3H12A | 9.4 |
| TGFBR1 | ZAP70 | 0 |
| TGFBR1 | LAG3 | 0 |
| TGFBR1 | TIGIT | 0.03 |
| TGFBR1 | TGFBR1 | 0.06 |
| TGFBR1 | CTLA4 | 0.01 |
| TGFBR1 | TGFBR2 | 0.07 |
| TGFBR1 | ZC3H12A | 3.33 |
| ZC3H12A | ZAP70 | 0.01 |
| ZC3H12A | LAG3 | 0.73 |
| ZC3H12A | TIGIT | 2.53 |
| ZC3H12A | CTLA4 | 0.61 |
| ZC3H12A | ZC3H12A | 1.14 |
| Havcr2 | Havcr2 | 0.02 |
| Havcr2 | LAG3 | 0.01 |
| Havcr2 | Olfr1389 | 0 |
| Havcr2 | Olfr453 | 0.01 |
| Havcr2 | PDCD1 | 0.02 |
| LAG3 | Olfr1389 | 0 |
| LAG3 | Olfr453 | 0.02 |
| LAG3 | PDCD1 | 0.02 |
| Olfr1389 | Olfr1389 | 0.01 |
| Olfr1389 | Olfr453 | 0 |
| Olfr1389 | PDCD1 | 0.02 |
| Olfr1389 | PTPN2 | 0.03 |
| Olfr1389 | ZC3H12A | 0.78 |
| Olfr453 | Olfr453 | 0.01 |
| Olfr453 | PDCD1 | 0.01 |
| Olfr453 | PTPN2 | 0.46 |
| Olfr453 | ZC3H12A | 1.58 |

### Example 3: Efficacy of Ptpn2/Socs1 Dual-Edited Transgenic T Cells in Murine Syngeneic Tumor Models

OT1 T cell and B16-Ova Tumor Cell Model: Anti-tumor efficacy of dual-edited Ptpn2/Socs1 CD8+ T cells was evaluated in mice using the B16Ova subcutaneous syngeneic tumor model. 6-8 week old female C57BL/6J mice from Jackson labs were injected subcutaneously in the right flank with 0.5×10⁶ B16-Ova tumor cells. When tumors in the entire cohort of mice reached an average volume of approximately 485 mm³ at 15 days after inoculation, the mice were randomized into five groups of 10 mice each and injected intravenously with edited murine OT1 CD8+ T cells via tail vein injection. Prior to injection, these cells were edited by electroporation with gRNA/Cas9 RNP complexes comprising (1) a non-targeting control gRNA; (2) a single gRNA targeting the PD1 gene (SEQ ID NO: 270); (3) a single gRNA targeting the Ptpn2 gene (SEQ ID NO: 202); (4) a single gRNA targeting the Socs1 gene (SEQ ID NO: 9); (5) 2 gRNAs, one targeting each of the Socs1 and Ptpn2 genes. Editing efficiency of the gRNA/Cas9 complex targeting the Ptpn2 and Socs1 genes was assessed by next-generation sequencing and determined to be 70% and 82%, respectively. Body weight and tumor volume was measured at least twice per week. Tumor volume was calculated as mean and standard error of the mean for each treatment group. The percentage tumor growth inhibition (TGI) was calculated according to the following formula:

% TGI=(Ptpn2/Socs1 TV_{final}−Ptpn2/Socs1 TVᵢₙᵢₜᵢₐₗ)/(Control TV_{final} Control TVᵢₙᵢₜᵢₐₗ),
where TV=mean tumor volume, final=Day 7, and initial=day of edited mouse OT1 CD8+ T cell transfer.

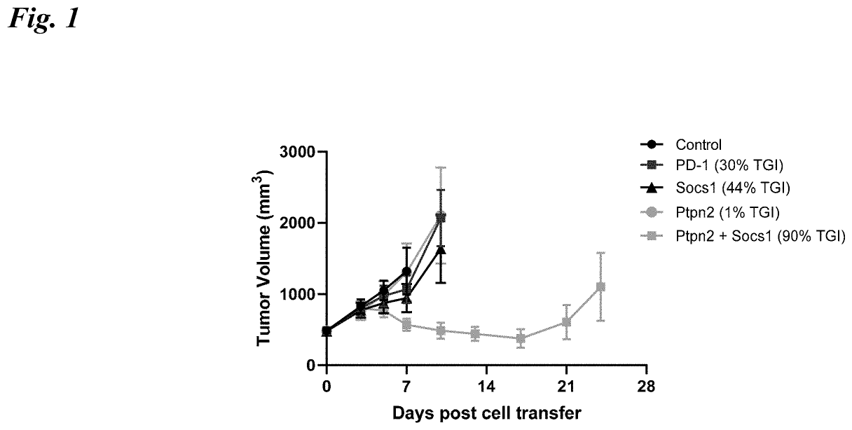

The data in FIG. 1 show that compared to a control guide, adoptive transfer of Ptpn2/Socs1 dual-edited mouse OT1 CD8+ T cells resulted in an anti-tumor response in the B16Ova subcutaneous mouse model. Further, the % TGI observed on Day 7 after T cell transfer was increased in the Ptpn2/Socs1 dual-edited group (TGI=90%) compared to either of the Ptpn2-single edited (TGI=1%) or Socs1-single edited (TGI=44%) groups. Additionally, the % TGI of the Ptpn2/Socs1 dual-edited group was also increased compared to the % TGI observed with PD1-edited T cells (TGI=30%). A summary of the efficacy of dual-edited and single-edited T cells in the B16-Ova model is provided in Table 20 below.

TABLE 20

| | PD-1 sensitive | PD-1 resistant |
|---|---|---|
| | B16-Ova, normal* | B16-Ova, large** |
| Gene Target | (melanoma) | (melanoma) |
| Ptpn2 | ++ | − |
| Socs1 | ++ | − |
| Dual Ptpn2/Socs1 | +++ | +++ |
| Pdcd-1 | ++ | − |

*100 mm³
**>500 mm³
NT = not tested; (−) = no efficacy observed; (+) = modest responses in majority of animals; (++) = strong responses in majority of animals; (+++) = strong responses, including some complete responses, in all animals treated

Subsequent studies in the PD-1 resistant large tumor B16Ova model were performed as described above with initial starting tumor volumes of approximately 343 mm³ at 15 days after inoculation. Mice that completely rejected the original large B16Ova tumor were then rechallenged subcutaneously in the left flank with either 0.5×10⁶ B16-Ova tumor cells (n=6) or 0.3×10⁶ B16F10 tumor cells (n=6) on day 106 after T cell transfer. Editing efficiency of the gRNA/Cas9 complex targeting the Ptpn2 and Socs1 genes was assessed by next-generation sequencing and determined to be 75.4% and 86.5%, respectively. Body weight and tumor volume was measured at least twice per week. Tumor volume was calculated as described above. At various timepoints before and after rechallenge the mice were bled via tail stick and samples were analyzed via flow cytometry to track the OT1 CD8+ T cells and their phenotype in peripheral blood.

A separate cohort of mice was inoculated and euthanized on day six. Tumor, spleen and blood was analyzed via flow cytometry for total OT1 population, cytokine production and other target related readouts.

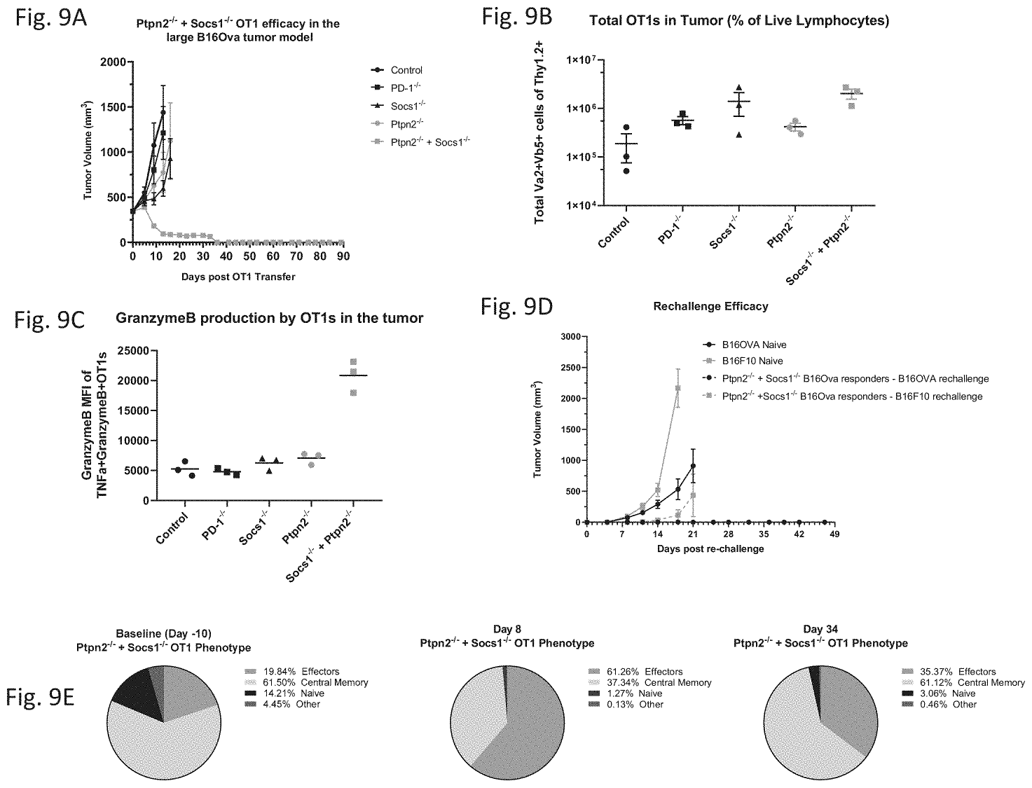

The data in FIG. 9A shows that adoptive transfer of Ptpn2/Socs1 dual-edited mouse OT1 CD8+ T cells resulted in eight out of eight mice achieving complete responses against the large B16OVA tumors. Mice treated with Ptpn2/Socs1 dual-edited mouse OT1 CD8+ T cells showed an increase in the number of OT1 cells in the B16Ova tumor on day six compared to single edited OT1 CD8 T cells (FIG. 9B) and also showed an increase in granzyme B production (FIG. 9C). FIG. 9D shows that all mice that previously had rejected a large B16Ova tumor were subsequently able to reject a second inoculation of B16Ova compared to naïve mice. Two out of six mice that previously had rejected a large B16Ova tumor were also able to completely reject a second inoculation with parental B16F10 that does not express neoantigen. Characterization of Ptpn2/Socs1 dual-edited mouse OT1 CD8+ T cells during the B16Ova rechallenge showed an expansion of these cells from a central memory phenotype to an effector phenotype eight days after B16Ova rechallenge. This was followed by a contraction back to a central memory phenotype (FIG. 9E).

PMEL T Cell and MC38-gp100 Tumor Cell Model:

Additional experiments are performed to assess the effect of Ptpn2/Socs1 dual-edited T cells in a MC38 subcutaneous syngeneic tumor model of colorectal cancer (which is insensitive to treatment with anti-PD1 antibodies). Briefly, 6-8 week old female C57BL/6J mice from Jackson labs are injected subcutaneously with 1×10⁶ MC38 tumor cells expressing gp100. When tumors reached a volume of approximately 100 mm³, mice are randomized into groups of 10 and injected intravenously with edited murine PMEL CD8⁺ T cells via tail vein. Prior to injection, these cells are edited by electroporation with gRNA/Cas9 RNP complexes comprising (1) a non-targeting control gRNA; (2) a single gRNA targeting the PD1 gene; (3) a single gRNA targeting the Ptpn2 gene; (4) a single gRNA targeting the Socs1 gene; (5) 2 gRNAs, one targeting each of the Socs1 and Ptpn2 genes. PMEL CD8⁺ T cells were edited according to the method described here in Example 4. The editing efficiency of the dual gRNA/Cas9 complex targeting the Socs1 and Ptpn2 genes was assessed using by NGS and was determined to be 65% and 47%, respectively. Body weight and tumor volume are measured at least twice per week. Tumor volume was calculated as mean and standard error of the mean for each treatment group and the % TGI for each group is calculated as described above. These experiments show an enhanced anti-tumor efficacy of Ptpn2/Socs1 dual-edited T cells compared to control or single-edited T cell treatment.

### Example 4: Efficacy of Ptpn2/Socs1 Dual-Edited Transgenic T Cells in a Murine Syngeneic Model of Metastatic Lung Cancer

Anti-tumor efficacy of Ptpn2/Socs1 dual-edited T cells was evaluated in mice using the aggressive metastatic B16-F10 syngeneic tumor model with disease manifesting as lung metastasis. Briefly, 6-8 week old female C57BL/6J mice from Jackson labs were injected intravenously with 0.5×10⁶ B16-F10 tumor cells. Prior to inoculation, mice were weighed and randomly assigned to treatment groups. 3 days post-tumor cell inoculation, mice were injected intravenously with edited mouse PMEL CD8+ T cells via tail vein. Prior to T cell injection, these cells were edited by electroporation with gRNA/Cas9 RNP complexes comprising (1) a non-targeting control gRNA; (2) a single gRNA targeting the Ptpn2 gene (SEQ ID NO: 202); (3) a single gRNA targeting the Socs1 gene (SEQ ID NO: 9); (4) 2 gRNAs, one targeting each of the Socs1 and Ptpn2 genes. The editing efficiency of the dual gRNA/Cas9 complex targeting the Socs1 and Ptpn2 genes was assessed using by NGS and was determined to be 65% and 47%, respectively. Body weight was monitored at least twice per week. At D15 post-tumor cell inoculation (D12 post-T cell transfer), lungs of each mouse were perfused and fixed with 10% para-formaldehyde. After overnight fixation, lungs were transferred to 70% EtOH for further preservation.

Tumor efficacy was evaluated by visually assessing the B16-F10 tumor burden which can be seen as black colonies of cancer cells on the lungs. Large numbers of metastatic colonies were observed in all lungs from the untreated group and from mice treated with control-edited PMEL CD8+ T cells, signifying significant disease progression in these groups. Partial efficacy was seen in mice treated with Socs1 single-edited cells with evidence of a partial reduction of metastatic burden, while Ptpn2 single-edited cells had a minimal efficacy. However, treatment with Ptpn2/Socs1 dual-edited cells resulted in strong anti-tumor efficacy with a near complete inhibition of tumor formation. A summary of the efficacy of dual-edited and single-edited T cells in the B16-F10 model is provided in Table 21 below.

TABLE 21

| Target Gene | PD-1 resistant - B16F10 (lung) |
|---|---|
| Ptpn2 | + |
| Socs1 | ++ |
| Dual Ptpn2/Socs1 | +++ |
| Pdcd1 | − |

(−) = no efficacy observed; (+) = modest responses in majority of animals; (++) = strong responses in majority of animals; (+++) = strong responses, including some complete responses, in all animals treated

### Example 5: Efficacy of Ptpn2/Socs1 Dual-Edited Transgenic T Cells in a Xenograft Model of Melanoma

Anti-tumor efficacy of Ptpn2/Socs1 dual-edited T cells is evaluated in mice using the A375 xenograft tumor model. Briefly, 6-8 week old NSG mice from Jackson labs are injected subcutaneously with 5×10⁶ A375 cells (expressing the NY-ESO-1 antigen). When tumors reach a volume of approximately 200 mm³, mice are randomized into groups of 8 and injected intravenously with up to 30×10⁶ edited cells, which were additionally lentivirally transduced to express TCR2, via tail vein. Prior to T cell injection, these cells are edited by electroporation with gRNA/Cas9 RNP complexes comprising (1) a non-targeting control gRNA; (2) a single gRNA targeting the PD1 gene; (3) a single gRNA targeting the Ptpn2 gene; (4) a single gRNA targeting the Socs1 gene; (5) 2 gRNAs, one targeting each of the Socs1 and Ptpn2 genes. Body weight and tumor volume are measured at least twice per week. Tumor volume is calculated as mean and standard error of the mean for each treatment group and % TGI will be calculated as described above. These data demonstrate that treatment with Ptpn2/Socs1 dual-edited T cells enhances anti-tumor efficacy in a NY-ESO-1tumor model compared to the anti-tumor efficacy observed after treatment with either Ptpn2-single edited or Socs1-single edited cells.

### Example 6: Efficacy of Ptpn2/Socs1 Dual-Edited Tumor Infiltrating Lymphocytes

Anti-tumor efficacy of Ptpn2/Socs1 dual-edited tumor infiltrating lymphocytes (TILs) was evaluated in an exploratory mouse model. Two mice cohorts were used in this experiment: a donor cohort of CD45.1 Pep Boy mice (B6. SJL-Ptprc^{a} Pepc^{b}/BoyJ) and a recipient cohort of CD45.2 C57BL/6J mice (Jackson labs), each comprised of 6-8 week old female mice.

To generate TILs, donor CD45.1 Pep Boy mice were injected subcutaneously with 0.5×10⁶ B16-Ova cells. On Day 14 post-tumor cell inoculation, tumors were harvested to generate edited CD45.1 Tumor Infiltrating Lymphocytes (TILs) to infuse into the second cohort of mice as described above in Example 1. These TIL cells were edited by electroporation of gRNA/Cas9 complexes comprise (1) a non-targeting control gRNA; (2) a single gRNA targeting the Ptpn2 gene; (3) a single gRNA targeting the Socs1 gene; or (4) 2 gRNAs, one targeting each of the Socs1 and Ptpn2 genes. The editing efficiency of the dual gRNA/Cas9 complex targeting the Socs1 and Ptpn2 genes was assessed using by NGS and was determined to be 77.8% and 87.6%, respectively. For details for Ptpn2++Socs1 editing see Example 10 and Example 15.

Recipient CD45.2 C57BL/6J mice were injected subcutaneously with 0.5×10⁶ B16-Ova tumor cells. When tumors reach a volume of approximately 100 mm³, mice were randomized into groups of 10 and injected intravenously with edited CD45.1 TILs via tail vein. In additional experiments, human IL-2 can be delivered simultaneously. Body weight and tumor volume were measured at least twice per week. Tumor volume was calculated as mean and standard error of the mean for each treatment group and the % TGI is calculated according to the following formula:

% TGI=(Ptpn2/Socs1 TV_{final})−Ptpn2/Socs1 TVᵢₙᵢₜᵢₐₗ)/(Control TV_{final}−Control TVᵢₙᵢₜᵢₐₗ),
where TV=mean tumor volume, final=Day 17, and initial=day of edited TIL transfer.

In this preliminary experiment using this exploratory model, treatment with Ptpn2/Socs1 dual edited TILs leads to enhanced tumor efficacy.

TABLE 22

| | Non-Lymphodepleted |
|---|---|
| Gene Target | mTIL model |
| Ptpn2 | − |
| Socs1 | − |
| Dual Ptpn2/Socs1 | − |
| Pdcd-1 | − |
| Dual ZC3H12A/Socs1 | +++ |

(−) = no efficacy observed; (+) = modest responses in majority of animals; (++) = strong responses in majority of animals; (+++) = strong responses, including some complete responses, in all animals treated

### Example 7: Efficacy of Zc3h12a/Socs1 Dual-Edited Transgenic T Cells in Murine Syngeneic Tumor Models

OT1 T cell and B16-Ova Tumor cell Model:

Anti-tumor efficacy of Zc3h12a/Socs1 dual-edited transgenic CD8+ T cells was evaluated in mice using the B16Ova subcutaneous syngeneic tumor model. 6-8 week old female C57BL/6J mice from Jackson labs were injected subcutaneously with 0.5×10⁶ B16-Ova tumor cells. When tumors in the entire cohort of mice reached an average volume of approximately 485 mm³, the mice were randomized into five groups of 10 mice each and injected intravenously with edited murine OT1 CD8+ T cells via tail vein injection. Prior to injection, these cells were edited by electroporation with gRNA/Cas9 RNP complexes comprising (1) a non-targeting control gRNA; (2) a single gRNA targeting the Zc3h12a gene (SEQ ID NO: 211); (3) a single gRNA targeting the Socs1 gene (SEQ ID NO: 9); or (4) 2 gRNAs, one targeting each of the Zc3h12a and Socs1 genes. The editing efficiency of the dual gRNA/Cas9 complex targeting the Zc3h12a and Socs1 genes was assessed by NGS and determined to be 86% and 84%, respectively. Body weight and tumor volume were measured at least twice per week. Tumor volume was calculated as mean and standard error of the mean for each treatment group. The percentage tumor growth inhibition (TGI) was calculated using the following formula:

% TGI=(Zc3h12a/Socs1 TV_{final})−Zc3h12a/Socs1 TVᵢₙᵢₜᵢₐₗ)/(Control TV_{final}−Control TVᵢₙᵢₜᵢₐₗ),
where TV=mean tumor volume, final=Day 10, and initial=day 0 of edited T cell transfer.

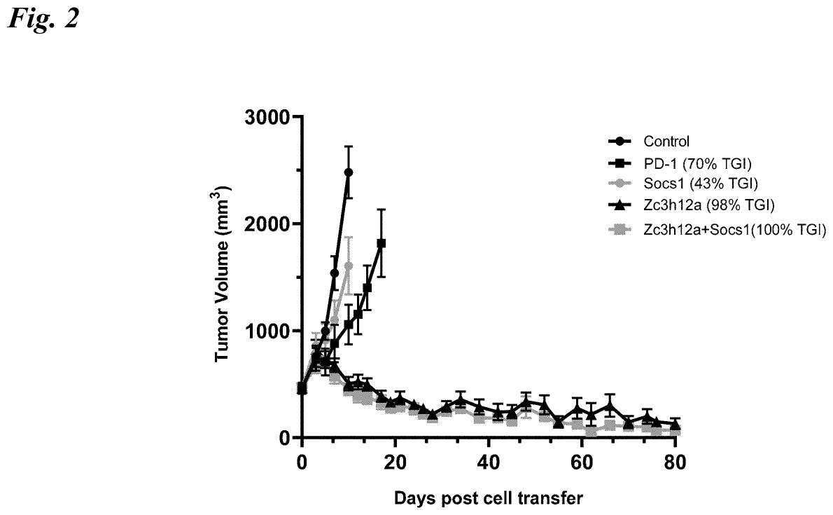

The data in FIG. 2 show that compared to a control guide, adoptive transfer of Zc3h12a/Socs1 dual-edited mouse OT1 CD8+ T cells resulted in an enhanced anti-tumor response in the B16Ova subcutaneous mouse model. This effect was maintained out to day 140 before the study was terminated.

PMEL T Cell and MC38-gp100 Tumor Cell Model:

Additional experiments are performed to assess the effect of Zc3h12a/Socs1 dual-edited T cells in a MC38 subcutaneous syngeneic tumor model of colorectal cancer (which is insensitive to treatment with anti-PD1 antibodies). Briefly, 6-8 week old female C57BL/6J mice from Jackson labs are injected subcutaneously with 1×10⁶ MC38 tumor cells expressing gp100. When tumors reach a volume of approximately 100 mm³, mice are randomized into groups of 10 and injected intravenously with edited murine PMEL CD8⁺ T cells via tail vein. Prior to injection, these cells are edited by electroporation with gRNA/Cas9 RNP complexes comprising (1) a non-targeting control gRNA; (2) a single gRNA targeting the PD1 gene; (3) a single gRNA targeting the Zc3h12a gene; (4) a single gRNA targeting the Socs1 gene; (5) 2 gRNAs, one targeting each of the Socs1 and Zc3h12a genes. Body weight and tumor volume will be measured at least twice per week. Tumor volume is calculated as mean and standard error of the mean for each treatment group and the % TGI for each group is calculated as described above. These experiments are expected to show an enhanced anti-tumor efficacy of Ptpn2/Socs1 dual-edited T cells compared to control or single-edited T cell treatment.

### Example 8: Efficacy of Zc3h/2a/Socs1 Dual-Edited Transgenic T Cells in a Murine Syngeneic Model of Metastatic Cancer

Anti-tumor efficacy of Zc3h12a/Socs1 dual-edited T cells are evaluated in mice using the aggressive metastatic B16-F10 syngeneic tumor model with disease manifesting as lung metastasis. Briefly, 6-8 week old female C57BL/6J mice from Jackson labs are injected intravenously with 0.5×10⁶ B16-F10 tumor cells. Prior to inoculation, mice are weighed and randomly assigned to treatment groups. 3 days post-tumor cell inoculation, mice are injected intravenously with edited mouse PMEL CD8+ T cells via tail vein. Prior to T cell injection, these cells are edited by electroporation with gRNA/Cas9 RNP complexes comprising (1) a non-targeting control gRNA; (2) a single gRNA targeting the Zc3h12a gene; (3) a single gRNA targeting the Socs1 gene; (4) 2 gRNAs, one targeting each of the Zc3h12a and Socs1 genes. The editing efficiency of the dual gRNA/Cas9 complex targeting the Zc3h12a and Socs1 genes is assessed using by NGS. Body weight is monitored at least twice per week. At D15 post-tumor cell inoculation (D12 post-T cell transfer), lungs of each mouse are perfused and fixed with 10% para-formaldehyde. After overnight fixation, lungs are transferred to 70% EtOH for further preservation. Tumor efficacy is evaluated by visually assessing the B16-F10 tumor burden which can be seen as black colonies of cancer cells on the lungs. These data are expected to show an enhanced anti-tumor efficacy of Zc3h12a/Socs1 dual-edited T cells compared to control or single-edited T cell treatment.

TABLE 23

| Target Gene | PD-1 resistant - B16F10 (lung) |
|---|---|
| Zc3h12a | +++ |
| Socs1 | ++ |
| Pdcd1 | − |

(−) = no efficacy observed; (+) = modest responses in majority of animals; (++) = strong responses in majority of animals; (+++) = strong responses, including some complete responses, in all animals treated

### Example 9: Efficacy of Zc3h12a/Socs1 Dual-Edited Transgenic T Cells in a Xenograft Model of Melanoma

Anti-tumor efficacy of Zc3h12a/Socs1 dual-edited T cells is evaluated in mice using the A375 xenograft tumor model. Briefly, 6-8 week old NSG mice from Jackson labs are injected subcutaneously with 5×10⁶ A375 cells (expressing the NY-ESO-1 antigen). When tumors reach a volume of approximately 400 mm³, mice are randomized into groups of 8 and injected intravenously with 30×10⁶ edited TCR2 cells via tail vein. Prior to T cell injection, these cells are edited by electroporation with gRNA/Cas9 RNP complexes comprising (1) a non-targeting control gRNA; (2) a single gRNA targeting the PD1 gene; (3) a single gRNA targeting the Zc3h12a gene; (4) a single gRNA targeting the Socs1 gene; (5) 2 gRNAs, one targeting each of the Socs1 and Zc3h12a genes. Body weight and tumor volume are measured at least twice per week. Tumor volume is calculated as mean and standard error of the mean for each treatment group and % TGI will be calculated as described above. These data demonstrate that treatment with Zc3h12a/Socs1 dual-edited T cells is expected to lead to enhanced anti-tumor efficacy in a NY-ESO-1 tumor model compared to the anti-tumor efficacy observed after treatment with either Zc3h12a-single edited or Socs1-single edited cells.

### Example 10: Efficacy of Zc3h12a/Socs1 Dual-Edited Tumor Infiltrating Lymphocytes

Anti-tumor efficacy of Zc3h12a/Socs1 dual-edited tumor infiltrating lymphocytes (TILs) was evaluated in non-lymphodepleted mice using the B16Ova subcutaneous syngeneic tumor model. Two mice cohorts were used in this experiment: a donor cohort of CD45.1 Pep Boy mice (B6. SJL-Ptprc^{a} Pepc^{b}/BoyJ) and a recipient cohort of CD45.2 C57BL/6J mice (Jackson labs), each comprised of 6-8 week old female mice.

To generate TILs, donor CD45.1 Pep Boy mice are injected subcutaneously with 0.5×10⁶ B16-Ova cells. On Day 14 post-tumor cell inoculation, tumors were harvested to generate edited CD45.1 Tumor Infiltrating Lymphocytes (TILs) to infuse into the second cohort of mice as described above in Example 1. These TIL cells were edited by electroporation of gRNA/Cas9 complexes comprise (1) a non-targeting control gRNA; (2) a single gRNA targeting the Zc3h12a gene (SEQ ID NO: 211); (3) a single gRNA targeting the Socs1 gene (SEQ ID NO: 9); or (4) 2 gRNAs, one targeting each of the Socs1 and Zc3h12a genes. The editing efficiency of the dual gRNA/Cas9 complex targeting the Zc3h12a and Socs1 genes was assessed using by NGS and was determined to be 82% and 84%, respectively.

Recipient CD45.2 C57BL/6J mice were injected subcutaneously with 0.5×10⁶ B16-Ova tumor cells. When tumors reached a volume of approximately 100 mm³ mice were randomized into groups of 10 and injected intravenously with edited CD45.1 TILs via tail vein. Body weight and tumor volume was measured at least twice per week. Tumor volume was calculated as mean and standard error of the mean for each treatment group and the % TGI is calculated according to the following formula:

% TGI=(Zc3h12a/Socs1 TV_{final})−Zc3h12a/Socs1 TVᵢₙᵢₜᵢₐₗ)/(Control TV_{final}−Control TVᵢₙᵢₜᵢₐₗ),
where TV=mean tumor volume, final=Day 17 and initial=day of edited TIL transfer.

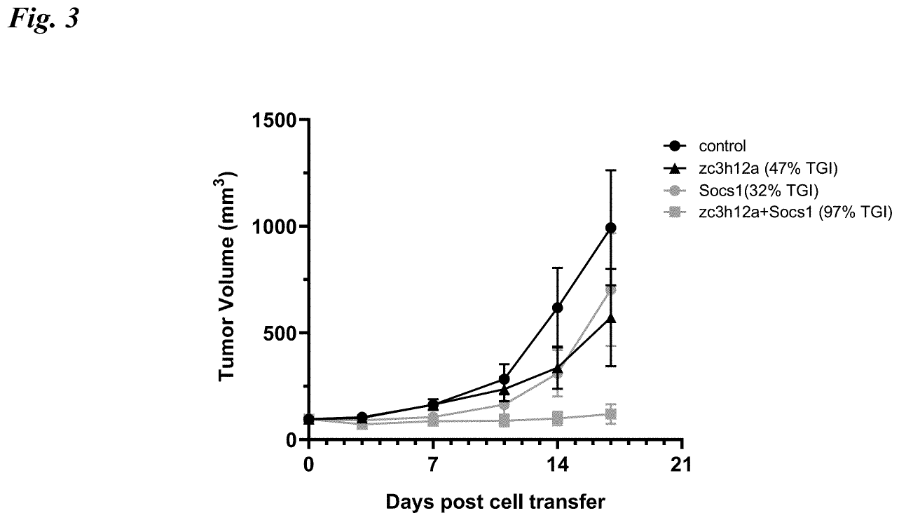

The data in FIG. 3 show that compared to a control guide, adoptive transfer of Zc3h12a/Socs1 dual-edited mouse TILs resulted in an enhanced anti-tumor response in the B16Ova subcutaneous mouse model (TGI=97%) compared to treatment with either Zc3h12a single-edited TILs (TGI=47%) or Socs1 single-edited TILs (TGI=32%).

### Example 11: Efficacy of PD1/Lag3 Dual-Edited Transgenic T Cells in a B16-Ova Murine Tumor Model

Anti-tumor efficacy of PD-1/Lag3 dual-edited T cells was evaluated in mice using the B16Ova subcutaneous syngeneic tumor model. 6-8 week old female C57BL/6J mice from Jackson labs were injected subcutaneously with 0.5×10⁶ B16Ova tumor cells. When tumors in the entire cohort of mice reached an average volume of approximately 485 mm³, the mice were randomized into groups of 10 and injected intravenously with edited mouse OT1 CD8+ T cells via tail vein. Prior to injection these cells were edited by electroporation with gRNA/Cas9 RNP complexes comprising (1) a non-targeting control gRNA; (2) a single gRNA targeting the PD1 gene (SEQ ID NO: 270); (3) a single gRNA targeting the Lag3 gene; (4) 2 gRNAs, one targeting each of the PD1 and Lag3 genes. The editing efficiency of the dual gRNA/Cas9 complex targeting the Pdcd1 and Lag3 genes was assessed using by NGS and was determined to be 58.8% and 89.4%, respectively. Body weight and tumor volume was measured at least twice per week. Tumor volume was calculated as mean and standard error of the mean for each treatment group. The percentage tumor growth inhibition (TGI) was calculated using the following formula:

% TGI=(PD1/Lag3 TV_{final})−PD1/Lag3 TVᵢₙᵢₜᵢₐₗ)/(Control TV_{final}−Control TVᵢₙᵢₜᵢₐₗ),
where TV=mean tumor volume, final=Day 10 and initial=day of edited mouse OT1 CD8+ T cell transfer.

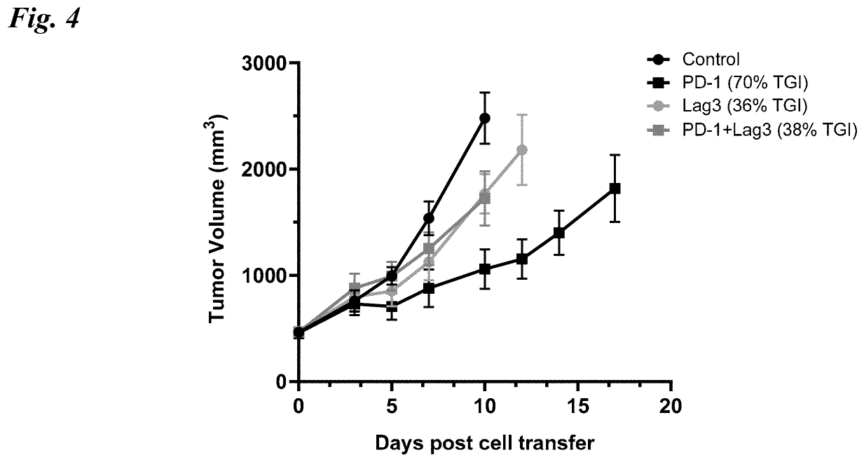

The data in FIG. 4 show adoptive transfer of PD-1 single-edited T cells resulted in a TGI of 70% and adoptive transfer of Lag3 single-edited T cells resulted in a TGI of 36%. Surprisingly, combination edits of PD1 and Lag3 did not result in enhanced tumor growth inhibition and demonstrated a TGI of 38%.

### Example 12: Validation of Dual-Edited CAR-T and TCR Transgenic T Cell Efficacy and Function

Experiments were performed to validate the effects of editing two of PTPN2, ZC3H12A, and/or SOCS1 on the anti-tumor efficacy of CAR T cells and T cells engineered to express an artificial TCR. The engineered T cells described in Table 24 were edited as described in Example 1 to reduce expression of PTPN2, ZC3H12A, and/or SOCS1. These edited T cells were then evaluated in subcutaneous xenograft models using the indicated cell type.

TABLE 24
Engineered Receptor Specificity and Target Cell Lines

| Receptor Specificity | Target Cell Line |
|---|---|
| CD19 | Raji, Daudi, NALM-6, NALM-16, RAMOS, JeKo1 |
| BCMA | Multiple Myeloma cell lines NCI-H929, |
| | U266-B1, and RPMI-8226 |
| NYESO | A375 |
| MART1 | SKMEL5, WM2664, IGR1 |
| HER2+ | BT474 |

Briefly, 6-8 week old female NSG mice from Jackson labs were injected subcutaneously with 1×10⁶ Raji cells. When tumors reached a volume of approximately 200 mm³, mice were randomized into groups of 8 and injected intravenously with 3×10⁶−10×10⁶ edited engineered CAR T cells targeting CD19 via tail vein. Prior to injection the adoptively transferred cells were edited with either a control gRNA or a gRNA targeting PTPN2, ZC3H12A, and/or SOCS1. Body weight and tumor volume were measured at least twice per week. Tumor volume was calculated as mean and standard error of the mean for each treatment group. The results of these experiments (Table 25) show enhanced anti-tumor efficacy of 10×10⁶ PTPN2^{-/-} and SOCS1^{-/-} dual edited engineered T cells or as compared to a control guide, measured by tumor volume and number of complete responses at the end of the study (eight out of eight for PTPN2^{-/-}/SOCS1^{-/-} CAR T cells vs one out of eight for control edited CAR T cells)

TABLE 25

| Target Gene | Raji - CD19 CAR T Model |
|---|---|
| PTPN2 + SOCS1 | +++ |
| Pdcd1 | − |

(−) = no efficacy observed; (+) = modest responses in majority of animals; (++) = strong responses in majority of animals; (+++) = strong responses, including some complete responses, in all animals treated

Additional experiments are performed to validate the effects of editing PTPN2, ZC3H12A, and/or SOCS1 on engineered T cell cytokine production. Briefly, the engineered T cells described in Table 25 above are generated from human T cells, and two or more of PTPN2, ZC3H12A, and SOCS1 are edited by electroporation using guide RNAs complexed to Cas9 in an RNP format. CAR-Ts are co-cultured with the corresponding cell line indicated in Table 22 in vitro at a 1:0, 0.3:1, 1:1, 3:1 and 10:1 ratio. After 24 hours, total cell counts of engineered T cells are determined, and supernatants saved for cytokine analyses. The results of these experiments are expected to show enhanced accumulation of and increased levels of cytokine production from dual-edited CAR T cells compared to control edited cells.

### Example 13: In Vitro Assessment of Dual-Edited Immune Cell Function

To assess for SOCS1-, PTPN2-, and ZC3H12A-dependent pharmacology, assays are developed that quantify the dependent biology of each target. These assays are also intended to be used to assess target-dependent pharmacology in double edited TILs. The activity of sgRNAs targeting SOCS1, PTPN2, and ZC3H12A in TILs are assessed in these assays. For example, cells in which both SOCS1 and PTPN2 are inactivated should demonstrate activity in assays measuring both SOCS1 and PTPN2 pharmacology.

In addition to the negative role of PTPN2 on T cell receptor (TCR) signaling, both SOCS1 and PTPN2 are negative regulators of JAK/STAT signaling. Therefore, SOCS1-dependent and PTPN2-dependent pharmacology can be measured by increases in JAK/STAT signaling.

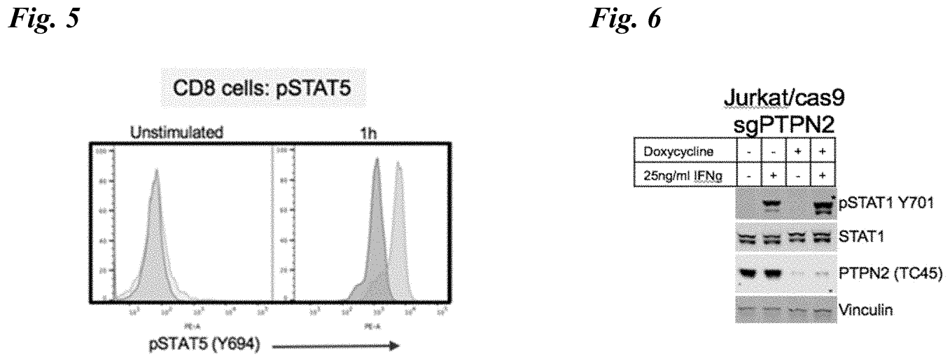

SOCS1 negatively regulates cytokine signaling in T cells, in part by inhibiting JAK1, a kinase involved in STAT5 phosphorylation and cytokine signal transduction. Upon IL-2 signaling through the IL-2 receptor complex, STAT5 is phosphorylated in a JAK1-dependent manner. Therefore, levels of pSTAT5 and activation of downstream signaling pathways upon IL-2 stimulation may serve as an assay measuring SOCS1-dependent pharmacology in TILs. Indeed, deletion of SOCS1 lead to an increase in pSTAT5 levels in primary human CD8 T cells in response to IL-2 signaling (FIG. 5).

PTPN2 also acts as a negative regulator of cytokine signaling, including IL-2 and IFNγ, by directly dephosphorylating STAT proteins such as pSTAT1 and pSTAT3. Therefore, levels of pSTAT1 and pSTAT3 and activation of downstream signaling pathways may serve as an assay measuring PTPN2-dependent pharmacology in TILs. Indeed, Cas9-mediated genetic knockdown of PTPN2 leads to increased pSTAT1 levels in Jurkat T cells in response to IFNγ stimulation (FIG. 6). PTPN2 is also a negative regulator of TCR signaling. Both LCK and FYN transmit positive signaling downstream of the TCR, and are direct targets of PTPN2 phosphatase activity following TCR activation. Therefore, the impact of genetic inactivation of PTPN2 on proximal T cell receptor signaling may be assessed by quantifying pLCK and pFYN following TCR stimulation.

In conclusion, direct assessment of SOCS1 and PTPN2 pharmacology in dual-edited cells can be conducted using 1) cytokine stimulation and pSTAT assays and 2) TCR activation and downstream signaling assays.

To determine the impact of genetic inactivation of SOCS1 and PTPN2 on cell function in vitro, multiple parameters may be assessed that correlate with T cell function. These include cytokine production (e.g., IL-6 and IL-12), baseline cell surface phenotypes and activated cell surface phenotypes, T cell differentiation state, and tumor-killing ability.

### Example 14: Manufacturing of Dual-Edited Tumor Infiltrating Lymphocytes

Dual-edited TILs are manufactured following established protocols used previously in FDA-approved clinical trials for the isolation and expansion of TILs.

Following removal of tumor tissue, the tumor is both fragmented into 2 mm³ pieces and mechanically/enzymatically homogenized and cultured in 6,000 IU/mL recombinant human IL-2 for up to 6 weeks or until the cell numbers reach or exceed 1×10⁸; this is defined as the pre-rapid expansion phase (pre-REP) of TIL manufacturing. Upon completion of the pre-REP stage TILs are electroporated with gRNA/Cas9 RNP complexes targeting SOCS1, PTPN2, and/or ZC3H12A under cGMP conditions. Cells may be also electroporated prior to or during the pre-REP process. Following electroporation, 50×10⁶ cells are transferred into a 1 L G-Rex™ culture flask with a 1:100 ratio of TIL:irradiated feeder cells for approximately 2 weeks. This portion of manufacturing is defined as the rapid expansion phase (REP). After the REP phase, TIL's are harvested, washed, and suspended in a solution for immediate infusion into the patient.

Using methods similar to those above, edited tumor infiltrating lymphocytes were generated at miniaturized research scale in three independent donors. SOCS1 single edited, PTPN2 single edited, ZC3H12A single edited, SOCS1/PTPN2 dual edited, and SOCS1/ZC3H12A dual edited cells were produced. Briefly, after the pre-REP expansion of TIL in IL-2, TILs were taken and resuspended in Maxcyte electroporation buffer (Maxcyte) at a concentration of 30M cells/ml. Per 20 μl of cells in electroporation buffer, 5 μl of RNP solution was added. Per 5 μl reaction, RNP solution was composed of 0.85 μl 61 μM sNLS-spCas9-sNLS (Aldevron), 1.75 μl of PBS, and 2.4 μl of 100 μM total sgRNA solution. sgRNA solutions were comprised of either 2.4 μl of a single sgRNA, or 1.2 μl each of 2 different sgRNAs. The guides used were as follows: SOCS1—GACGCCTGCGGATTCTACTG (SEQ ID 25), PTPN2—GGAAACTTGGCCACTCTATG (SEQ ID 190), and ZC3H12A—CAGGACGCTGTGGATCTCCG (SEQ ID NO: 219).

Cell/RNP solutions were loaded into Maxcyte processing assemblies (Cat # OC-25X3 or OC-100X2), and subsequently electroporated using a Maxcyte STX, using the program “Optimization #9”. Cells were recovered from the processing assemblies and added to 2× the volume complete REP media (50:50 mix of AIMV media (Gibco #12055) and RPMI 1640 (Gibco #11875), supplemented with 5% heat inactivated human AB serum (Valley Biomedical). Cells were allowed to recover at 37 C for 20 minutes.

Subsequently, TILs were seeded into the REP by transferring them to either 6-well (10 cm² surface area per well) or 24-well (2 cm² surface area per well) Grex flasks at a density of 50,000 TIL per cm². Flasks additionally contained irradiated PBMC feeder cells at a density of 5M per cm², 6000U/ml of recombinant human IL-2, and 30 ng/ml OKT3. REP was carried on for 14 days, during which cells were fed IL-2, fresh media containing IL-2, and/or cells were split. At day 14 of the REP, cells were harvested and editing efficiency was determined by amplicon sequencing of the genomic DNA of the cut site. Editing efficiencies were as noted in Table 26 (numbers reflect the percentage of DNA reads that demonstrated a mutation from the expected wild-type sequence):

TABLE 26

| | | Target | % | Target | % |
|---|---|---|---|---|---|
| Donor | Sample | Locus 1 | Edited | Locus 2 | Edited |
| A | ZC3H12A/SOCS1 | SOCS1 | 98% | ZC3H12A | 99% |
| | dual edit | | | | |
| | SOCS1/PTPN2 dual | SOCS1 | 98% | PTPN2 | 97% |
| | edit | | | | |
| B | ZC3H12A/SOCS1 | SOCS1 | 98% | ZC3H12A | 99% |
| | dual edit | | | | |
| | SOCS1/PTPN2 dual | SOCS1 | 96% | PTPN2 | 92% |
| | edit | | | | |
| C | ZC3H12A/SOCS1 | SOCS1 | 98% | ZC3H12A | 98% |
| | dual edit | | | | |
| | SOCS1/PTPN2 dual | SOCS1 | 84% | PTPN2 | 71% |
| | edit | | | | |

### Example 15: Efficacy of Dual-Edited Tumor Infiltrating Lymphocytes in Lymphodepleted System

Anti-tumor efficacy of Ptpn2/Socs1, Ptpn2/Zc3h12a, or Socs1/Zc3h12A dual-edited tumor infiltrating lymphocytes (TILs) was evaluated in mice using the B16Ova subcutaneous syngeneic tumor model with lymphodepletion in contrast to Example 6 and 10. Two mice cohorts were used in this experiment: a donor cohort of CD45.1 Pep Boy mice (B6.SJL-Ptprc^{a} Pepc^{b}/BoyJ) and a recipient cohort of CD45.2 C57BL/6J mice (Jackson labs), each comprised of 6-8 week old female mice.

To generate TILs, donor CD45.1 Pep Boy mice were injected subcutaneously with 0.5×10⁶ B16-Ova cells. On Day 14 post-tumor cell inoculation, tumors were harvested to generate edited CD45.1 Tumor Infiltrating Lymphocytes (TILs) to infuse into the second cohort of mice as described above in Example 1. These TIL cells were edited by electroporation of gRNA/Cas9 complexes comprise (1) a non-targeting control gRNA; (2) a single gRNA targeting the Ptpn2, Socs1, or (3) 2 gRNAs, one targeting each of the Socs1 and Ptpn2 genes or the Socs1 and Zc3h12a genes. The editing efficiency of the dual gRNA/Cas9 complex targeting the Socs1 and Ptpn2 genes was assessed using by NGS and was determined to be 85% and 71%, respectively. The editing efficiency of the dual gRNA/Cas9 complex targeting the Socs1 and Zc3h12a genes was assessed using NGS and was determined to be 94% and 90%, respectively. In additional experiments, TIL can be edited by electroporation of gRNA/Cas9 complexes targeting each of the Ptpn2 and Zc3h12a genes.

Recipient CD45.2 C57BL/6J mice were injected subcutaneously with 0.5×10⁶ B16-Ova tumor cells. When tumors reach a volume of approximately 100 mm³ mice were randomized into groups of 10 and injected intraperitoneal with cyclophosphamide (200 mg/kg) to induce lymphodepletion. The following day mice were injected intravenously with edited CD45.1 TILs via tail vein. In additional experiments, mice can be injected intraperitoneally with recombinant human IL-2 (720,000 IU/Kg) twice daily for up to a maximum of 4 days. Body weight and tumor volume were measured at least twice per week. Tumor volume is calculated as mean and standard error of the mean for each treatment group and the % TGI is calculated on Day 17 according to the following formula:

% TGI=(Combo TV_{final})−Combo TVᵢₙᵢₜᵢₐₗ)/(Control TV_{final}−Control TVᵢₙᵢₜᵢₐₗ),

where TV=mean tumor volume, final=Day 17 and initial=day of edited TIL transfer.

These data demonstrate that treatment with dual-edited TILs leads to enhanced anti-tumor efficacy compared to the anti-tumor efficacy observed after treatment with single edited TILs in a lymphodepleted system.

TABLE 27

| | Lymphodepleted mTIL |
|---|---|
| Gene Target | model |
| Ptpn2 | + |
| Socs1 | − |
| Dual Ptpn2/Socs1 | + |
| Pdcd-1 | − |
| Dual ZC3H12A/Socs1 | ++ |

(−) = no efficacy observed; (+) = modest responses in majority of animals; (++) = strong responses in the majority of animals; (+++) = strong responses, including some complete responses, in all animals treated

### Example 16: Functional Characterization of SOCS1/PTPN2Dual-Edited TIL

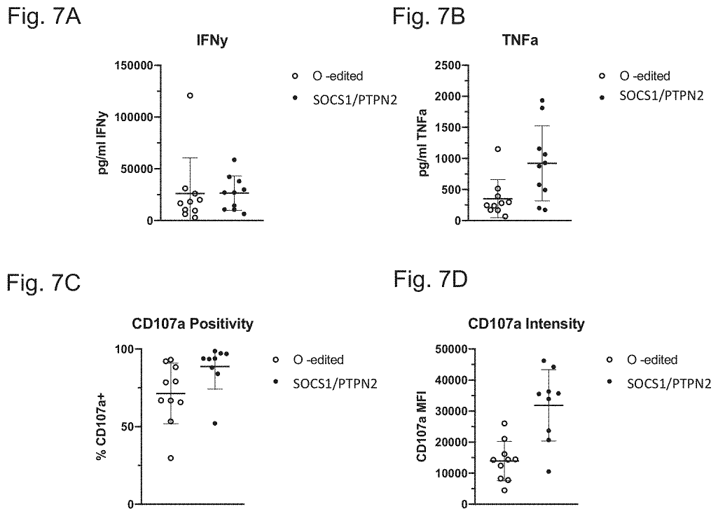

SOCS1 I PTPN2 dual-edited TILs, and control TILs (edited at the OR1A1 locus, which is not expressed in T cells) were generated using methods as described in Example 14. The ability of TILs to produce inflammatory cytokines was assessed. Briefly, 200,000 viable TILs from 5 unique donors were seeded into the wells of a 96-well plate. The volume of media in the well was 200 μl, comprised of 180 μl of REP media (a 50:50 mix of AIM V (Gibco) and RPMI 1640 (Gibco), supplemented with 5% heat inactivated human AB serum (Valley Biomedical)) and 20 μl of anti-CD3 activation tetramer (Stemcell Technologies, custom reagent). The TILs were incubated at 37 degrees Celsius for 18 hours in a 5% CO₂ humidified chamber. After incubation, culture supernatants were harvested and the levels of IFNγ and TNFα in the supernatants were measured using by V-plex cytokine plates and a Quickplex SQ 120 machine (Mesoscale Diagnostics). Dual SOCS1/PTPN2-edited TIL demonstrated a comparable ability to produce IFNγ (FIG. 7A) and an increased ability to produce TNFα (FIG. 7B) compared to controlled edited TILs.

The ability of SOCS1/PTPN2 dual-edited TILs to undergo degranulation upon stimulation was also assessed. 500,000 TILs were stimulated in a 96 well plate with 1/500 dilution of Cell Stimulation Cocktail (Invitrogen) in the presence of golgiplug (BD) and fluorescent anti-CD107a antibody (BD) for 4 hours. Cells were subsequently stained for T cell markers, and CD107a positivity and fluorescence intensity on T cells was assessed by flow cytometry. Dual SOCS1/PTPN2-edited TILs demonstrated increase in degranulation (FIG. 7C) and CD107a intensity (FIG. 7D) as compared to control edited TIL.

### Example 17—Efficacy, Mechanism of Action and Rechallenge of Ptpn2/Socs1 Dual-Edited Transgenic T Cells in Murine Syngeneic Tumor Models

OT1 T Cell and B16-Ova Tumor Cell Model:

Anti-tumor efficacy of dual-edited Ptpn2/Socs1 CD8+ T cells was evaluated in mice using the B16Ova subcutaneous syngeneic tumor model. 6-8 week old female C57BL/6J mice from Jackson labs were injected subcutaneously in the right flank with 0.5×10⁶ B16-Ova tumor cells. When tumors in the entire cohort of mice reached an average volume of approximately 100 mm³, the mice were randomized into five groups of 10 to 20 mice each and injected intravenously with edited murine OT1 CD8+ T cells via tail vein injection. Prior to injection, these cells were edited by electroporation with gRNA/Cas9 RNP complexes comprising (1) a non-targeting control gRNA; (2) a single gRNA targeting the PD1 gene (SEQ ID NO: 270); (3) a single gRNA targeting the Ptpn2 gene (SEQ ID NO: 202); (4) a single gRNA targeting the Socs1 gene (SEQ ID NO: 9); (5) 2 gRNAs, one targeting each of the Socs1 and Ptpn2 genes. Editing efficiency of the gRNA/Cas9 complex targeting the Ptpn2 and Socs1 genes was assessed by next-generation sequencing and determined to be 80.3% and 87.6%, respectively. Body weight and tumor volume was measured at least twice per week. Tumor volume was calculated as mean and standard error of the mean for each treatment group. The percentage tumor growth inhibition (TGI) was calculated according to the following formula:

% TGI=(Ptpn2/Socs1 TV_{final}−Ptpn2/Socs1 TV initial)/(Control TV_{final} Control TVᵢₙᵢₜᵢₐₗ),
where TV=mean tumor volume, final=Day 7, and initial=day of edited mouse OT1 CD8+ T cell transfer.

Mice that completely rejected the original large B16Ova tumor were then rechallenged subcutaneously in the left flank with either 0.5×10⁶ B16-Ova tumor cells (n=6) or 0.3×10⁶ B16F10 tumor cells (n=5) on day 76. Body weight and tumor volume was measured at least twice per week. Tumor volume was calculated as described above. At various timepoints before and after rechallenge the mice were bled via tail stick and samples were analyzed via flow cytometry to track the OT1 CD8+ T cells and their phenotype in peripheral blood.

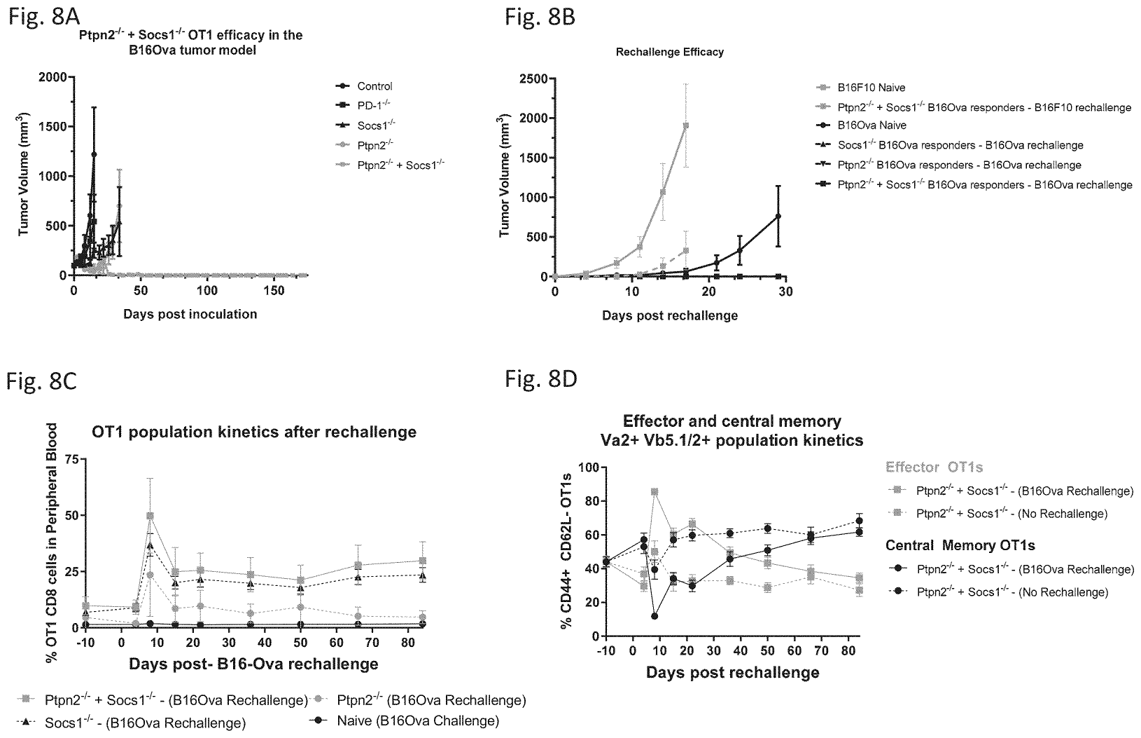

The data in FIG. 8A shows that adoptive transfer of Ptpn2/Socs1 dual-edited mouse OT1 CD8+ T cells resulted in seventeen out of eighteen mice achieving complete responses against the B160VA tumors. FIG. 8B shows that all mice that previously had rejected a B16Ova tumor were subsequently able to reject a second inoculation of B16Ova compared to naïve mice. Two out of five mice that previously had rejected a B16Ova tumor were also able to completely reject a second inoculation with parental B16F10 that does not express neoantigen. FIG. 8C OT1 shows CD8+ T cells underwent a rapid expansion in peripheral blood eight days after rechallenge. FIG. 8D displays characterization of these same OT1s shifting from a central memory phenotype prior to rechallenge to an effector phenotype eight days after the second inoculation of B16Ova. This was followed by a contraction back to a central memory phenotype out to day 84 post rechallenge.

### Example 18. Increased Potency of Ptpn2/Socs1 Dual-Edited Mouse T Cells

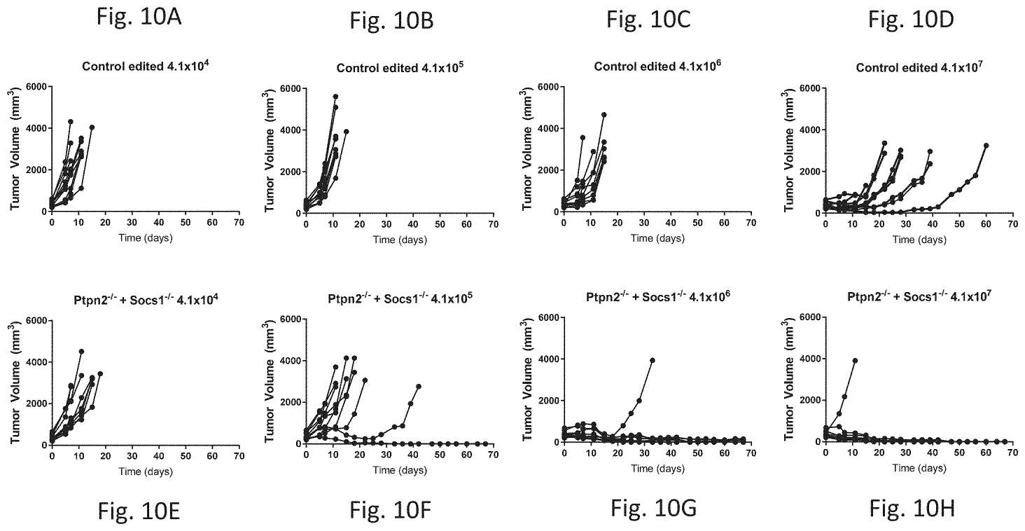

To assess the relative potency of Ptpn2/Socs1 dual-edited mouse OT1 CD8+ T cells in the PD-1 resistant large tumor B16Ova model, four different doses were tested versus their control edited equivalents. These studies were initiated as described above in Example 3 with initial starting tumor volumes of approximately 355 mm³ at which point either Ptpn2/Socs1 dual-edited or control edited mouse OT1 CD8+ T cells were adoptively transferred intravenously at doses of 4.1×10⁴, 4.1×10⁵, 4.1×10⁶, or 4.1×10⁷ cells per mouse. Editing efficiency of the gRNA/Cas9 complex targeting the Ptpn2 and Socs1 genes was assessed by next-generation sequencing and determined to be 66.4% and 86.5%, respectively. Body weight and tumor volume was measured at least twice per week. Tumor volume was calculated as described above. As shown in FIGS. 10A-10H, adoptive transfer of control edited mouse OT1 CD8+ T cells resulted in delayed tumor growth only at the highest dose, 4.1×10⁷ T-cells per mouse, with no complete responses observed. In mice dosed with an equivalent number of Ptpn2/Socs1 dual-edited mouse OT1 CD8+ T cells nine out of ten complete tumor regressions were observed. Additionally, at lower doses of Ptpn2/Socs1 dual-edited mouse OT1 CD8+ T cells significant anti-tumor activity was observed with one out of ten mice showing complete tumor response in the 4.1×10⁵ group. Taken together, these data demonstrate that Ptpn2/Socs1 dual-edited mouse T cells were approximately 10-100× as potent as control edited mouse OT1 CD8+ T cells.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

1. A modified immune effector cell comprising a gene-regulating system capable of reducing expression and/or function of at least two endogenous target genes selected from SOCS1, PTPN2, and ZC3H12A, wherein the reduced expression and/or function of the at least two endogenous target genes enhances an effector function of the immune effector cell.

2-5. (canceled)

6. The modified immune effector cell of claim 1, wherein the gene-regulating system comprises (i) a nucleic acid molecule; (ii) an enzymatic protein; or (iii) a nucleic acid molecule and an enzymatic protein.

7-9. (canceled)

10. The modified immune effector cell of claim 6, wherein the gene-regulating system comprises a nucleic acid molecule and an enzymatic protein, wherein the nucleic acid molecule is a guide RNA (gRNA) molecule and the enzymatic protein is a Cas protein or Cas ortholog.

11-15. (canceled)

16. The modified immune effector cell of claim 10, wherein the at least two endogenous genes are SOCS1 and PTPN2, and wherein the gene-regulating system comprises at least one SOCS1-targeting gRNA molecule comprising a targeting domain sequence complementary to a nucleic acid sequence defined by any one of the set of genome coordinates shown in Tables 3 and 4 and at least one PTPN2-targeting gRNA molecule comprising a targeting domain sequence complementary to a nucleic acid sequence defined by any one of the set of genome coordinates shown in Tables 5 and 6.

17. The modified immune effector cell of claim 10, wherein the at least two endogenous genes are SOCS1 and PTPN2, and wherein the gene-regulating system comprises at least one SOCS1-targeting gRNA molecule comprising a targeting domain sequence that binds to a nucleic acid sequence defined by any one of the set of genome coordinates shown in Tables 3 and 4 and at least one PTPN2-targeting gRNA molecule comprising a targeting domain sequence that binds to a nucleic acid sequence defined by any one of the set of genome coordinates shown in Tables 5 and 6.

18. The modified immune effector cell of claim 10, wherein the at least two endogenous genes are SOCS1 and PTPN2, and wherein the gene-regulating system comprises at least one SOCS1-targeting gRNA molecule comprising a targeting domain sequence that binds to a target DNA sequence selected from the group consisting of SEQ ID NOs: 7-151 and at least one PTPN2-targeting gRNA molecule comprising a targeting domain sequence that binds to a target DNA sequence selected from the group consisting of SEQ ID NOs: 185-207.

19. The modified immune effector cell of claim 10, wherein the at least two endogenous genes are SOCS1 and PTPN2, and wherein the gene-regulating system comprises at least one SOCS1-targeting gRNA molecule comprising a targeting domain sequence encoded by a nucleic acid sequence selected from the group consisting of SEQ ID NOs: 7-151 and at least one PTPN2-targeting gRNA molecule comprising a targeting domain sequence encoded by a nucleic acid sequence selected from the group consisting of SEQ ID NOs: 185-207.

20. The modified immune effector cell of claim 10, wherein the at least two endogenous genes are SOCS1 and ZC3H12A, and wherein the gene-regulating system comprises at least one SOCS1-targeting gRNA molecule comprising a targeting domain sequence complementary to a nucleic acid sequence defined by any one of the set of genome coordinates shown in Tables 3 and 4, and at least one ZC3H12A-targeting gRNA molecule comprising a targeting domain sequence complementary to a nucleic acid sequence defined by any one of the set of genome coordinates shown in Tables 7 and 8.

21. The modified immune effector cell of claim 10, wherein the at least two endogenous genes are SOCS1 and ZC3H12A, and wherein the gene-regulating system comprises at least one SOCS1-targeting gRNA molecule comprising a targeting domain sequence that binds to a nucleic acid sequence defined by any one of the set of genome coordinates shown in Tables 3 and 4 and at least one ZC3H12A-targeting gRNA molecule comprising a targeting domain sequence that binds to a nucleic acid sequence defined by any one of the set of genome coordinates shown in Tables 7 and 8.

22. The modified immune effector cell of claim 10, wherein the at least two endogenous genes are SOCS1 and ZC3H12A, and wherein the gene-regulating system comprises at least one SOCS1-targeting gRNA molecule comprising a targeting domain sequence that binds to a target DNA sequence selected from the group consisting of SEQ ID NOs: 7-151 and at least one ZC3H12A-targeting gRNA molecule comprising a targeting domain sequence that binds to a target DNA sequence selected from the group consisting of SEQ ID NOs: 208-230, SEQ ID NOs: 376-812, or SEQ ID NOs: 376-575.

23. The modified immune effector cell of claim 10, wherein the at least two endogenous genes are SOCS1 and ZC3H12A, and wherein the gene-regulating system comprises at least one SOCS1-targeting gRNA molecule comprising a targeting domain sequence encoded by a nucleic acid sequence selected from the group consisting of SEQ ID NOs: 7-151 and at least one ZC3H12A-targeting gRNA molecule comprising a targeting domain sequence encoded by a nucleic acid sequence selected from the group consisting of SEQ ID NOs: 208-230, SEQ ID NOs: 376-812, or SEQ ID NOs: 376-575.

24. The modified immune effector cell of claim 10, wherein the at least two endogenous genes are PTPN2 and ZC3H12A, and wherein the gene-regulating system comprises at least one PTPN2-targeting gRNA molecule comprising a targeting domain sequence complementary to a nucleic acid sequence defined by any one of the set of genome coordinates shown in Tables 5 and 6 and at least one ZC3H12A-targeting gRNA molecule comprising a targeting domain sequence complementary to a nucleic acid sequence defined by any one of the set of genome coordinates shown in Tables 7 and 8.

25-44. (canceled)

45. A modified immune effector cell, comprising reduced expression and/or function of at least two endogenous genes selected from SOCS1, PTPN2, and ZC3H12A, wherein the reduced expression and/or function of the at least two endogenous genes enhances an effector function of the immune effector cell.

46-58. (canceled)

59. A modified immune effector cell, comprising one or more exogenous polynucleotides encoding at least two nucleic acid inhibitors of at least two endogenous target genes selected from SOCS1, PTPN2, and ZC3H12A.

60-83. (canceled)

84. A gene-regulating system capable of reducing expression of at least two endogenous target genes in a cell selected from SOCS1, PTPN2, and ZC3H12A, comprising (i) a nucleic acid molecule; (ii) an enzymatic protein; or (iii) a nucleic acid molecule and an enzymatic protein.

85-138. (canceled)

139. A composition comprising a plurality of gRNA molecules, wherein the plurality of gRNA molecules comprises at least one gRNA molecule targeting a first target gene and at least one gRNA molecule targeting a second target gene, wherein the first and second target gene are selected from SOCS1, PTPN2, and ZC3H12A.

140-160. (canceled)

161. A polynucleotide molecule encoding a plurality of gRNA molecules, wherein the plurality of gRNA molecules comprises at least one gRNA molecule targeting a first target gene and at least one gRNA molecule targeting a second target gene, wherein the first and second target gene are selected from SOCS1, PTPN2, and ZC3H12A.

162-176. (canceled)

177. A polynucleotide molecule encoding a plurality of siRNA or shRNA molecules, wherein the plurality of siRNA or shRNA molecules comprises at least one siRNA or shRNA molecule targeting a first target gene and at least one siRNA or shRNA molecule targeting a second target gene, wherein the first and second target gene are selected from SOCS1, PTPN2, and ZC3H12A.

178-186. (canceled)

187. A polynucleotide molecule encoding at least one TALEN, zinc finger, or meganuclease protein targeting a first target gene and at least one TALEN, zinc finger, or meganuclease protein targeting a second target gene, wherein the first and second target gene are selected from SOCS1, PTPN2, and ZC3H12A.

188-197. (canceled)

198. A kit comprising the polynucleotide of claim 161.

199. A method of producing a modified immune effector cell comprising introducing the gene-regulating system of claim 84.

200-210. (canceled)

211. A method of treating a disease or disorder in a subject in need thereof comprising administering to the subject an effective amount of the modified immune effector cells of claim 1.

212-224. (canceled)

225. A method of killing a cancerous cell comprising exposing the cancerous cell to the modified immune effector cell of claim 1, wherein exposure to the modified immune effector cell results in increased killing of the cancerous cells compared to exposure to an immune effector cell that has not been modified.

226. (canceled)

227. A method of enhancing one or more effector functions of an immune effector cell comprising introducing a gene-regulating system into the immune effector cell, wherein the gene-regulating system is capable of reducing the expression and/or function of at least two endogenous target genes selected from SOCS1, PTPN2, and ZC3H12A.

228-240. (canceled)

241. A kit comprising the polynucleotide molecule of claim 177.
